(19) United States
(12) Patent Application Publication
(10) Pub. No.: US 2006/0167044 A1
(43) Pub. Date: Jul. 27, 2006

(54) Piperidine derivatives and their use as anti-inflammatory agents

(76) Inventors: Damian O. Arnaiz, El Sobrante, CA (US); Yuo-Ling Chou, Lafayette, CA (US); Monica J. Kochanny, Benicia, CA (US); Wheeseong Lee, Orinda, CA (US); Shou-Fu Lu, San Ramon, CA (US); Anne Mengel, Berlin (DE); Gary Phillips, Pleasant Hill, CA (US); Guo Ping Wei, San Ramon, CA (US); Hongyi Yu, Richmond, CA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/305,322

(22) Filed: Dec. 19, 2005

Related U.S. Application Data
(60) Provisional application No. 60/638,033, filed on Dec. 20, 2004.

(51) Int. Cl. A61K 31/4709; A61K 31/454; C07D 401/02; C07D 409/02

(52) U.S. Cl. 514/310; 514/314; 514/318; 514/326; 546/148; 546/176; 546/194; 546/212

(57) ABSTRACT

Piperidine derivatives of the following formulae I and II: 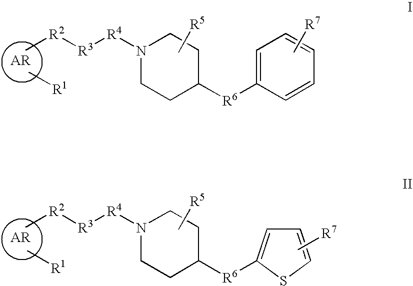 and their pharmaceutically acceptable salts, which are functional antagonists of the CC chemokine receptor CCR1 and are thus useful as anti-inflammatory agents, a pharmaceutical compositions containing said piperidine derivatives or their pharmaceutically acceptable salts, and methods of their use.

This application claims the benefit of the filing date of U.S. Provisional Application Ser. No. 60/638,033 filed Dec. 20, 2004 which is incorporated by reference herein.

### FIELD OF THE INVENTION

The present invention is directed to piperidine derivatives and their pharmaceutically acceptable salts, which are functional antagonists of the CC chemokine receptor CCR1 and are thus useful as anti-inflammatory agents. It also relates to pharmaceutical compositions containing the derivatives or their pharmaceutically acceptable salts, and methods of their use.

### BACKGROUND OF THE INVENTION

An important component of the inflammatory process involves the migration and activation of select populations of leukocytes from the circulation and their accumulation in the affected tissue. Leukocyte trafficking is regulated by members of a large family of chemotactic cytokines known as chemokines. Chemokines are characterized by a distinctive pattern of four conserved cysteine residues. They are divided into two major (CXC and CC) and two minor (C and CX3C) groups dependent on the number and spacing of the first two conserved cysteine residues. Although originally identified on the basis of their ability to regulate the trafficking of immune cells the biological role of chemokines goes well beyond this simple description of their function as chemoattractants, and they have been shown to be involved in a number of biological processes, including growth regulation, hematopoiesis, embryologic development, angiogenesis and HIV-1 infection. (See, Horuk, R. 2001. Chemokine Receptors. Growth factor reviews 12:313-335.)

Chemokines mediate their biological effects by binding to cell surface receptors which belong to the GPCR superfamily. Receptor binding initiates a cascade of intracellular events mediated by the receptor associated heterotrimeric G proteins. These G-protein subunits trigger various effector enzymes which leads to the activation not only of chemotaxis but also to a wide range of functions in different leukocytes such as an increase in the respiratory burst, degranulation, phagocytosis and lipid mediator synthesis. (See, Baggiolini, M. 1998 Nature 392:565-568) Chemokines have been shown to be associated with a number of autoinflammatory diseases including multiple sclerosis, rheumatoid arthritis, diabetes, endometriosis, transplant rejection, renal fibrosis, multiple myeloma, etc. (see, Gerard, C., and B. J. Rollins. 2001 Nat Immunol 2:108-115). Evidence reviewed below is mounting that chemokines may play a major role in the pathophysiology of these diseases and thus chemokine receptor antagonists could prove to be useful therapeutics in treating these and other proinflammatory diseases.

Insight into the physiological and pathophysiological roles of CCR1 has been provided by studies with potent CCR1 antagonists (see, Liang, M., et al., 2000, J Biol Chem 275:19000-19008; Horuk, R., et al., 2001 J Biol Chem 276:4199-4204; and Horuk, R., et al., 2001 Immunol Lett 76:193-201), and confirmed by targeted gene disruption studies (see Gao, J. L., et al., 1997, J Exp Med 185:1959-1968; and Rottman, J. B., et al., 2000, Eur J Immunol 30:2372-2377).

Two recent targeted gene disruption studies (cited above) with CCR1 (−/−) mice have confirmed the roles of CCR1 in the pathophysiology of multiple sclerosis and organ transplant rejection. In the first study, Rottman et al demonstrated, in an EAE model of multiple sclerosis, that CCR1 (−/−) mice had a significantly reduced incidence of disease compared to wild type mice. The spinal cords of the wild type mice showed non-suppurative myelitis while those from the CCR1 knockouts were minimally inflamed. Taken together with the CCR1 antagonist studies discussed below these data strongly argue that CCR1 plays a role in the pathogenesis of EAE and further suggest a role for CCR1 in the pathophysiology of the human disease, multiple sclerosis. In the second study Gao et al reported a significant prolongation of allograft survival in CCR1 (−/−) mice in 4 separate models of cardiac allograft rejection. In one model, levels of cyclosporin that had marginal effects in CCR1 (+/+) mice resulted in permanent allograft acceptance in CCR1 (−/−) recipients.

Three studies with potent CCR1 receptor antagonists (cited above) have illuminated the role of CCR1 in the pathophysiology of multiple sclerosis and organ transplant rejection. Several potent non-peptide CCR1 antagonists have been reported. A potent member of this class of compounds, BX 471, displaced the CCR1 ligands, CCL3 (MIP-1q, CCL5 (RANTES) and CCL7 (MCP-3), with high affinity and was a potent functional antagonist based on its ability to inhibit a number of CCR1-mediated effects including Ca²⁺ mobilization, increase in extracellular acidification rate, CD11b expression and leukocyte migration. In addition, BX 471 demonstrated a greater than 10,000 fold selectivity for CCR1 compared with 28 different GPCR's.

In a rat EAE model of multiple sclerosis BX 471 decreased the clinical score (see, Liang, M., et al., 2000, J Biol Chem 275:19000-19008). BX 471 was also efficacious in a rat heterotopic heart transplant rejection model. Animals treated with BX 471 and a sub-therapeutic dose of cyclosporin, 2.5 mg/kg, which is by itself ineffective in prolonging transplant rejection, was much more efficacious in prolonging transplantation rejection than animals treated with either cyclosporin or BX 471 alone (see Horuk, R., et al., 2001 J Biol Chem 276:4199-4204). Immunohistology of the rat hearts for infiltrating monocytes confirmed these data. Three days after transplantation the extent of monocytic graft infiltration was significantly reduced by the combined therapy of BX 471 and cyclosporin. Thus, BX 471 given in combination with cyclosporin resulted in a clear increase in efficacy in heart transplantation compared to cyclosporin alone. These data were in line with the observed effects of BX 471 in dose-responsively blocking the firm adhesion of monocytes triggered by CCL5 (RANTES) on inflamed endothelium. Together, these data demonstrate a significant role for CCR1 in allograft rejection.

Several studies have demonstrtated that BX 471 is effective in animal models of renal fibrosis (see, Anders, H. J., et al., 2004, J Am Soc Nephrol 15:1504-1513; Anders, H. J., et al., 2002 J Clin Invest 109:251-259; and Eis, V., et. al., 2004 J Am Soc Nephrol 15:337-347) In the study reported in J Clin Invest kidneys from unilateral urether obstructed (UUO) mice treated with BX471 revealed a 40-60% reduction of interstitial macrophage and lymphocyte infiltrate compared with UUO kidneys from untreated animals. Treated mice also showed a marked reduction of CCR1 and CCR5 mRNA levels, and FACS analysis showed a comparable reduction of CD8+/CCR5+T cells. Markers of renal fibrosis, such as interstitial fibroblasts, interstitial volume, mRNA and protein expression for collagen 1, were all significantly reduced by BX471-treatment compared with vehicle controls. In summary, blockade of CCR1 substantially reduces cell accumulation and renal fibrosis after UUO. Most interestingly, late onset of treatment during active disease was also found to be effective and this is the first report that a chemokine receptor antagonist is active therapeutically.

Additionally, U.S. Pat. No. 6,676,926 discloses that radio-labeled analogues of CCR1 inhibitors may be used as imaging agents for the diagnosis of Alzheimer's disease. United States Provisional Patent Application Serial No. 60/548950, filed Mar. 2, 2004 discloses that CCR1 inhibitors may be used to treat endometriosis.

Related Disclosures

Other piperidine compounds having CCR1 inhibitor activity are disclosed in WO 04/009550, and WO 04/009588.

### SUMMARY OF THE INVENTION

This invention is directed to compounds or their pharmaceutically acceptable salts which are functional antagonists of the CC chemokine receptor CCR1 and are therefore useful as pharmacological agents for the treatment of inflammatory disorders in humans.

Accordingly, in one aspect, this invention provides compounds of the following formulae I and II: 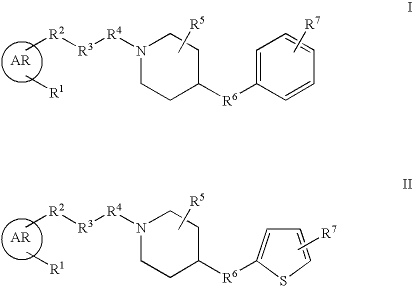
enantiomers, diastereomers, tautomers, salts and solvates thereof wherein 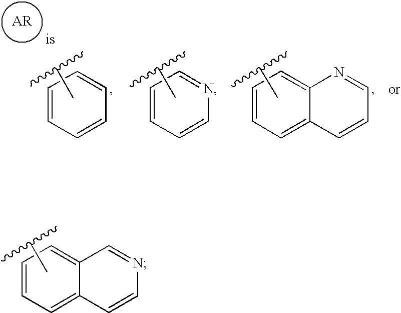
R¹ is one or more groups independently selected from
   (a) hydrogen, halo, nitro, alkyl, alkoxy, haloalkyl, cycloalkyl, (cycloalkyl)alkyl, alkenyl, alkynyl, aryl, (aryl)alkyl, heteroaryl, (heteroaryl)alkyl, heterocyclo, or (heterocyclo)alkyl,
   (b) —(O)_{q}—(Y)ₚ—CN,
      —(O)_{q}—(Y)ₚ—OR
      —(O)_{q*}—(Y)ₚ—C(═O)R¹⁰,
      —(O)_{q}—(Y)ₚ—C(═O)OR¹⁰,
      —(O)_{q}—(Y)ₚ—C(═O)—C(═O)OR¹⁰,
      —(O)_{q*}—(Y)ₚ—C(═O)—NR¹¹R¹²,
      —(O)_{q*}—(Y)ₚ—C(═O)—NR^{11a}—S(═O)ₜR^{10a},
      —(O)_{q}—(Y)ₚ—NH—OH,
      —(O)_{q}—(Y)ₚ—NH—NH₂,
      —(O)_{q}—(Y)ₚ—NR^{11a}R¹⁰,
      —(O)_{q}—(Y)ₚ—NR^{11a}—C(═O)R¹⁰,
      —(O)_{q}—(Y)ₚ—NR^{11a}C(═O)OR¹⁰,
      —(O)_{q}—(Y)ₚ—NR^{11a}—SO₂R¹⁰,
      —(O)_{q}—(Y)ₚ—N(R^{11a})—SO₂NR¹¹R¹²,
      —(O)_{q}—(Y)ₚ—NR^{11a}—C(═O)—NR¹¹R¹²,
      —(O)_{q}—(Y)ₚ—NR^{11a}—(Y)—C(═O)OR¹⁰,
      —(O)_{q}—(Y)ₚ—NR^{11a}—C(═O)—(Y)—NR¹¹R¹²,
      —(O)_{q}—(Y)ₚNR^{11a}—C(═O)—(Y)—C(═O)—NR¹¹R¹²
      —(O)_{q}—(Y)ₚ—SR¹⁰,
      —(O)_{q}—(Y)ₚ—SO₃H,
      —(O)_{q}—(Y)ₚ—S(═O)ₜR^{10a},
      —(O)_{q}—(Y)ₚ—S(═O)ₜNR¹¹R¹²,
      —(O)_{q}—(Y)ₚ—S(═O)ₜ—NR^{11a}C(═O)R^{10a}, or
      —(O)_{q}—(Y)ₚ—P(═O)(OH)OR¹⁰;
p is 0 or 1;
q* is 0 or 1;
q is 0 or 1, provided that q is not 1 when p is 0;
t is 1 or 2;
Y at each occurrence is independently
   a) alkylene optionally substituted independently with one or more halogen, —OH or —NR¹³R¹⁴ groups; or
   b) alkenylene optionally substituted independently with one or more —OH or —NR¹³R¹⁴ groups, and;
R² is —O—, —S—, —N(R⁸)—, —N(R⁸)—C(═O)—, —C(R⁹)₂— or a bond;
R³ is alkylene or alkenylene either of which may be optionally independently substituted by one or more aryl, hydroxy, oxo, —C(═O)OR¹⁰, or —N(R⁸)₂;
R⁴ is —C(═O)—, —OC(═O)—, —C(═S)—, —CH₂— or a bond;
R⁵ is one or more groups independently selected from hydrogen, oxo, halo, alkyl, alkenyl, cycloalkyl, haloalkyl, (cycloalkyl)alkyl, (aryl)alkyl, (heterocyclo)alkyl, (heteroaryl)alkyl, -(alkyl)ₚ—CN, -(alkyl)ₚ—OR¹⁰, -(alkyl)ₚ—C(═O)R¹⁰, -(alkyl)ₚ—C(═O)OR¹⁰, -(alkyl)ₚ—S(═O)ₜR^{10a}, -(alkyl)ₚ—C(═O)—NR¹¹R¹², -(alkyl)ₚ—NR¹¹R¹², -(alkyl)ₚ—NR^{11a}—C(═O)NR¹¹R¹², -(alkyl)ₚ—NR^{11a}—C(═O)R¹⁰, or -(alkyl)ₚ—NR^{11a}—C(═O)OR¹⁰;
R⁶ is —C(═O)—, —C(═S)—, —C(R⁹)₂—, ═C(R⁹)—, —S—, —S(═O)ₜ—;
R⁷ is one or more groups independently selected from hydrogen, halo, alkyl, cycloalkyl, alkenyl, -(alkyl)ₚ—CN, -(alkyl)ₚ—OR¹⁰, -(alkyl)ₚ—C(═O)OR¹⁰, -(alkyl)ₚ—NR^{11a}—C(═O)R¹⁰, -(alkyl)ₚ—NR^{11a}—C(═O)OR¹⁰, -(alkyl)ₚ—C(═O)—NR¹¹R¹² or -(alkyl)ₚ—NR^{11a}—C(═O)—NR¹¹R¹²;
each R⁸ is independently selected from hydrogen, alkyl, cycloalkyl, (cycloalkyl)alkyl, aryl, (aryl)alkyl, heteroaryl, (heteroaryl)alkyl, heterocyclo, (heterocyclo)alkyl, -(alkyl)ₚ—C(═O)R¹⁰, -(alkyl)ₚ—C(═O)OR¹⁰, or -(alkyl)ₚ—C(═O)—NR¹¹R¹²;
each R⁹ is independently selected from hydrogen, halo, alkyl, cycloalkyl, haloalkyl, (cycloalkyl)alkyl, aryl, (aryl)alkyl, -(alkyl)ₚ—OR¹⁰, -(alkyl)ₚ—C(═O)R¹⁰, -(alkyl)ₚ—O—C(═O)R¹⁰, -(alkyl)ₚ—C(═O)OR¹⁰, -(alkyl)ₚ—NR¹¹R¹², -(alkyl)ₚ—NR^{11a}—C(═O)R¹⁰, -(alkyl)ₚ—NR^{11a}—C(═O)OR¹⁰, -(alkyl)ₚ—NR^{11a}—S(═O)ₜR^{10a} or -(alkyl)ₚ—C(═O)—NR¹¹R¹²;
each R¹⁰ is independently selected from
   (a) hydrogen, or
   (b) alkyl, haloalkyl, cycloalkyl, (cycloalkyl)alkyl, aryl, (aryl)alkyl, heterocyclo, (heterocyclo)alkyl, heteroaryl or (heteroaryl)alkyl any of which may be optionally independently substituted with one or more Z groups;
R^{10a} is alkyl, haloalkyl, cycloalkyl, (cycloalkyl)alkyl, aryl, (aryl)alkyl, heterocyclo, (heterocyclo)alkyl, heteroaryl or (heteroaryl)alkyl any of which may be optionally independently substituted with one or more Z groups;
each R¹¹, R^{11a} and R¹² is independently selected from
   (a) hydrogen, hydroxy, NH₂ or
   (b) —C(═NH)—NH₂, or
   (c) alkyl, haloalkyl, (amino)alkyl, (hydroxy)alkyl, (alkoxy)alkyl, (aryloxy)alkyl, cycloalkyl, (cycloalkyl)alkyl, aryl, (aryl)alkyl, heterocyclo, (heterocyclo)alkyl, heteroaryl or (heteroaryl)alkyl any of which may be optionally independently substituted with one or more Z groups;
or R¹¹ and R¹² together with the nitrogen atom to which they are bonded may combine to form a heterocyclo ring optionally independently substituted with one or more Z groups;
R¹³ and R¹⁴ are independently hydrogen or alkyl;
Z at each occurrence is independently
   (1) V, where V is
      (i) alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocylco)alkyl, heteroaryl, or (heteroaryl)alkyl;
      (ii) a group (i) which is itself substituted by one or more of the same or different groups (i); or
      (iii) a group (i) or (ii) which is independently substituted by one or more (preferably 1 to 3) of the following groups (2) to (13),
   (2) —OH or —OV,
   (3) —SH or —SV,
   (4) —C(O)H, —C(O)OH, —C(O)V, —C(O)OV, or —O—C(O)V,
   (5) —SO₃H, —S(O)ₘV, or S(O)ₘN(V¹)V, where m is 1 or 2,
   (6) halo,
   (7) cyano,
   (8) nitro,
   (9) —U¹—NV²V³,
   (10) —U¹—N(V¹)—U²—NV²V³,
   (11) —U¹—N(V⁴)—U²—V,
   (12) —U¹—N(V⁴)—U²—H,
   (13) oxo;
U¹ and U² are each independently
   (1) a single bond,
   (2) —U³—S(O)ₜ—U⁴—,
   (3) —U³—C(O)—U⁴—,
   (4) —U³—C(S)—U⁴—,
   (5) —U³—O—U⁴—,
   (6) —U³—S—U⁴—,
   (7) —U³—O—C(O)—U⁴—,
   (8) —U³—C(O)—O—U⁴—,
   (9) —U³—C(═NV^{1a})—U⁴—, or
   (10) —U³—C(O)—C(O)—U⁴—;
V¹, V^{1a}, V², V³ and V⁴
   (1) are each independently hydrogen or a group provided in the definition of Z; or
   (2) V² and V³ may together be alkylene or alkenylene, completing a 3- to 8-membered saturated or unsaturated ring together with the atoms to which they are attached, which ring is unsubstituted or substituted with one or more groups listed in the definition of Z, or
   (3) V² or V³, together with V¹, may be alkylene or alkenylene completing a 3- to 8-membered saturated or unsaturated ring together with the nitrogen atoms to which they are attached, which ring is unsubstituted or substituted with one or more groups listed in the definition of Z; and
U³ and U⁴ are each independently
   (1) a single bond,
   (2) alkylene,
   (3) alkenylene, or
   (4) alkynylene;
      Particularly preferred are compounds of formula 1 and 11 according to the invention wherein 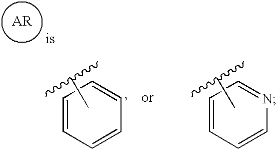
      R¹ is one or more groups independently selected from hydrogen, alkyl, hydroxy, alkoxy, cyano, halo, -(alkyl)ₚ—OR¹⁰, -(alkyl)ₚ—NR^{11a}—C(═O)—NR¹¹R¹², -(alkyl)ₚ—C(═O)OR¹⁰, -(alkyl)ₚ—C(═O)R¹⁰, -(alkyl)ₙ—CN, -(alkyl)ₚ—C(═O)—NR¹¹R¹², heteroaryl, (heteroaryl)alkyl, (heterocyclo)alkyl, -(alkyl)ₚ—NR¹¹R¹², -(alkyl)ₚ—NR^{11a}—C(═O)R¹⁰, -(alkyl)ₚ—NR^{11a}—CH₂—C(═O)OR¹⁰, or -(alkyl)ₚ—NR^{11a}—SO₂—R¹⁰;
      R⁵ is one or more groups independently selected from hydrogen, alkyl, alkenyl, keto, —CN, —C(═O)OR¹⁰, haloalkyl, (heterocyclo)alkyl, —(CH₂)ₚ—OR¹⁰, —(CH₂)ₚ—NR¹¹R¹², or —C(═O)R¹⁰;
      R⁶ is —CH₂—, —CHF—, —CH(OH)—, or —C(═O)—; and
      R⁷ is one or two same or different halo groups; and Y, p, q, R¹⁰, R¹¹, R^{11a}, and R¹² have the meaning defined above.
      Further preferred are the compounds of formulae I and II wherein
      R² is —O—;
      R³ is alkylene or alkenylene either of which may be optionally independently substituted by one or more aryl, hydroxy, oxo or —N(R⁸)₂; and
      R⁴ is —C(═O); and
      R⁸ has the meaning as previously defined.

A further aspect of the present invention refers to a compound of formula (I) or (II) as described above for use as a medicament.

Aim of the invention is also the use of a compound of formula (I) or (II) as described above, for the production of a medicament for the treatment of inflammatory disorders.

In another aspect, this invention provides pharmaceutical compositions useful in treating an inflammatory disorder in a human in need of such treatment, which composition comprises a therapeutically effective amount of a compound of formula (I) or (II) as described above, and a pharmaceutically acceptable excipient.

In another aspect, this invention provides a method of treating an inflammatory disorder in a human, which method comprises administering to a human in need of such treatment a therapeutically effective amount of a compound of formula (I) or (II) as described above.

Further aim of the present invention is a process for the preparation of a compound of formula (I) or (II) as described above.

### DETAILED DESCRIPTION OF THE INVENTION

Definitions

As used in the specification and appended claims, unless specified to the contrary, the following terms have the meaning indicated:

The term “alkyl” is used herein at all occurrences (as a group per se or a part of a group) to mean straight or branched chain alkyl groups of 1 to 6 carbon atoms, unless the chain length is otherwise indicated, including, but not limited to methyl, ethyl, n-propyl, isopropyl, n-butyl, sec-butyl, isobutyl, tert-butyl, and the like. Alkyl groups may also be substituted one or more times by halogen, aryl, substituted aryl, hydroxy, methoxy, amino, nitro, carboxy, or cyano or any other group within the definition of “Z” herein.

The term “lower alkyl” as used herein by itself or as part of another group refers to straight or branched chain alkyl groups of 1 to 6 carbon atoms, unless the chain length is otherwise indicated, including, but not limited to methyl, ethyl, n-propyl, isopropyl, n-butyl, sec-butyl, isobutyl, tert-butyl, and the like.

The term “alkenyl” as used herein by itself or as part of another group refers to straight or branched chain radicals of 2 to 8 carbons, (more preferably 2 to 6 carbons in the normal chain), which include one to 4 double bonds in the normal chain (preferably one to two double bonds) provided that two unsaturated bonds are not adjacent to each other, such as vinyl, 2-propenyl, 3-butenyl, 2-butenyl, 4-pentenyl, 3-pentenyl, 2-hexenyl, 3-hexenyl, 2-heptenyl, 3-heptenyl, 4-heptenyl, 3-octenyl, and the like.

The term “alkynyl” as used herein by itself or as part of another group refers to straight or branched chain hydrocarbon groups having 2 to 8 carbon atoms, (more preferably 2 to 6 carbon atoms), and at least one triple carbon to carbon bond, such as ethynyl, 2-propynyl, 3-butynyl, 2-butynyl, 4-pentynyl, 3-pentynyl, 2-hexynyl, 3-hexynyl, 2-heptynyl, 3-heptynyl, 4-heptynyl, 3-octynyl, and the like.

Where alkyl groups as defined above have single bonds for attachment to two other groups, they are termed “alkylene” groups (which may also be designated by “-(alkyl)-” as used herein). Similarly, where alkenyl groups as defined above and alkynyl groups as defined above, respectively, have single bonds for attachment to two other groups, they are termed “alkenylene groups” and “alkynylene groups” respectively.

The term “amino” as used herein by itself or as part of another group refers to a group —NR^{a}R^{b} where R^{a} and R^{b} are independently hydrogen, alkyl, cycloalkyl, (cycloalkyl)alkyl, aryl, (aryl)alkyl, heterocyclo, (heterocyclo)alkyl, heteroaryl or (heteroaryl)alkyl any of which may be optionally independently substituted with one or more groups falling within the definition of “Z” herein.

The term “cycloalkyl” as used herein by itself or as part of another group refers to saturated and partially unsaturated (containing 1 or 2 double bonds) cyclic hydrocarbon groups containing 1 to 3 rings, including monocyclicalkyl, bicyclicalkyl and tricyclicalkyl, containing a total of 3 to 20 carbons forming the rings, preferably 3 to 7 carbons, forming the ring. The rings of multi-ring cycloalkyls may be either fused, bridged and/or joined through one or more spiro union to 1 or 2 aromatic, cycloalkyl or heterocyclo rings. Exemplary cycloalkyl groups include cyclopropyl, cyclobutyl, cyclopentyl, cyclohexyl, cycloheptyl, cyclooctyl, cyclodecyl, cyclododecyl, cyclopentenyl, cyclohexenyl, cycloheptenyl, cyclooctenyl, cyclohexadienyl, cycloheptadienyl, 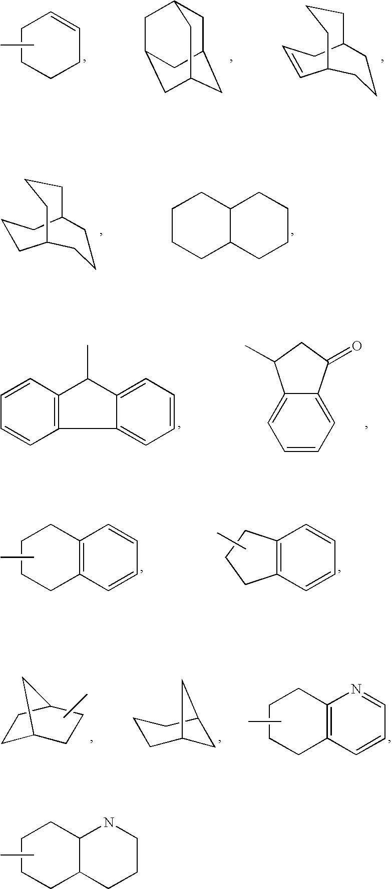
and the like. Cycloalkyl groups may optionally be substituted with one or more groups within the definition of “Z” herein.

“Alkoxy” means —O-alkyl groups in which the alkyl portion (substituted or unsubstituted) is in accordance with the previous definition. Suitable alkoxy groups include methoxy, ethoxy, propoxy and butoxy.

The terms “halo” or “halogen” are used interchangeably herein at all occurrences to mean radicals derived from the elements chlorine, fluorine, iodine or bromine. “Halogenated” is analogous and refers to a degree of halogen substitutions from single to full (per) substitution. The term “haloalkyl” represents a straight or branched alkyl chain substituted by 1 to 5 halo atoms, which can be attached to the same or different carbon atoms, e.g., —CH₂F, —CHF₂, —CF₃, F₃CCH₂— and —CF₂CF₃.

The term “heteroaryl” as used herein by itself or as part of another group refers to monocyclic and bicyclic aromatic rings containing from 5 to 10 atoms, which includes 1 to 4 hetero atoms such as nitrogen, oxygen or sulfur, and such rings fused to an aryl, cycloalkyl, heteroaryl or heterocyclo ring, where the nitrogen and sulfur heteroatoms may optionally be oxidized and the nitrogen heteroatoms may optionally be quaternized. Examples of heteroaryl groups include pyrrolyl, pyrazolyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl, indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, cinnolinyl, quinoxalinyl, indazolyl, pyrrolopyridyl, furopyridyl, tetrahydroquinolinyl, carbazolyl, benzidolyl, phenanthrollinyl, acridinyl, phenanthridinyl, xanthenyl 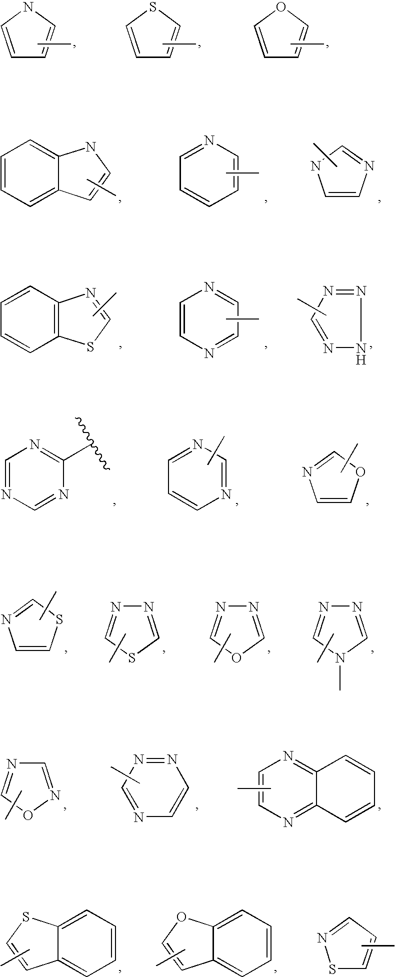
and the like. Heteroaryl groups may optionally be substituted with one or more groups within the definition of “Z” herein.
The terms “heterocyclic” or “heterocyclo” as used herein by itself or as part of another group refer to optionally substituted, fully saturated or partially unsaturated cyclic groups (for example, 3 to 13 member monocyclic, 7 to 17 member bicyclic, or 10 to 20 member tricyclic ring systems, preferably containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valance allows. The rings of multi-ring heterocycles may be either fused, bridged and/or joined through one or more spiro unions to 1 or 2 aromatic, heteroaryl or cycloalkyl rings. Exemplary heterocyclic groups include azetidinyl, pyrrolidinyl, pyrazolinyl, oxetanyl, imidazolinyl, oxazolidinyl, isoxazolinyl, thiazolidinyl, isothiazolidinyl, tetrahydrofuranyl, piperidinyl, piperazinyl, 2-oxopiperazinyl, 2-oxopiperidinyl, 2-oxopyrrolidinyl, 2-oxoazepinyl, azepinyl, 4-piperidonyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, benzodioxolyl, dihydroisoindolyl, 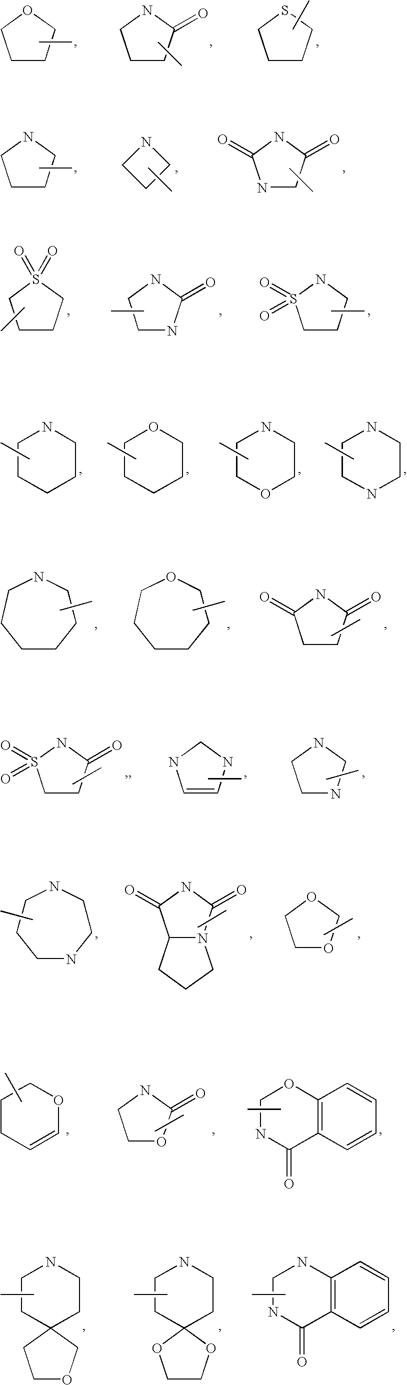 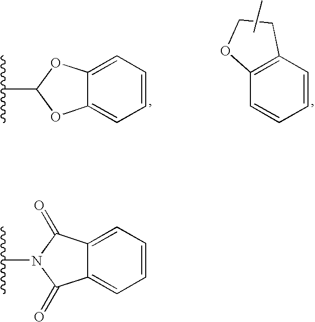
and the like. Heterocyclo groups may optionally be substituted with one or more groups within the definition of “Z” herein.

The terms “ar” or “aryl” as used herein by itself or as part of another group refer to aromatic hydrocarbon monocyclic, bicyclic or tricyclic ring groups containing 6 to 14 carbons in the ring portion (such as phenyl, biphenyl, naphthyl (including 1-naphthyl and 2-naphthyl) and anthracenyl) and may optionally include one to three additional rings (either cycloalkyl, heterocyclo or heteroaryl) fused thereto. Examples include: 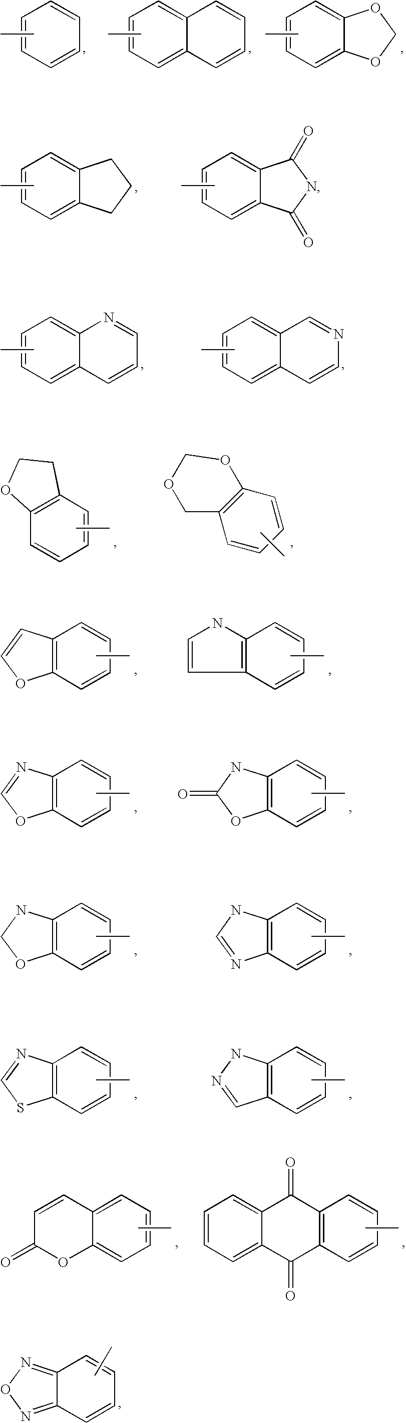
and the like. Aryl groups may optionally be substituted with one or more groups within the definition of “Z” herein.
The term “arylalkyl”, “aralkyl”, “(aryl)alkyl” or “(ar)alkyl” refers to a residue in which an aryl moiety is attached to the parent structure via an alkyl residue, wherein the aryl and alkyl portions are in accordance with the descriptions above. Similarly, terms such as “(heteroaryl)alkyl”, “(heterocyclo)alkyl”, and “(cycloalkyl)alkyl” refer respectively to heteroaryl, heterocyclo and cycloalkyl moieties that are attached to the parent structure via an alkyl residue.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, propionic acid, pyruvic acid, maleic acid, malonic acid, succinic acid, fumaric acid, tartaric acid, citric acid, benzoic acid, mandelic acid, methanesulfonic acid, ethanesulfonic acid, p-toluenesulfonic acid, salicylic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, zinc, aluminum salts and the like. Preferred inorganic salts are the ammonium, sodium, lithium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

“THF” refers to tetrahydrofuran.

“Therapeutically effective amount” refers to that amount of a compound of formula (I) or (II) which, when administered to a human in need of such administration, is sufficient to effect treatment, as defined below, for inflammatory disorders which are alleviated by functional antagonism of the chemokine receptor CCR1, in particular, for inflammatory disorders characterized by migration, accumulation and activation of leukocytes to the affected tissue. The amount of a compound of formula (I) or (II) which constitutes a “therapeutically effective amount” will vary depending on the compound, the disorder and its severity, and the age of the human to be treated, but can be determined routinely by one of ordinary skill in the art having regard to his own knowledge and to this disclosure.

“Treating” or “treatment” as used herein cover the treatment of an inflammatory disorder in a human; and include:

(i) preventing the disorder from occurring in a human, in particular, when such human is predisposed to the disorder but has not yet been diagnosed as having it;

(ii) inhibiting the disorder, i.e., arresting its development; or

(iii) relieving the disorder, i.e., causing regression of the disorder.

It is understood from the above definitions and examples that for radicals containing a substituted alkyl group any substitution thereon can occur on any carbon of the alkyl group.

The compounds of the invention, or their pharmaceutically acceptable salts, may have asymmetric carbon atoms in their structure. The compounds of the invention and their pharmaceutically acceptable salts may therefore exist as single stereoisomers, racemates, and as mixtures of enantiomers and diastereomers. All such single stereoisomers, racemates and mixtures thereof are intended to be within the scope of this invention. Absolute configuration of certain carbon atoms within the compounds, if known, are indicated by the appropriate absolute descriptor R or S.

Utility and Administration

A. Utility

The compounds of the invention are functional antagonists of the CC chemokine receptor CCR1 and are therefore useful as anti-inflammatory agents. In particular, the compounds are useful in treating inflammatory disorders such as multiple sclerosis, leukoencephalopathy, encephalomyelitis, Alzheimer's disease, Guillian-Barre syndrome, acute cell-mediated renal transplant rejection, allograft rejection, rheumatoid arthritis, atherosclerosis, uricaria, angioderma, allergic conjunctivitis, atopic dermatitis, psoriasis, allergic contact dermatitis, drug or insect sting allergy or systemic anaphylaxis. Of particular interest to the invention is the use of the compounds to treat multiple sclerosis.

The compounds of the present invention are further useful in treating endometriosis, fibrosis particularly renal fibrosis, heart transplant rejection, myocarditis, and multiple myeloma. Additionally, radio-labeled analogues of these compounds may also be used as imaging agents for the diagnosis of Alzheimer's disease, as disclosed in U.S. Pat. No. 6,676,926.

B. Testing

To demonstrate that the compounds are functional antagonists of CCR1 several assays in which they inhibit the ability of the CCR1 ligands MIP-1α and RANTES to activate the receptor may be employed. One assay utilizes a microphysiometer, which uses a silicon-based light addressable potentiometric sensor to continuously monitor subtle changes in extracellular pH levels. These changes result from the generation of acidic metabolites excreted by living cells into their immediate microenvironment during basal and stimulated conditions. It has been previously demonstrated by microphysiometry that THP-1 cells, which have been shown to express the chemokine receptors, CCR1 and CCR2, respond dose-responsively to their respective chemokines, including MIP-1α, RANTES and MCP-1 (a ligand for CCR2). See, e.g., Hirst, M. et al., “Chemokine receptors,” Journal of NIH Research (1995), Vol. 80.

Another assay which may be used to demonstrate the ability of the compounds to inhibit the activity of MIP-1α and RANTES is based on the measurement of intracellular Ca²⁺ concentrations and/or increases in intracellular [³H] inositol phosphate release from MIP-1α and RANTES stimulated cells. Ligand binding to the CCR1 receptor results in G-protein induced activation of phospholipase C, which leads to the conversion of phosphatidyl inositol phosphate to inositol phosphate and diacyglycerol. Inositol phosphate in turn binds to a receptor located at intracellular sites to release Ca²⁺ into the cytoplasm. In addition to Ca²⁺ concentration increases due to release from intracellular stores, binding of inositol phosphate to its receptor leads to an increased flux of extracellular calcium across the membrane and into the cell. Thus the activation of the CCR1 receptor by MIP-1α and RANTES and, subsequently, inhibition of the activation by the compounds of the invention can be determined by assaying for an increase in free intracellular Ca²⁺ levels. Typically this can be achieved by the use of calcium-sensitive fluorescent probes such as quin-2, fura-2 and indo-1. Alternatively, functional activation or inhibition of the activation of the CCR1 receptor can be measured by quantitation of [³H] inositol phosphate release from the cell pre-labeled with [³H] inositol.

Standard in vitro binding assays may be employed to demonstrate the affinity of the compounds for the CCR1 receptor (thereby inhibiting the activity of MIP-1α and RANTES by competitive binding to the receptor). See, e.g., Neote, K. et al., Cell (1993), Vol. 72, pp. 415-425. One particular assay employs the use of HEK293 cells which have been stablely transfected to express human CCR1 receptor.

The compounds of the invention exemplified herein have been tested using in vitro assay techniques, and have demonstrated their affinity to bind to the CCR1 receptor.

Standard in vivo assays which may be employed to demonstrate the compounds usefulness as anti-inflammatory agents are the animal model for experimental autoimmune encephalomyelitis (EAE) model for multiple sclerosis and the adjuvant-induced arthritis (AIA) model for rheumatoid arthritis.

C. General Administration

Administration of the compounds of the invention, or their pharmaceutically acceptable salts, in pure form or in an appropriate pharmaceutical composition, can be carried out via any of the accepted moues of administration or agents for serving similar utilities. Thus, administration can be, for example, orally, nasally, parenterally, topically, transdermally, or rectally, sublingually, intramuscular, subcutaneously, or intravenously in the form of solid, semi-solid, lyophilized powder, or liquid dosage forms, such as for example, tablets, suppositories, pills, soft elastic and hard gelatin capsules, powders, solutions, suspensions, or aerosols, or the like, preferably in unit dosage forms suitable for simple administration of precise dosages. The compositions will include a conventional pharmaceutical carrier or excipient and a compound of the invention as the/an active agent, and, in addition, may include other medicinal agents, pharmaceutical agents, carriers, adjuvants, etc.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of one or more suitable pharmaceutical excipient(s). Preferably, the composition will be about 5% to 75% by weight of a compound(s) of the invention, or a pharmaceutically acceptable salt thereof, with the rest being suitable pharmaceutical excipients.

The preferred route of administration is oral, using a convenient daily dosage regimen which can be adjusted according to the degree of severity of the disease-state to be treated. For such oral administration, a pharmaceutically acceptable composition containing a compound(s) of the invention, or a pharmaceutically acceptable salt thereof, is formed by the incorporation of any of the normally employed excipients. Such excipients include non-toxic and chemically compatible fillers, binders, disintegrants, buffers, preservatives, anti-oxidants, lubricants, flavorings, thickeners, coloring agents, emulsifiers, and the like, for example, pharmaceutical grades of mannitol, lactose, starch, pregelatinized starch, magnesium stearate, sodium saccharine, talcum, cellulose ether derivatives, glucose, gelatin, sucrose, citrate, cyclodextrin, propyl gallate, and the like. Such compositions take the form of solutions, suspensions, tablets, pills, capsules, powders, sustained release formulations and the like.

Preferably such compositions will take the form of capsule, caplet or tablet and therefore will also contain a diluent such as lactose, sucrose, dicalcium phosphate, and the like; a disintegrant such as croscarmellose sodium or derivatives thereof; a lubricant such as magnesium stearate and the like; and a binder such as a starch, gum acacia, polyvinylpyrrolidone, gelatin, cellulose ether derivatives, and the like.

The compounds of the invention, or their pharmaceutically acceptable salts, may also be formulated into a suppository using, for example, about 0.5% to about 50% active ingredient disposed in a carrier that slowly dissolves within the body, e.g., polyoxyethylene glycols and polyethylene glycols (PEG), e.g., PEG 1000 (96%) and PEG 4000 (4%), and propylene glycol.

Liquid pharmaceutically administrable compositions can, for example, be prepared by dissolving, dispersing, etc., a compound(s) of the invention (about 0.5% to about 20%), or a pharmaceutically acceptable salt thereof, and optional pharmaceutical adjuvants in a carrier, such as, for example, water, saline, aqueous dextrose, aqueous cyclodextrin, glycerol, ethanol and the like, to thereby form a solution or suspension.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of an inflammatory disorder alleviated by the inhibition of the activity of the CC chemokine receptor CCR1.

The compounds of the invention, or their pharmaceutically acceptable salts, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. Generally, a therapeutically effective daily dose is from about 0.014 mg to about 14.0 mg/kg of body weight per day of a compound of the invention, or a pharmaceutically acceptable salt thereof; preferably, from about 0.14 mg to about 10.0 mg/kg of body weight per day; and most preferably, from about 1.4 mg to about 7.0 mg/kg of body weight per day. For example, for administration to a 70 kg person, the dosage range would be from about 1.0 mg to about 1.0 gram per day of a compound of the invention, or a pharmaceutically acceptable salt thereof, preferably from about 10 mg to about 700 mg per day, and most preferably from about 100 mg to about 500 mg per day.

The following examples illustrate various pharmaceutical compositions comprising compounds of the present invention together pharmaceutically acceptable vehicles, carriers, or excipients therefor:

Oral Formulation

This example illustrates the preparation of representative pharmaceutical compositions for oral administration containing a compound of the invention, as a single stereoisomer, a mixture of stereoisomers, or as a racemic mixture of stereoisomers; or as a hydrate thereof, or as a pharmaceutically acceptable salt thereof:

| | | |
|---|---|---|
| A. | Ingredients | % wt./wt. |

| | |
|---|---|
| Compound of the invention | 20.0% |
| Lactose | 79.5% |
| Magnesium stearate | 0.5% |

The above ingredients are mixed and dispensed into hard-shell gelatin capsules containing 100 mg each, one capsule would approximate a total daily dosage.

| | | |
|---|---|---|
| B. | Ingredients | % wt./wt. |

| | |
|---|---|
| Compound of the invention | 20.0% |
| Magnesium stearate | 0.9% |
| Starch | 8.6% |
| Lactose | 69.6% |
| PVP (polyvinylpyrrolidine) | 0.9% |

The above ingredients with the exception of the magnesium stearate are combined and granulated using water as a granulating liquid. The formulation is then dried, mixed with the magnesium stearate and formed into tablets with an appropriate tableting, machine.

| | |
|---|---|
| C. | Ingredients |

| | | |
|---|---|---|
| Compound of the invention | 0.1 | g |
| Propylene glycol | 20.0 | g |
| Polyethylene glycol 400 | 20.0 | g |
| Polysorbate 80 | 1.0 | g |
| Water | q.s. 100 | mL |

The compound of the invention is dissolved in propylene glycol, polyethylene glycol 400 and polysorbate 80. A sufficient quantity of water is then added with stirring to provide 100 mL of the solution which is filtered and bottled.

| | | |
|---|---|---|
| D. | Ingredients | % wt./wt. |

| | |
|---|---|
| Compound of the invention | 20.0% |
| Peanut Oil | 78.0% |
| Span 60 | 2.0% |

The above ingredients are melted, mixed and filled into soft elastic capsules.

| | | |
|---|---|---|
| E. | Ingredients | % wt./wt. |

| | |
|---|---|
| Compound of the invention | 1.0% |
| Methyl or carboxymethyl cellulose | 2.0% |
| 0.9% saline | q.s. 100 mL |

The compound of the invention is dissolved in the cellulose/saline solution, filtered and bottled for use.

Parenteral Formulation

This example illustrates the preparation of a representative pharmaceutical formulation for parenteral administration containing a compound of the invention, as a single stereoisomer, a mixture of stereoisomers, or as a racemic mixture of stereoisomers; or as a pharmaceutically acceptable salt thereof:

| Ingredients | | |
|---|---|---|
| Compound of the invention | 0.02 | g |
| Propylene glycol | 20.0 | g |
| Polyethylene glycol 400 | 20.0 | g |
| Polysorbate 80 | 1.0 | g |
| 0.9% Saline solution | q.s. 100 | mL |

The compound of the invention is dissolved in propylene glycol, polyethylene glycol 400 and polysorbate 80. A sufficient quantity of 0.9% saline solution is then added with stirring to provide 100 mL of the I.V. solution which is filtered through a 0.2 m membrane filter and packaged under sterile conditions.

Suppository Formulation

This example illustrates the preparation of a representative pharmaceutical composition in suppository form containing a compound of the invention, as a single stereoisomer, a mixture of stereoisomers, or as a racemic mixture of stereoisomers; or as a pharmaceutically acceptable salt thereof:

| Ingredients | % wt./wt. |
|---|---|
| Compound of the invention | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

Insufflation Formulation

This example illustrates the preparation of a representative pharmaceutical formulation for insufflation containing a compound of the invention, as a single stereoisomer, a mixture of stereoisomers, or as a racemic mixture of stereoisomers; or as a hydrate thereof, or as a pharmaceutically acceptable salt thereof:

| Ingredients | % wt./wt. |
|---|---|
| Micronized compound of the invention | 1.0% |
| Micronized lactose | 99.0% |

The ingredients are milled, mixed, and packaged in an insufflator equipped with a dosing pump.

Nebulized Formulation

This example illustrates the preparation of a representative pharmaceutical formulation in nebulized form containing a compound of the invention, as a single stereoisomer, a mixture of stereoisomers, or as a racemic mixture of stereoisomers; or as a pharmaceutically acceptable salt thereof:

| Ingredients | % wt./wt. |
|---|---|
| Compound of the invention | 0.005% |
| Water | 89.995% |
| Ethanol | 10.000% |

The compound of the invention is dissolved in ethanol and blended with water. The formulation is then packaged in a nebulizer equipped with a dosing pump.

Aerosol Formulation

This example illustrates the preparation of a representative pharmaceutical formulation in aerosol form containing a compound of the invention, as a single stereoisomer, a mixture of stereoisomers, or as a racemic mixture of stereoisomers; or as a pharmaceutically acceptable salt thereof:

| Ingredients | % wt./wt. |
|---|---|
| Compound of the invention | 0.10% |
| Propellant 11/12 | 98.90% |
| Oleic acid | 1.00% |

The compound of the invention is dispersed in oleic acid and the propellants. The resulting mixture is then poured into an aerosol container fitted with a metering valve.

Preferred Embodiments

Preferred compounds of the present invention include compounds of the following formulae Ia and IIa: 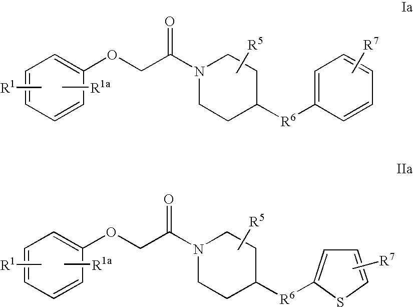
where
R¹ is one or more groups independently selected from hydrogen, alkyl, haloalkyl, heterocyclo, hydroxy, alkoxy, cyano, halo or —(O)_{q}—(Y)ₚ—NR¹¹R¹²;
R^{1a} is heteroaryl, (heteroaryl)alkyl, (heterocyclo)alkyl,
   —(O)_{q}—(Y)ₚ—CN,
   —(O)_{q}—(Y)ₚ—OR¹⁰,
   —(O)_{q*}—(Y)ₚ—C(═O)OR¹⁰,
   —(O)_{q*}—(Y)ₚ—C(═O)R¹⁰,
   —(O)_{q*}—(Y)ₚ—C(═O)—NR¹¹R¹²,
   —(O)_{q}—(Y)ₚ—NR^{11a}—C(═O)—NR¹¹R¹²,
   —(O)_{q}—(Y)ₚ—NR^{11a}—(Y)—C(═O)OR¹⁰,
   —(O)_{q}—(Y)ₚ—NR^{11a}—SO₂R¹⁰, or
   —(O)_{q}—(Y)ₚ—NR^{11a}—C(═O)—(Y)—NR¹¹R¹²
R⁵ is one or more groups independently selected from hydrogen, alkyl, alkenyl, keto, —CN, —C(═O)OR¹⁰, haloalkyl, (heterocyclo)alkyl, —(CH₂)ₚ—OR¹⁰, —(CH₂)ₙ—NR¹¹R¹², or —C(═O)R¹⁰;
R⁶ is —CH₂—, —CHF—, —CH(OH)—, or —C(═O)—; and
R⁷ is one or two same or different halo groups.
   Further preferred forms of embodiment according to the present invention are compounds of formulae Ia and IIa where
R¹ is halo; and
R^{1a} is —(CH₂)—OR¹⁰, —(CH₂)—NR¹¹R¹², —(CH₂)—NR^{11a}—C(═O)—NR¹¹R¹², —(CH₂)—C(═O)OR¹⁰, —(CH₂)—C(═O)R¹⁰, —(CH₂)CN, —(CH₂)—C(═O)—NR¹¹R¹², —(CH₂)—NR^{11a}—C(═O)R¹⁰, —CH₂—NR^{11a}—CH₂—C(═O)OR¹⁰, —(CH₂)—NR^{11a}—SO₂—R¹⁰, —NR^{11a}—SO₂—R¹⁰, —C(═O)OR¹⁰, —CH═CH—C(═O)OR¹⁰, or —(CH₂)₂—C(═O)OR¹⁰, and
R¹⁰, R¹¹, R^{11a}, and R¹² have the meaning as defined above.
   Particularly preferred are compounds of formulae Ia and IIa where
q and q* is 0,
p is 0 or 1,
Y is CH₂ or CH═CH or CH₂CH₂,
R¹⁰ is hydrogen or lower alkyl,
R^{11a} is hydrogen,
R¹¹ is hydrogen or lower alkyl,
R¹² is hydrogen, lower alkyl, haloalkyl or alkyl-CO₂R¹⁰
or NR¹¹R¹² is pyrrolidine or piperidine.

Preferred compounds of formula Ia include compounds of the following formulae Ib, Ic, and Id: 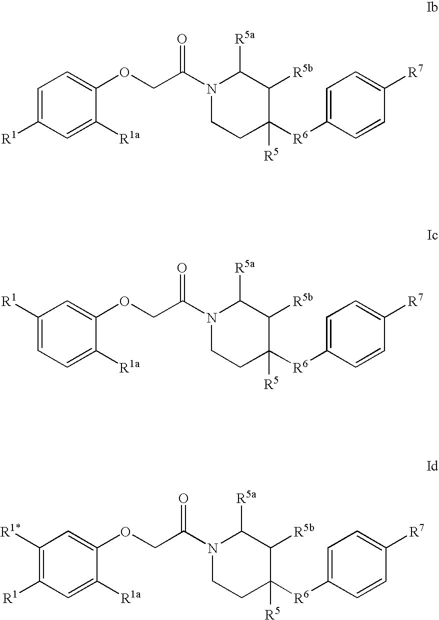
where
R¹, R^{1a}, R⁵, R⁶, and R⁷ are as defined for formula Ia;
R¹* is hydrogen, alkyl, haloalkyl, heterocyclo, hydroxy, alkoxy, cyano, halo or —(O)_{q}—(Y)ₚ—NR¹¹R¹²;
R^{5a} is hydrogen or alkyl; and
R^{5b} is hydrogen, alkyl, keto, or hydroxy.
   Further preferred forms of embodiment according to the present invention are compounds of formulae Ib, Ic and Id where
R¹ is halo; and
R^{1a} is —(CH₂)—OR¹⁰, —(CH₂)—NR¹¹R¹², —(CH₂)13 NR^{11a}—C(═O)—NR¹¹R¹², —(CH₂)—C(═O)OR¹⁰, —(CH₂)—C(═O)R¹⁰, —(CH₂)CN, —(CH₂)—C(═O)—NR¹¹R¹², —(CH₂)—NR^{11a}—C(═O)R¹⁰, —CH₂—NR^{11a}—CH₂—C(═O)OR¹⁰, —(CH₂)—NR^{11a}—SO₂—R¹⁰, —NR^{11a}—SO₂—R¹⁰, —C(═O)OR¹⁰, —CH═CH—C(═O)OR¹⁰, or —(CH₂)₂—C(═O)OR¹⁰, and
R¹⁰, R¹¹, R^{11a}, and R¹² have the meaning as defined above.
   Particularly preferred are compounds of formulae Ib, Ic and Id where
q is 0,
p is 0 or 1,
Y is CH₂ or CH═CH or CH₂CH₂,
R¹⁰ is hydrogen or lower alkyl,
R^{11a} is hydrogen,
R¹¹ is hydrogen or lower alkyl,
R¹² is hydrogen, lower alkyl, haloalkyl or alkyl-CO₂R¹⁰
or NR¹¹R¹² is pyrrolidine or piperidine.
   Preferred compounds of formula IIa include compounds of the following formulae IIb, IIc, and IId: 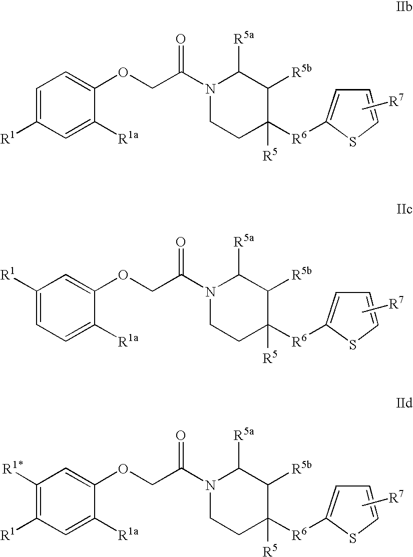
   where
R¹, R^{1a}, R⁵, R⁶, and R⁷ are as defined for formula Ia;
R¹* is hydrogen, alkyl, haloalkyl, heterocyclo, hydroxy, alkoxy, cyano, halo or —(O)_{q}—(Y)ₚ—NR¹¹R¹²;
R^{5a} is hydrogen or alkyl; and
R^{5b} is hydrogen, alkyl, keto, or hydroxy.
Further preferred forms of embodiment according to the present invention are compounds of formulae IIb, IIc and IId where
R¹ is halo; and
R^{1a} is —(CH₂)—OR¹⁰, —(CH₂)—NR¹¹R¹², —(CH₂)—NR^{11a}—C(═O)—NR¹¹R¹², —(CH₂)—C(═O)OR¹⁰, —(CH₂)—C(═O)R¹⁰, —(CH₂)—CN, —(CH₂)—C(═O)—NR¹¹R¹², —(CH₂)—NR^{11a}—C(═O)R¹⁰, —CH₂—NR^{11a}—CH₂—C(═O)OR¹⁰, —(CH₂)—NR^{11a}—SO₂—R¹⁰, —NR^{11a}—SO₂—R¹⁰, —C(═O)OR¹⁰, —CH═CH—C(═O)OR¹⁰, or —(CH₂)₂—C(═O)OR¹⁰, and
R¹⁰, R¹¹, R^{11a}, and R¹² have the meaning as defined above.
   Particularly preferred are compounds of formulae IIb, IIc and IId where
q is 0,
p is 0 or 1,
Y is CH₂ or CH═CH or CH₂CH₂,
R¹⁰ is hydrogen or lower alkyl,
R^{11a} is hydrogen,
R¹¹ is hydrogen or lower alkyl,
R¹² is hydrogen, lower alkyl, haloalkyl or alkyl-CO₂R¹⁰
or NR¹¹R¹² is pyrrolidine or piperidine.
Further preferred forms of embodiment according to the present invention are the compounds according to examples 1 to 2XX as described in the following.
   Most preferred forms of embodiments of the invention are the compounds:
N-[5-chloro-2-[2-[4-cyano-4-[(4-fluorophenyl)methyl]-1-piperidinyl]-2-oxoethoxy]phenyl]urea
N-[5-chloro-2-[2-[4-[(4-fluorophenyl)methyl]-4-[(2-hydroxypropyl)amino]-1-piperidinyl]-2-oxoethoxy]phenyl]urea
N-[5-chloro-2-[2-[4-[(4-fluorophenyl)methyl]-4-hydroxy-1-piperidinyl]-2-oxoethoxy]phenyl]urea
[[[1-[[2-[(aminocarbonyl)amino]-4-chlorophenoxy]acetyl]-4-[(4-fluorophenyl)methyl]-4-piperidinyl]methyl]amino]acetic acid
N-[5-chloro-2-[2-[4-cyano-4-(4-fluorobenzoyl)-1-piperidinyl]-2-oxoethoxy]phenyl]urea
N-[5-chloro-2-[2-[4-cyano-4-[fluoro(4-fluorophenyl)methyl]-1-piperidinyl]-2-oxoethoxy]phenyl]urea
5-chloro-2-[2-[4-cyano-4-[(4-fluorophenyl)methyl]-1-piperidinyl]-2-oxoethoxy]benzoic acid
N-[[5-chloro-2-[2-[4-cyano-4-[(4-fluorophenyl)methyl]-1-piperidinyl]-2-oxoethoxy]phenyl]methyl]methanesulfonamide
N-[5-chloro-2-[2-[4-cyano-4-[(4-fluorophenyl)methyl]-1-piperidinyl]-2-oxoethoxy]phenyl]methanesulfonamide
5-bromo-2-[2-[4-cyano-4-[(4-fluorophenyl)methyl]-1-piperidinyl]-2-oxoethoxy]benzeneacetic acid
5-bromo-2-[2-[4-[(4-fluorophenyl)methyl]-4-(1-piperidinylmethyl)-1-piperidinyl]-2-oxoethoxy]benzeneacetic acid
5-chloro-2-[2-[4-cyano-4-[(4-fluorophenyl)methyl]-1-piperidinyl]-2-oxoethoxy]benzeneacetic acid
5-bromo-2-[2-[4-[(4-fluorophenyl)methyl]-4-(1-pyrrolidinylmethyl)-1-piperidinyl]-2-oxoethoxy]benzeneacetic acid
3-[5-chloro-2-[2-[4-cyano-4-[(4-fluorophenyl)methyl]-1-piperidinyl]-2-oxoethoxy]phenyl]-2-propenoic acid
5-chloro-2-[2-[4-cyano-4-[(4-fluorophenyl)methyl]-1-piperidinyl]-2-oxoethoxy]benzenepropanoic acid
5-bromo-2-[2-[4-[(4-fluorophenyl)methyl]-4-[(methylamino)methyl]-1-piperidinyl]-2-oxoethoxy]benzeneacetic acid
[[5-bromo-2-[2-[4-cyano-4-[(4-fluorophenyl)methyl]-1-piperidinyl]-2-oxoethoxy]phenyl]methyl]phosphonic acid ethyl ester
5-bromo-2-[2-[4-(2-cyanoethyl)-4-[(4-fluorophenyl)methyl]-1-piperidinyl]-2-oxoethoxy]benzeneacetic acid
5-chloro-2-[2-[4-cyano-4-[(4-fluorophenyl)methyl]-1-piperidinyl]-2-oxoethoxy]benzenemethanesulfonic acid
5-bromo-2-[2-[4-[(4-fluorophenyl)methyl]-4-[[(2,2,2-trifluoroethyl)amino]methyl]-1-piperidinyl]-2-oxoethoxy]benzeneacetic acid
5-bromo-2-[2-[4-cyano-4-[(4-fluorophenyl)methyl]-1-piperidinyl]-2-oxoethoxy]-4-(dimethylamino)benzeneacetic acid
5-chloro-2-[2-[4-cyano-4-[(4-fluorophenyl)methyl]-1-piperidinyl]-2-oxoethoxy]benzeneacetic acid
5-chloro-2-[2-[4-cyano-4-[(4-fluorophenyl)methyl]-1-piperidinyl]-2-oxoethoxy]-4-methylbenzeneacetic acid
5-chloro-2-[(1E)-3-[4-cyano-4-[(4-fluorophenyl)methyl]-1-piperidinyl]-3-oxo-1-propenyl]benzeneacetic acid
5-bromo-2-[2-[4-cyano-4-[fluoro(4-fluorophenyl)methyl]-1-piperidinyl]-2-oxoethoxy]benzeneacetic acid
5-bromo-2-[2-[4-[(5-chloro-2-thienyl)methyl]-4-cyano-1-piperidinyl]-2-oxoethoxy]benzeneacetic acid
5-bromo-2-[2-[4-[(4-chlorophenyl)methyl]-4-cyano-1-piperidinyl]-2-oxoethoxy]benzeneacetic acid
   Preparation of Compounds of the Invention

The following Reaction Schemes are directed to the preparation of compounds of formula I. It is understood that those compounds of the invention which are not specifically prepared in the following Reaction Schemes may be prepared by similar synthetic processes with the appropriately substituted starting materials and reagents. It is also understood that in the following descriptions, combinations of the various substituents on the depicted formulae are permissible only if such combinations result in stable compounds.

For the purposes of convenience only, preparation of compounds of the invention where Ar is only phenyl are illustrated below. It is understood that other Ar groups may be prepared in a similar manner.

It is also understood that during the preparation of the compounds of the invention, as described below, additional reactive groups (for example, hydroxy, amino or carboxy groups) on the intermediate compounds utilized in the preparation may be protected as needed by the appropriate protecting group by treating the intermediate compound prior to the desired reaction with the appropriate protecting group precursor by methods known to those of ordinary skill in the art. The protecting groups may then be removed as desired by methods known to those of ordinary skill in the art, for example, by acidic or basic hydrolysis. Such protecting groups and methods are described in detail in Greene, T. W. and Wuts, P. G. M., “Protective Groups in Organic Synthesis”, 2nd Edition, 1991, John Wiley & Sons.

Scheme I

Compounds where R² is —O—, R³ is alkylene, and R⁴ is —C(═O)—, can be made according to the following Scheme 1:

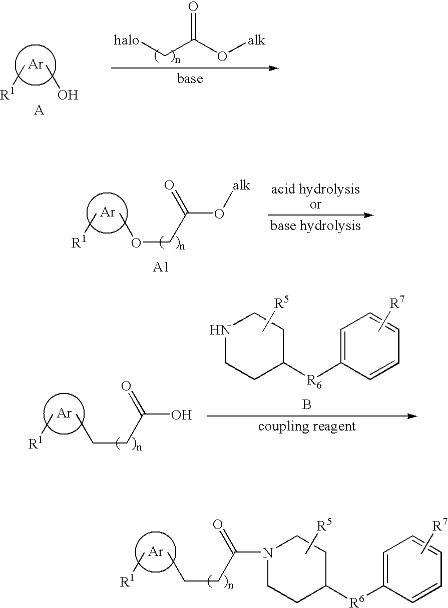

Precursor A is reacted with the desired haloalkylester to generate Precursor A1, which is then hydrolyzed to its acid form and coupled with Precursor B to generate the desired end product.

Scheme 2

The compounds generated according to Scheme 1 can alternatively be synthesized according to the following scheme:

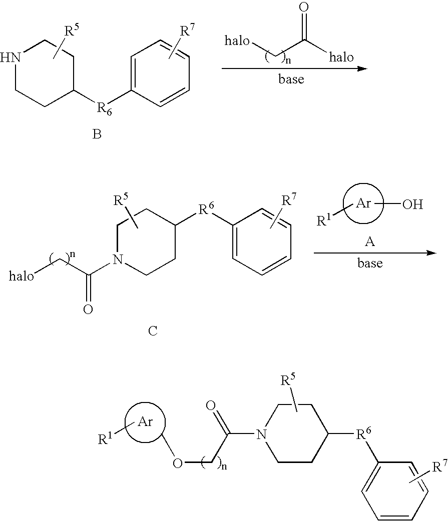

Precursor B is reacted with the desired haloalkylcarbonylhalide to generate Precursor C, which is then coupled with Precursor A to generate the desired end product.

Scheme 3

Compounds where R² is —O—, R³ is a hydroxyl-substituted alkylene, and R⁴ is a bond or —CH₂— can be made according to the following scheme:

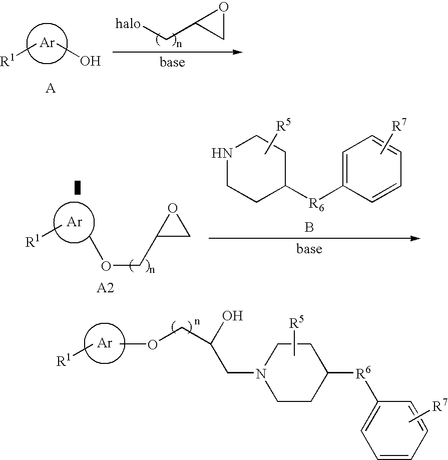

Precursor A is reacted with the desired haloalkyl-oxirane to generate Precursor A2, which is then coupled with Precursor B to generate the desired end product.

Schemes 4

Precursor B compounds containing an R⁵ substituent linked via a carbon atom to the 4-position of the piperidine ring can be made according to the following general scheme: 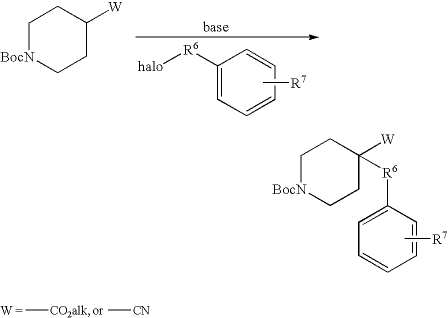
The W functionality, linked to the piperidine ring via a carbon atom in the end product of the above reaction Scheme 4, can then be further transformed into any of the various functional R⁵ groups that are linked via a carbon atom using synthetic methods and techniques well known to those of skill in the art.
Scheme 5

Precursor B compounds containing an R⁵ or R^{5a} alkyl substituent linked to the 2-position of the piperidine ring can be made according to the following general scheme: 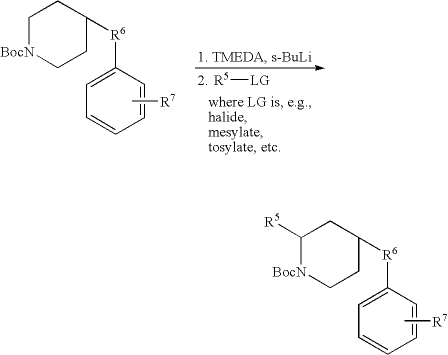
Scheme 6

Precursor B compounds containing an R⁵ or R^{5b} alkyl substituent linked to the 3-position of the piperidine ring can be made according to the following general scheme: 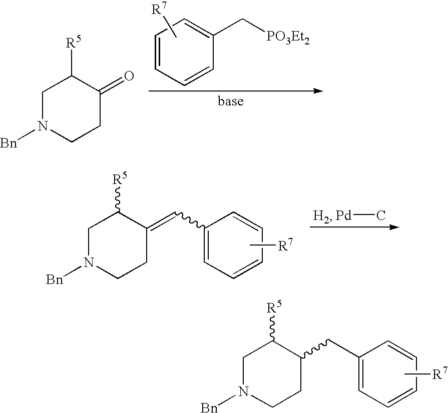
Scheme 7

Precursor B compounds containing an R⁵ substituent linked via an oxygen atom to the 4-position of the piperidine ring can be made according to the following general scheme: 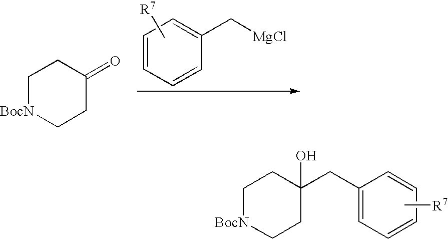
Scheme 8

Precursor B compounds containing an R⁵ or R^{5b} substituent linked via an oxygen atom to the 3-position of the piperidine ring can be made according to the following general scheme: 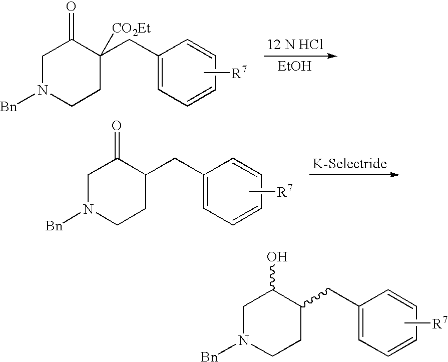
Scheme 9

Precursor B compounds containing an R⁵ substituent linked via a nitrogen atom to the 4-position of the piperidine ring can be made according to the following general scheme:

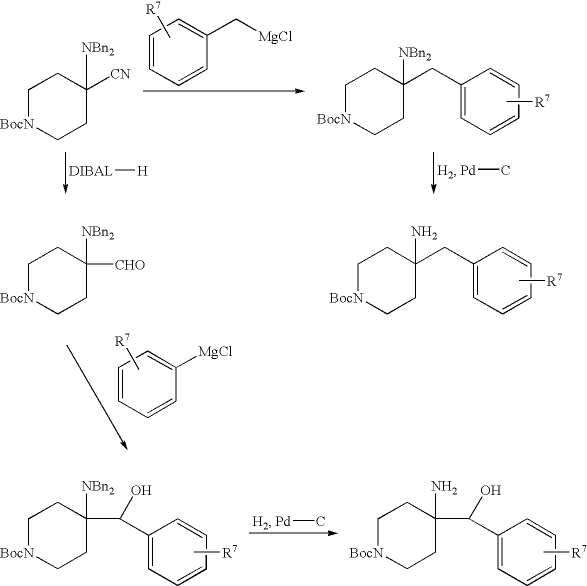

### EXAMPLES

### Example 1

### N-[5-chloro-2-[2-oxo-2-[4-(phenylmethyl)-1-piperidinyl]ethoxy]phenyl]urea

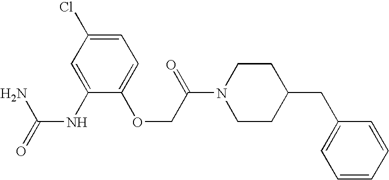

### A. [2-[(aminocarbonyl)amino]-4-chlorophenoxy]acetic acid 1,1-dimethylethyl ester

To a solution of N-(5-chloro-2-hydroxyphenyl)urea (25 g, 134 mmol) in dimethylsulfoxide (250 mL) was added tert-butyl bromoacetate (20.5 mL, 140 mmol) and potassium carbonate (37.5 g, 270 mmol), and the mixture stirred at ambient temperature overnight. The mixture was poured onto ice water and the resulting solid collected by filtration and washed with water. Recrystallization (hexane-dichloromethane-ethyl acetate) afforded Intermediate 1a as a light yellow crystalline solid.

### B. [2-[(aminocarbonyl)amino]-4-chlorophenoxy]acetic acid

Intermediate 1a (5 g, 16.5 mmol) was dissolved in 1:1 v/v trifluoroacetic acid-dichloromethane (50 mL) and stirred at ambient temperature overnight. The mixture was concentrated and dried under vacuum to afford Intermediate 1 b as a white solid.

### C. N-[5-chloro-2-[2-oxo-2-[4-(phenylmethyl)-1-piperidinyl]ethoxy]phenyl]urea

To a solution of Intermediate 1b (100 mg, 0.41 mmol) in dimethylformamide (5 mL) was added triethylamine (2.0 mL) and 4-(phenylmethyl)piperidine (60 mg, 0.34 mmol). HATU (155 mg, 0.41 mmol) was added and the mixture was stirred at ambient temperature overnight. The mixture was poured onto ice water and the resulting solid collected by filtration. Purification by reverse phase HPLC afforded Compound 1 as a light yellow solid. ¹H NMR (400 MHz, DMSO-d₆): δ/ppm=0.95 (m, 1H), 1.12 (m, 1H), 1.50-1.51 (m, 2H), 1.74 (m, 1H), 2.94 (br, 1H), 3.75 (br, 1H), 4.35 (br, 1H), 4.89 (m, 2H), 6.76-6.86 (m, 2H), 7.10-7.20 (m, 3H), 7.22-7.30 (m, 2H), 8.11 (br, 1H), 8.16 (d, 1H).

LRMS M+H, 402.2

### Example 2

### 1-[[2-[(aminocarbonyl)amino]-4-chlorophenoxy]acetyl]-4-[(4-fluorophenyl)methyl]4-piperidinecarboxylic acid methyl ester

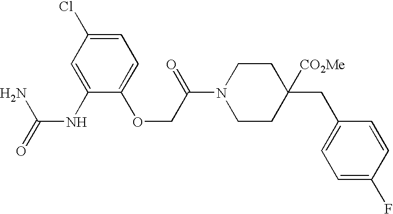

### A. 4-[(4-fluorophenyl)methyl]-1,4-piperidinedicarboxylic acid 1-(1,1-dimethylethyl) 4-methyl ester

To a solution of 1,4-piperidinedicarboxylic acid 1-(1,1-dimethylethyl) 4-methyl ester (10 g, 41 mmol) in tetrahydrofuran (20 mL) at −78° C. was added lithium diisopropylamide (2.0 M solution in tetrahydrofuran-heptane-ethylbenzene, 23 mL, 45 mmol). The mixture was stirred at −30° C. for 30 minutes, then recooled to −78° C. 4-Fluorobenzyl bromide (10.2 mL, 82 mmol) was added, and the mixture allowed to warm to ambient temperature and stirred for 2 days. The reaction was quenched with water, concentrated to remove tetrahydrofuran and extracted with dichloromethane. The extracts were dried over sodium sulfate, concentrated and purified by chromatography on silica to afford Intermediate 2a as a light yellow oil.

### B. 1-[[2-[(aminocarbonyl)amino]-4-chlorophenoxy]acetyl]-4-[(4-fluorophenyl)methyl]-4-piperidinecarboxylic acid methyl ester

Intermediate 2a (200 mg, 0.57 mmol) was deprotected by treatment with trifluoroacetic acid in dichloromethane at ambient temperature for 30 minutes. After concentration, the crude product was reacted with Intermediate 1 b in a similar manner to that described for Compound 1. The product was purified by recrystallization to afford Compound 2 as a white solid. ¹H NMR (400 MHz, DMSO-d₆): δ/ppm=1.35 (m, 1H), 1.48 (m, 1H), 1.92 (m, 2H), 2.64 (m, 1H), 2.78 (m, 2H), 3.00 (m, 1H), 3.60 (s, 3H), 3.72 (m, 1H), 4.10 (m, 1H), 4.88 (m, 2H), 6.36 (br, 2H), 6.78-6.86 (m, 2H), 7.00-7.12 (m, 4H), 8.12 (br, 1H), 8.16 (m, 1H).

LRMS M+H, 478.1

### Example 3

### N-[5-chloro-2-[2-[4-[(4-fluorophenyl)methyl]-1-piperidinyl]-2-oxoethoxy]phenyl]urea

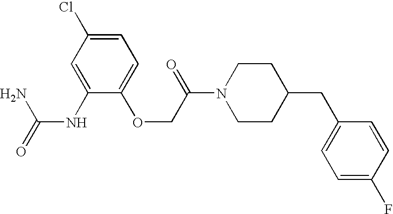 Prepared in a similar manner to Compound 1. ¹H NMR (400 MHz, DMSO-d₆+TFA): δ/ppm=1.00 (m, 1H), 1.14 (m, 1H), 1.54-1.62 (m, 2H), 1.74 (m, 1H), 2.46-2.60 (m, 4H), 2.96. (m, 1H), 3.80 (m, 1H), 4.30 (m, 1H), 4.90 (m, 2H), 6.83 (m, 2H), 7.08 (m, 2H), 7.18 (m, 2H), 8.16 (br, 1H), 8.20 (d, 1H).
LRMS M+H, 419.6

### Example 4

### N-[5-chloro-2-[2-[4-cyano-4-[(4-fluorophenyl)methyl]-1-piperidinyl]-2-oxoethoxy]phenyl]urea

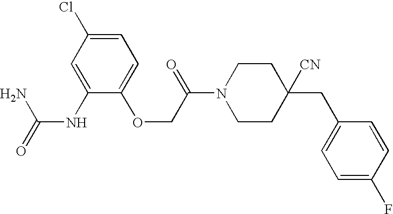 Prepared in a similar manner to Compound 1. Piperidine intermediate prepared in a similar manner to Intermediate 2a. ¹H NMR (400 MHz, CDCl₃): δ/ppm=1.27-1.44 (m, 2H), 1.58 (m, 3H), 1.79-1.87 (m, 2H), 2.80 (m, 3H), 3.27 (t, 1H), 3.74-3.93 (m, 1H), 4.62 (t, 1H), 4.95 (m, 1H), 5.28 (br, 2H), 6.80 (m, 2H), 7.04 (m, 2H), 7.20 (m, 2H), 8.20 (d, 1H), 8.38 (d, 1H).
LRMS M: 444

### Example 5

### N-[5-chloro-2-[2-[4-cyano-4-[(4-fluorophenyl)methyl]-1-piperidinyl]-1-methyl-2-oxoethoxy]phenyl]urea

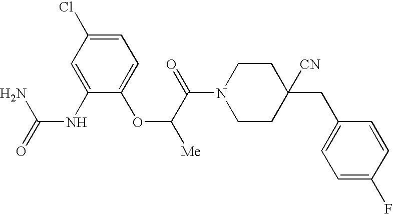 Prepared in a similar manner to Compound 1. ¹H NMR (400 MHz, CDCl₃): δ/ppm=1.27 (m, 3H), 1.44 (t, 1H), 1.79 (d, 0.6H), 1.87 (m, 1.4H), 2.37 (br, 1H), 2.80 (m, 3H), 3.27 (t, 1H), 3.74 (d, 0.6H), 3.93 (d, 0.4H), 4.62 (t, 1H), 4.95 (m, 1H), 5.28 (br, 2H), 6.80 (m, 2H), 7.04 (m, 2H), 7.20 (m, 2H), 8.20 (d, 1H), 8.38 (d, 1H).
LRMS M+H 458

### Example 6

### N-[5-chloro-2-[2-[4-[(4-fluorophenyl)methyl]-4-methyl-1-piperidinyl]-2-oxoethoxy]phenyl]urea

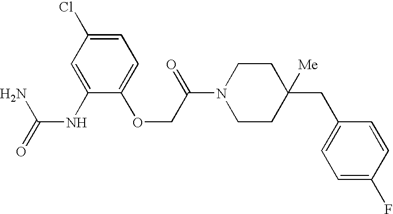

### A. 4-[(4-fluorophenyl)methyl]-4-(hydroxymethyl)cyclohexanecarboxylic acid 1,1-dimethylethyl ester

To a solution of Intermediate 2a (500 mg, 1.4 mmol) in tetrahydrofuran (10 mL) at 0° C. was added lithium aluminum hydride (60 mg, 1.6 mmol). The mixture was warmed to ambient temperature and stirred for 1 hour, then the reaction quenched by addition of water and aqueous sodium hydroxide (15% w/w). Extraction and purification by chromatography on silica afforded Intermediate 6a as a colorless oil.

### B. 4-[(4-fluorophenyl)methyl]-4-[[[(4-methyl phenyl)sulfonyl]oxy]methyl]-1-piperidinecarboxylic acid 1,1-dimethylethyl ester

To a solution of Intermediate 6a (430 mg, 1.3 mmol) in dichloromethane (5 mL) were added p-toluenesulfonyl chloride (1.04 g, 5.5 mmol), triethylamine (1.4 mL, 10.0 mmol) and catalytic dimethylaminopyridine. The mixture was stirred at ambient temperature for 7 days, then concentrated in vacuo. Extraction and purification by chromatography on silica afforded Intermediate 6b as a brown oil.

### C. 4-[(4-fluorophenyl)methyl]-4-methyl-1-piperidinecarboxylic acid 1,1-dimethylethyl ester

To a solution of Intermediate 6b (350 mg, 0.7 mmol) in DMSO (10 mL) was added sodium borohydride (166 mg, 4.4 mmol). The mixture was heated to 130° C. for 3 hours, then cooled and poured onto ice water. Extraction and purification by chromatography on silica afforded Intermediate 6c as a colorless oil.

### D. N-[5-chloro-2-[2-[4-[(4-fluorophenyl)methyl]-4-methyl-1-piperidinyl]-2-oxoethoxy]phenyl]urea

The tert-butoxycarbonyl protecting group was removed from Intermediate 6c, and the crude product reacted with Intermediate 1b in a similar manner as described for Compound 2. ¹H NMR (400 MHz, DMSO-d₆): δ/ppm=0.85 (s, 3H), 1.18-1.46 (m, 4H), 2.73 (s, 2H), 3.12 (m, 1H), 3.26 (m, 1H), 3.56 (m, 1H), 3.80 (m, 1H), 4.88 (m, 2H), 6.34 (br, 2H), 6.77-6.84 (m, 2H), 7.04-7.16 (m, 4H), 8.10 (br, 1H), 8.17 (d, 1H).

LRMS M+H: 434

### Example 7

### N-[5-chloro-2-[2-[cis-4-[(4-fluorophenyl)methyl]-2-methylpiperidinyl]-2-oxoethoxy]phenyl]urea

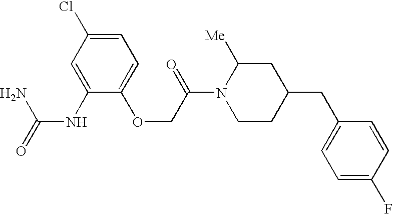

### A. 4-[(4-fluorophenyl)methyl]-2-methyl-1-piperidinecarboxylic acid 1,1-dimethylethyl ester

To a solution of 4-[(4-fluorophenyl)methyl]-1-piperidinecarboxylic acid 1,1-dimethylethyl ester (1.0 g, 3.4 mmol) in ether (20 mL) at −78° C. was added TMEDA (1.8 mL, 12 mmol), followed by dropwise addition of sec-butyllithium (1.4 M solution in cyclohexane, 8.6 mL, 12 mmol). The mixture was stirred at −78° C. for 3 hours. Iodomethane (0.21 mL, 3.4 mmol) was then added dropwise, and stirring continued for 30 minutes. The reaction was quenched by addition of water. Extraction and purification by chromatography on silica afforded Intermediate 7a as the racemic cis-substituted compound, contaminated with unreacted piperidine starting material.

### B. N-[5-chloro-2-[2-[cis-4-[(4-fluorophenyl)methyl]-2-methylpiperidinyl]-2-oxoethoxy]phenyl]urea

The tert-butoxycarbonyl protecting group was removed from Intermediate 7a, and the crude product reacted with Intermediate 1b in a similar manner as described for Compound 2 to afford Compound 7 (racemic cis) as a pale yellow solid. ¹H NMR (400 MHz, DMSO-d₆+TFA): δ/ppm=1.09 (d, 3H), 1.18 (m, 2H), 1.68 (m, 3H), 2.52 (m, 2H), 3.08 (m, 1H), 3.64 (m, 1H), 3.98 (m, 1H), 4.84 (s, 2H), 6.76-6.82 (m, 2H), 7.04 (m, 2H), 7.14 (m, 2H), 8.13 (br, 1H), 8.16 (d, 1H).

LRMS M+H: 434

### Example 8

### N-[5-chloro-2-[2-[4-[(4-fluorophenyl)methyl]-3-methyl-1-piperidinyl]-2-oxoethoxy]phenyl]urea

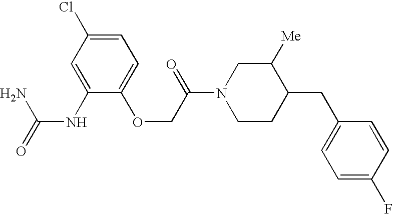

### A. 4-[(4-fluorophenyl)methylene]-3-methyl-1-(phenylmethyl)piperidine

To a solution of [(4-fluorophenyl)methyl]phosphonic acid diethyl ester (2.0 g, 8.1 mmol) in tetrahydrofuran (10 mL) at −78° C. was added potassium bis(trimethylsilyl)amide (0.5 M in toluene, 16 mL, 8.1 mmol). After 30 minutes, a solution of 3-methyl-1-(phenylmethyl)-4-piperidinone (1.5 g, 7.4 mmol) in tetrahydrofuran (10 mL) was added dropwise. The mixture was warmed to ambient temperature and stirred overnight. The reaction was quenched by addition of water. Extraction and purification by chromatography on silica afforded Intermediate 8a (1.42 g, mixture of isomers) as a colorless oil.

### B. 4-[(4-fluorophenyl)methyl]-3-methyl-piperidine

A mixture of Intermediate 8a (360 mg, 1.4 mmol) and 10% Pd-C (catalytic) in methanol (100 mL) was hydrogenated at 45 psi overnight. The mixture was filtered and the filtrate concentrated to dryness to afford Intermediate 8b (227 mg) as a colorless oil.

### C. N-[5-chloro-2-[2-[4-[(4-fluorophenyl)methyl]-3-methyl-1-piperidinyl]-2-oxoethoxy]phenyl]urea

Intermediate 8b was reacted with Intermediate 1 b in a similar manner to that described for Compound 1 to afford Compound 8 as a mixture of cis and trans isomers. ¹H NMR (400 MHz, DMSO-d₆+TFA): δ/ppm=0.72-1.06 (m, 3H), 1.20-1.49 (m, 2H), 1.74 (m, 1H), 1.95 (m, 1H), 2.45-2.58 (m, 2H), 2.60-3.25 (m, 2H), 3.55-3.90 (m, 1H), 4.00-4.30 (m, 1H), 4.85-5.10 (m, 2H), 6.80-6.90 (m, 2H), 7.10 (m, 2H), 7.24 (m, 2H), 8.16-8.24 (m, 2H).

LRMS M+H, 434.2

### Example 9

### N-[5-chloro-2-[2-[4-[(4-fluorophenyl)methyl]-4-methoxy-1-piperidinyl]-2-oxoethoxy]phenyl]urea

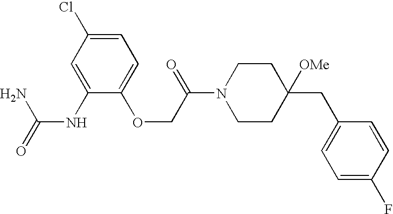

### A. 4-[(4-fluorophenyl)methyl]-4-hydroxy-1-piperidinecarboxylic acid 1,1-dimethylethyl ester

To 4-fluorobenzylmagnesium chloride (0.5 M in tetrahydrofuran, 50 mL, 12.5 mmol) was added a solution of 4-oxo-1-piperidinecarboxylic acid 1,1-dimethylethyl ester (2.3 g, 11 mmol) in tetrahydrofuran (15 mL). The mixture was stirred at ambient temperature for 3 hours, then the reaction quenched by addition of water. Extraction and purification by chromatography on silica afforded Intermediate 9a as a colorless oil.

### B. 4-[(4-fluorophenyl)methyl]4-methoxy-1-piperidinecarboxylic acid 1,1-dimethylethyl ester

A solution of Intermediate 9a (540 mg, 1.7 mmol) in dimethylformamide (5 mL) was added treated with sodium hydride (96 mg, 2.4 mmol) at ambient temperature for 30 minutes. Iodomethane (0.15 mL, 2.4 mmol) was then added and the mixture stirred overnight. Addition of water, extraction and purification by chromatography on silica afforded Intermediate 9b (50 mg) as a yellow oil.

### C. N-[5-chloro-2-[2-[4-[(4-fluorophenyl)methyl]-4-methoxy-1-piperidinyl]-2-oxoethoxy]phenyl]urea

The tert-butoxycarbonyl protecting group was removed from Intermediate 9b, and the crude product reacted with Intermediate 1 b in a similar manner as described for Compound 2. Recrystallization afforded Compound 9 as a white solid. ¹H NMR (400 MHz, DMSO-d₆): δ/ppm=1.26 (m, 1H), 1.42 (m, 1H), 1.60 (m, 2H), 2.66-2.82 (m, 3H), 3.14 (m, 1H), 3.54 (m, 1H), 4.01 (m, 1H), 4.86 (m, 2H), 6.34 (br, 2H), 6.76-6.84 (m, 2H), 7.06 (m, 2H), 7.16 (m, 2H), 8.08 (br, 1H), 8.16 (d, 1H).

LRMS M+H, 450.2

### Example 10

### N-[5-chloro-2-[2-[4-[(2,4-difluorophenyl)methyl]-4-hydroxy-1-piperidinyl]-2-oxoethoxy]phenyl]urea

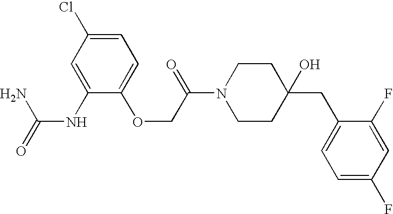 Prepared in a similar manner to Compound 1. ¹H NMR (400 MHz, DMSO-d₆): δ/ppm=1.32 (m, 3H), 1.42 (m, 4H), 2.65 (s, 2H), 2.87 (m, 1H), 3.24 (m, 1H), 3.54 (d, 1H), 4.03 (d, 1H), 4.85 (m, 2H), 6.76 (d, 2H), 6.91 (dt, 1H) 6.99 (dt, 1H), 7.27 (q, 1H), 8.14 (t, 2H).

### Example 11

### 1-[[2-[(aminocarbonyl)amino]4-chlorophenoxy]acetyl]-4-[(4-fluorophenyl)methyl]-3-oxo-4-piperidinecarboxylic acid ethyl ester

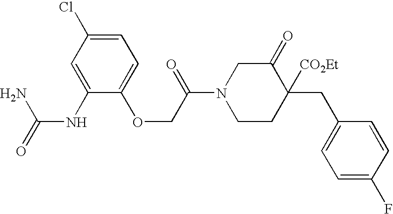 Prepared in a similar manner to Compound 1 from Intermediate 1 b and 4-[(4-fluorophenyl)methyl]-3-oxo-4-piperidinecarboxylic acid ethyl ester (prepared in a similar manner to Intermediate 2a). Purification by reverse phase HPLC afforded Compound 11 as a light yellow solid. ¹H NMR (400 MHz, DMSO-d₆+TFA): δ/ppm=1.01 (t, 3H), 1.72-1.90 (m, 1H), 2.22-2.38 (m, 1H), 2.82-2.92 (m, 1H), 3.14 (m, 1H), 3.40-3.70 (m, 2H), 3.84-4.10 (m, 3H), 4.28-4.42 (m, 1H), 4.72-4.92 (m, 2H), 6.72-6.82 (m, 2H), 7.02 (m, 2H), 7.10 (m, 2H), 8.10 (br, 1H), 8.16 (d, 1H).
LRMS M+H, 506.1

### Example 12

### N-[5-chloro-2-[2-[(3R,4R)-4-[(4-fluorophenyl)methyl]-3-hydroxypiperidinyl]-2-oxoethoxy]phenyl]urea

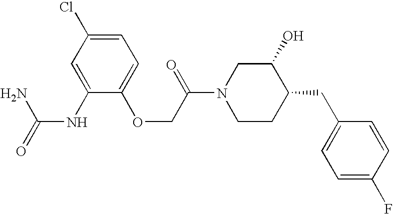

### A. 4-[(4-fluorophenyl)methyl]-1-(phenylmethyl)-3-piperidinone

A solution of 4-[(4-fluorophenyl)methyl]-1-(phenylmethyl)-1,4-piperidinedicarboxylic acid ethyl ester (14.7 g, 40 mmol, prepared in a similar manner to Intermediate 2a) in 12 N hydrochloric acid (100 mL) and ethanol (100 mL) was heated at 50° C. for 2 hours. The mixture was filtered and the filtrate concentrated to ca. 140 mL, heated at reflux for 24 hours, then concentrated in vacuo. The residue was dissolved in ethyl acetate and washed with 10 N sodium hydroxide. Purification by chromatography on silica afforded Intermediate 12a (9.8 g) as a light yellow solid.

### B. (3R,4R)-4-[(4-fluorophenyl)methyl]-1-(phenylmethyl)-3-piperidinol

To a solution of Intermediate 12a (900 mg, 3.0 mmol) in tetrahydrofuran (20 mL) at −78° C. was added dropwise a solution of K-Selectride (1.0 M in tetrahydrofuran, 4.5 mL, 4.5 mmol). The mixture was stirred at −78° C. for 4 hours, then the reaction was quenched by addition of 10 N sodium hydroxide (1 mL), followed by 6 N hydrochloric acid (1.5 mL). Extraction and purification by chromatography on silica afforded Intermediate 12b (760 mg) as a colorless oil. Ratio of cis:trans isomers ca. 5.8:1 by ¹H NMR.

### C. N-[5-chloro-2-[2-[(3R,4R)-4-[(4-fluorophenyl)methyl]-3-hydroxypiperidinyl]-2-oxoethoxy]phenyl]urea

Intermediate 12b was deprotected with hydrogen and catalytic palladium on charcoal. Reaction with Intermediate 1b in a similar manner to that described for Compound 1, and purification by recrystallization afforded Compound 12 as a white solid. ¹H NMR (400 MHz, DMSO-d₆+TFA): δ/ppm=1.20 (m, 1H), 1.36-1.76 (m, 2H), 2.36-2.50 (m, 2H), 2.60 (m, 1H), 2.86-3.04 (m, 1H), 3.46-3.52 (m, 1H), 3.60-3.72 (m, 1H), 4.22-4.32 (m, 1H), 4.84-5.00 (m, 2H), 6.74-6.82 (m, 2H), 6.98 (m, 2H), 7.16 (m, 2H), 8.13 (d, 1H), 8.20 (br, 1H).

LRMS M+H, 436.1

### Example 13

### N-[5-chloro-2-[2-[4-[(4-fluorophenyl)methyl]-4-[(2-hydroxypropyl)amino]-1-piperidinyl]-2-oxoethoxy]phenyl]urea

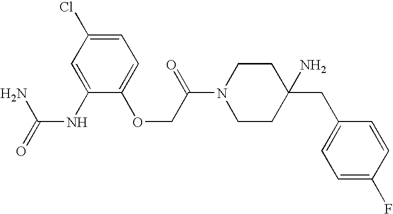

### A. 4-[bis(phenylmethyl)amino]-4-[(4-fluorophenyl)methyl]-1-piperidinecarboxylic acid 1,1-dimethylethyl ester

To 4-[bis(phenylmethyl)amino]-4-cyano-1-piperidinecarboxylic acid 1,1-dimethylethyl ester (1 g, 2.5 mmol) was added a solution of 4-fluorobenzylmagnesium chloride (0.25 M in tetrahydrofuran, 41 mL, 10 mmol). The mixture was stirred at ambient temperature for 5 hours, then the reaction quenched by addition of water. Extraction and purification by chromatography on silica afforded Intermediate 13a (730 mg) as a white solid.

### B. 4-amino-4-[(4-fluorophenyl)methyl]-1-piperidinecarboxylic acid 1,1-dimethylethyl ester

A mixture of Intermediate 13a (730 mg, 1.5 mmol) and 10% Pd-C (catalytic) in methanol-ethyl acetate (200 mL, 1:1 v/v) was hydrogenated at 45 psi overnight. The mixture was filtered and the filtrate concentrated to afford Intermediate 13b (270 mg) as a colorless oil.

### C. N-[5-chloro-2-[2-[4-[(4-fluorophenyl)methyl]4-[(2-hydroxypropyl)amino]-1-piperidinyl]-2-oxoethoxy]phenyl]urea

The tert-butoxycarbonyl protecting group was removed from Intermediate 13b, and the crude product reacted with Intermediate 1b in a similar manner as described for Compound 2. Purification by chromatography on silica and reverse phase HPLC afforded Compound 13 as a white solid (82 mg). ¹H NMR (400 MHz, DMSO-d₆): δ/ppm=1.66 (m, 4H), 2.96 (s, 2H), 3.60 (m, 4H), 4.96 (s, 2H), 6.38 (s, 2H), 6.80 (m, 2H), 7.24 (m, 4H), 7.96 (m, 2H), 8.10 (s, 1H), 8.18 (s, 1H).

LRMS M+H, 435.1

### Example 14

### N-[5-chloro-2-[2-[4-[(4-fluorophenyl)methyl]-4-[(2-hydroxypropyl)amino]-1-piperidinyl]-2-oxoethoxy]phenyl]urea

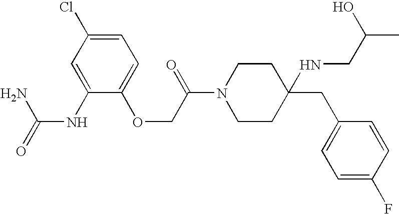

### A. 4-[(4-fluorophenyl)methyl]-4-[(2-hydroxypropyl)amino]-1-piperidinecarboxylic acid 1,1-dimethylethyl ester

A mixture of Intermediate 13a (730 mg, 1.5 mmol) and 10% Pd-C (catalytic) in methanol-ethyl acetate (200 mL, 1:1 v/v) was hydrogenated at 45 psi overnight. The mixture was filtered and the filtrate concentrated to dryness. A mixture of the resulting amine (308 mg, 1.0 mmol), propylene oxide (0.21 mL, 3.0 mmol) and 3 drops of water was heated at 75° C. in a sealed tube for 2 days. The mixture was concentrated and purified by chromatography on silica to afford Intermediate 14a (270 mg) as a colorless oil.

### B. N-[5-chloro-2-[2-[4-[(4-fluorophenyl)methyl]-4-[(2-hydroxypropyl)amino]-1-piperidinyl]-2-oxoethoxy]phenyl]urea

The tert-butoxycarbonyl protecting group was removed from Intermediate 14a, and the crude product reacted with Intermediate 1b in a similar manner as described for Compound 2. Purification by reverse phase HPLC afforded Compound 14 as a white solid (208 mg). ¹H NMR (400 MHz, DMSO-d₆+TFA): δ/ppm=1.00 (d, 3H), 1.60 (m, 4H), 2.74 (m, 1H), 2.90 (m, 1H), 2.95-3.10 (m, 3H), 3.20 (m, 1H), 3.36 (m, 1H), 3.60 (m, 1H), 3.75 (m, 2H), 4.80 (m, 2H), 6.62-6.70 (m, 2H), 7.02 (m, 2H), 7.16 (m, 2H), 7.83 (m, 1H), 8.00 (br, 1H), 8.05 (d, 1H), 8.12 (m, 1H).

LRMS M+H, 493.1

### Example 15

### N-[5-chloro-2-[2-[4-(4-fluorobenzoyl)-4-methyl-1-piperidinyl]-2-oxoethoxy]phenyl]urea

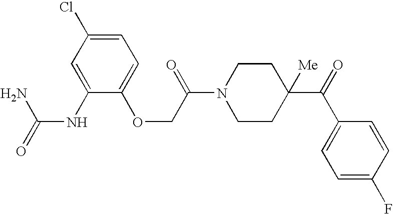

### A. 4-(4-fluorobenzoyl)-4-methyl-1-piperidinecarboxylic acid 1,1-dimethylethyl ester

A solution of 4-(4-fluorobenzoyl)-1-piperidinecarboxylic acid 1,1-dimethylethyl ester (1.0 g, 3.3 mmol) in dimethylformamide (15 mL) at 0° C. was treated with sodium hydride (312 mg, 7.8 mmol) for 30 minutes. Iodomethane (0.64 mL, 10.4 mmol) was added and the mixture stirred at ambient temperature for 3 days. Addition to ice water, extraction and purification by chromatography on silica afforded Intermediate 15a as a yellow oil.

### B. N-[5-chloro-2-[2-[4-(4-fluorobenzoyl)-4-methyl-1-piperidinyl]-2-oxoethoxy]phenyl]urea

The tert-butoxycarbonyl protecting group was removed from Intermediate 15a, and the crude product reacted with Intermediate 1b in a similar manner as described for Compound 2. Purification by recrystallization afforded Compound 15 as a white solid. ¹H NMR (400 MHz, DMSO-d₆): δ/ppm=1.38 (s, 3H), 1.48-1.64 (m, 2H), 2.06 (m, 2H), 3.08-3.22 (m, 2H), 3.49 (m, 1H), 3.62 (m, 1H), 4.88 (m, 2H), 6.34 (br, 2H), 6.78-6.84 (m, 2H), 7.28 (m, 2H), 7.82 (m, 2H), 8.09 (br, 1H), 8.16 (d, 1H).

LRMS M+H, 448.1

### Example 16

### N-[5-chloro-2-[2-[4-ethyl-4-(4-fluorobenzoyl)-1-piperidinyl]-2-oxoethoxy]phenyl]urea

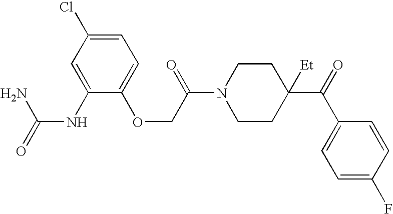 Prepared in a similar manner to Compound 1 from Intermediate 1b and (4-ethyl-4-piperidinyl)(4-fluorophenyl)methanone. Purification by recrystallization afforded Compound 16 as a white solid. ¹H NMR (400 MHz, DMSO-d₆): δ/ppm=0.72 (t, 3H), 1.48 (m, 1H), 1.58 (m, 1H), 1.90 (q, 2H), 2.14 (m, 2H), 2.86 (m, 1H), 3.06 (m, 1H), 3.56 (m, 1H), 3.82 (m, 1H), 4.86 (m, 2H), 6.34 (br, 2H), 6.78-6.84 (m, 2H), 7.28 (m, 2H), 7.82 (m, 2H), 8.08 (br, 1H), 8.15 (d, 1H).
LRMS M+H, 462.2

### Examples 17 and 18

### N-[2-[2-[4-[(acetyloxy)(4-fluorophenyl)methyl]-4-methyl-1-piperidinyl]-2-oxoethoxy]-5-chlorophenyl]urea and

### N-[5-chloro-2-[2-[4-[(4-fluorophenyl)hydroxymethyl]-4-methyl-1-piperidinyl]-2-oxoethoxy]phenyl]urea

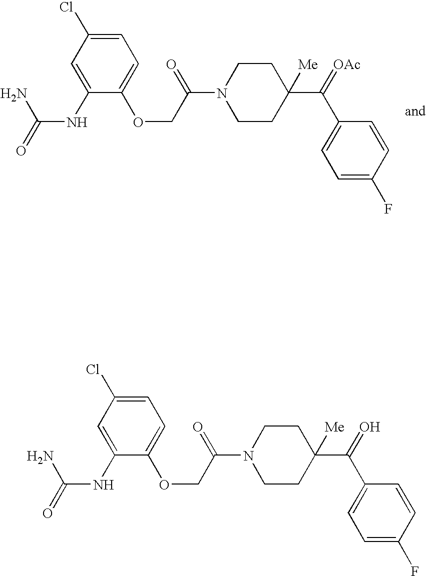

### A. 4-[(acetyloxy)(4-fluorophenyl)methyl]-4-methyl-1-piperidinecarboxylic acid 1,1-dimethylethyl ester and

### 4-[(4-fluorophenyl)hydroxymethyl]-4-methyl-1-piperidinecarboxylic acid 1,1-dimethylethyl ester

A mixture of Intermediate 15a (530 mg, 1.65 mmol), 10% Pd-C (250 mg) and 70% perchloric acid (0.3 mL) in acetic acid (20 mL) was hydrogenated at 45 psi for 5.5 hours. The mixture was filtered and concentrated. The residue was diluted with water (10 mL) and adjusted to pH 14 with 15% aqueous sodium hydroxide. Tetrahydrofuran (20 mL) and di-tert-butyl dicarbonate (540 mg, 2.5 mmol) were added and the mixture was stirred at ambient temperature overnight. Extraction, drying over sodium sulfate and concentration afforded a mixture of the title compounds (Intermediates 17a (acetate) and 18a (alcohol)).

### B. N-[2-[2-[4-[(acetyloxy)(4-fluorophenyl)methyl]-4-methyl-1-piperidinyl]-2-oxoethoxy]-5-chlorophenyl]urea and

### N-[5-chloro-2-[2-[4-[(4-fluorophenyl)hydroxymethyl]-4-methyl-1-piperidinyl]-2-oxoethoxy]phenyl]urea

The crude mixture of Intermediates 17a and 18a was deprotected, and the product mixture reacted with Intermediate 1b in a similar manner as described for Compound 2. Separation of the products by chromatography on silica, followed by recrystallization afforded Compounds 17 (278 mg) and 18 (170 mg) as white solids.

Compound 17: ¹H NMR (400 MHz, DMSO-d₆): δ/ppm=0.92 (m, 3H), 1.04 (m, 1H), 1.30-1.58 (m, 3H), 2.02 (s, 3H), 2.04 (s, 3H), 2.80 (s, 1H), 3.15 (m, 1H), 3.66 (m, 1H), 4.05 (m, 1H), 4.80-5.00 (m, 2H), 4.44 (s, 1H), 6.36 (br, 2H), 6.78-6.84 (m, 2H), 7.14 (m, 2H), 7.30 (m, 2H), 8.12 (s, 1H), 8.18 (s, 1H).

LRMS M+H, 492.1

Compound 18: ¹H NMR (400 MHz, DMSO-d₆): δ/ppm=0.81 (s, 3H), 0.96 (m, 1H), 1.34-1.62 (m, 3H), 2.80 (m, 1H), 3.12 (m, 1H), 3.62 (m, 1H), 4.02 (m, 1H), 4.26 (m, 1H), 4.80-4.96 (m, 2H), 5.32 (m, 1H), 6.34 (br, 2H), 6.76-6.84 (m, 2H), 7.10 (m, 2H), 7.28 (m, 2H), 8.12 (br, 1H), 8.18 (m, 1H).

LRMS M+H, 450.2

### Example 19

### N-[5-chloro-2-[2-[4-cyano-4-[fluoro(4-fluorophenyl)methyl]-1-piperidinyl]-2-oxoethoxy]phenyl]urea

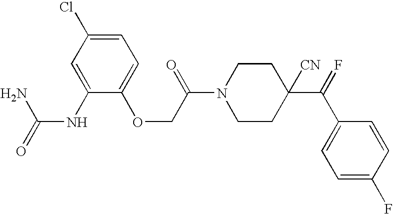

### A. 4-cyano-4-[(4-fluorophenyl)hydroxymethyl]-1-piperidinecarboxylic acid 1,1-dimethylethyl ester

A solution of 4-cyano-4-(4-fluorobenzoyl)-1-piperidinecarboxylic acid 1,1-dimethylethyl ester (prepared in a similar manner to Intermediate 2a) (4.0 mmol) in methanol-dichloromethane (100 mL, 1:1 v/v) was treated with sodium borohydride (227 mg, 6.0 mmol) at ambient temperature for 30 minutes. The mixture was quenched by addition of water. Concentration to remove organic solvents, extraction and purification by chromatography on silica afforded Intermediate 19a (349 mg) as a white solid.

### B. 4-cyano-4-[fluoro(4-fluorophenyl)methyl]-1-piperidinecarboxylic acid 1,1-dimethylethyl ester

To a solution of Intermediate 19a (300 mg, 0.9 mmol) in dichloromethane (5 mL) at −78° C. was added (diethylamino)sulfur trifluoride (DAST, 0.16 mL, 1.1 mmol). The mixture was allowed to warm to ambient temperature and stirred for 30 minutes, then recooled to −78° C. and quenched by addition of methanol. Concentration and purification by chromatography on silica afforded intermediate 19b (282 mg) as a yellow solid.

### C. N-[5-chloro-2-[2-[4-cyano-4-[fluoro(4-fluorophenyl)methyl]-1-piperidinyl]-2-oxoethoxy]phenyl]urea

The tert-butoxycarbonyl protecting group was removed from Intermediate 19b, and the crude product reacted with Intermediate 1b in a similar manner as described for Compound 2. Purification by recrystallization afforded Compound 19 as a white solid (184 mg). ¹H NMR (400 MHz, DMSO-d₆): δ/ppm=1.34 (m, 1H), 1.52-1.86 (m, 2H), 2.18 (m, 1H), 2.66 (m, 1H), 3.12 (m, 1H), 3.92 (m, 1H), 4.42 (m, 1H), 4.92 (m, 2H), 5.74 (s, 1H), 5.62 (s, 1H), 6.35 (br, 2H), 6.82 (m, 2H), 7.32 (m, 2H), 7.48 (m, 2H), 8.08 (br, 1H), 8.17 (m, 1H).

LRMS M+H, 463.1

### Example 20

### N-[5-chloro-2-[2-[4-cyano-4-(4-fluorobenzoyl)-1-piperidinyl]-2-oxoethoxy]phenyl]urea

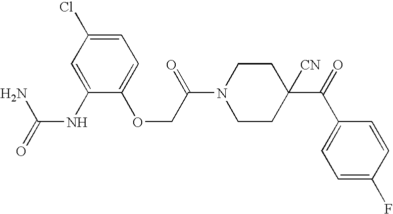 The tert-butoxycarbonyl protecting group was removed 4-cyano-4-(4-fluorobenzoyl)-1-piperidinecarboxylic acid 1,1-dimethylethyl ester, and the crude product reacted with Intermediate 1b in a similar manner as described for Compound 2. Purification by chromatography on silica afforded Compound 20 (147 mg). ¹H NMR (400 MHz, DMSO-d₆): δ/ppm=1.86 (m, 1H), 2.08 (m, 1H), 2.32 (m, 2H), 2.94 (m, 1H), 3.36 (m, 1H), 3.92 (m, 1H), 4.40 (m, 1H), 5.00 (m, 2H), 6.32 (m, 2H), 6.84 (m, 2H), 7.42 (m. 2H). 8.10 (s, 1H), 8.20 (m, 3H).
LRMS M+H, 459.1

### Example 21

### N-[2-[2-[4-amino-4-[(4-fluorophenyl)hydroxymethyl]-1-piperidinyl]-2-oxoethoxy]-5-chlorophenyl]urea

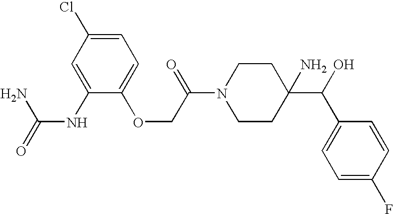

### A. 4-[bis(phenylmethyl)amino]-4-formyl-1-piperidinecarboxylic acid 1,1-dimethylethyl ester

To a solution of 4-[bis(phenylmethyl)amino]-4-cyano-1-piperidinecarboxylic acid 1,1-dimethylethyl ester (2.0 g, 4.9 mmol) in dichloromethane (20 mL) at −78° C. was added a solution of diisobutyl aluminum hydride (1.0 M in dichloromethane, 10 mL, 10 mmol). The mixture was stirred at −78° C. for 30 minutes, then warmed to −20° C. and stirred for an additional 1 hour. The reaction was quenched by addition of 6 N hydrochloric acid (5 mL) at −20° C. and stirred at ambient temperature overnight. The pH was adjusted to 13 by addition of 10 N sodium hydroxide (3.5 mL). Extraction and purification by chromatography on silica afforded Intermediate 21a (730 mg) as a colorless oil.

### B. 4-[bis(phenylmethyl)amino]-4-[(4-fluorophenyl)hydroxymethyl]-1-piperidinecarboxylic acid 1,1-dimethylethyl ester

To 4-fluorophenylmagnesium chloride (1.0 M in tetrahydrofuran, 4.0 mL, 4.0 mmol) was added a solution of Intermediate 21a (1.06 g, 2.6 mmol) in tetrahydrofuran (10 mL) at 0° C. The mixture was stirred at ambient temperature for 15 minutes, then the reaction quenched by addition of water. Extraction and purification by chromatography on silica afforded Intermediate 21b (1.15 g) as a white solid.

### C. 4-amino-4-[(4-fluorophenyl)hydroxymethyl]-1-piperidinecarboxylic acid 1,1-dimethylethyl ester

A mixture of Intermediate 21b (450 mg, 0.9 mmol) and 10% Pd-C (catalytic) in methanol (100 mL) was hydrogenated at 45 psi overnight. The mixture was filtered and the filtrate concentrated to dryness. Purification by chromatography on silica afforded Intermediate 21c (183 mg) as a white solid.

### D. N-[2-[2-[4-amino-4-[(4-fluorophenyl)hydroxymethyl]-1-piperidinyl]-2-oxoethoxy]-5-chlorophenyl]urea

The tert-butoxycarbonyl protecting group was removed from Intermediate 21c, and the crude product reacted with Intermediate 1b in a similar manner as described for Compound 2. Purification by reverse phase HPLC afforded Compound 21 as a white solid (159 mg). ¹H NMR (400 MHz, DMSO-d₆+TFA): δ/ppm=1.40-1.86 (m, 4H), 3.00-3.54 (m, 2H), 3.72 (m, 1H), 3.94 (m, 1H), 4.70 (m, 1H), 4.88 (m, 2H), 6.74 (m, 2H), 7.16 (m, 2H), 7.36 (m, 2H), 7.92 (br, 3H), 8.12 (br, 1H), 8.18 (m, 1H).

LRMS M+H, 451.1

### Example 22

### 5-chloro-2-[2-[4-cyano-4-[(4-fluorophenyl)methyl]-1-piperidinyl]-2-oxoethoxy]benzoic acid methyl ester

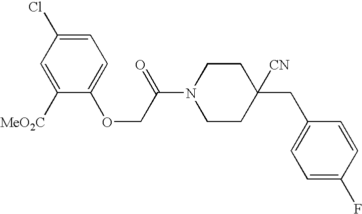 The tert-butoxycarbonyl protecting group was removed from Intermediate 36a, and the crude product reacted with 2-(carboxymethoxy)-5-chlorobenzoic acid methyl ester (prepared in a similar manner to Intermediate 1b) in a similar manner as described for Compound 2. Purification by reverse phase HPLC afforded Compound 22 as a white solid. ¹H NMR (400 MHz, DMSO-d₆): δ/ppm=1.47 (t, 1H), 1.68 (t, 1H), 1.78, (m, 2H), 2.67 (t, 1H), 2.88 (s, 2H), 3.10 (t, 1H), 3.77 (s, 3H), 3.85 (d, 1H), 4.33 (d, 1H), 4.85 (d, 1H), 5.06 (d, 1H), 7.02 (d, 1H), 7.15 (t, 2H), 7.30 (m, 2H), 7.51 (dd, 1H), 7.81 (s, 1H).
LRMS M+H: 444

### Example 23

### N-[2-[2-[4-cyano-4-[(4-fluorophenyl)methyl]-1-piperidinyl]-2-oxoethoxy]-4-methoxyphenyl]urea

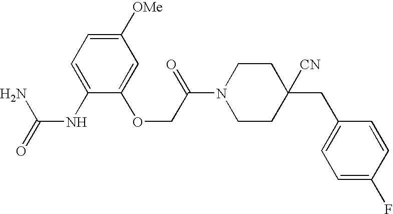 Prepared from 2-[(aminocarbonyl)amino-5-methoxyphenoxy]acetic acid and Intermediate 36a. Purification by recrystallization afforded Compound 23 as a white solid. ¹H NMR (400 MHz, DMSO-d₆): δ/ppm=1.49 (m, 1H), 1.64 (m, 1H), 1.74-1.83 (m, 2H), 2.78 (m, 1H), 2.90 (m, 2H), 3.11 (m, 1H), 3.65 (s, 3H), 3.88 (m, 1H), 4.37 (m, 1H), 4.86 (m, 2H), 6.04 (br, 2H), 6.42 (dd, 1H), 6.48 (d, 1H), 7.17 (m, 2H), 7.30 (m, 2H), 7.67 (br, 1H), 7.81 (d, 1H).
LRMS M+H, 441.2

### Example 24

### 4-[(4-fluorophenyl)methyl]-1-[(3,4,5-trimethoxyphenoxy)acetyl]-4-piperidinecarbonitrile

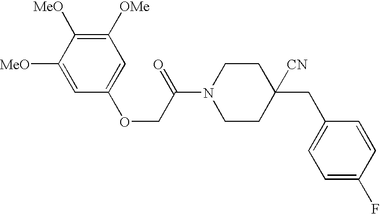 Prepared from (3,4,5-trimethoxyphenoxy)acetic acid and Intermediate 36a. Purification by chromatography on silica afforded Compound 24 (300 mg) as a white solid. ¹H NMR (400 MHz, CDCl₃): δ/ppm=1.65 (m, 2H), 1.90 (d, 2H), 2.81 (m, 2H), 2.88 (t, 1H), 3.36 (t, 1H), 3.78 (s, 3H), 3.83 (s, 6H), 4.10 (d, 1H), 4.85 (m, 3H), 6.20 (s, 2H), 7.04 (t, 2H), 7.20 (m, 2H).
LRMS M+H: 442

### Example 25

### 1-[(4-chloro-2-formylphenoxy)acetyl]-4-[(4-fluorophenyl)methyl]-4-piperidinecarbonitrile

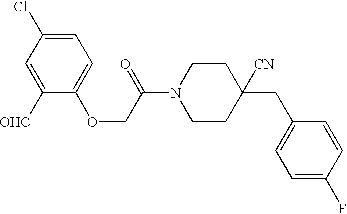 Prepared from (4-chloro-2-formylphenoxy)acetic acid and Intermediate 36a. Purification by chromatography on silica and reverse phase HPLC afforded Compound 25 (325 mg). ¹H NMR (400 MHz, CDCl₃): δ/ppm=1.45-1.51 (m, 2H), 1.94-1.98 (m, 2H), 2.85 (s, 2H), 2.96-2.90 (m, 1H), 3.38-3.45 (m, 1H), 3.97-4.01 (m, 1H), 4.63-4.66 (m, 1H), 4.81 (d, 1H), 4.88 (d, 1H), 6.96 (d, 1H), 7.03-7.07 (m, 2H), 7.20-7.23 (m, 2H), 7.49 (dd, 1H), 7.81, (d, 1H), 10.39 (s, 1H).
LRMS M+H, 415.1

### Example 26

### 2-[2-[4-amino-4-[(4-fluorophenyl)methyl]-1-piperidinyl]-2-oxoethoxy]-5-chlorobenzoic acid methyl ester

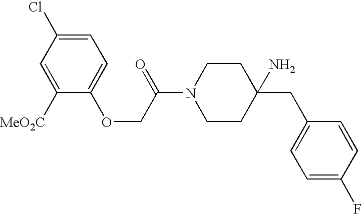 Prepared from 2-(carboxymethoxy)-5-chlorobenzoic acid methyl ester and Intermediate 13b. Purification by chromatography on silica afforded Compound 26 (676 mg) as a brown solid. ¹H NMR (400 MHz, CDCl₃): δ/ppm=1.30-1.40 (m, 2H), 1.44-1.58 (m, 2H), 2.60 (s, 2H), 3.15 (m, 1H), 3.50 (m, 1H), 3.80-3.93 (m, 1H), 3.88 (s, 3H), 4.18 (m, 1H), 4.78 (m, 2H), 6.96-7.10 (m, 5H), 7.39 (m, 1H), 7.78 (m, 1H).
LRMS M+H, 435.1

### Example 27

### N-[5-chloro-2-[2-[4-(4-fluorobenzoyl)-1-piperidinyl]-2-oxoethoxy]phenyl]urea

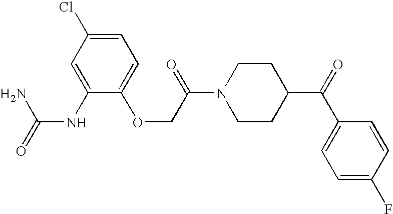 Prepared in a similar manner to Compound 1. ¹H NMR (400 MHz, CDCl₃): δ/ppm=1.56-2.00 (m, 4H), 3.00 (m, 1H), 3.25 (m, 1H), 3.52 (m, 1H), 3.74 (m, 1H), 4.50 (m, 1H), 4.74 (m, 2H), 4.85 (br, 1H), 6.82 (d, 1H), 6.88 (dd, 1H), 7.14-7.20 (m, 2H), 7.94-8.02 (m, 2H), 8.28 (d, 1H), 8.95 (br, 1H).
LRMS M+H, 434.1

### Example 28

### N-[5-chloro-2-[2-[4-[(4-fluorophenyl)thio]-1-piperidinyl]-2-oxoethoxy]phenyl]urea

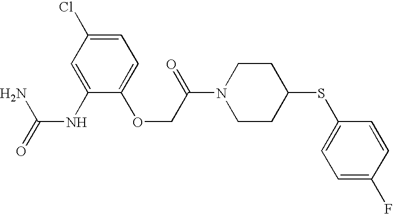 Prepared in a similar manner to Compound 1. ¹H NMR (400 MHz, DMSO-d₆): δ/ppm=1.26 (m, 1H), 1.42 (m, 1H), 1.86 (m, 2H), 2.82 (m, 1H), 3.08-3.18 (m, 1H), 3.38 (m, 1H), 3.76 (m, 1H), 4.12 (m, 1H), 4.90 (m, 2H), 6.34 (br, 2H), 6.78-6.84 (m, 2H), 7.18 (m, 2H), 7.46 (m, 2H), 8.10 (br, 1H), 8.16 (d, 1H).
LRMS M: 437

### Example 29

### N-[5-chloro-2-[2-[4-(4-chlorobenzoyl)-1-piperidinyl]-2-oxoethoxy]phenyl]urea

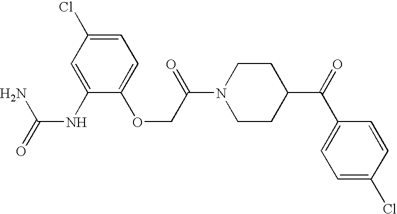 Prepared in a similar manner to Compound 1. ¹H NMR (400 MHz, DMSO-d₆): δ/ppm=1.34 (m, 1H), 1.55 (m, 1H), 1.76-1.84 (m, 2H), 2.80 (m, 1H), 3.20 (m, 1H), 3.70 (m, 1H), 3.84 (m, 1H), 4.30 (m, 1H), 4.94 (s, 2H), 6.34 (br, 2H), 6.83 (m, 2H), 7.60 (m, 2H), 8.00 (m, 2H), 8.12 (br, 1H), 8.17 (m, 1H).
LRMS M+H, 450.1

### Example 30

### N-[2-[2-[4-(4-bromobenzoyl)-1-piperidinyl]-2-oxoethoxy]-5-chlorophenyl]urea

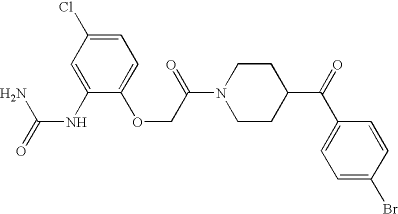 Prepared in a similar manner to Compound 1. ¹H NMR (400 MHz, DMSO-d₆): δ/ppm=1.34 (m, 1H), 1.55 (m, 1H), 1.80 (m, 2H), 2.80 (m, 1H), 3.20 (m, 1H), 3.70 (m, 1H), 3.84 (m, 1H), 4.30 (m, 1H), 4.94 (s, 2H), 6.34 (br, 2H), 6.84 (m, 2H), 7.74 (m, 2H), 7.92 (m, 2H), 8.12 (br, 1H), 8.17 (m, 1H).
LRMS M+H, 496.0

### Example 31

### N-[5-chloro-2-[2-[4-[(4-fluorophenyl)methylene]-1-piperidinyl]-2-oxoethoxy]phenyl]urea

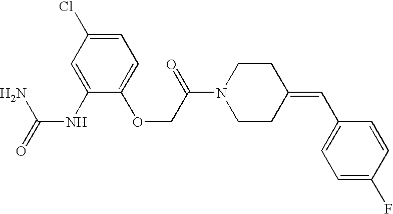

### A. 4-[(4-fluorophenyl)methylene]-1-piperidinecarboxylic acid 1,1-dimethylethyl ester

To a solution of [(4-fluorophenyl)methyl]phosphonic acid diethyl ester (2.7 g, 11 mmol) in tetrahydrofuran (20 mL) at −78° C. was added potassium bis(trimethylsilyl)amide (0.5 M in toluene, 22 mL, 11 mmol). After 30 minutes, a solution of 4-oxo-1-piperidinecarboxylic acid 1,1-dimethylethyl ester (2.0 g, 10 mmol) in tetrahydrofuran (10 mL) was added dropwise. The mixture was warmed to ambient temperature and stirred for 1 hour, then the reaction quenched by addition of water. Extraction and purification by chromatography on silica afforded Intermediate 31a (1.45 g) as a white solid.

### B. N-[5-chloro-2-[2-[4-[(4-fluorophenyl)methylene]-1-piperidinyl]-2-oxoethoxy]phenyl]urea

The tert-butoxycarbonyl protecting group was removed from Intermediate 31a, and the crude product reacted with Intermediate 1b in a similar manner as described for Compound 2. Purification by recrystallization afforded Compound 31 as a white solid (570 mg). ¹H NMR (400 MHz, DMSO-d₆): δ/ppm=2.28 (m, 1H), 2.36 (m, 2H), 2.45 (m, 1H), 3.44 (m, 2H), 3.52 (m, 2H), 4.96 (m, 2H), 6.16 (m, 3H), 6.78 (m, 2H), 7.14 (m, 2H), 7.24 (m, 2H), 8.13 (m, 1H), 8.17 (m, 1H).

LRMS M+H, 418.1

### Example 32

### N-[5-chloro-2-[2-[4-[(4-fluorophenyl)methyl]-4-(hydroxymethyl)-1-piperidinyl]-2-oxoethoxy]phenyl]urea

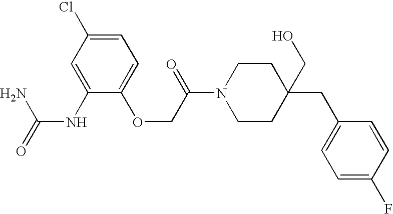 Prepared in a similar manner to Compound 1. ¹H NMR (400 MHz, DMSO-d₆): δ/ppm=1.36 (m, 4H), 2.60 (s, 2H), 3.16 (m, 2H), 3.32 (m, 2H), 3.56 (m, 2H), 4.88 (m, 2H), 6.34 (s, 2H), 6.80 (m, 2H), 7.08 (m, 2H), 7.18 (m, 2H), 8.10 (s, 1H), 8.16 (s, 1H).
LRMS M+H, 450.2

### Example 33

### N-[[1-[2-[2-[(aminocarbonyl)amino]-4-chlorophenoxy]acetyl]-4-[(4-fluorophenyl)methyl]-4-piperidinyl]methyl]-2,2,2-trifluoroacetamide

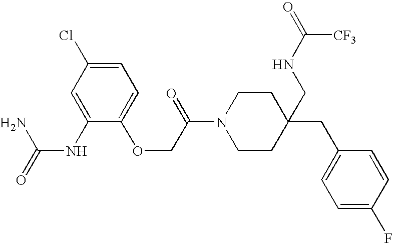

### A. 4-(aminomethyl)-4-[(4-fluorophenyl)methyl]-1-piperidinecarboxylic acid 1,1-dimethylethyl ester

A mixture of 4-cyano-4-[(4-fluorophenyl)methyl]-1-piperidinecarboxylic acid 1,1-dimethylethyl ester (prepared in a similar manner to Intermediate 2a) (650 mg, 2.0 mmol) and Raney nickel (1 mL) in methanol (20 mL) was cooled to 0° C. and saturated with ammonia gas. The mixture was hydrogenated at ambient temperature for 2 hours, filtered and the filtrate concentrated to dryness to afford Intermediate 33a (700 mg).

### B. 4-[(4-fluorophenyl)methyl]-4-[[(trifluoroacetyl)amino]methyl]-1-piperidinecarboxylic acid, 1,1-dimethylethyl ester

To a solution of Intermediate 33a (650 mg, 2.0 mmol) in dichloromethane was added triethylamine (0.40 g, 4.0 mmol), followed by trifluoroacetic anhydride (440 mg, 2.1 mmol). The mixture was stirred at ambient temperature for 2 hours. Extraction and purification by chromatography on silica afforded intermediate 33b (612 mg).

### C. N-[[1-[2-[2-[(aminocarbonyl)amino]-4-chlorophenoxy]acetyl]-4-[(4-fluorophenyl)methyl]-4-piperidinyl]methyl]-2,2,2-trifluoroacetamide

The tert-butoxycarbonyl protecting group was removed from Intermediate 33b, and the crude product reacted with Intermediate 1b in a similar manner as described for Compound 2. Purification by chromatography on silica afforded Compound 33 (597 mg). ¹H NMR (400 MHz, DMSO-d₆): δ/ppm=1.28 (m, 4H), 2.68 (s, 2H), 3.12 (m, 2H), 3.40 (m, 2H), 3.52 (m, 1H), 3.62 (m, 1H), 4.88 (s, 2H), 6.36 (s, 1H), 6.76 (m, 2H), 7.10 (m, 2H), 7.20 (m, 2H), 8.10 (s, 1H), 8.16 (s, 1H), 9.36 (m, 1H).

### Example 34

### N-[5-chloro-2-[2-[4-[(4-fluorophenyl)methyl]-4-hydroxy-1-piperidinyl]-2-oxoethoxy]phenyl]urea

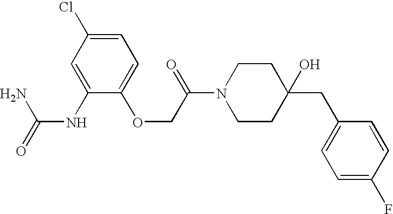 The tert-butoxycarbonyl protecting group was removed from Intermediate 9a, and the crude product reacted with Intermediate 1b in a similar manner as described for Compound 2. Purification by chromatography on silica afforded Compound 34. ¹H NMR (400 MHz, DMSO-d₆): δ/ppm=1.34 (m, 3H), 1.44 (m, 1H), 2.64 (s, 2H), 2.90 (m, 1H), 3.24 (m, 1H), 3.54 (m, 1H), 4.00 (m, 1H), 4.48 (s, 1H), 4.88 (m, 2H), 6.34 (s, 2H), 6.80 (m, 2H), 7.04 (m, 2H), 7.18 (m, 2H), 8.10 (s, 1H), 8.16 (s, 1H).
LRMS M+H, 436.1

### Example 35

### N-[5-chloro-2-[2-[4-fluoro-4-[(4-fluorophenyl)methyl]-1-piperidinyl]-2-oxoethoxy]phenyl]urea

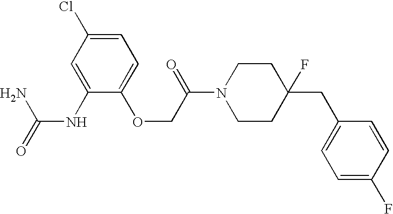 Prepared from Intermediate 1 b and 4-fluoro-4-[(4-fluorophenyl)methyl]piperidine (prepared in a similar manner to intermediate 19b). Purification by reverse phase HPLC afforded Compound 35. ¹H NMR (400 MHz, DMSO-d₆): δ/ppm=1.90 (m, 1H), 2.02 (m, 1H), 3.30 (m, 2H), 3.48 (m, 3H), 3.90 (m, 1H), 3.98 (m, 1H), 4.92 (m, 2H), 5.42 (s, 1H), 6.38 (s, 2H), 6.80 (m, 2H), 7.10 (m, 2H), 7.20 (m, 2H), 8.12 (s, 1H), 8.18 (s, 1H).
LRMS M+H 438.1

### Example 36

### N-[5-chloro-2-[2-[4-[(4-fluorophenyl)methyl]-4-formyl-1-piperidinyl]-2-oxoethoxy]phenyl]urea

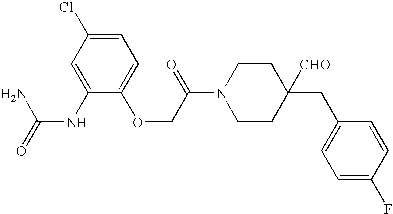

### A. 4-cyano-4-[(4-fluorophenyl)methyl]-1-piperidinecarboxylic acid 1,1-dimethylethyl ester

To a solution of 4-cyano-1-piperidinecarboxylic acid 1,1-dimethylethyl ester (5.0 g, 24 mmol) in tetrahydrofuran at −78° C. was added lithium diisopropylamide (1.8 M solution in tetrahydrofuran-heptane-ethylbenzene, 15 mL, 27 mmol) and the mixture stirred at −78° C. for 1 hour. 4-Fluorobenzyl bromide (6.0 mL, 48 mmol) was added, and the mixture allowed to warm to ambient temperature. The reaction was quenched with water. Extraction and purification by chromatography on silica afforded Intermediate 36a (8.7 g).

### B. 4-[(4-fluorophenyl)methyl]4-formyl-1-piperidinecarboxylic acid 1,1-dimethylethyl ester

To a solution of Intermediate 36a (477 mg, 1.5 mmol) in dichloromethane at −78° C. was added a solution of diisobutyl aluminum hydride (1.0 M in dichloromethane, 2.4 mL, 2.4 mmol). The mixture was stirred at −78° C. for 30 minutes, then warmed to −20° C. and stirred for an additional 2 hours. The reaction was quenched by addition of 10% hydrcohloric acid and stirred at ambient temperature overnight. The pH was adjusted to 13 by addition of 10% sodium hydroxide. Extraction and purification by chromatography on silica afforded Intermediate 36b (233 mg).

### C. N-[5-chloro-2-[2-[4-[(4-fluorophenyl)methyl]-4-formyl-1-piperidinyl]-2-oxoethoxy]phenyl]urea

The tert-butoxycarbonyl protecting group was removed from Intermediate 36b, and the crude product reacted with Intermediate 1b in a similar manner as described for Compound 2. Purification by reverse phase HPLC afforded Compound 36 (49 mg). ¹H NMR (400 MHz, DMSO-d₆): δ/ppm=1.50 (m, 2H), 1.82 (m, 2H), 2.68 (m, 2H), 2.90 (s, 1H), 3.08 (m, 2H), 4.02 (m, 1H), 4.34 (m, 1H), 4.90 (m, 2H), 6.36 (s, 1H), 6.80 (m, 2H), 7.10 (m, 2H), 7.18 (m, 1H), 7.30 (m, 1H), 8.14 (m, 2H), 9.60 (s, 1H).

LRMS M+H, 448.1

### Example 37

### N-[5-chloro-2-[2-[4-[(dimethylamino)methyl]-4-[(4-fluorophenyl)methyl]-1-piperidinyl]-2-oxoethoxy]phenyl]urea

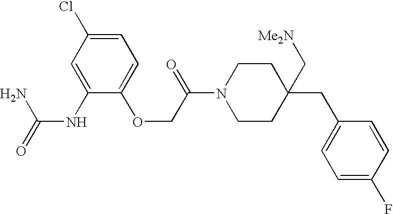

### A. 4-[(dimethylamino)methyl]-4-[(4-fluorophenyl)methyl]-1-piperidinecarboxylic acid 1,1-dimethylethyl ester

To a solution of Intermediate 33a (500 mg, 1.5 mmol) in dichloromethane was added triethylamine (300 mg, 3.0 mmol), followed by iodomethane (420 mg, 3.0 mmol). The mixture was stirred at ambient temperature for 2 hours. Extraction and purification by chromatography on silica afforded Intermediate 37a (353 mg).

### B. N-[5-chloro-2-[2-[4-[(dimethylamino)methyl]-4-[(4-fluorophenyl)methyl]-1-piperidinyl]-2-oxoethoxy]phenyl]urea

The tert-butoxycarbonyl protecting group was removed from Intermediate 37a, and the crude product reacted with Intermediate 1b in a similar manner as described for Compound 2. Purification by reverse phase HPLC afforded Compound 37 (144 mg). ¹H NMR (400 MHz, DMSO-d₆): δ/ppm=1.42 (m, 4H), 2.64 (m, 2H), 2.76 (m, 1H), 2.90 (m, 4H), 3.20 (m, 2H), 3.56 (m, 1H), 4.88 (m, 2H), 6.38 (s, 1H), 6.76 (m, 2H), 7.18 (m, 4H), 8.14 (m, 2H), 8.30 (s, 1H).

LRMS M+H, 477.2

### Example 38

### N-[5-chloro-2-[2-[4-(dimethylamino)-4-[(4-fluorophenyl)methyl]-1-piperidinyl]-2-oxoethoxy]phenyl]urea

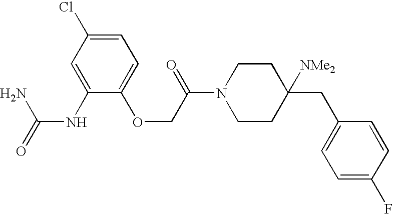 Prepared in a similar manner to Compound 1. ¹H NMR (400 MHz, DMSO-d₆): δ/ppm=1.70 (m, 2H), 1.98 (m, 2H), 2.78 (s, 6H), 3.18 (m, 2H), 3.48 (m, 4H), 4.88 (m, 2H), 6.76 (m, 2H), 7.18 (m, 2H), 7.34 (m, 2H), 8.10 (s, 1H), 8.16 (s, 1H), 9.10 (s, 1H).
LRMS M+H, 463.2

### Example 39

### N-[5-chloro-2-[2-[4-[(dipropylamino)methyl]-4-[(4-fluorophenyl)methyl]-1-piperidinyl]-2-oxoethoxy]phenyl]urea

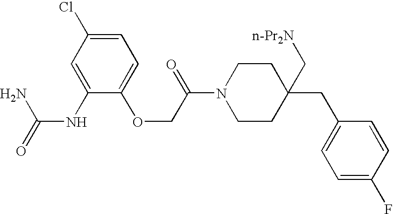 Prepared in a similar manner to Compound 1. ¹H NMR (400 MHz, DMSO-d₆): δ/ppm=0.86 (t, 6H), 1.35 (m, 2H), 1.70 (m, 2H), 2.90 (s, 2H), 3.04 (m, 2H), 3.38 (m, 2H), 3.66 (m, 4H), 4.90 (m, 2H), 6.40 (s, 1H), 6.80 (m, 2H), 7.18 (m, 4H), 8.10 (s, 1H), 8.20 (s, 1H), 8.80 (m, 1H).
LRMS M+H, 533.2

### Example 40

### N-[5-chloro-2-[2-[4-[(diethylamino)methyl]-4-[(4-fluorophenyl)methyl]-1-piperidinyl]-2-oxoethoxy]phenyl]urea

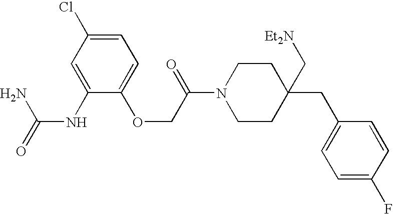 Prepared in a similar manner to Compound 1. ¹H NMR (400 MHz, DMSO-d₆): δ/ppm=1.20 (t, 6H), 1.32 (m, 1H), 1.42 (m, 1H), 1.60 (m, 2H), 2.90 (s, 2H), 3.16 (m, 6H), 4.90 (m, 2H), 6.42 (s, 1H), 6.80 (m, 2H), 7.22 (m, 4H), 8.12 (s, 1H), 8.20 (s, 1H), 8.50 (m, 1H).
LRMS M+H, 505.2

### Example 41

### N-[1-[2-[2-[(aminocarbonyl)amino]-4-chlorophenoxy]acetyl]-4-[(4-fluorophenyl)methyl]-4-piperidinyl]acetamide

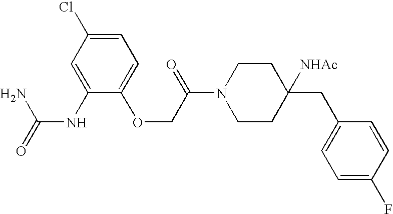 Prepared from Intermediate 1b and N-[4-[(4-fluorophenyl)methyl]-4-piperidinyl]acetamide (prepared in a similar manner to Intermediate 33b). Purification by reverse phase HPLC afforded Compound 41. ¹H NMR (400 MHz, DMSO-d₆): δ/ppm=1.27 (m, 1H), 1.44 (m, 1H), 1.82 (s, 3H), 2.02 (m, 2H), 2.74 (m, 1H), 2.92 (m, 2H), 3.10 (m, 1H), 3.60 (m, 2H), 4.06 (m, 2H), 4.92 (m, 2H), 6.36 (s, 2H), 6.82 (m, 2H), 7.08 (m, 4H), 8.12 (s, 1H), 8.18 (s, 1H).

### Example 42

### N-[1-[[2-[(aminocarbonyl)amino]-4-chlorophenoxy]acetyl]-4-[(4-fluorophenyl)methyl]-4-piperidinyl]urea

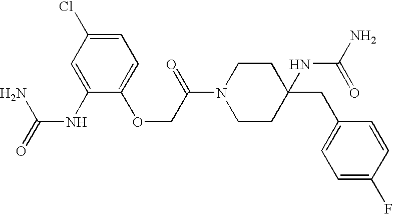

### A. 4-[(aminocarbonyl)amino]-4-[(4-fluorophenyl)methyl]-1-piperidinecarboxylic acid 1,1-dimethylethyl ester

To a solution of Intermediate 13b (154 mg, 0.5 mmol) in tetrahydrofuran was added trimethylsilyl isocyanate (287 mg, 2.5 mmol). The reaction was heated at 50° C. overnight. Isolation and purification by chromatography on silica afforded Intermediate 42a (131 mg).

### B. N-[1-[[2-[(aminocarbonyl)amino]-4-chlorophenoxy]acetyl]-4-[(4-fluorophenyl)methyl]-4-piperidinyl]urea

The tert-butoxycarbonyl protecting group was removed from Intermediate 42a, and the crude product reacted with Intermediate 1b in a similar manner as described for Compound 2. Purification by reverse phase HPLC afforded Compound 42 (47 mg). ¹H NMR (400 MHz, DMSO-d₆): δ/ppm=1.30 (m, 1H), 1.42 (m, 1H), 1.88 (m, 2H), 2.76 (m, 1H), 2.92 (s, 2H), 3.10 (m, 1H), 3.60 (m, 2H), 4.90 (m, 2H), 5.70 (s, 1H), 6.36 (s, 2H), 6.80 (m, 2H), 7.10 (m, 4H), 8.12 (s, 1H), 8.18 (s, 1H).

LRMS M+H, 478.2

### Example 43

### [[1-[[2-[(aminocarbonyl)amino]-4-chlorophenoxy]acetyl]-4-[(4-fluorophenyl)methyl]-4-piperidinyl]amino]acetic acid ethyl ester

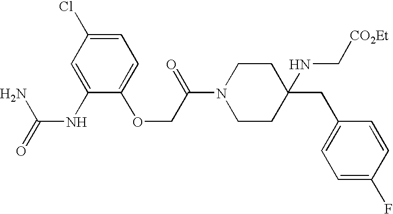

### A. 4-[(2-ethoxy-2-oxoethyl)amino]-4-[(4-fluorophenyl)methyl]-1-piperidinecarboxylic acid 1,1-dimethylethyl ester

To a solution of Intermediate 13b (308 mg, 1.0 mmol) in dimethylformamide (5 mL) were added ethyl bromoacetate (167 mg, 1.0 mmol), cesium carbonate (138 mg, 1.3 mmol) and sodium iodide (165 mg, 1.1 mmol). The mixture was heated at 60° C. overnight. Extraction and purification by chromatography on silica afforded Intermediate 43a (363 mg) as an oil.

### B. [[1-[[2-[(aminocarbonyl)amino]-4-chlorophenoxy]acetyl]-4-[(4-fluorophenyl)methyl]-4-piperidinyl]amino]acetic acid ethyl ester

The tert-butoxycarbonyl protecting group was removed from Intermediate 43a (350 mg, 0.9 mmol), and the crude product reacted with Intermediate 1b (217 mg, 0.9 mmol) in a similar manner as described for Compound 2. Purification by reverse phase HPLC afforded Compound 43 (429 mg). ¹H NMR (400 MHz, DMSO-d₆): δ/ppm=0.88 (m, 1H), 1.26 (m, 6H), 1.44 (m, 3H), 2.61 (m, 2H), 3.10 (m, 1H), 3.24 (m, 1H), 3.43 (s, 3H), 4.20 (m, 3H), 4.64 (s, 2H), 4.96 (s, 2H), 6.82 (m, 2H), 6.96 (m, 4H), 7.25 (s, 1H), 8.21 (s, 1H), 9.14 (s, 1H).

### Example 44

### N-[5-chloro-2-[2-[4-[(4-fluorophenyl)methyl]-4-[(2-hydroxyethyl)amino]-1-piperidinyl]-2-oxoethoxy]phenyl]urea

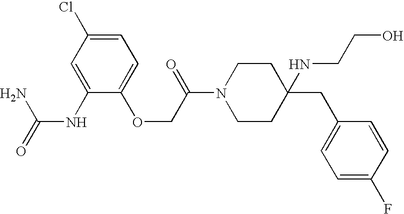 Prepared in a similar manner to Compound 43. ¹H NMR (400 MHz, DMSO-d₆): δ/ppm=1.74 (m, 4H), 3.12 (m, 3H), 3.32 (m, 1H), 3.50 (m, 1H), 3.64 (m, 3H), 3.72 (m, 1H), 3.90 (m, 1H), 4.92 (m, 2H), 6.36 (m, 2H), 6.78 (m, 2H), 7.22 (m, 4H), 8.10 (s, 1H), 8.20 (m, 2H).
LRMS M+H, 479.1 (478.2 calcd)

### Example 45

### [[[1-[[2-[(aminocarbonyl)amino]-4-chlorophenoxy]acetyl]-4-[(4-fluorophenyl)methyl]-4-piperidinyl]methyl]amino]acetic acid ethyl ester

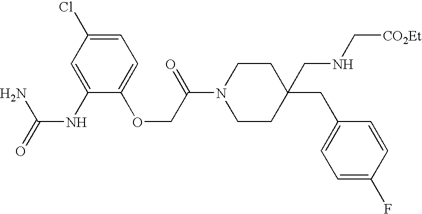

### A. 4-[[(2-ethoxy-2-oxoethyl)amino]methyl]-4-[(4-fluorophenyl)methyl]-1-piperidinecarboxylic acid 1,1-dimethylethyl ester

To a solution of Intermediate 36b (2.75 g, 8.5 mmol) in 1,2-dichloroethane (50 mL) was added glycine ethyl ester (1.2 g, 8.5 mmol) and several drops of acetic acid. After stirring overnight at ambient temperature, sodium triacetoxyborohydride (2.3 g, 11 mmol) was added and the mixture again stirred overnight. The reaction was diluted with dichloromethane (50 mL) and water (30 mL), and the aqueous layer adjusted to ca. pH 9. Extraction and purification by chromatography on silica afforded Intermediate 45a (1.1 g).

### B. [[[1-[[2-[(aminocarbonyl)amino]-4-chlorophenoxy]acetyl]-4-[(4-fluorophenyl)methyl]-4-piperidinyl]methyl]amino]acetic acid ethyl ester

The tert-butoxycarbonyl protecting group was removed from Intermediate 45a (450 mg, 1.1 mmol), and the crude product reacted with Intermediate 1b (270 mg, 1.1 mmol) in a similar manner as described for Compound 2. Purification by reverse phase HPLC afforded Compound 45 (396 mg). ¹H NMR (400 MHz, DMSO-d₆): δ/ppm=1.26 (t, 3H), 1.70 (m, 6H), 2.52 (m, 2H), 2.72 (s, 2H), 3.40 (s, 2H), 3.48 (s, 2H), 3.56 (m, 1H), 3.72 (m, 1H), 4.22 (m, 2H), 4.66 (m, 2H), 5.06 (m, 1H), 5.30 (s, 1H), 6.82 (m, 2H), 7.00 (m, 3H), 7.15 (m, 2H), 8.20 (s, 1H), 8.96 (s, 1H).

LRMS M+H, 535.2

### Example 46

### N-[5-chloro-2-[2-[4-[(4-fluorophenyl)methyl]-4-(4-morpholinylmethyl)-1-piperidinyl]-2-oxoethoxy]phenyl]urea

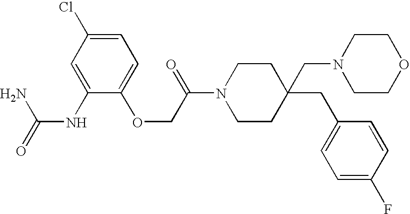 Prepared in a similar manner to Compound 45. ¹H NMR (400 MHz, DMSO-d₆): δ/ppm=1.36 (m, 2H), 1.60 (m, 2H), 2.90 (m, 2H), 3.24 (m, 4H), 3.92 (m, 4H), 4.86 (m, 2H), 6.36 (s, 2H), 6.78 (m, 2H), 7.18 (m, 4H), 8.08 (s, 1H), 8.20 (s, 1H).
LRMS M+H, 519.2

### Example 47

### N-[5-chloro-2-[2-[4-[(4-fluorophenyl)methyl]-4-(methylamino)-1-piperidinyl]-2-oxoethoxy]phenyl]urea

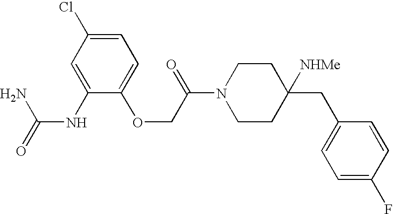

### A. 4-[(4-fluorophenyl)methyl]-4-(methylamino)-1-piperidinecarboxylic acid 1,1-dimethylethyl ester

Intermediate 47a was prepared from Intermediate 13b and formaldehyde in a similar manner to that described for Intermediate 45a.

### B. N-[5-chloro-2-[2-[4-[(4-fluorophenyl)methyl]-4-(methylamino)-1-piperidinyl]-2-oxoethoxy]phenyl]urea

The tert-butoxycarbonyl protecting group was removed from Intermediate 47a (220 mg, 0.65 mmol), and the crude product reacted with Intermediate 1b (167 mg, 0.65 mmol) in a similar manner as described for Compound 2. Purification by chromatography on silica afforded Compound 47 (25 mg). ¹H NMR (400 MHz, DMSO-d₆): δ/ppm=1.70 (m, 4H), 2.58 (s, 3H), 3.10 (s, 2H), 3.58 (m, 4H), 4.92 (s, 2H), 6.78 (m, 2H), 7.24 (m, 4H), 8.10 (s, 1H), 8.18 (s, 1H), 8.44 (s, 1H).

LRMS M+H, 449.2

### Example 48

### N-[5-chloro-2-[2-[4-ethenyl-4-[(4-fluorophenyl)methyl]-1-piperidinyl]-2-oxoethoxy]phenyl]urea

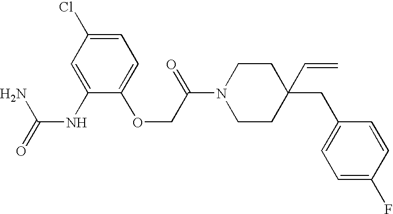

### A. 4-ethenyl-4-[(4-fluorophenyl)methyl]-1-piperidinecarboxylic acid 1,1-dimethylethyl ester

To a suspension of methyltriphenylphosphonium bromide (790 mg, 2.2 mmol) in tetrahydrofuran (20 mL) was added sodium hydride (60% suspension in mineral oil, 96 mg, 2.4 mmol). After stirring for 45 minutes at ambient temperature, the resulting solution was added to Intermediate 36b (650 mg, 2.0 mmol). The mixture was stirred at ambient temperature overnight, then concentrated in vacuo. Extraction and purification by chromatography on silica afforded Intermediate 48a (340 mg).

### B. N-[5-chloro-2-[2-[4-ethenyl-4-[(4-fluorophenyl)methyl]-1-piperidinyl]-2-oxoethoxy]phenyl]urea

The tert-butoxycarbonyl protecting group was removed from Intermediate 48a, and the crude product reacted with Intermediate 1 b in a similar manner as described for Compound 2. Purification by reverse phase HPLC afforded Compound 48 (86 mg). ¹H NMR (400 MHz, DMSO-d₆): δ/ppm=1.38 (m, 1H), 1.54 (m, 3H), 2.56 (s, 2H), 2.90 (m, 1H), 3.12 (m, 1H), 3.60 (m, 1H), 3.96 (m, 1H), 4.86 (m, 2H), 5.18 (d, 1H), 5.62 (m, 1H), 6.34 (s, 1H), 6.80 (m, 2H), 7.06 (m, 4H), 8.10 (s, 1H), 8.16 (s, 1H).

### Example 49

### N-[5-chloro-2-[2-[4-ethyl-4-[(4-fluorophenyl)methyl]-1-piperidinyl]-2-oxoethoxy]phenyl]urea

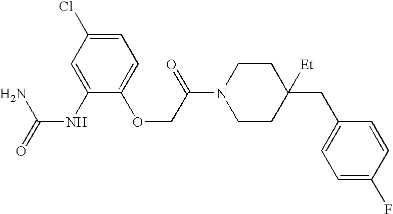

### A. 4-ethyl-4-[(4-fluorophenyl)methyl]-1-piperidinecarboxylic acid 1,1-dimethylethyl ester

A mixture of Intermediate 48a (250 mg, 0.78 mmol) and 10% Pd-C (catalytic) in ethanol (20 mL) was hydrogenated at 60 psi overnight. The mixture was filtered and the filtrate concentrated to dryness to afford Intermediate 49a (270 mg) as a colorless oil.

### B. N-[5-chloro-2-[2-[4-ethyl-4-[(4-fluorophenyl)methyl]-1-piperidinyl]-2-oxoethoxy]phenyl]urea

The tert-butoxycarbonyl protecting group was removed from Intermediate 49a, and the crude product reacted with Intermediate 1b in a similar manner as described for Compound 2. Purification by reverse phase HPLC afforded Compound 49 (110 mg). ¹H NMR (400 MHz, DMSO-d₆): δ/ppm=0.84 (t, 3H), 1.24 (m, 6H), 2.54 (s, 2H), 3.30 (m, 2H), 3.60 (m, 2H), 4.90 (m, 2H), 6.36 (s, 1H), 6.80 (m, 2H), 7.10 (m, 4H), 8.10 (s, 1H), 8.20 (s, 1H).

### Example 50

### N-[5-chloro-2-[2-[4-[(4-fluorophenyl)methyl]-4-(1-piperidinylmethyl)-1-piperidinyl]-2-oxoethoxy]phenyl]urea

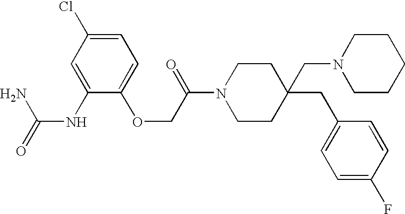

### A. 4-[(4-fluorophenyl)methyl]-4-(1-piperidinylmethyl)-1-piperidinecarboxylic acid 1,1-dimethylethyl ester

To a solution of Intermediate 33a (750 mg, 2.3 mmol) in dimethylformamide (5 mL) were added 1,5-dibromopentane (530 mg, 2.3 mmol), cesium carbonate (984 mg, 3.0 mmol) and sodium iodide (349 mg, 2.5 mmol). The mixture was heated at 60° C. overnight. Extraction and purification by chromatography on silica afforded Intermediate 50a (310 mg) as an oil.

### B. N-[5-chloro-2-[2-[4-[(4-fluorophenyl)methyl]-4-(1-piperidinylmethyl)-1-piperidinyl]-2-oxoethoxy]phenyl]urea

The tert-butoxycarbonyl protecting group was removed from Intermediate 50a, and the crude product reacted with Intermediate 1b in a similar manner as described for Compound 2. Purification by reverse phase HPLC afforded Compound 50 (75 mg). ¹H NMR (400 MHz, DMSO-d₆): δ/ppm=1.35 (m, 3H), 1.60 (m, 3H), 1.78 (m, 4H), 2.90 (m, 2H), 3.08 (m, 2H), 3.17 (m, 1H), 3.39 (m, 4H), 3.60 (m, 3H), 4.88 (m, 2H), 6.34 (s, 1H), 6.78 (m, 2H), 7.20 (m, 4H), 8.08 (s, 1H), 8.16 (s, 1H), 8.50 (s, 1H).

LRMS M+H, 517.2

### Example 51

### N-[2-[2-[4-(1-azetidinylmethyl)-4-[(4-fluorophenyl)methyl]-1-piperidinyl]-2-oxoethoxy]-5-chlorophenyl]urea

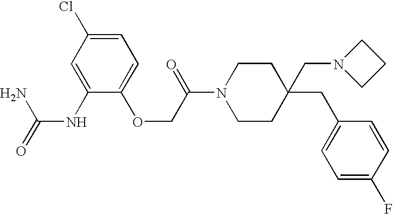 Prepared in a similar manner to Compound 50. ¹H NMR (400 MHz, DMSO-d₆): δ/ppm=1.22 (m, 1H), 1.37 (m, 3H), 2.22 (m, 1H), 2.74 (m, 2H), 3.18 (m, 2H), 3.39 (m, 2H), 3.56 (m, 2H), 4.20 (m, 5H), 4.86 (m, 2H), 6.34 (s, 1H), 6.78 (m, 2H), 7.14 (m, 4H), 8.08 (s, 1H), 8.16 (s, 1H), 9.56 (s, 1H).
LRMS M+H, 489.2

### Example 52

### N-[5-chloro-2-[2-[4-[fluoro(4-fluorophenyl)methyl]-4-(1-pyrrolidinylmethyl)-1-piperidinyl]-2-oxoethoxy]phenyl]urea

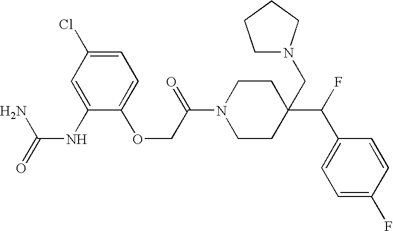

### A. 4-(aminomethyl)-4-[fluoro(4-fluorophenyl)methyl]-1-piperidinecarboxylic acid 1,1-dimethylethyl ester

Reduction of Intermediate 19b with Raney nickel in a similar manner to that described for Intermediate 33a afforded Intermediate 52a.

### B. 4-[fluoro(4-fluorophenyl)methyl]-4-(1-pyrrolidinylmethyl)-1-piperidinecarboxylic acid 1,1-dimethylethyl ester

Intermediate 52a was converted to Intermediate 52b in a similar manner to that described for Intermediate 50a.

### C. N-[5-chloro-2-[2-[4-[fluoro(4-fluorophenyl)methyl]-4-(1-pyrrolidinylmethyl)-1-piperidinyl]-2-oxoethoxy]phenyl]urea

The tert-butoxycarbonyl protecting group was removed from Intermediate 52b, and the crude product reacted with Intermediate 1b in a similar manner as described for Compound 2. Purification by reverse phase HPLC afforded Compound 52 (75 mg). ¹H NMR (400 MHz, DMSO-d₆) δ/ppm=1.25 (m, 3H), 1.52 (m, 3H), 1.68 (m, 4H), 2.60 (m, 4H), 3.02 (m, 1H), 3.22 (m, 1H), 3.60 (m, 1H), 3.82 (m, 1H), 4.82 (m, 2H), 5.86 (d, 1H), 6.36 (s, 1H), 6.74 (m, 2H), 7.35 (m, 4H), 8.10 (s, 1H), 8.18 (s, 1H).

LRMS M+H, 521.2

### Example 53

### N-[5-chloro-2-[2-[4-[fluoro(4-fluorophenyl)methyl]-4-(1-piperidinylmethyl)-1-piperidinyl]-2-oxoethoxy]phenyl]urea

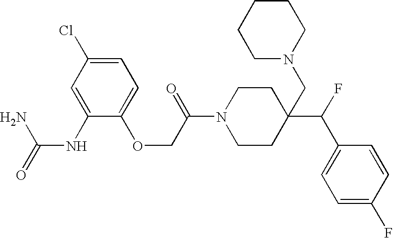 Prepared in a similar manner to Compound 52. ¹H NMR (400 MHz, DMSO-d₆): δ/ppm=1.10 (m, 1H), 1.30 (m, 1H), 1.56 (m, 2H), 1.70 (m, 2H), 1.82 (m, 4H), 3.12 (m, 2H), 3.42 (m, 5H), 3.65 (m, 1H), 3.82 (m, 2H), 4.82 (m, 2H), 5.95 (d, 1H), 6.36 (s, 1H), 6.70 (m, 2H), 7.35 (m, 4H), 8.02 (s, 1H), 8.18 (s, 1H), 8.68 (s, 1H).

### Example 54

### N-[5-chloro-2-[2-[4-[(dimethylamino)methyl]-4-[fluoro(4-fluorophenyl)methyl]-1-piperidinyl]-2-oxoethoxy]phenyl]urea

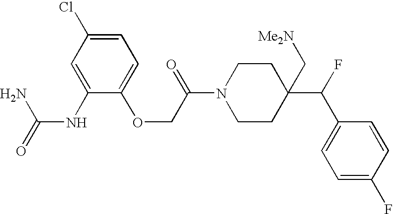 Prepared in a similar manner to Compound 52. ¹H NMR (400 MHz, DMSO-d₆): δ/ppm=1.30 (m, 2H), 1.62 (m, 1H), 1.80 (m, 1H), 2.95 (s, 6H), 3.12 (m, 1H), 3.32 (m, 1H), 3.45 (m, 2H), 3.66 (m, 1H), 3.82 (m, 1H), 4.86 (m, 2H), 5.95 (d, 1H), 6.32 (s, 1H), 6.70 (m, 1H), 6.78 (m, 1H), 7.30 (m, 4H), 8.02 (s, 1H), 8.18 (s, 1H), 9.24 (s, 1H).
LRMS M+H, 495.2

### Example 55

### N-[5-chloro-2-[2-[4-[(4-fluorophenyl)methyl]-4-(1H-pyrrol-1-ylmethyl)-1-piperidinyl]-2-oxoethoxy]phenyl]urea

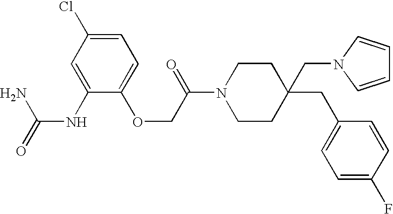

### A. 4-[(4-fluorophenyl)methyl]-4-(1H-pyrrol-1-ylmethyl)-1-piperidinecarboxylic acid 1,1-dimethylethyl ester

To a solution of Intermediate 33a (443 mg, 1.4 mmol) and sodium acetate (112 mg, 1.4 mmol) in acetic acid (15 mL) at 75° C. was added tetrahydro-2,5-dimethoxyfuran (236 mg, 1.8 mmol). Heating was continued overnight. Extraction and purification by chromatography on silica afforded Intermediate 55a (281 mg).

### B. N-[5-chloro-2-[2-[4-[(4-fluorophenyl)methyl]-4-(1H-pyrrol-1-ylmethyl)-1-piperidinyl]-2-oxoethoxy]phenyl]urea

The tert-butoxycarbonyl protecting group was removed from Intermediate 55a, and the crude product reacted with Intermediate 1b in a similar manner as described for Compound 2. Purification by reverse phase HPLC afforded Compound 55 (79 mg). ¹H NMR (400 MHz, DMSO-d₆): δ/ppm=1.30 (m, 2H), 1.38 (m, 2H), 2.74 (s, 2H), 3.60 (m, 4H), 3.94 (m, 2H), 4.88 (s, 2H), 6.06 (s, 2H), 6.40 (s, 1H), 6.70 (m, 3H), 6.80 (m, 1H), 7.20 (m, 4H), 8.10 (s, 1H), 8.22 (s, 1H).

### Examples 56 and 57

### N-[5-chloro-2-[3-[4-(4-fluorobenzoyl)-1-piperidinyl]-2-hydroxypropoxy]phenyl]urea and

### [1-[3-(2-amino-4-chlorophenoxy)-2-hydroxypropyl]-4-piperidinyl](4-fluorophenyl)methanone

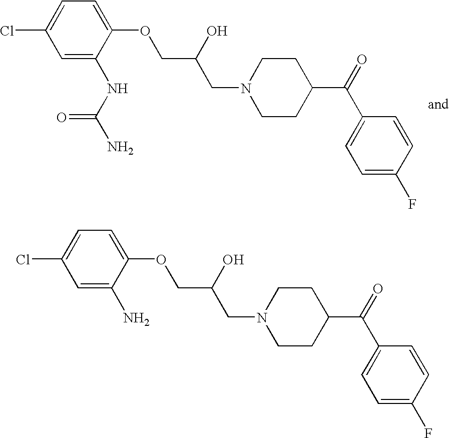

### A. N-[5-chloro-2-(oxiranylmethoxy)phenyl]urea

To a solution of N-(5-chloro-2-hydroxyphenyl)urea (5 g, 27 mmol) in dimethylformamide (20 mL) were added epibromohydrin (4.8 mL, 56 mmol) and potassium carbonate (1.4 g, 54 mmol), and the mixture stirred at ambient temperature for 3 days. The mixture was poured into ice water and the resulting solid collected by filtration and washed with water. Recrystallization (dichloromethane-methanol) afforded Intermediate 56a as a light yellow crystalline solid.

### B. N-[5-chloro-2-[3-[4-(4-fluorobenzoyl)-1-piperidinyl]-2-hydroxypropoxy]phenyl]urea and

### [1-[3-(2-amino-4-chlorophenoxy)-2-hydroxypropyl]-4-piperidinyl](4-fluorophenyl)methanone

To a solution of Intermediate 56a (500 mg, 2.1 mmol) and (4-fluorophenyl)-4-piperidinylmethanone (540 mg, 2.2 mmol) in dimethylformamide (10 mL) was added potassium carbonate (725 mg, 5.2 mmol). The mixture was heated at 110° C. in a sealed tube for 2 days. After cooling to ambient temperature, the mixture was poured into ice water. Extraction and purification by reverse phase HPLC afforded Compounds 56 and 57 as yellow solids.

Compound 56: ¹H NMR (400 MHz, DMSO-d₆+TFA): δ/ppm=1.50-1.85 (m, 4H), 2.80-3.80 (m, 9H), 4.42 (m, 1H), 6.60-6.74 (m, 2H), 7.04-7.14 (m, 2H), 7.68 (br, 1H), 7.78-7.86 (m, 2H), 7.90 (m, 1H), 9.18 (br, 1H).

LRMS M+H, 450.2

Compound 57: ¹H NMR (400 MHz, DMSO-d₆): δ/ppm=1.8-2.1 (m, 4H), 3.05-3.90 (m, 9H), 4.30 (m, 1H), 6.48 (dd, 1H), 6.62 (m, 1H), 6.74 (d, 1H), 7.38 (dd, 2H), 8.10 (m, 2H).

LRMS M+H, 407.2

### Example 58

### N-[5-chloro-2-[2-[4-(4-fluorobenzoyl)-1-piperidinyl]ethoxy]phenyl]urea

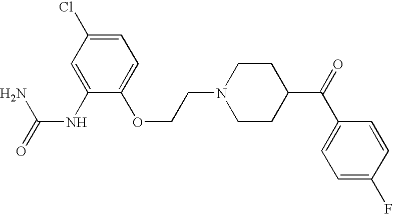

### A. N-[2-(2-bromoethoxy)-5-chlorophenyl]urea

To a mixture of N-(5-chloro-2-hydroxyphenyl)urea (1.0 g, 5.4 mmol) and potassium carbonate (1.5 g, 11 mmol) in dimethylformamide (20 mL) was added dropwise 1,2-dibromoethane (1.5 mL, 17 mmol), and the mixture stirred at ambient temperature for 3 days. The mixture was poured into ice water and the resulting solid collected by filtration and washed with water. The solid was dissolved in dichloromethane (20 mL), filtered and concentrated to afford Intermediate 58a as a light yellow crystalline solid.

### B. N-[5-chloro-2-[2-[4-(4-fluorobenzoyl)-1-piperidinyl]ethoxy]phenyl]urea

(4-Fluorophenyl)-4-piperidinylmethanone (250 mg, 1.0 mmol) was treated with aqueous cesium carbonate (650 mg, 2.0 mmol), extracted into ether, dried and concentrated. The residue was dissolved in acetonitrile (10 mL) and Intermediate 58a (200 mg, 0.6 mmol) was added. The mixture was stirred at ambient temperature for 5 days. Concentration and purification by reverse phase HPLC afforded Compound 58 (53 mg). ¹H NMR (400 MHz, DMSO-d₆): δ/ppm=1.82 (m, 1.7H), 2.01 (m, 2.3H), 3.20 (m, 2H), 3.38 (m, 2H), 3.65 (m, 3H), 4.18 (m, 2H), 6.90 (m, 1H), 7.00 (d, 1H), 7.30 (t, 2H), 7.95 (s, 1H), 8.04 (m, 2H), 8.10 (s, 1H), 9.55 (br, 1H).

LRMS M+H 419

### Example 59

### 1-[(4-chloro-2-cyanophenoxy)acetyl]-4-[(4-fluorophenyl)methyl]-4-piperidinecarbonitrile

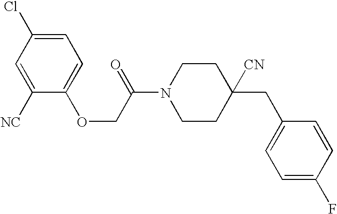

### A. 4-[(4-fluorophenyl)methyl]-4-piperidinecarbonitrile monohydrochloride

To a solution of 4-cyano-4-[(4-fluorophenyl)methyl]-1-piperidinecarboxylic acid 1,1-dimethylethyl ester (19.5 g, 62 mmol) in tetrahydrofuran (40 mL) was added hydrochloric acid (4.0 M solution in dioxane, 40 mL, 160 mmol) and the mixture stirred at ambient temperature for 5 hours. The precipitate was collected by filtration, washed with ether and dried to afford Intermediate 59a (15 g) as a white solid.

### B. 1-(chloroacetyl)-4-[(4-fluorophenyl)methyl]-4-piperidinecarbonitrile

To a solution of Intermediate 59a (590 mg, 2.3 mmol) and triethylamine (0.74 mL, 5.3 mmol) in dichloromethane (2 mL) at −78° C. was added a solution of chloroacetyl chloride (0.20 mL, 2.5 mmol) in dichloromethane (2 mL). The mixture was stirred allowed to warm to ambient temperature over 30 minutes and stirred for an additional 2 hours. The mixture was washed with 1 N hydrochloric acid, water and saturated aqueous sodium bicarbonate, dried and concentrated to afford Intermediate 59b (626 mg), which was used without further purification.

### C. 1-[(4-chloro-2-cyanophenoxy)acetyl]-4-[(4-fluorophenyl)methyl]-4-piperidinecarbonitrile

To a solution of Intermediate 59b (209 mg, 0.80 mmol) in dimethylsulfoxide (5 mL) were added potassium carbonate (196 mg, 1.4 mmol) and 5-chloro-2-hydroxybenzonitrile (109 mg, 0.7 mmol). The mixture was heated at 60° C. overnight. Extraction and purification by reverse phase HPLC afforded Compound 59 (175 mg). ¹H NMR (400 MHz, CDCl₃): δ/ppm=1.52 (dt, 1H), 1.75 (dt, 1H), 1.94-2.02 (m, 2H), 2.86 (s, 2H), 2.91-2.98 (m, 1H), 3.37-3.43 (m, 1H), 4.07-4.11 (m, 1H), 4.62-4.65 (m, 1H), 4.80 (d, 1H), 4.88 (d, 1H), 7.0 (d, 1H), 7.02-7.06 (m, 2H), 7.23-7.27 (m, 2H), 7.52 (dd, 1H), 7.58 (d, 1H).

LRMS M+H, 412.1

### Example 60

### 1-[[4-chloro-2-(1H-pyrazol-5-yl)phenoxy]acetyl]-4-[(4-fluorophenyl)methyl]-4-piperidinecarbonitrile

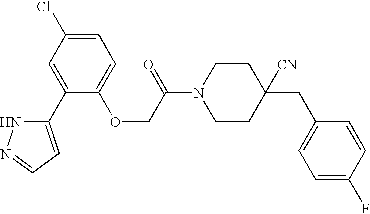 Prepared in a similar manner to Compound 59. ¹H NMR (400 MHz, CDCl₃): δ/ppm=1.55-1.65 (m, 2H), 1.92-1.96 (m, 2H), 2.85-2.95 (m, 3H), 3.35-3.41 (m, 1H), 3.81 (d, 1H), 4.99 (d, 1H), 4.99 (d, 1H), 5.03 (d, 1H), 6.82 (d, 1H), 7.00-7.08 (m, 3H), 7.22-7.25 (m, 2H), 7.37 (dd, 1H), 7.68 (d, 1H), 7.86 (d, 1H).
LRMS M+H, 453.1

### Examples 61 and 62

### 1-[[4-chloro-2-(5-isoxazolyl)phenoxy]acetyl]-4-[(4-fluorophenyl)methyl]-4-piperidinecarbonitrile and

### 1-[[4-chloro-2-(cyanoacetyl)phenoxy]acetyl]-4-[(4-fluorophenyl)methyl]-4-piperidinecarbonitrile

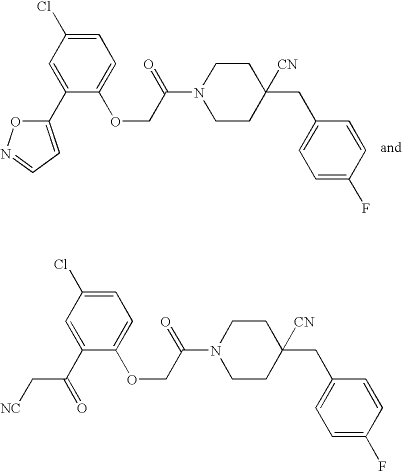 Treatment of Intermediate 59b with 4-chloro-2-(5-isoxazolyl)phenol in a similar manner to that described for Compound 59 afforded a mixture of Compounds 61 and 62. The products were separated by reverse phase HPLC.
Compound 61 (41 mg): ¹H NMR (400 MHz, CDCl₃): δ/ppm=1.40-1.49 (m, 2H), 1.85-1.97 (m, 2H), 2.80-2.86 (m, 2H), 2.90-2.96 (m, 1H), 3.34-3.41 (m, 1H), 3.88-3.91 (m, 1H), 4.67-4.70 (m, 1H), 4.80 (d, 1H), 4.86 (d, 1H), 6.95 (d, 1H), 7.02-7.06 (m, 2H), 7.10 (d, 1H), 7.17-7.20 (m, 2H), 7.35 (dd, 1H), 7.98 (d, 1H), 8.34 (d, 1H).
LRMS M+H, 454.1
Compound 62 (10 mg): ¹H NMR (400 MHz, CDCl₃): δ/ppm=1.50-1.60 (m, 2H), 1.98 (d, 2H), 2.88 (d, 2H), 2.92-3.98 (m, 1H), 3.41-3.47 (m, 1H), 3.72-3.76 (m, 1H), 4.2 (d, 1H), 4.38 (d, 1H), 4.65-4.69 (m, 1H), 4.88 (s, 2H), 6.88 (d, 1H), 7.04-7.08 (m, 2H), 7.23-7.26 (m, 2H), 7.49 (dd, 1H), 7.81 (d, 1H).
LRMS M+H, 454.1

### Example 63

### N-[[5-chloro-2-[2-[4-cyano-4-[(4-fluorophenyl)methyl]-1-piperidinyl]-2-oxoethoxy]phenyl]methyl]methanesulfonamide

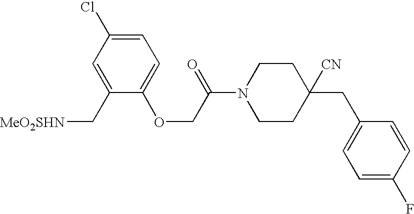

### A. N-[(5-chloro-2-methoxyphenyl)methyl]methanesulfonamide

To a solution of 5-chloro-2-methoxybenzenemethanamine (1.0 g, 4.8 mmol, Yu, et. al., Synthesis, 2003, 3, 403) in dichloromethane (30 mL) were added methanesulfonyl chloride (0.41 mL, 5.3 mmol) and triethylamine (1.3 mL, 9.3 mmol) and the mixture stirred at ambient temperature overnight. Extraction and recrystallization afforded Intermediate 63a (960 mg) as a white solid.

### B. N-[(5-chloro-2-hydroxyphenyl)methyl]methanesulfonamide

To a solution of Intermediate 63a (465 mg, 1.9 mmol) in dichloromethane (10 mL) at −78° C. was added boron tribromide (1.0 M in dichloromethane, 2.8 mL, 2.8 mmol) dropwise. The mixture was allowed to warm slowly to ambient temperature while stirring for 3 hours. The reaction was then cooled to 0° C. and quenched with methanol (3 mL), followed by addition of dichloromethane (10 mL), 1 M sodium hydroxide (2 mL) and 1 M hydrochloric acid (3 mL). Extraction and recrystallization afforded Intermediate 63b (230 mg).

### C. N-[[5-chloro-2-[2-[4-cyano-4-[(4-fluorophenyl)methyl]-1-piperidinyl]-2-oxoethoxy]phenyl]methyl]methanesulfonamide

Reaction of Intermediate 63b (160 mg, 0.68 mmol) with Intermediate 59b (200 mg, 0.68 mmol) in a similar manner to that described for Compound 59 and purification by MPLC afforded Compound 63. ¹H NMR (400 MHz, DMSO-d₆): δ/ppm=1.47-1.53 (m, 1H), 1.65-1.71 (m, 1H), 1.78 (m, 2H), 2.67 (t, 1H), 2.87 (s, 3H), 2.91 (s, 2H), 3.11 (t, 1H), 3.87 (d, 1H), 4.15 (d, 2H), 4.34 (d, 1H), 4.90 (d, 1H), 4.98 (d, 1H), 6.96 (d, 1H), 7.14-7.18 (m, 2H), 7.26 (dd, 1H), 7.29-7.32 (m, 2H), 7.35 (d, 1H), 7.45 (m, 1H).

LRMS M+H, 494.1

### Example 64

### 1-[(2-bromo-4-chlorophenoxy)acetyl]-4-[(4-fluorophenyl)methyl]-4-piperidinecarbonitrile

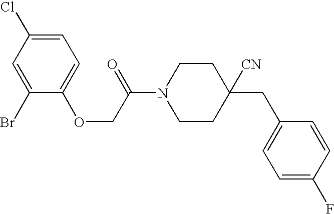 Prepared in a similar manner to Compound 59. ¹H NMR (400 MHz, CDCl₃): δ/ppm=1.42 (dd, 1H), 1.50-1.57 (m, 1H), 1.88-1.95 (m, 2H), 2.82 (d, 2H), 2.89-2.93 (m, 1H), 3.35 (dt, 1H), 4.16-4.20 (m, 1H), 4.59-4.63 (m, 1H), 4.68 (d, 1H), 4.80 (d, 1H), 6.91 (d, 1H), 7.01-7.06 (m, 2H), 7.19-7.22 (m, 2H), 7.24 (dd, 1H), 7.56 (d, 1H).
LRMS M+H, 466.9

### Example 65

### N-[2-[2-[4-cyano-4-[(4-fluorophenyl)methyl]-1-piperidinyl]-2-oxoethoxy]phenyl]urea

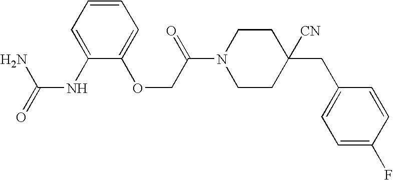 Prepared in a similar manner to Compound 59. ¹H NMR (400 MHz, CDCl₃) δ/ppm=1.40 (m, 2H), 1.80 (m, 2H), 2.75 (s, 2H), 2.80 (m, 1H), 3.25 (m, 1H), 3.45 (d, 1H), 4.60 (m, 1H), 4.65 (s, 2H), 6.80-7.00 (m, 6H), 7.15 (m, 2H), 8.05 (d, 1H), 8.45 (s, 1H).
LRMS M+H: 410

### Example 66

### 1-[[(5-chloro-8-quinolinyl)oxy]acetyl]-4-[(4-fluorophenyl)methyl]-4-piperidinecarbonitrile

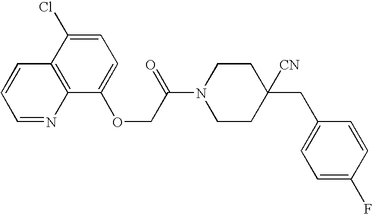 Prepared in a similar manner to Compound 59. ¹H NMR (400 MHz, DMSO-d₆): δ/ppm=1.50 (m, 1H), 1.70-1.85 (m, 3H), 2.70 (m, 1H), 2.90 (s, 2H), 3.15 (m, 1H), 4.00 (m, 1H), 4.35 (m, 1H), 5.10 (m, 2H), 7.12-7.20 (m, 3H), 7.30 (m, 2H), 7.68 (d, 1H), 7.77 (dd, 1H), 8.58 (dd, 1H), 8.98 (dd, 1H).
LRMS M+H, 438.1

### Example 67

### 5-chloro-2-[2-[4-(4-fluorobenzoyl)-1-piperidinyl]-2-oxoethoxy]benzamide

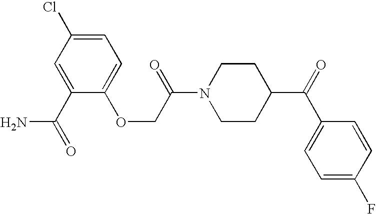 Prepared in a similar manner to Compound 59. ¹H NMR (400 MHz, DMSO-d₆): /ppm=1.40 (m, 1H), 1.60 (m, 1H), 1.80 (d, 2H), 2.85 (t, 1H), 3.21 (d, 1H), 3.77 (t, 1H), 3.82 (d, 1H), 4.35 (d, 1H), 5.06 (d, 1H), 5.12 (d, 1H), 7.24 (d, 1H), 7.38 (m, 2H), 7.52 (d, 1H), 7.70 (s, 1H), 7.82 (s, 1H), 8.09 (dd, 2H), 8.74 (s, 1H).
LRMS M+H: 418

### Example 68

### 5-chloro-2-[2-[4-(4-fluorobenzoyl)-1-piperidinyl]-2-oxoethoxy]benzoic acid methyl ester

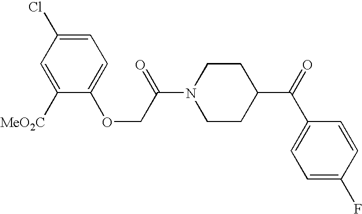 Prepared in a similar manner to Compound 59. ¹H NMR (400 MHz, CDCl₃): δ/ppm=1.64 (m, 1H), 1.78 (m, 1H), 1.92 (m, 2H), 2.93 (t, 1H), 3.30 (t, 1H), 3.47 (m, 1H), 3.86 (s, 3H), 4.22 (d, 1H), 4.47 (d, 1H), 4.79 (s, 2H), 7.02 (d, 1H), 7.14 (m, 2H), 7.40 (d, 1H), 7.78 (s, 1H), 7.95 (m, 2H).
LRMS M+H 433

### Example 69

### [1-[(2-amino-4-chlorophenoxy)acetyl]-4-piperidinyl](4-fluorophenyl)methanone

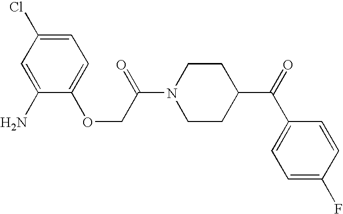 Prepared in a similar manner to Compound 59. ¹H NMR (400 MHz, CDCl₃): δ/ppm=1.70 (m, 1H), 1.90 (m, 3H), 2.95 (t, 1H), 3.24 (t, 1H), 3.48 (m, 1H), 3.95 (d, 1H), 4.06 (s, 2H), 4.53 (d, 1H), 4.73 (s, 2H), 6.62 (m, 1H), 6.72 (m, 2H), 7.16 (t, 2H), 7.95 (dd, 2H).

### Example 70

### 4-[(4-fluorophenyl)methyl]-1-[[(5-nitro-8-quinolinyl)oxy]acetyl]-4-piperidinecarbonitrile

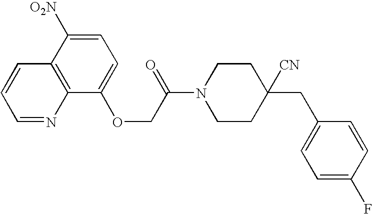 Prepared in a similar manner to Compound 59. ¹H NMR (400 MHz, DMSO-d₆): δ/ppm=1.54 (m, 1H), 1.78 (m, 3H), 2.68 (m, 1H), 2.94 (s, 2H), 3.17 (m, 1H), 3.96 (m, 1H), 4.34 (m, 1H), 5.26 (m, 2H), 7.20 (m, 2H), 7.32 (m, 3H), 7.84 (m, 1H), 8.48 (m, 1H), 9.00 (m, 2H).
LRMS M+H, 449.1

### Example 71

### 1-[(4-chlorophenoxy)acetyl]-4-[(4-fluorophenyl)methyl]-4-piperidinecarbonitrile

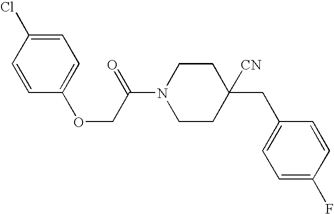 Prepared in a similar manner to Compound 59. ¹H NMR (400 MHz, DMSO-d₆): δ/ppm=1.50 (m, 1H), 1.68 (m, 1H), 1.76 (m, 2H), 2.50 (m, 1H), 2.92 (s, 2H), 3.10 (m, 1H), 3.86 (m, 1H), 4.34 (m, 1H), 4.82 (m, 2H), 6.90 (m, 2H), 7.16 (m, 2H), 7.30 (m, 4H).
LRMS (M+H): 387.0

### Example 72

### 4-[(4-fluorophenyl)methyl]-1-[(4-quinolinyloxy)acetyl]-4-piperidinecarbonitrile

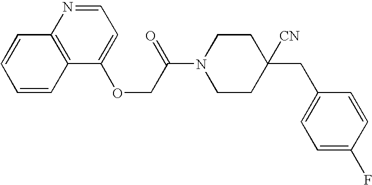 Prepared in a similar manner to Compound 59. ¹H NMR (400 MHz, DMSO-d₆+TFA): δ/ppm=1.55 (m, 1H), 1.70-1.90 (m, 3H), 2.75 (m, 1H), 2.95 (s, 2H), 3.20 (m, 1H), 3.90 (m, 1H), 4.85 (m, 1H), 5.60 (m, 2H), 7.16 (m, 2H), 7.32 (m, 2H), 7.56 (m, 1H), 7.79 (m, 1H), 8.08-8.20 (m, 2H), 8.40 (m, 1H), 9.16 (m, 1H).
LRMS M+H, 404.1

### Example 73

### 4-[(4-fluorophenyl)methyl]-1-[(7-isoquinolinyloxy)acetyl]-4-piperidinecarbonitrile

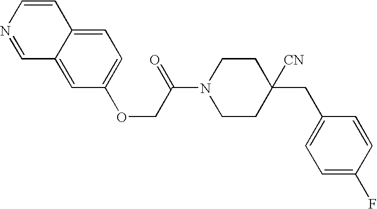 Prepared in a similar manner to Compound 59. ¹H NMR (400 MHz, DMSO-d₆+TFA): δ/ppm=1.55 (m, 1H), 1.70-1.90 (m, 3H), 2.75 (m, 1H), 2.95 (s, 2H), 3.20 (m, 1H), 3.95 (m, 1H), 4.40 (m, 1H), 5.15 (m, 2H), 7.16 (m, 2H), 7.32 (m, 2H), 7.82 (m, 1H), 7.92 (m, 1H), 8.28 (m, 1H), 8.46 (m, 1H), 8.56 (m, 1H), 9.67 (s, 1H).
LRMS M+H, 404.1

### Example 74

### 4-[(4-fluorophenyl)methyl]-1-[[(2-hydroxy-8-quinolinyl)oxy]acetyl]-4-piperidinecarbonitrile

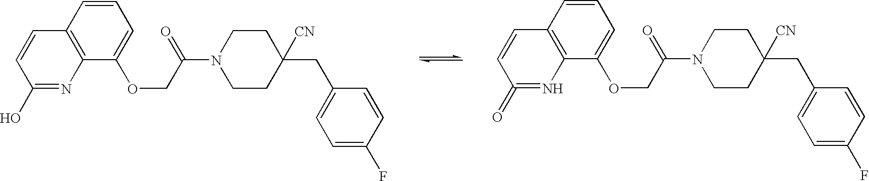 Prepared in a similar manner to Compound 59. ¹H NMR (400 MHz, DMSO-d₆+TFA): δ/ppm=1.50 (m, 1H), 1.66 (m, 1H), 1.74-1.82 (m, 2H), 2.70 (m, 1H), 2.89 (s, 2H), 3.14 (m, 1H), 3.94 (m, 1H), 4.38 (m, 1H), 4.98 (m, 2H), 6.54 (m, 1H), 7.04-7.18 (m, 4H), 7.24-7.32 (m, 3H), 7.89 (m, 1H).
LRMS M+H, 420.1.

### Example 75

### 4-[(4-fluorophenyl)methyl]-1-[(6-quinolinyloxy)acetyl]-4-piperidinecarbonitrile

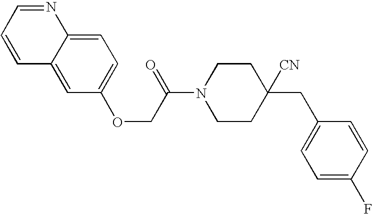 Prepared in a similar manner to Compound 59. ¹H NMR (400 MHz, DMSO-d₆+TFA): δ/ppm=1.54 (m, 1H), 1.70-1.90 (m, 3H), 2.70 (m, 1H), 2.95 (s, 2H), 3.20 (m, 1H), 3.92 (m, 1H), 4.48 (m, 1H), 5.10 (m, 2H), 7.16 (m, 2H), 7.32 (m, 2H), 7.72 (m, 1H), 7.84 (m, 1H), 8.04 (m, 1H), 8.20 (m, 1H), 9.00 (m, 1H), 9.16 (m, 1H).
LRMS M+H, 404.1

### Example 76

### 4-[(4-fluorophenyl)methyl]-1-[(5-isoquinolinyloxy)acetyl]-4-piperidinecarbonitrile

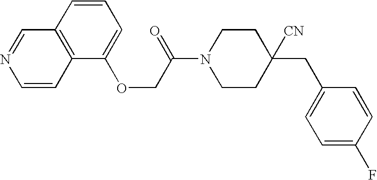 Prepared in a similar manner to Compound 59. ¹H NMR (400 MHz, DMSO-d₆+TFA): δ/ppm=1.55 (m, 1H), 1.70-1.90 (m, 3H), 2.70 (m, 1H), 2.95 (s, 2H), 3.20 (m, 1H), 3.95 (m, 1H), 4.38 (m, 1H), 5.25 (m, 2H), 7.16 (m, 2H), 7.32 (m, 2H), 7.62 (m, 1H), 7.92 (m, 1H), 8.08 (m, 1H), 8.57 (m, 1H), 8.68 (m, 1H), 9.88 (s, 1H).
LRMS M+H, 404.1

### Example 77

### 4-[(4-fluorophenyl)methyl]-1-[(6-isoquinolinyloxy)acetyl]-4-piperidinecarbonitrile

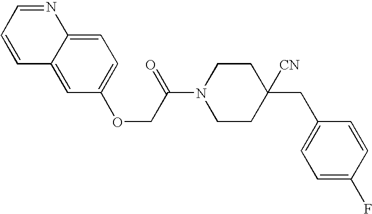 Prepared in a similar manner to Compound 59. ¹H NMR (400 MHz, DMSO-d₆+TFA): δ/ppm=1.52 (m, 1H), 1.70-1.90 (m, 3H), 2.70 (m, 1H), 2.90 (s, 2H), 3.18 (m, 1H), 3.90 (m, 1H), 4.35 (m, 1H), 5.20 (m, 2H), 7.13 (m, 2H), 7.30 (m, 2H), 7.62 (s, 1H), 7.65 (m, 1H), 8.20 (m, 1H), 8.42 (m, 1H), 8.50 (m, 1H), 9.66 (s, 1H).
LRMS M+H, 404.1

### Example 78

### 4-[(4-fluorophenyl)methyl]-1-[[(5-fluoro-8-quinolinyl)oxy]acetyl]-4-piperidinecarbonitrile

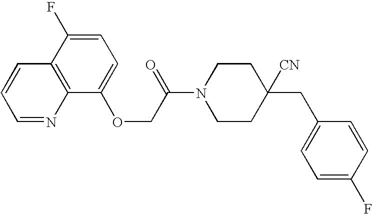 Prepared in a similar manner to Compound 59. ¹H NMR (400 MHz, DMSO-d₆+TFA): δ/ppm=1.50 (m, 1H), 1.62-1.85 (m, 3H), 2.70 (m, 1H), 2.90 (s, 2H), 3.18 (m, 1H), 3.90 (m, 1H), 4.35 (m, 1H), 5.28 (m, 2H), 7.12 (m, 2H), 7.28 (m, 2H), 7.50 (m, 1H), 7.62 (m, 1H), 8.08 (m, 1H), 9.08 (m, 1H), 9.20 (m, 1H).
LRMS M+H, 422.1

### Example 79

### 1-[[(2-amino-8-quinolinyl)oxy]acetyl]-4-[(4-fluorophenyl)methyl]-4-piperidinecarbonitrile

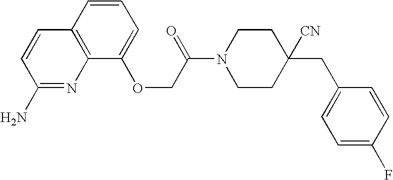 Prepared in a similar manner to Compound 59. ¹H NMR (500 MHz, DMSO-d₆+D₂O): δ/ppm=1.49 (m, 1H), 1.65-1.80 (m, 3H), 2.68 (m, 1H), 2.85 (m, 2H), 3.10 (m, 1H), 4.03 (m, 1H), 4.35 (m, 1H), 4.85 (m, 2H), 6.74 (m, 1H), 6.87 (m, 1H), 6.98 (m, 1H), 7.13 (m, 2H), 7.20 (m, 1H), 7.27 (m, 2H), 7.83 (m, 1H).
LRMS M+H, 419.2

### Example 80

### N-[[5-chloro-2-[2-[4-cyano-4-[(4-fluorophenyl)methyl]-1-piperidinyl]-2-oxoethoxy]phenyl]methyl]-1-propanesulfonamide

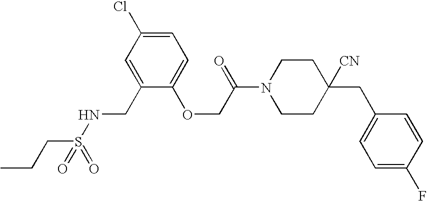 Prepared in a similar manner to Compound 59. ¹H NMR (400 MHz, CDCl₃): δ/ppm=0.98 (t, 3H), 1.51-1.63 (m, 2H), 1.72-1.81 (m, 2H), 1.95 (t, 2H), 2.87 (s, 2H), 2.90-2.94 (m, 3H), 3.99 (t, 1H), 3.80 (d, 1H), 4.24 (d, 1H), 4.28 (d, 1H), 4.64 (d, 1H), 4.78 (s, 2H), 6.39 (br, 1H), 6.79 (d, 1H), 7.05 (t, 2H), 7.22-7.28 (m, 4H).
LRMS M+H, 522.1

### Example 81

### 4-[(4-fluorophenyl)methyl]-1-[[4-(trifluoromethyl)phenoxy]acetyl]-4-piperidinecarbonitrile

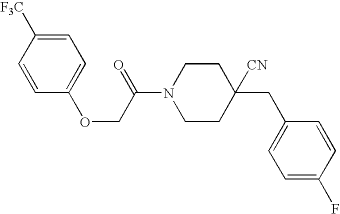 Prepared in a similar manner to Compound 59. ¹H NMR (400 MHz, CDCl₃): δ/ppm=1.41-1.52 (m, 2H), 1.90-1.95 (m, 2H), 2.82 (s, 2H), 2.89 (t, 1H), 3.37 (t, 1H), 4.03 (d, 1H), 4.63 (d, 1H), 4.71 (d, 1H), 4.77 (d, 1H), 7.00-7.07 (m, 4H), 7.20-7.24 (m, 2H), 7.56 (d, 2H).
LRMS M+H, 421.1

### Example 82

### 5-chloro-2-[2-[4-cyano-4-[(4-fluorophenyl)methyl]-1-piperidinyl]-2-oxoethoxy]-N-methylbenzenesulfonamide

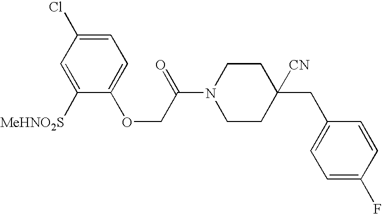 Prepared in a similar manner to Compound 59. ¹H NMR (400 MHz, CDCl₃): δ/ppm=1.56 (dt, 1H), 1.69 (dt, 1H), 1.92-1.97 (m, 2H), 2.57 (d, 3H), 2.88-2.91 (m, 3H), 3.34 (t, 1H), 3.74 (d, 1H), 4.62 (d, 1H), 4.72 (d, 1H), 4.97 (d, 1H), 6.79 (d, 1H), 6.89 (q, 1H), 7.06-7.09 (m, 2H), 7.25-7.28 (m, 2H), 7.33 (dd, 1H), 7.88 (d, 1H).
LRMS M+H, 480.0

### Example 83

### N-[5-chloro-2-[2-[4-cyano-4-[(4-fluorophenyl)methyl]-1-piperidinyl]-2-oxoethoxy]phenyl]methanesulfonamide

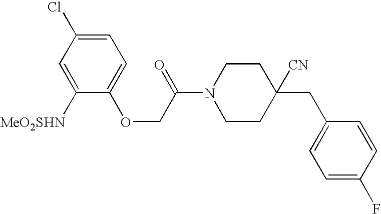 Prepared in a similar manner to Compound 59. ¹H NMR (400 MHz, CDCl₃): δ/ppm=1.50 (dt, 2H), 1.94 (d, 2H), 2.81-2.91 (m, 3H), 3.03 (s, 3H), 3.35 (t, 1H), 3.64 (d, 1H), 4.65 (d, 1H), 4.79 (s, 2H), 6.89 (d, 1H), 7.03-7.08 (m, 3H), 7.22-7.26 (m, 2H), 7.58 (d, 1H), 8.53 (s, 1H).
LRMS M+H, 480.1

### Example 84

### 5-chloro-2-[2-[4-cyano-4-[(4-fluorophenyl)methyl]-1-piperidinyl]-2-oxoethoxy]benzenesulfonamide

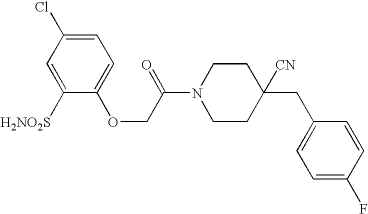 Prepared in a similar manner to Compound 59. ¹H NMR (400 MHz, DMSO-d₆): δ/ppm=1.50-1.56 (m, 1H), 1.70-1.73 (m, 1H), 1.80 (d, 2H), 2.71 (t, 1H), 2.91 (s, 2H), 3.11 (t, 1H), 3.87 (d, 1H), 4.38 (d, 1H), 5.04 (d, 1H), 5.14 (d, 1H), 7.15-7.19 (m, 2H), 7.27-7.32 (m, 3H), 7.49 (s, 2H), 7.64-7.66 (m, 2H).
LRMS M+H, 466.1

### Example 85

### 5-bromo-2-[2-[4-cyano-4-[(4-fluorophenyl)methyl]-1-piperidinyl]-2-oxoethoxy]benzeneacetic acid methyl ester

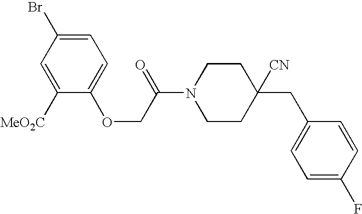 Prepared in a similar manner to Compound 59. ¹H NMR (400 MHz, CDCl₃): δ/ppm=1.50 (m, 2H), 1.90 (m, 2H), 2.84 (s, 2H), 2.90 (m, 1H), 3.30 (t, 1H), 3.60 (br, 2H), 3.65 (s, 3H), 4.0 (m, 1H), 4.65-4.70 (m, 3H), 6.80 (d, 1H), 7.05 (t, 2H), 7.25 (m, 2H), 7.35 (m, 2H).
LRMS M+H: 503

### Example 86

### 4-[(4-fluorophenyl)methyl]-1-[(4-formyl-3,5-dimethoxyphenoxy)acetyl]-4-piperidinecarbonitrile

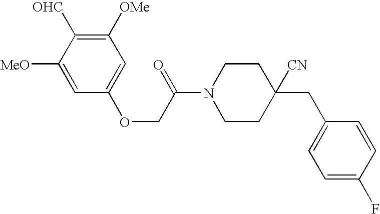 Prepared in a similar manner to Compound 59. ¹H NMR (400 MHz, CDCl₃): δ/ppm=1.43-1.46 (m, 2H), 1.94 (d, 2H), 2.83 (s, 2H), 2.91 (t, 1H), 3.39 (t, 1H), 3.88 (s, 6H), 4.07 (d, 1H), 4.64 (d, 1H), 4.72 (d, 1H), 4.78 (d, 1H), 6.17 (s, 2H), 7.02-7.06 (m, 2H), 7.20-7.23 (m, 2H), 10.37 (s, 1H).
LRMS M+H, 441.1

### Example 87

### N-[5-chloro-2-[2-[4-cyano-4-[(4-fluorophenyl)methyl]-1-piperidinyl]-2-oxoethoxy]-4-methylphenyl]acetamide

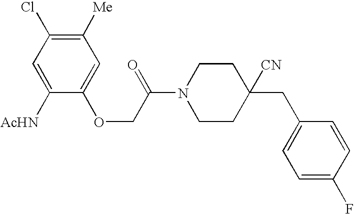 Prepared in a similar manner to Compound 59. ¹H NMR (400 MHz, CDCl₃): δ/ppm=1.46-1.53 (m, 2H), 1.93 (d, 2H), 2.21 (s, 3H), 2.29 (s, 3H), 2.81-2.93 (m, 3H), 3.34 (t, 1H), 3.66 (d, 1H), 4.67 (d, 1H), 4.74 (s, 2H), 6.78 (s, 1H), 7.02-7.07 (m, 2H), 7.21-7.24 (m, 2H), 8.36 (s, 1H), 9.33 (s, 1H).
LRMS M+H, 458.1

### Example 88

### N-[2-[2-[4-cyano-4-[(4-fluorophenyl)methyl]-1-piperidinyl]-2-oxoethoxy]-4-methylphenyl]acetamide

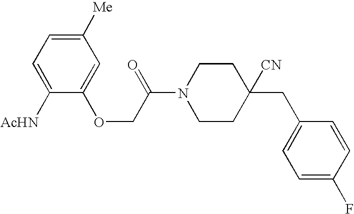 Prepared in a similar manner to Compound 59. ¹H NMR (400 MHz, CDCl₃): δ/ppm=1.45-1.51 (m, 2H), 1.90-1.95 (m, 2H), 2.21 (s, 3H), 2.29 (s, 3H), 2.80-2.93 (m, 3H), 3.34 (t, 1H), 3.70 (d, 1H), 4.68 (d, 1H), 4.76 (s, 2H), 6.74 (s, 1H), 6.86 (d, 1H), 7.02-7.07 (m, 2H), 7.21-7.24 (m, 2H), 8.17 (d, 1H), 9.11 (s, 1H).
LRMS M+H, 424.1

### Example 89

### 4-[(4-fluorophenyl)methyl]-1-[(4-nitrophenoxy)acetyl]-4-piperidinecarbonitrile

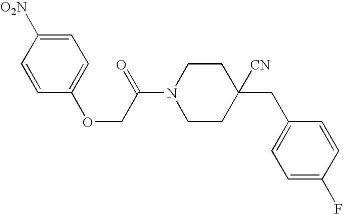 Prepared in a similar manner to Compound 59. ¹H NMR (400 MHz, CDCl₃): δ/ppm=1.49 (dq, 2H), 1.97 (d, 2H), 2.85 (s, 2H), 2.88-2.96 (m, 1H), 3.40 (t, 1H), 3.98 (d, 1H), 4.64 (d, 1H), 4.78 (d, 1H), 4.84 (d, 1H), 7.00-7.07 (m, 4H), 7.21-7.27 (m, 2H), 8.19-8.23 (m, 2H).
LRMS M+H, 398.1

### Example 90

### 4-[(4-fluorophenyl)methyl]-1-[(2-methoxy-4-nitrophenoxy)acetyl]-4-piperidinecarbonitrile

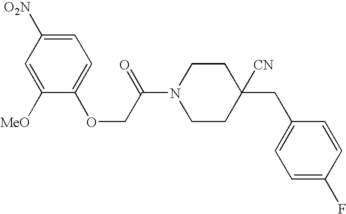 Prepared in a similar manner to Compound 59. ¹H NMR (400 MHz, CDCl₃): δ/ppm=1.44-1.57 (m, 2H), 1.91-1.96 (m, 2H), 2.84 (s, 2H), 2.87-2.93 (m, 1H), 3.38 (t, 1H), 3.96 (s, 3H), 4.06 (d, 1H), 4.62 (d, 1H), 4.83 (d, 1H), 4.87 (d, 1H), 6.96 (d, 1H), 7.02-7.07 (m, 2H), 7.21-7.24 (m, 2H), 7.78 (d, 1H), 7.87 (dd, 1H).
LRMS M+H, 428.1

### Example 91

### 4-[(4-fluorophenyl)methyl]-1-[(3-nitrophenoxy)acetyl]-4-piperidinecarbonitrile

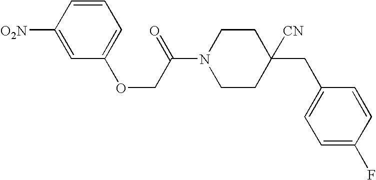 Prepared in a similar manner to Compound 59. ¹H NMR (400 MHz, CDCl₃): δ/ppm=1.48-1.55 (m, 2H), 1.94 (t, 2H), 2.85 (s, 2H), 2.90 (t, 1H), 3.40 (s, 1H), 3.95 (d, 1H), 4.64 (d, 1H), 4.74 (d, 1H), 4.82 (d, 1H), 7.01-7.05 (m, 2H), 7.21-7.25 (m, 2H), 7.28-7.31 (m, 1H), 7.46 (t, 1H), 7.72 (t, 1H), 7.87 (dd, 1H).
LRMS M+H, 398.0

### Example 92

### 1-[(4-chloro-3-nitrophenoxy)acetyl]-4-[(4-fluorophenyl)methyl]-4-piperidinecarbonitrile

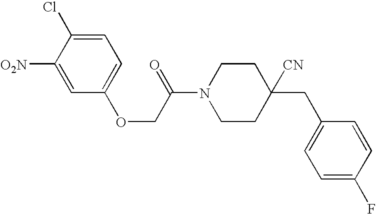 Prepared in a similar manner to Compound 59. ¹H NMR (400 MHz, CDCl₃): δ/ppm=1.51 (dq, 2H), 1.94 (t, 2H), 2.85 (s, 2H), 2.89 (t, 1H), 3.39 (t, 1H), 3.90 (d, 1H), 4.62 (d, 1H), 4.71 (d, 1H), 4.78 (d, 1H), 7.01-7.05 (m, 2H), 7.13 (dd, 1H), 7.21-7.25 (m, 2H), 7.41 (d, 1H), 7.44 (d, 1H).
LRMS M+H, 432.0

### Example 93

### 4-[(4-fluorophenyl)methyl]-1-[(4-formyl-2-methylphenoxy)acetyl]-4-piperidinecarbonitrile

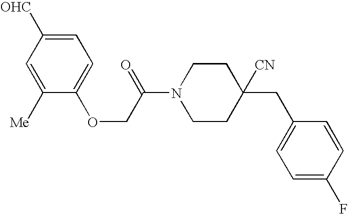 Prepared in a similar manner to Compound 59. ¹H NMR (400 MHz, CDCl₃): δ/ppm=1.44 (dt, 2H), 1.92 (d, 2H), 2.28 (s, 3H), 2.81 (s, 2H), 2.87-2.92 (m, 1H), 3.37 (t, 1H), 4.06 (d, 1H), 4.63 (d, 1H), 4.76 (d, 1H), 4.83 (d, 1H), 6.93 (d, 1H), 7.00-7.04 (m, 2H), 7.17-7.20 (m, 2H), 7.66-7.70 (m, 2H), 9.86 (s, 1H).
LRMS M+H, 395.1

### Example 94

### 2-[2-[4-cyano-4-[(4-fluorophenyl)methyl]-1-piperidinyl]-2-oxoethoxy]-benzenepropanoic acid methyl ester

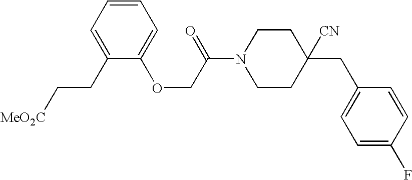 Prepared in a similar manner to Compound 59. ¹H NMR (400 MHz, CDCl₃): δ/ppm=1.44-1.50 (m, 2H), 1.90 (d, 2H), 2.59-2.63 (m, 2H), 2.82 (s, 2H), 2.85-3.00 (m, 3H), 3.34 (t, 1H), 3.66 (s, 3H), 4.10-4.14 (m, 1H), 4.64 (d, 2H), 4.75 (d, 1H), 6.84 (d, 1H), 6.92-6.96 (m, 1H), 7.90-7.04 (m, 2H), 7.16 (d, 2H), 7.20-7.25 (m, 2H).
LRMS M+H, 439.1

### Example 95

### 1-[(4-cyano-3-fluorophenoxy)acetyl]-4-[(4-fluorophenyl)methyl]-4-piperidinecarbonitrile

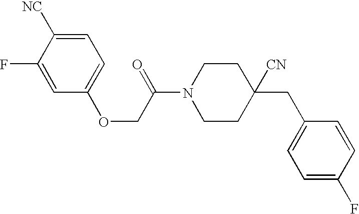 Prepared in a similar manner to Compound 59. ¹H NMR (400 MHz, CDCl₃): δ/ppm=1.43-1.52 (m, 2H), 1.94 (t, 2H), 2.84 (s, 2H), 2.89 (t, 1H), 3.38 (t, 1H), 3.90 (d, 1H), 4.61 (d, 1H), 4.72 (d, 1H), 4.77 (d, 1H), 6.75-6.82 (m, 2H), 7.03 (t, 2H), 7.20-7.25 (m, 2H), 7.53 (t, 1H).
LRMS M+H, 396.1

### Example 96

### 1-[(2-amino-4-chlorophenoxy)acetyl]-α-(4-fluorophenyl)-4-methyl-4-piperidinemethanol

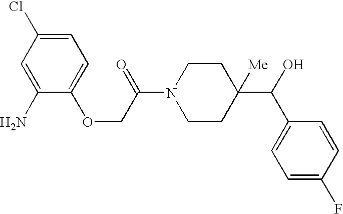 The tert-butoxycarbonyl protecting group was removed from Intermediate 15a, and the product reacted with chloroacetyl chloride and 2-amino-4-chlorophenol in a similar manner as described for Compound 59. The resulting Intermediate 96a (2.8 g, 6.9 mmol) was dissolved in ethanol (15 mL) and treated with sodium borohydride (287 mg, 7.6 mmol) at ambient temperature for 2 hours. Extraction and purification by chromatography on silica afforded Compound 96 (1.3 g) as a light brown solid. ¹H NMR (400 MHz, CDCL₃): δ/ppm=0.97 (s, 3H), 1.12 (t, 1H), 1.60 (m, 3H), 2.18 (s, 1H), 2.83 (q, 1H), 3.19 (m, 1H), 3.65 (t, 1H), 4.08 (br, 2H), 4.34 (m, 2H), 4.65 (m, 2H), 6.62 (m, 1H), 6.70 (m, 2H), 7.01 (t, 2H), 7.25 (m, 2H).
LRMS M+H: 406

### Example 97

### N-[5-chloro-2-[2-[4-cyano-4-[(4-fluorophenyl)hydroxymethyl]-1-piperidinyl]-2-oxoethoxy]phenyl]urea

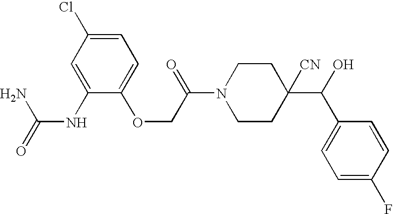 A solution of Compound 20 (114 mg, 0.25 mmol) in 1:1 v/v dichloromethane-methanol (50 mL) was treated with sodium borohydride (28 mg, 0.74 mmol) at ambient temperature for 30 minutes. The reaction was quenched by addition of water. Extraction and purification by reverse phase afforded Compound 97 (46 mg) as a white solid. ¹H NMR (400 MHz, DMSO-d₆+TFA): δ/ppm=1.28 (m, 1H), 1.46 (m, 1H), 1.62 (m, 1H), 2.16 (m, 1H), 2.62 (m, 1H), 3.08 (m, 1H), 3.88 (m, 1H), 4.38 (m, 1H), 4.50 (m, 1H), 4.78-5.02 (m, 2H), 6.78-6.84 (m, 2H), 7.16 (m, 2H), 7.42 (m, 2H), 8.11 (br, 1H), 8.16 (s, 1H).
LRMS M+H, 461.1

### Example 98

### N-[5-chloro-2-[2-[4-[(4-fluorophenyl)hydroxymethyl]-1-piperidinyl]-2-oxoethoxy]phenyl]urea

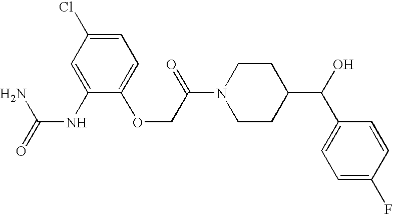 Prepared from Compound 27 in a similar manner to Compound 97. ¹H NMR (400 MHz, DMSO-d₆): δ/ppm=1.00 (m, 1H), 1.10-1.30 (m, 2H), 1.60-1.80 (m, 2H), 2.40 (m, 1H), 2.90 (m, 1H), 3.80 (m, 1H), 4.30 (m, 2H), 4.90 (m, 2H), 6.30 (br, 2H), 6.80 (m, 2H), 7.15 (m, 2H), 7.30 (m, 2H), 8.10 (br, 1H), 8.19 (d, 1H).
LRMS.M+H, 436.2

### Example 99

### 1-[[4-chloro-2-(hydroxymethyl)phenoxy]acetyl]-4-[(4-fluorophenyl)methyl]-4-piperidinecarbonitrile

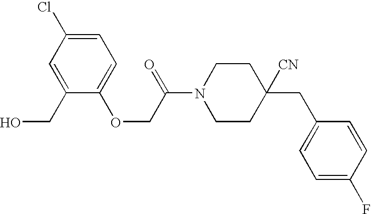 Prepared from Compound 25 in a similar manner to Compound 97. ¹H NMR (400 MHz, DMSO-d₆): δ/ppm=1.49-1.52 (m, 1H), 1.66-1.69 (m, 1H), 1.75 (br, 2H), 2.62-2.69 (m, 1H), 2.90 (s, 2H), 3.06-3.12 (m, 1H), 3.86 (d, 1H), 4.33 (d, 1H), 4.49 (dd, 2H), 4.83 (d, 1H), 4.90 (d, 1H), 5.18-5.22 (m, 1H), 6.80-6.89 (m, 1H), 7.15-7.19 (m, 3H), 7.29-7.33 (m, 3H).
LRMS M+H, 419.2

### Example 100

### 5-chloro-2-[2-[4-cyano-4-[(4-fluorophenyl)methyl]-1-piperidinyl]-2-oxoethoxy]benzoic acid

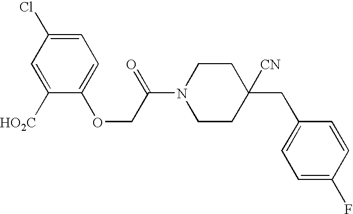 To a solution of Compound 22 (2.6 g, 5.8 mmol) in methanol (50 mL) at 0° C. was added dropwise a solution of sodium hydroxide (0.7 g, 18 mmol) in water (20 mL). The solution was warmed to ambient temperature and stirred for 4 hours, then diluted with water (30 mL) and concentrated to remove methanol. The resulting solution was cooled in an ice-water bath and acidified to pH 2 with 4 N hydrochloric acid. The resulting precipitate was collected by filtration, washed with water and dried to afford 2.2 g of solid. Purification by reverse phase HPLC afforded Compound 100. ¹H NMR (400 MHz, DMSO-d₆): δ/ppm=1.47 (t, 1H), 1.68 (t, 1H), 1.78, (m, 2H), 2.67 (t, 1H), 2.88 (s, 2H), 3.10 (t, 1H), 3.85 (d, 1H), 4.33 (d, 1H), 4.90 (d, 1H), 5.12 (d, 1H), 7.12 (m, 3H), 7.28 (m, 2H), 7.50 (dd, 1H), 7.61 (s, 1H).
LRMS M+H: 430

### Example 101

### 5-chloro-2-[2-[4-(4-fluorobenzoyl)-1-pi peridinyl]-2-oxoethoxy]-benzoic acid

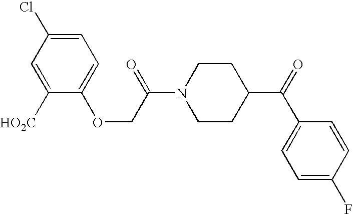 Prepared from Compound 68 in a similar manner to Compound 100. ¹H NMR (400 MHz, DMSO-d₆): δ/ppm=1.36 (q, 1H), 1.55 (q, 1H), 1.78 (d, 2H), 2.80 (t, 1H), 3.18 (t, 1H), 3.65 (m, 1H), 3.80 (d, 1H), 4.27 (d, 1H), 5.00 (d, 1H), 5.03 (d, 1H), 7.10 (d, 1H), 7.32 (t, 2H), 7.50 (dd, 1H), 7.58 (d, 1H), 8.04 (m, 2H).
LRMS M+H 419

### Example 102

### 2-[2-[4-amino-4-[(4-fluorophenyl)methyl]-1-piperidinyl]-2-oxoethoxy]-5-chlorobenzoic acid

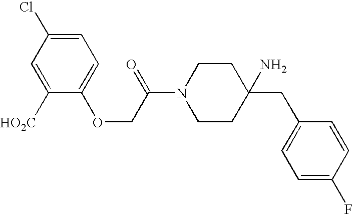 Prepared from Compound 26 in a similar manner to Compound 100. ¹H NMR (400 MHz, DMSO-d₆+TFA); δ/ppm=1.80-2.10 (m, 4H), 3.20 (s, 2H), 3.85 (m, 4H), 5.28 (m, 2H), 7.30-7.42 (m, 3H), 7.44-7.52 (m, 2H), 7.70 (m, 1H), 7.86 (m, 1H), 8.16 (br, 3H).
LRMS M+H, 421.1

### Example 103

### [[[1-[[2-[(aminocarbonyl)amino]-4-chlorophenoxy]acetyl]-4-[(4-fluorophenyl)methyl]-4-piperidinyl]methyl]amino]acetic acid

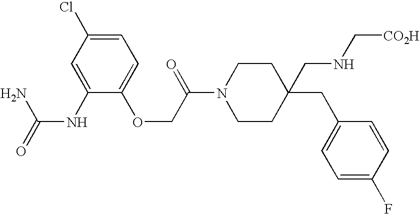 To a solution of Compound 45 (160 mg, 0.3 mmol) in 4:1 v/v tetrahydrofuran-water (5 mL) was added lithium hydroxide monohydrate (25 mg, 0.6 mmol) dissolved in water. The mixture was stirred at ambient temperature for 30 minutes, then concentrated, acidified with trifluoroacetic acid, and purified by reverse phase HPLC to afford Compound 103 (70 mg). ¹H NMR (400 MHz, DMSO-d₆): δ/ppm=1.22 (m, 1H), 1.36 (m, 1H), 1.50 (m, 2H), 2.80 (s, 2H), 3.02 (s, 2H), 3.20 (m, 2H), 3.58 (m, 1H), 3.66 (m, 1H), 3.94 (m, 1H), 4.88 (m, 2H), 6.36 (s, 1H), 6.74 (m, 2H), 7.20 (m, 4H), 8.10 (s, 1H), 8.20 (s, 1H).
LRMS M+H: 507

### Example 104

### [[1-[[2-[(aminocarbonyl)amino]-4-chlorophenoxy]acetyl]-4-[(4-fluorophenyl)methyl]-4-piperidinyl]amino]acetic acid

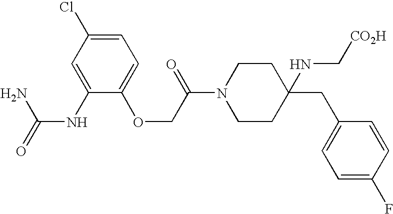 Prepared from Compound 43 in a similar manner to Compound 103. ¹H NMR (400 MHz, DMSO-d₆): δ/ppm=1.76 (m, 3H), 1.90 (m, 1H), 3.18 (m, 2H), 3.36 (m, 1H), 3.50 (m, 1H), 3.70 (m, 1H), 3.90 (m, 3H), 4.92 (s, 2H), 6.80 (m, 2H), 7.24 (m, 4H), 8.10 (s, 1H), 8.18 (s, 1H).
LRMS M+H, 493.1

### Example 105

### 5-bromo-2-[2-[4-cyano-4-[(4-fluorophenyl)methyl]-1-piperidinyl]-2-oxoethoxy]benzeneacetic acid

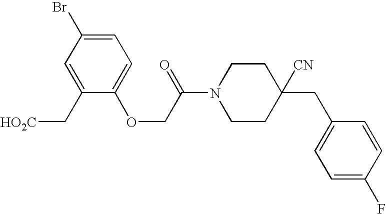 Prepared from Compound 85 in a similar manner to Compound 103. ¹H NMR (400 MHz, CDCl₃): δ/ppm=1.50 (m, 2H), 1.90 (m, 2H), 2.84 (s, 2H), 2.90 (m, 1H), 3.35 (t, 1H), 3.65 (s, 2H), 3.92 (m, 1H), 4.60-4.75 (m, 3H), 6.78 (d, 1H), 7.05 (t, 2H), 7.25 (m, 2H), 7.38 (m, 2H).
LRMS M+H: 489

### Example 106

### N-[2-[2-[4-(aminomethyl)-4-[(4-fluorophenyl)methyl]-1-piperidinyl]-2-oxoethoxy]-5-chlorophenyl]urea

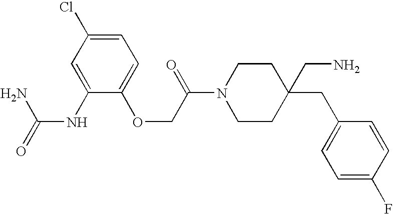 To a solution of Compound 33 (163 mg, 0.3 mmol) in methanol (15 mL) was added potassium carbonate (81 mg, 0.6 mmol) and the mixture heated at 50° C. overnight. Extraction and purification by reverse phase HPLC afforded Compound 106 (77 mg). ¹H NMR (400 MHz, DMSO-d₆): δ/ppm=1.42 (m, 4H), 2.76 (m, 4H), 3.12 (m, 1H), 3.30 (m, 1H), 3.64 (m, 2H), 4.88 (m, 2H), 6.34 (s, 2H), 6.80 (m, 2H), 7.18 (m, 4H), 7.90 (m, 2H), 8.10 (s, 1H), 8.16 (s, 1H).

### Example 107

### 5-chloro-2-[2-[4-(4-fluorobenzoyl)-1-pi peridinyl]-2-oxoethoxy]-N-[2-(2-hydroxyethoxy)ethyl]benzamide

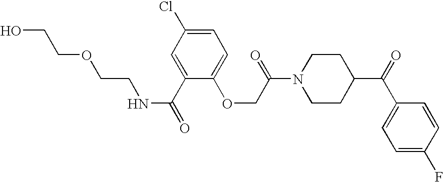 To a solution of Compound 101 (200 mg, 0.48 mmol) in dimethylformamide (20 mL) was added 2-(2-aminoethoxy)ethanol (50 mg, 0.48 mmol), HATU (180 mg, 0.48 mmol) and Hunig's base (0.4 mL, 2.4 mmol). The mixture was stirred at ambient temperature overnight, then diluted with water. The resulting solid was collected by filtration, dissolved in ethyl acetate, dried and concentrated. The product was purified by crystallization to afford Compound 107 (260 mg) as a white solid. ¹H NMR (400 MHz, DMSO-d₆): δ/ppm=1.40 (q, 1H), 1.60 (q, 1H), 1.81 (d, 2H), 2.85 (t, 1H), 3.20 (t, 1H), 3.44 (m, 6H), 3.55 (t, 2H), 3.72 (m, 1H), 3.82 (d, 1H), 4.37 (d, 1H), 4.54 (t, 1H), 5.09 (q, 2H), 7.25 (d, 1H), 7.35 (t, 2H), 7.52 (dd, 1H), 7.82 (d, 1H), 8.09 (m, 2H), 9.29 (t, 1H).

### Example 108

### 5-chloro-N-(cyanomethyl)-2-[2-[4-(4-fluorobenzoyl)-1-piperidinyl]-2-oxoethoxy]benzamide

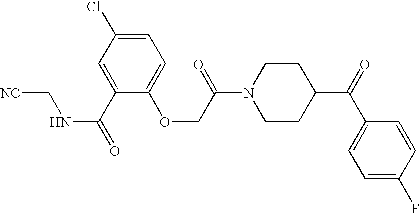 Prepared in a similar manner to Compound 107. ¹H NMR (400 MHz, DMSO-d₆): δ/ppm=1.40 (m, 1H), 1.61 (m, 1H), 1.82 (d, 2H), 2.87 (t, 1H), 3.21 (t, 1H), 3.73 (m, 1H), 3.81 (d, 1H), 4.36 (m, 3H), 5.14 (q, 2H), 7.27 (d, 1H), 7.36 (t, 2H), 7.59 (dd, 1H), 7.84 (d, 1H), 8.09 (m, 2H), 9.29 (t, 1H).

### Example 109

### 5-chloro-2-[2-[4-cyano-4-[(4-fluorophenyl)methyl]-1-piperidinyl]-2-oxoethoxy]-N-(1-methyl-4-piperidinyl)benzamide

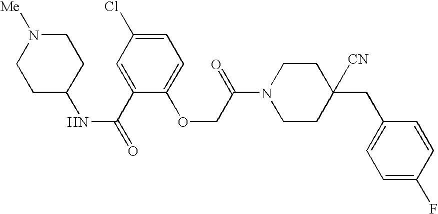 Prepared in a similar manner to Compound 107. ¹H NMR (400 MHz, DMSO-d₆): δ/ppm=1.50 (t, 1H), 1.78-2.02 (m, 6H), 2.75 (m, 4H), 2.90 (m, 2H), 3.08 (m, 2H), 3.30 (m, 1H), 3.45 (d, 2H), 3.86 (m, 1H), 4.04 (m, 1H), 4.42 (m, 1H), 4.97 (d, 1H), 5.13 (d, 1H), 7.14 (m, 2H), 7.30 (m, 3H), 7.53 (m, 1H), 7.86 (m, 1H), 9.12-9.40 (m, 2H).
LRMS M+H: 526

### Example 110

### 5-chloro-2-[2-[4-cyano-4-[(4-fluorophenyl)methyl]-1-piperidinyl]-2-oxoethoxy]-N-[2-(1-pyrrolidinyl)ethyl]benzamide

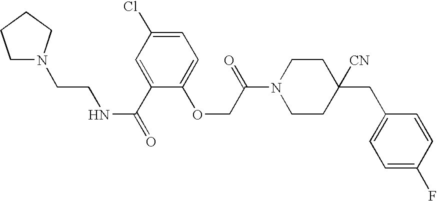 Prepared in a similar manner to Compound 107. ¹H NMR (400 MHz, DMSO-d₆): δ/ppm=1.51 (t, 1H), 1.68 (t, 1H), 1.82 (m, 4H), 1.97 (m, 2H), 2.75 (t, 1H), 2.88 (s, 2H), 3.08 (m, 3H), 3.38 (m, 2H), 3.63 (m, 4H), 3.85 (d, 1H), 4.40 (d, 1H), 5.02 (d, 1H), 5.13 (d, 1H), 7.13 (m, 2H), 7.28 (m, 3H), 7.57 (d, 1H), 7.87 (s, 1H), 9.38 (br, 1H), 9.63 (t, 1H).
LRMS M+H: 526

### Example 111

### 5-chloro-2-[2-[4-cyano-4-[(4-fluorophenyl)methyl]-1-piperidinyl]-2-oxoethoxy]-N-[2-hydroxy-1-(hydroxymethyl)ethyl]benzamide

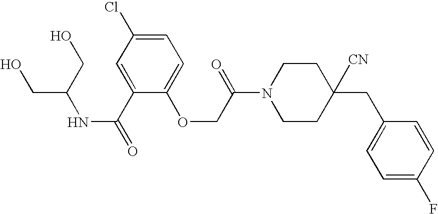 Prepared in a similar manner to Compound 107. ¹H NMR (400 MHz, DMSO-d₆): δ/ppm=1.48 (t, 1H), 1.76 (m, 3H), 2.70 (t, 1H), 2.86 (s, 2H), 3.08 (t, 1H), 3.50 (m, 4H), 3.85 (d, 1H), 3.98 (m, 1H), 4.40 (m, 1H), 4.98 (m, 1H), 5.08 (d, 1H), 7.15 (m, 2H), 7.24 (m, 1H), 7.30 (m, 2H), 7.48 (m, 1H), 7.85 (t, 1H), 8.87 (m, 1H).
LRMS M+H: 503

### Example 112

### 5-chloro-2-[2-[4-cyano-4-[(4-fluorophenyl)methyl]-1-piperidinyl]-2-oxoethoxy]-N-[2-(2-hydroxyethoxy)ethyl]benzamide

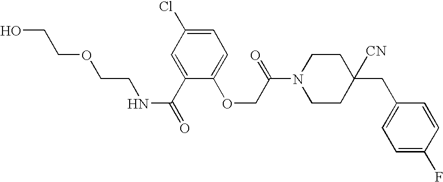 Prepared in a similar manner to Compound 107. ¹H NMR (400 MHz, DMSO-d₆): δ/ppm=1.59 (t, 1H), 1.78 (t, 1H), 1.83 (m, 2H), 2.75 (t, 1H), 2.84 (s, 2H), 3.14 (t, 1H), 3.47 (m, 6H), 3.60 (m, 2H), 3.88 (d, 1H), 4.44 (d, 1H), 5.08 (d, 1H), 5.15 (d, 1H), 7.20 (m, 2H), 7.28 (d, 1H), 7.34 (m, 2H), 7.52 (d, 1H), 7.83 (s, 1H), 9.28 (t, 1H).
LRMS M+H: 517

### Example 113

### 5-chloro-2-[2-[4-cyano-4-[(4-fluorophenyl)methyl]-1-piperidinyl]-2-oxoethoxy]-N-(1,2,2,6,6-pentamethyl-4-piperidinyl)benzamide

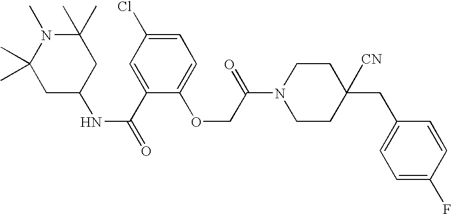 Prepared in a similar manner to Compound 107. ¹H NMR (400 MHz, DMSO-d₆): δ/ppm=1.44 (s, 12H), 1.60 (t, 1H), 1.76 (t, 1H), 1.85 (m, 4H), 2.14 (m, 2H), 2.78 (m, 4H), 2.95 (s, 2H), 3.15 (t, 1H), 3.92 (d, 1H), 4.47 (m, 1H), 4.53 (d, 1H), 5.08 (d, 1H), 5.18 (d, 1H), 7.20 (t, 2H), 7.35 (m, 3H), 7.59 (d, 1H), 7.87 (s, 1H), 8.67 (br, 1H), 9.41 (d, 1H).
LRMS M+H: 582

### Example 114

### 5-chloro-2-[2-[4-cyano-4-[(4-fluorophenyl)methyl]-1-piperidinyl]-2-oxoethoxy]-N-(6-methoxy-3-pyridinyl)benzamide

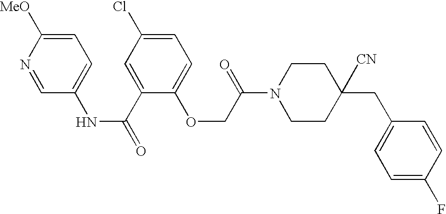 Prepared in a similar manner to Compound 107. ¹H NMR (400 MHz, DMSO-d₆): δ/ppm=1.60 (t, 1H), 1.78 (t, 1H), 1.85 (m, 2H), 2.80 (t, 1H), 2.94 (s, 2H), 3.17 (t, 1H), 3.88 (s, 3H), 3.92 (d, 1H), 4.53 (d, 1H), 5.15 (d, 1H), 5.25 (d, 1H), 6.93 (d, 1H), 7.20 (t, 2H), 7.35 (m, 2H), 7.40 (d, 1H), 7.63 (d, 1H), 7.98 (s, 1H), 8.30 (d, 1H), 8.74 (s, 1H), 11.30 (s, 1H).
LRMS M+H: 536

### Example 115

[[5-chloro-2-[2-[4-cyano-4-[(4-fluorophenyl)methyl]-1-piperidinyl]-2-oxoethoxy]benzoyl]amino]acetic acid 1,1-dimethylethyl ester 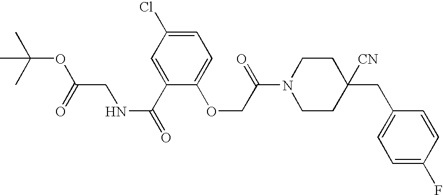
Prepared in a similar manner to Compound 107. ¹H NMR (400 MHz, DMSO-d₆): δ/ppm=1.42 (s, 9H), 1.55 (t, 1H), 1.76 (t, 1H), 1.83 (m, 2H), 2.75 (t, 1H), 2.92 (s, 2H), 3.12 (t, 1H), 3.90 (m, 3H), 4.42 (d, 1H), 5.08 (d, 1H), 5.15 (d, 1H), 7.20 (t, 2H), 7.28 (d, 1H), 7.32 (m, 2H), 7.60 (d, 1H), 7.85 (s, 1H), 9.55 (t, 1H).
LRMS M+H: 543

### Example 116

### 4-[[5-chloro-2-[2-[4-cyano-4-[(4-fluorophenyl)methyl]-1-piperidinyl]-2-oxoethoxy]benzoyl]amino]-1-piperidinecarboxylic acid 1,1-dimethylethyl ester

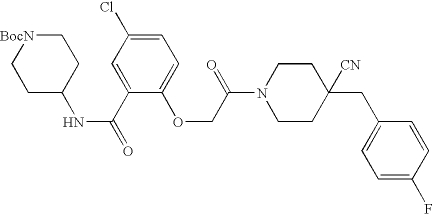 Prepared in a similar manner to Compound 107. ¹H NMR (400 MHz, DMSO-d₆): δ/ppm=0.84 (m, 2H), 1.26 (m, 2H), 1.42 (s, 9H), 1.56 (m, 2H), 1.78 (m, 4H), 2.82 (m, 5H), 3.08 (m, 1H), 3.88 (m, 2H), 3.98 (m, 1H), 4.42 (m, 1H), 5.04 (m, 2H), 7.20 (m, 2H), 7.34 (m, 3H), 7.58 (m, 1H), 7.90 (s, 1H), 9.28 (s, 1H).

### Example 117

### 5-chloro-2-[2-[4-cyano-4-[(4-fluorophenyl)methyl]-1-piperidinyl]-2-oxoethoxy]-N-(4-piperidinyl)benzamide

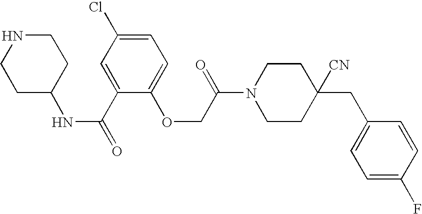 Prepared by treatment of Compound 116 (281 mg, 0.46 mmol) in dichloromethane (1 mL) with hydrochloric acid (4.0 M in dioxane, 1.0 mL, 4.0 mmol) at ambient temperature for 2 hours. ¹H NMR (400 MHz, DMSO-d₆): δ/ppm=1.20 (m, 1H), 1.48 (m, 3H), 1.76 (m, 5H), 2.72 (m, 1H), 2.92 (m, 4H), 3.10 (m, 1H), 3.86 (m, 2H), 4.46 (m, 1H), 5.12 (m, 2H), 7.20 (m, 2H), 7.32 (m, 3H), 7.56 (d, 1H), 7.86 (s, 1H), 9.16 (d, 1H).
LRMS M+H, 513.2

### Example 118

### [[5-chloro-2-[2-[4-(4-fluorobenzoyl)-1-piperidinyl]-2-oxoethoxy]benzoyl]amino]acetic acid

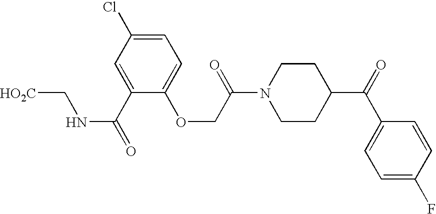 Prepared from Compound 101 and glycine ethyl ester hydrochloride in a similar manner to Compound 107, followed by hydrolysis of the ethyl ester and purification by reverse phase HPLC. ¹H NMR (400 MHz, DMSO-d₆): δ/ppm=1.40 (m, 1H), 1.60 (m, 1H), 1.82 (m, 2H), 2.04 (s, 2H), 2.86 (m, 1H), 3.20 (m, 2H), 3.72 (m, 1H), 3.80 (m, 1H), 3.94 (m, 2H), 4.36 (m, 1H), 5.10 (m, 2H), 7.24 (m, 2H), 7.36 (m, 2H), 7.56 (m, 2H), 7.88 (s, 1H), 8.10 (m, 2H), 9.66 (m, 1H).
LRMS M+H, 450.2

### Example 119

### [[2-[2-[4-amino-4-[(4-fluorophenyl)methyl]-1-piperidinyl]-2-oxoethoxy]-5-chlorobenzoyl]amino]acetic acid

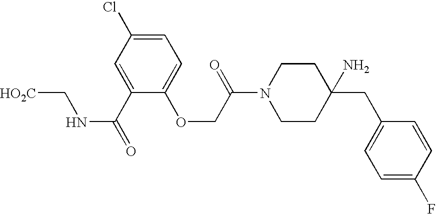 Prepared from Compound 102 and glycine tert-butyl ester hydrochloride in a similar manner to Compound 107, followed by hydrolysis of the ester and purification by reverse phase HPLC. ¹H NMR (400 MHz, DMSO-d₆+TFA): δ/ppm=1.55-1.85 (m, 4H), 2.96 (s, 2H), 3.60 (m, 4H), 3.95 (d, 2H), 5.10 (m, 2H), 7.10-7.28 (m, 5H), 7.52 (dd, 1H), 7.84 (d, 1H), 7.94 (br, 3H), 9.52 (t, 1H), 8.18 (m, 1H).
LRMS M+H, 478.1

### Example 120

### [[5-chloro-2-[2-[4-cyano-4-[(4-fluorophenyl)methyl]-1-piperidinyl]-2-oxoethoxy]benzoyl]amino]acetic acid

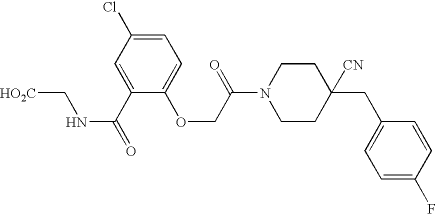 Prepared by hydrolysis of Compound 115 with trifluoroacetic acid. ¹H NMR (400 MHz, DMSO-d₆): δ/ppm=1.58 (t, 1H), 1.76 (t, 1H), 1.83 (m, 2H), 2.75 (t, 1H), 2.95 (s, 2H), 3.15 (t, 1H), 3.92 (d, 1H), 4.00 (d, 2H), 4.43 (d, 1H), 5.08 (d, 1H), 5.18 (d, 1H), 7.19 (t, 2H), 7.28 (d, 1H), 7.32 (m, 2H), 7.59 (d, 1H), 7.87 (s, 1H), 9.58 (t, 1H).
LRMS M+H: 487

### Example 121

### [[[5-chloro-2-[2-[4-cyano-4-[(4-fluorophenyl)methyl]-1-piperidinyl]-2-oxoethoxy]phenyl]methyl]amino]acetic acid

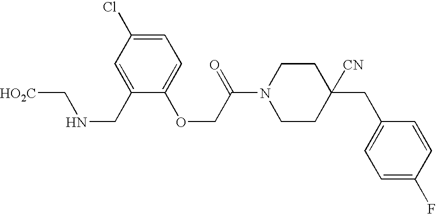 Prepared from Compound 169 in a similar manner to Compound 120. ¹H NMR (400 MHz, DMSO-d₆): δ/ppm=1.47-1.51 (m, 1H), 1.67-1.70 (m, 1H), 1.78-1.85 (m, 2H), 2.68 (t, 1H), 2.92 (s, 2H), 3.12 (t, 1H), 3.85 (br, 2H), 4.19 (s, 2H), 4.29 (d, 1H), 5.03 (d, 1H), 5.14 (d, 1H), 7.15-7.20 (m, 3H), 7.30 (m, 2H), 7.43-7.48 (m, 2H), 9.26 (br, 2H).
LRMS M+H, 474.1

### Example 122

### 5-chloro-2-[2-[4-(4-fluorobenzoyl)-1-piperidinyl]-2-oxoethoxy]-N-(4-piperidinyl)benzamide

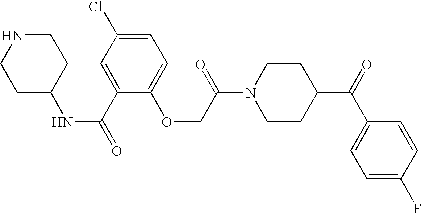

### A. 4-[[5-chloro-2-[2-[4-(4-fluorobenzoyl)-1-piperidinyl]-2-oxoethoxy]benzoyl]amino]-1-piperidinecarboxylic acid 1,1-dimethylethyl ester

To a solution of Compound 101 (126 mg, 0.3 mmol) in dimethylformamide (5 mL) at 0° C. was added triethylamine (120 mg, 1.2 mmol), followed after 5 minutes by 1-tert-butoxycarbonyl-4-aminopiperidine (60 mg, 0.3 mmol) and HATU (140 mg, 0.36 mmol). The mixture was stirred at ambient temperature overnight. The mixture was poured onto ice water and the resulting solid was collected by filtration. Purification by chromatography on silica afforded Intermediate 122a (172 mg).

### B. 5-chloro-2-[2-[4-(4-fluorobenzoyl)-1-pi peridinyl]-2-oxoethoxy]-N-(4-piperidinyl)benzamide

To a solution of Intermediate 122a (120 mg, 0.2 mmol) in dichloromethane (1 mL) at 0° C. was added trifluoroacetic acid (0.5 mL) and the mixture stirred for 15 minutes. The mixture was concentrated to dryness, ether added and concentration repeated to afford Compound 122 (131 mg). ¹H NMR (400 MHz, DMSO-d₆): δ/ppm=1.40 (m, 1H), 1.62 (m, 1H), 1.80 (m, 4H), 2.00 (m, 2H), 2.90 (m, 1H), 3.04 (m, 2H), 3.30 (m, 3H), 3.74 (m, 1H), 3.88 (m, 1H), 4.10 (m, 1H), 4.44 (m, 1H), 5.10 (m, 2H), 7.36 (m, 3H), 7.58 (d, 1H), 7.88 (s, 1H), 8.10 (m, 2H), 8.30 (m, 1H), 8.50 (m, 1H), 9.40 (d, 1H).

### Example 123

### 5-chloro-2-[2-[4-(4-fluorobenzoyl)-1-pi peridinyl]-2-oxoethoxy]-N-(4-piperidinylmethyl)benzamide

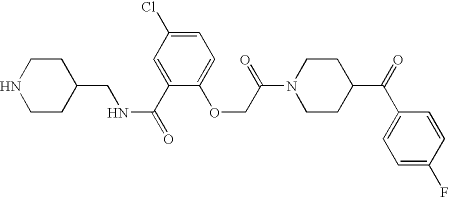 Prepared in a similar manner to compound 122. ¹H NMR (400 MHz, DMSO-d₆): δ/ppm=1.36 (m, 4H), 1.60 (m, 1H), 1.84 (m, 5H), 2.82 (m, 2H), 3.26 (m, 4H), 3.40 (m, 1H), 3.80 (m, 1H), 4.38 (m, 1H), 5.08 (m, 2H), 7.36 (m, 3H), 7.54 (m, 1H), 7.80 (m, 1H), 8.10 (m, 2H), 8.40 (m, 1H), 9.42 (m, 1H).

### Example 124

### [1-[[2-[(4-amino-1-piperidinyl)carbonyl]-4-chlorophenoxy]acetyl]-4-piperidinyl](4-fluorophenyl)methanone

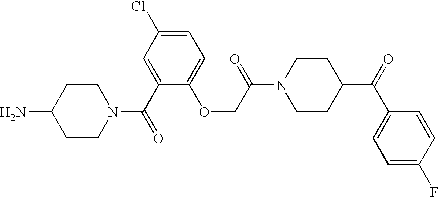 Prepared in a similar manner to Compound 122. ¹H NMR (400 MHz, DMSO-d₆): δ/ppm=1.44 (m, 4H), 1.76 (m, 3H), 1.86 (m, 1H), 2.78 (m, 2H), 3.12 (m, 3H), 3.54-3.84 (m, 7H), 4.30 (m, 1H), 4.48 (m, 1H), 4.96 (m, 2H), 6.90 (m, 1H), 7.16 (m, 1H), 7.36 (m, 3H), 7.84 (m, 2H), 8.08 (m, 2H).

### Example 125

### N-(3-aminopropyl)-5-chloro-2-[2-[4-cyano-4-[(4-fluorophenyl)methyl]-1-piperidinyl]-2-oxoethoxy]benzamide

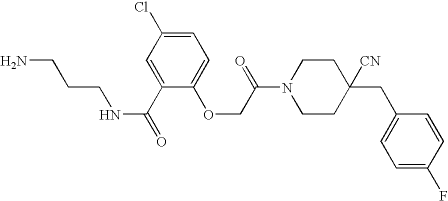 Prepared in a similar manner to Compound 122. ¹H NMR (400 MHz, DMSO-d₆): δ/ppm=1.55 (t, 1H), 1.75 (t, 1H), 1.84, (m, 4H), 2.79 (t, 1H), 2.88 (m, 2H), 2.93 (s, 2H), 3.14 (t, 1H), 3.23 (m, 2H), 3.92 (d, 1H), 4.26 (d, 1H), 5.03 (d, 1H), 5.17 (d, 1H), 7.20 (t, 2H), 7.35 (m, 3H), 7.58 (d, 1H), 7.73 (br, 3H), 7.68 (s, 1H), 9.27 (t, 1H).
LRMS M+H: 486

### Example 126

### 5-chloro-2-[2-[4-cyano-4-[(4-fluorophenyl)methyl]-1-piperidinyl]-2-oxoethoxy]-N-(3-piperidinylmethyl)benzamide

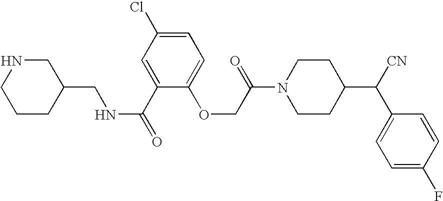 Prepared in a similar manner to Compound 122. ¹H NMR (400 MHz, DMSO-d₆): δ/ppm=1.37 (q, 2H), 1.55 (t, 1H), 1.76 (t, 1H), 1.83 (m, 5H), 2.78 (t, 1H), 2.86 (q, 2H), 2.95 (s, 2H), 3.14 (t, 1H), 3.27 (m, 4H), 3.88 (d, 1H), 4.44 (d, 1H), 5.08 (d, 1H), 5.15 (d, 1H), 7.20 (t, 2H), 7.24 (m, 3H), 7.57 (d, 1H), 7.87 (s, 1H), 8.18 (br, 1H), 8.51 (br, 1H), 9.44 (t, 1H).
LRMS M+H: 526

### Example 127

### N-(3-aminopropyl)-5-chloro-2-[2-[4-(4-fluorobenzoyl)-1-piperidinyl]-2-oxoethoxy]benzamide

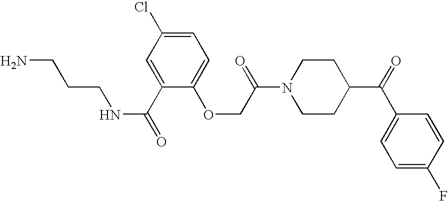 To a solution of Compound 101 (210 mg, 0.5 mmol) and triethylamine (0.3 mL, 2.0 mmol) in dichloromethane (3 mL) at −30° C. was added isobutyl chloroformate (70 mg, 0.5 mmol), followed after 10 minutes by 1-tert-butoxycarbonyl-1,3-propanediamine (90 mg, 0.6 mmol). The mixture was stirred for 2 hours, then purified directly by chromatography on silica. The product was dissolved in dichloromethane (4 mL) and trifluoroacetic acid (2 mL) was added and the mixture stirred for 1 hour. The mixture was concentrated to dryness, water added and the solution frozen and lyophilized to afford Compound 127. ¹H NMR (400 MHz, DMSO-d₆): δ/ppm=1.40 (q, 1H), 1.60 (q, 1H), 1.81 (m, 4H), 2.83 (m, 3H), 3.20 (t, 1H), 3.38 (m, 2H), 3.75 (m, 1H), 3.82 (d, 1H), 4.38 (d, 1H), 5.03 (d, 1H), 5.11 (d, 1H), 7.28 (d, 1H), 7.38 (m, 2H), 7.55 (d, 1H), 7.65 (br, 2H), 7.82 (s, 1H), 8.09 (dd, 2H), 9.48 (t, 1H).
LRMS M+H: 475

### Example 128

### N-(2-aminoethyl)-5-chloro-2-[2-[4-(fluorobenzoyl)-1-piperidinyl]-2-oxoethoxy]benzamide

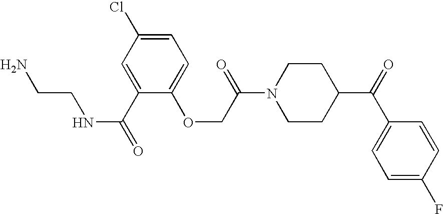 Prepared in a similar manner to Compound 127. ¹H NMR (400 MHz, DMSO-d₆): δ/ppm=1.40 (q, 1H), 1.60 (q, 1H), 1.81 (m, 2H), 2.86 (m, 1H), 3.00 (m, 2H), 3.20 (t, 1H), 3.58 (m, 2H), 3.75 (m, 1H), 3.82 (d, 1H), 4.38 (d, 1H), 5.03 (d, 1H), 5.11 (d, 1H), 7.28 (d, 1H), 7.38 (m, 2H), 7.59 (d, 1H), 7.75 (br, 2H), 7.82 (s, 1H), 8.09 (dd, 2H), 9.60 (t, 1H).
LRMS M+H: 461

### Example 129

### 5-chloro-2-[2-[4-(4-fluorobenzoyl)-1-piperidinyl]-2-oxoethoxy]-N-(3-piperidinyl)benzamide

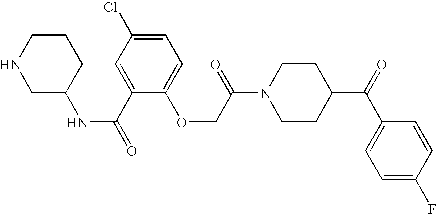 Prepared in a similar manner to Compound 127. ¹H NMR (400 MHz, DMSO-d₆): δ/ppm=1.20 (m, 1H), 1.62 (m, 3H), 1.85 (m, 4H), 2.83 (m, 3H), 3.20 (m, 2H), 3.38 (d, 1H), 3.73 (t, 1H), 3.81 (d, 1H), 4.18 (m, 1H), 4.40 (d, 1H), 5.00 (d, 1H), 5.14 (d, 1H), 7.30 (d, 1H), 7.36 (t, 2H), 7.56 (dd, 1H), 7.83 (s, 1H), 8.09 (dd, 2H), 8.70 (m, 2H), 9.43 (t, 1H).
LRMS M+H: 501

### Example 130

### 5-chloro-2-[2-[4-(4-fluorobenzoyl)-1-pi peridinyl]-2-oxoethoxy]-N-(3-piperidinylmethyl)benzamide

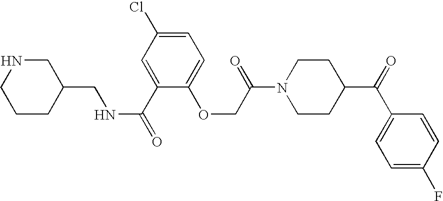 Prepared in a similar manner to Compound 127. ¹H NMR (400 MHz, DMSO-d₆): δ/ppm=1.34 (m, 3H), 1.60 (q, 1H), 1.82 (m, 5H), 2.83 (m, 3H), 3.22 (m, 5H), 3.72 (t, 1H), 3.82 (d, 1H), 4.35 (d, 1H), 5.07 (d, 1H), 5.14 (d, 1H), 7.28 (d, 1H), 7.36 (t, 2H), 7.56 (d, 1H), 7.80 (s, 1H), 8.07 (m, 2H), 8.20 (m, 1H), 8.55 (m, 1H), 9.43 (t, 1H).
LRMS M+H: 515

### Example 131

### 5-chloro-2-[2-[4-(4-fluorobenzoyl)-1-piperidinyl]-2-oxoethoxy]-N-[2-(4-morpholinyl)ethyl]benzamide

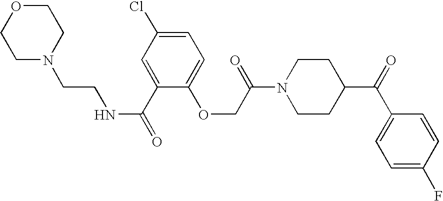 To a solution of Compound 101 (210 mg, 0.5 mmol) and triethylamine (0.3 mL, 2.0 mmol) in dichloromethane (3 mL) at −30° C. was added isobutyl chloroformate (70 mg, 0.5 mmol), followed after 10 minutes by 4-(2-aminoethyl)morpholine (150 mg, 1.0 mmol). The mixture was stirred for 2 hours, then purified directly by chromatography on silica to afford Compound 131. ¹H NMR (400 MHz, DMSO-d₆): δ/ppm=1.38 (q, 1H), 1.60 (q, 1H), 1.81 (d, 2H), 2.38 (s, 4H), 2.82 (t, 1H), 3.28 (d, 3H), 3.40 (m, 2H), 3.50 (m, 4H), 3.70 (m, 1H), 3.80 (d, 1H), 4.37 (d, 1H), 5.03 (d, 1H), 5.11 (d, 1H), 7.22 (d, 1H), 7.35 (m, 2H), 7.50 (d, 1H), 7.80 (s, 1H), 8.09 (dd, 2H), 9.24 (t, 1H).
LRMS M+H: 531

### Example 132

### 5-chloro-2-[2-[4-(4-fluorobenzoyl)-1-piperidinyl]-2-oxoethoxy]-N-(1-methyl-4-piperidinyl)benzamide

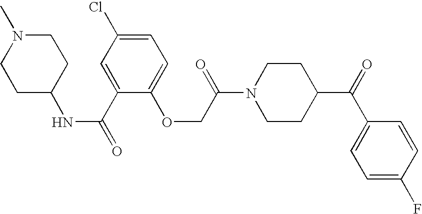 Prepared in a similar manner to Compound 131. ¹H NMR (400 MHz, DMSO-d₆): δ/ppm=1.16 (t, 2H), 1.40 (q, 1H), 1.62 (m, 1H), 1.70 (m, 3H), 1.83 (m, 4H), 2.40 (s, 3H), 2.85 (t, 1H), 2.96 (m, 3H), 3.22 (m, 1H), 3.75 (m, 1H), 3.84 (m, 2H), 4.40 (d, 1H), 5.00 (d, 1H), 5.14 (d, 1H), 7.30 (d, 1H), 7.36 (t, 2H), 7.56 (d, 1H), 7.83 (s, 1H), 8.09 (d, 2H), 9.26 (t, 1H).
LRMS M+H: 515

### Example 133

### 5-chloro-2-[2-[4-(4-fluorobenzoyl)-1-piperidinyl]-2-oxoethoxy]-N-hydroxybenzamide

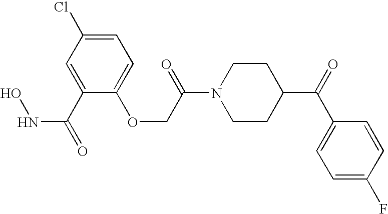 Prepared in a similar manner to Compound 131. ¹H NMR (400 MHz, DMSO-d₆): δ/ppm=1.40 (m, 1H), 1.60 (m, 1H), 1.80 (d, 2H), 2.85 (t, 1H), 3.21 (d, 1H), 3.77 (t, 1H), 3.82 (d, 1H), 4.38 (d, 1H), 5.06 (d, 1H), 5.12 (d, 1H), 7.24 (d, 1H), 7.38 (m, 2H), 7.52 (d, 1H), 7.82 (s, 1H), 8.09 (m, 2H), 9.22 (s, 1H), 11.68 (s, 1H).
LRMS M+H: 434

### Example 134

### 5-chloro-2-[2-[4-(4-fluorobenzoyl)-1-pi peridinyl]-2-oxoethoxy]-N-(2-hydroxyethyl)benzamide

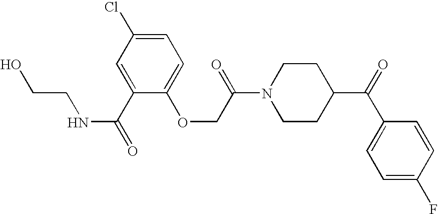 Prepared in a similar manner to Compound 131. ¹H NMR (400 MHz, DMSO-d₆): δ/ppm=1.40 (m, 1H), 1.60 (m, 1H), 1.80 (d, 2H), 2.85 (t, 1H), 3.21 (d, 1H), 3.38 (m, 2H), 3.50 (m, 2H), 3.74 (m, 1H), 3.82 (d, 1H), 4.38 (d, 1H), 4.67 (t, 1H), 5.05 (d, 1H), 5.11 (d, 1H), 7.24 (d, 1H), 7.38 (m, 2H), 7.52 (d, 1H), 7.82 (s, 1H), 8.09 (m, 2H), 9.22 (t, 1H).
LRMS M+H: 462

### Example 135

### 5-chloro-2-[2-[4-(4-fluorobenzoyl)-1-piperidinyl]-2-oxoethoxy]-N-(2-hydroxyethyl)-N-methylbenzamide

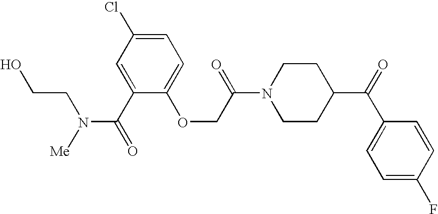 Prepared in a similar manner to Compound 131. ¹H NMR (400 MHz, DMSO-d₆): δ/ppm=1.38 (m, 1H), 1.60 (m, 1H), 1.80 (d, 2H), 2.80 (m, 1H), 2.82 (s, 1.5H, amide rotamer), 2.92 (s, 1.5H, amide rotamer), 3.02 (m, 1H), 3.19 (t, 1H), 3.38 (m, 2H), 3.53 (m, 1H), 3.71 (t, 1H), 3.82 (d, 1H), 4.28 (d, 1H), 4.90 (m, 2H), 6.92 (dd, 1H), 7.19 (dd, 1H), 7.36 (m, 3H), 8.09 (dd, 2H).
LRMS M+H: 476

### Example 136

### 5-chloro-2-[2-[4-(4-fluorobenzoyl)-1-piperidinyl]-2-oxoethoxy]-N-[2-hydroxy-1-(hydroxymethyl)ethyl]benzamide

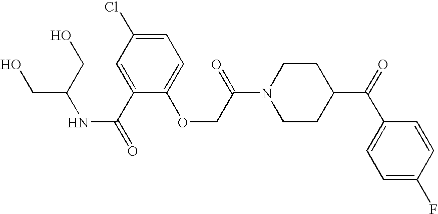 Prepared in a similar manner to Compound 131. ¹H NMR (400 MHz, DMSO-d₆): δ/ppm=1.39 (q, 1H), 1.60 (q, 1H), 1.80 (d, 2H), 2.84 (t, 1H), 3.23 (t, 1H), 3.28 (d, 3H), 3.71 (m, 1H), 3.82 (d, 1H), 3.95 (m, 1H), 4.35 (d, 1H), 4.62 (t, 1H), 5.00 (d, 1H), 5.14 (d, 1H), 7.23 (d, 1H), 7.34 (m, 2H), 7.52 (d, 1H), 7.84 (s, 1H), 8.09 (dd, 2H), 8.91 (d, 1H).
LRMS M+H: 492

### Example 137

### 5-chloro-2-[2-[4-(4-fluorobenzoyl)-1-piperidinyl]-2-oxoethoxy]-N-[2-(1H-imidazol-4-yl)ethyl]benzamide

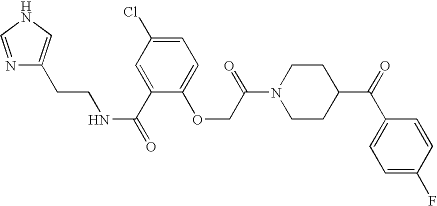 Prepared in a similar manner to Compound 131. ¹H NMR (400 MHz, DMSO-d₆): δ/ppm=1.40 (q, 1H), 1.60 (q, 1H), 1.80 (d, 2H), 2.78 (br, 1H), 2.81 (t, 1H), 3.21 (t, 1H), 3.28 (d, 3H), 3.50 (m, 2H), 3.71 (m, 1H), 3.82 (d, 1H), 4.19 (d, 1H), 5.00 (d, 1H), 5.14 (d, 1H), 7.23 (d, 1H), 7.37 (m, 2H), 7.52 (m, 1H), 7.80 (s, 1H), 8.09 (t, 2H), 9.38 (t, 1H).
LRMS M+H: 512

### Example 138

### 5-chloro-2-[2-[4-(4-fluorobenzoyl)-1-piperidinyl]-2-oxoethoxy]benzoic acid hydrazide

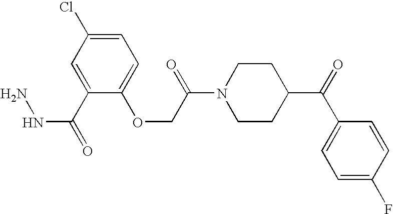 Prepared in a similar manner to Compound 131. ¹H NMR (400 MHz, DMSO-d₆): δ/ppm=1.40 (q, 1H), 1.60 (q, 1H), 1.72 (m, 2H), 1.80 (d, 2H), 2.88 (t, 1H), 3.21 (t, 1H), 3.55 (m, 2H), 3.78 (m, 2H), 4.38 (d, 1H), 5.12 (d, 1H), 5.24 (d, 1H), 7.33 (t, 2H), 7.63 (d, 1H), 7.82 (s, 1H), 8.09 (t, 2H), 11.78 (s, 1H).
LRMS M+H: 433

### Example 139

### 5-chloro-N-[2-(dimethylamino)ethyl]-2-[2-[4-(4-fluorobenzoyl)-1-piperidinyl]-2-oxoethoxy]benzamide

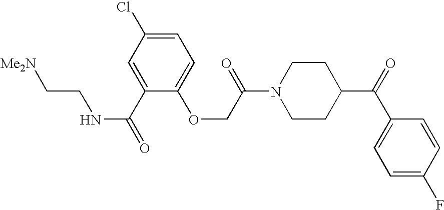 Prepared in a similar manner to Compound 131. ¹H NMR (400 MHz, DMSO-d₆): δ/ppm=1.40 (q, 1H), 1.60 (q, 1H), 1.80 (d, 2H), 2.81 (s, 6H), 2.82 (t, 1H), 3.21 (t, 1H), 3.32 (m, 2H), 3.65 (m, 2H), 3.73 (m, 1H), 3.83 (d, 1H), 4.38 (d, 1H), 5.14 (dd, 2H), 7.28 (d, 1H), 7.36 (t, 2H), 7.60 (dd, 1H), 7.83 (s, 1H), 8.09 (dd, 2H), 9.65 (t, 1H).
LRMS M+H: 489

### Example 140

### 5-chloro-N-[3-(dimethylamino)propyl]-2-[2-[4-(4-fluorobenzoyl)-1-pi peridinyl]-2-oxoethoxy]benzamide

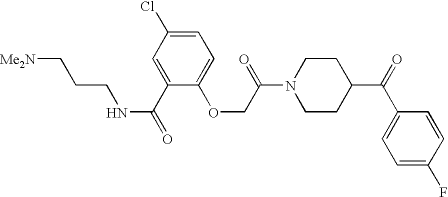 Prepared in a similar manner to Compound 131. ¹H NMR (400 MHz, DMSO-d₆): δ/ppm=1.40 (q, 1H), 1.60 (q, 1H), 1.82 (d, 2H), 1.95 (m, 2H), 2.72 (s, 3H), 2.83 (t, 1H), 3.05 (m, 1H), 3.20 (t, 1H), 3.38 (q, 2H), 3.73 (m, 1H), 3.83 (d, 1H), 4.38 (d, 1H), 5.00 (d, 1H), 5.14 (d, 1H), 7.28 (d, 1H), 7.36 (t, 2H), 7.56 (dd, 1H), 7.83 (s, 1H), 8.09 (dd, 2H), 9.43 (t, 1H), 10.05 (br, 1H).
LRMS M+H: 503

### Example 141

### 2-amino-N-[5-chloro-2-[2-[4-(4-fluorobenzoyl)-1-piperidinyl]-2-oxoethoxy]phenyl]acetamide

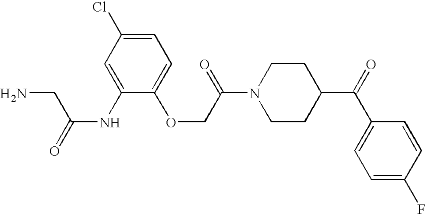

### A. [2-[[5-chloro-2-[2-[4-(4-fluorobenzoyl)-1-piperidinyl]-2-oxoethoxy]phenyl]amino]-2-oxoethyl]carbamic acid 1,1-dimethylethyl ester

To a solution of Compound 69 (200 mg, 0.5 mmol), tert-butoxycarbonylglycine (90 mg, 0.5 mmol) and HATU (200 mg, 0.5 mmol) in dichloromethane (8 mL) was added triethylamine (0.14 mL, 1.0 mmol). The mixture was stirred at ambient temperature for 2 days, then diluted with dichloromethane (50 mL) and washed with 0.2 N hydrochloric acid (50 mL), water (100 mL) and saturated sodium bicarbonate, dried and concentrated. Drying under vacuum afforded Intermediate 141a (270 mg).

### B. 2-amino-N-[5-chloro-2-[2-[4-(4-fluorobenzoyl)-1-piperidinyl]-2-oxoethoxy]phenyl]acetamide

Intermediate 141a (250 mg, 0.46 mmol) was dissolved in 4 N hydrochloric acid in dioxane (5 mL) and stirred at ambient temperature for 2 hours. Ether (30 mL) was added and the solid collected by filtration. Purification by reverse phase HPLC afforded Compound 141 (95 mg). ¹H NMR (400 MHz, DMSO-d₆): δ/ppm=1.40 (q, 1H), 1.62 (q, 1H), 1.82 (d, 2H), 2.85 (t, 1H), 3.24 (t, 1H), 3.73 (t, 1H), 3.86 (d, 1H), 3.92 (m, 2H), 4.35 (d, 1H), 5.01 (d, 1H), 5.04 (d, 1H), 7.03 (d, 1H), 7.12 (d, 1H), 7.38 (t, 2H), 8.13 (m, 5H), 10.15 (s, 1H).

LRMS M+H: 447

### Example 142

### 3-amino-N-[5-chloro-2-[2-[4-(4-fluorobenzoyl)-1-piperidinyl]-2-oxoethoxy]phenyl]propanamide

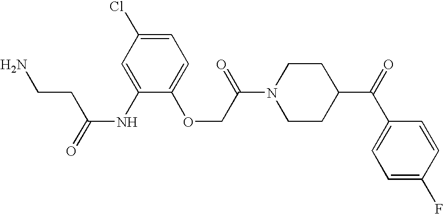 Prepared in a similar manner to Compound 141. ¹H NMR (400 MHz, DMSO-d₆): δ/ppm=1.37 (q, 1H), 1.55 (q, 1H), 1.80 (d, 2H), 2.80 (m, 3H), 3.07 (m, 2H), 3.18 (t, 1H), 3.68 (t, 1H), 3.79 (d, 1H), 4.32 (d, 1H), 4.92 (d, 1H), 4.98 (d, 1H), 7.00 (d, 1H), 7.08 (d, 1H), 7.34 (t, 2H), 7.70 (br, 3H), 8.13 (m, 3H), 9.96 (s, 1H).
LRMS M+H: 461

### Example 143

### (2S)-N-[5-chloro-2-[2-[4-(4-fluorobenzoyl)-1-piperidinyl]-2-oxoethoxy]phenyl]-2-pyrrolidinecarboxamide

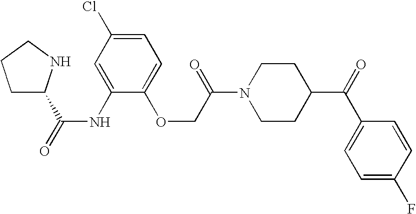 Prepared in a similar manner to Compound 141. ¹H NMR (400 MHz, DMSO-d₆): δ/ppm=1.40 (m, 1H), 1.60 (q, 1H), 1.83 (d, 2H), 1.95 (m, 3H), 2.40 (m, 1H), 2.84 (t, 1H), 3.28 (m, 3H), 3.73 (t, 1H), 3.83 (d, 1H), 4.37 (d, 1H), 4.55 (m, 1H), 5.01 (d, 1H), 5.07 (d, 1H), 7.07 (d, 1H), 7.17 (d, 1H), 7.38 (t, 2H), 8.08 (m, 3H), 8.68 (m, 1H), 9.32 (m, 1H), 10.34 (d, 1H).
LRMS M+H: 487

### Example 144

### (α⁴S)-α-amino-N-[5-chloro-2-[2-[4-(4-fluorobenzoyl)-1-piperidinyl]-2-oxoethoxy]phenyl]-1H-imidazole-4-propanamide

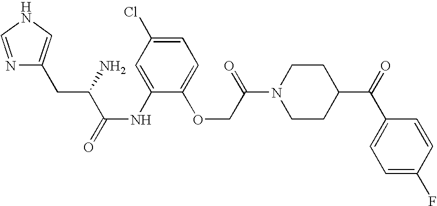 Prepared in a similar manner to Compound 141. ¹H NMR (400 MHz, DMSO-d₆): δ/ppm=1.38 (m, 1H), 1.58 (m, 1H), 1.80, (m, 2H), 2.80 (t, 1H), 3.12 (m, 3H), 3.71 (t, 1H), 3.80 (d, 1H), 4.32 (d, 1H), 4.27 (m, 1H), 5.00 (m, 2H), 7.03 (d, 1H), 7.13 (d, 1H), 7.34 (t, 2H), 7.47 (s, 1H), 7.97 (m, 1H), 8.07 (m, 2H), 8.43 (br, 3H), 9.00 (s, 1H), 10.12 (d, 1H).
LRMS M+H: 527

### Example 145

### (2S)-2-amino-5-[(aminoiminomethyl)amino]-N-[5-chloro-2-[2-[4-(4-fluorobenzoyl)-1-piperidinyl]-2-oxoethoxy]phenyl]pentanamide

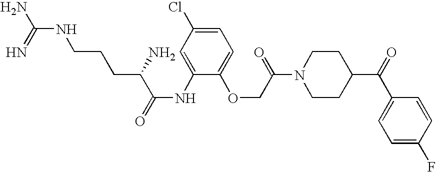 Prepared in a similar manner to Compound 141. ¹H NMR (400 MHz, DMSO-d₆): δ/ppm=1.78 (q, 1H), 1.95 (m, 3H), 2.20 (m, 4H), 3.22 (t, 1H), 3.48 (m, 2H), 3.60 (t, 1H), 4.10 (m, 1H), 4.21 (d, 1H), 4.60 (m, 1H), 4.74 (d, 1H), 5.42 (s, 2H), 7.42 (d, 1H), 7.34 (m, 1H), 7.75 (m, 2H), 8.00 (m, 1H), 8.45 (m, 3H), 8.68 (m, 3H), 10.64 (d, 1H).
LRMS M+H: 546

### Example 146

### (2S)-2,5-diamino-N-[5-chloro-2-[2-[4-(4-fluorobenzoyl)-1-piperidinyl]-2-oxoethoxy]phenyl]pentanamide

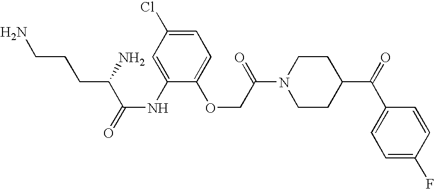 Prepared in a similar manner to Compound 141. ¹H NMR (400 MHz, DMSO-d₆): δ/ppm=1.38 (q, 1H), 1.64 (m, 3H), 1.85 (m, 4H), 2.82 (m, 3H), 3.22 (t, 1H), 3.78 (t, 1H), 3.82 (d, 1H), 4.23 (m, 1H), 4.34 (d, 1H), 5.04 (s, 2H), 7.04 (d, 1H), 7.18 (d, 1H), 7.40 (t, 2H), 7.75 (br, 3H), 8.02 (m, 1H), 8.10 (dd, 2H), 8.35 (br, 3H), 10.28 (s, 1H).
LRMS M+H: 504

### Example 147

(2S)-2-amino-N-[5-chloro-2-[2-[4-(4-fluorobenzoyl)-1-piperidinyl]-2-oxoethoxy]phenyl]-3-hydroxypropanamide 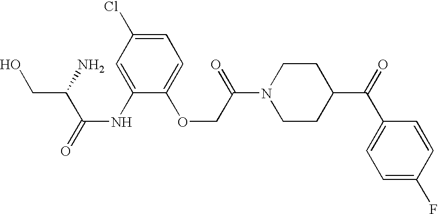
Prepared in a similar manner to Compound 141. ¹H NMR (400 MHz, DMSO-d₆): δ/ppm=1.38 (q, 1H), 1.56 (q, 1H), 1.80 (d, 2H), 2.82 (t, 1H), 3.20 (t, 1H), 3.62 (br, 2H), 3.70 (m, 2H), 3.82 (m, 2H), 4.20 (m, 1H), 4.33 (d, 1H), 5.04 (s, 2H), 5.60 (br, 1H), 7.02 (d, 1H), 7.14 (dd, 1H), 7.36 (t, 2H), 8.06 (m, 3H), 8.25 (m, 2H), 10.18 (s, 1H).
LRMS M+H: 477

### Example 148

### (2S)-2-amino-N′-[5-chloro-2-[2-[4-(4-fluorobenzoyl)-1-piperidinyl]-2-oxoethoxy]phenyl]pentanediamide

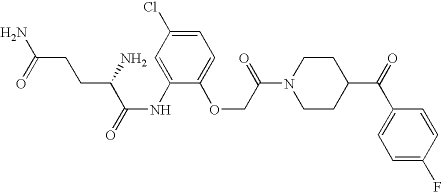 Prepared in a similar manner to Compound 141. ¹H NMR (400 MHz, DMSO-d₆): δ/ppm=1.38 (q, 1H), 1.62 (m, 3H), 1.81 (d, 2H), 2.82 (t, 1H), 3.22 (m, 4H), 3.78 (m, 1H), 3.82 (d, 1H), 4.33 (d, 1H), 4.54 (m, 1H), 5.04 (t, 2H), 7.04 (d, 1H), 7.18 (d, 1H), 7.38 (m, 2H), 8.04 (m, 1H), 8.10 (dd, 2H), 8.56 (br, 2H), 10.18 (s, 1H).
LRMS M+H: 518

### Example 149

### N-[5-chloro-2-[2-[4-[(4-fluorophenyl)hydroxymethyl]-4-methyl-1-piperidinyl]-2-oxoethoxy]phenyl]-4-piperidineacetamide

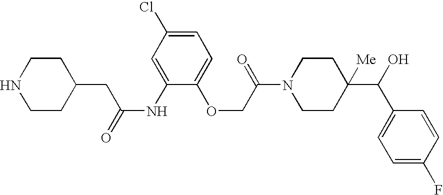 Prepared from Compound 96 in a similar manner to Compound 141. ¹H NMR (400 MHz, DMSO-d₆): δ/ppm=0.86 (s, 3H), 1.02 (m, 1H), 1.42 (m, 4H), 1.61 (m, 1H), 1.84 (d, 2H), 2.08 (m, 1H), 2.41 (d, 2H), 2.88 (m, 3H), 3.13 (q, 1H), 3.26 (d, 2H), 3.60 (d, 1H), 4.08 (m, 1H), 4.28 (d, 1H), 4.95 (m, 2H), 7.02 (m, 2H), 7.10 (t, 2H), 7.30 (m, 2H), 8.15 (s, 1H), 8.20 (br, 1H), 8.50 (br, 1H), 9.80 (s, 1H).
LRMS M+H: 531

### Example 150

### (α⁴S)-α-amino-N-[5-chloro-2-[2-[4-[(4-fluorophenyl)hydroxymethyl]-4-methyl-1-piperidinyl]-2-oxoethoxy]phenyl]-1H-imidazole-4-propanamide

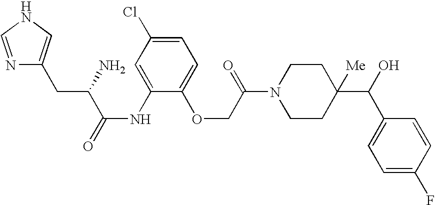 Prepared from Compound 96 in a similar manner to Compound 141. ¹H NMR (400 MHz, DMSO-d₆): δ/ppm=0.86 (s, 3H), 1.05 (m, 1H), 1.24 (m, 2H), 1.42 (m, 2H), 1.61 (m, 1H), 1.77 (m, 1H), 1.99 (m, 1H), 2.82 (m, 1H), 3.13 (m, 1H), 3.28 (d, 2H), 3.60 (d, 1H), 4.08 (m, 1H), 4.34 (s, 1H), 4.50 (m, 1H), 4.99 (m, 2H), 7.01 (m, 1H), 7.15 (m, 3H), 7.32 (m, 2H), 7.53 (s, 1H), 8.00 (s, 1H), 8.14 (br, 1H), 8.48 (br, 3H), 9.03 (s, 1H), 10.22 (d. 1H).
LRMS M+H: 543

### Example 151

### N-[5-chloro-2-[2-[4-(4-fluorobenzoyl)-1-piperidinyl]-2-oxoethoxy]phenyl]-N′-[2-(1H-imidazol-4-yl)ethyl]urea

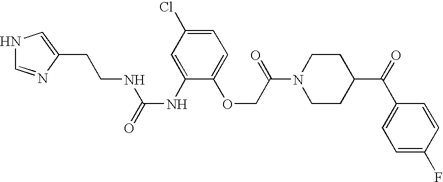

### A. [5-chloro-2-[2-[4-(4-fluorobenzoyl)-1-piperidinyl]-2-oxoethoxy]phenyl]carbamic acid phenyl ester

To a solution of phenyl chloroformate (170 mg, 1.1 mmol) in dichloromethane (10 mL) at 0° C. was added Compound 69 (390 mg, 1.0 mmol) and the mixture stirred until a homogeneous solution formed. The mixture was cooled to −20° C., and a solution of triethylamine (0.20 mL, 1.4 mmol) in dichloromethane (1 mL) added dropwise. The mixture was stirred at 0° C. for 5 hours, then warmed to ambient temperature and stirred overnight. The mixture was diluted with dichloromethane (35 mL) and washed with 0.5 N hydrochloric acid (30 mL) and water (2×70 mL). Removal of the solvent afforded the crude phenyl carbamate Intermediate 151a (550 mg).

### B. N-[5-chloro-2-[2-[4-(4-fluorobenzoyl)-1-piperidinyl]-2-oxoethoxy]phenyl]-N′-[2-(1H-imidazol-4-yl)ethyl]urea

Histamine (37 mg, 0.3 mmol) was added to a solution of Intermediate 151a (100 mg, 0.0.2 mmol) in acetonitrile (2 mL) and stirred at ambient temperature for 3 days. Acidification with 1 N hydrochloric acid (0.5 mL) and purification by reverse phase HPLC afforded Compound 151 (103 mg). ¹H NMR (400 MHz, DMSO-d₆): δ/ppm=1.42 (q, 1H), 1.61 (q, 1H), 1.84 (d, 2H), 2.83 (m, 3H), 3.15 (t, 1H), 3.43 (m, 2H), 3.75 (t, 1H), 3.88 (d, 1H), 4.38 (d, 1H), 5.00 (s, 2H), 6.97 (s, 2H), 7.18 (br, 1H), 7.37 (t, 2H), 7.48 (s, 1H), 8.13 (m, 2H), 8.20 (s, 2H), 9.01 (s, 1H).

LRMS M+H: 527

### Example 152

### N-[5-chloro-2-[2-[4-(4-fluorobenzoyl)-1-piperidinyl]-2-oxoethoxy]phenyl]-N′-[(2R)-2-hydroxypropyl]urea

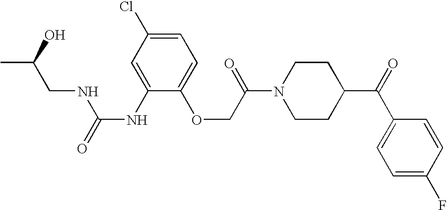 Prepared in a similar manner to Compound 151. ¹H NMR (400 MHz, DMSO-d₆): δ/ppm=1.00 (d, 3H), 1.30 (q, 1H), 1.55 (q, 1H), 1.78 (m, 2H), 2.79 (t, 1H), 2.94 (dd, 1H), 3.05 (dd, 1H), 3.20 (t, 1H), 3.65 (m, 2H), 3.83 (t, 1H), 4.30 (d, 1H), 4.90 (s, 2H), 6.80 (t, 2H), 7.10 (br, 1H), 7.31 (t, 2H), 8.03 (t, 2H), 8.17 (s, 1H), 8.26 (s, 1H).
LRMS M+H: 491

### Example 153

### N-[5-chloro-2-[2-[4-(4-fluorobenzoyl)-1-piperidinyl]-2-oxoethoxy]phenyl]-N′-[(2S)-2-hydroxypropyl]urea

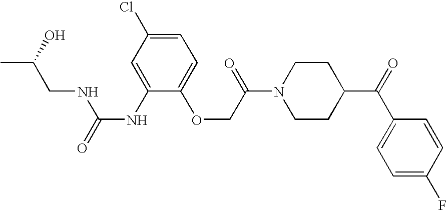 Prepared in a similar manner to Compound 151. ¹H NMR (400 MHz, DMSO-d₆): δ/ppm=15=1.00 (d, 3H), 1.30 (q, 1H), 1.55 (q, 1H), 1.78 (m, 2H), 2.79 (t, 1H), 2.94 (dd, 1H), 3.05 (dd, 1H), 3.20 (t, 1H), 3.65 (m, 2H), 3.83 (t, 1H), 4.30 (d, 1H), 4.90 (s, 2H), 6.80 (t, 2H), 7.10 (br, 1H), 7.31 (t, 2H), 8.03 (t, 2H), 8.17 (s, 1H), 8.26 (s, 1H).
LRMS M+H: 491

### Example 154

### N-[5-chloro-2-[2-[4-[(4-fluorophenyl)hydroxymethyl]-4-methyl-1-piperidinyl]-2-oxoethoxy]phenyl]-N′-[2-(1H-imidazol-4-yl)ethyl]urea

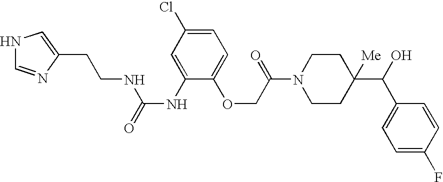 Prepared from Compound 96 in a similar manner to Compound 151. ¹H NMR (400 MHz, DMSO-d₆): δ/ppm=0.81 (s, 3H), 0.96 (m, 1H), 1.40 (m, 2H), 1.55 (q, 1H), 2.80 (m, 3H), 3.11 (q, 1H), 3.38 (m, 2H), 3.60 (d, 1H), 4.02 (m, 1H), 4.25 (d, 1H), 4.87 (m, 2H), 6.80 (s, 2H), 7.10 (m, 3H), 7.25 (m, 2H), 7.44 (s, 1H), 8.13 (s, 1H), 8.98 (s, 1H).
LRMS M+H: 543

### Example 155

### N-[5-chloro-2-[2-[4-[fluoro(4-fluorophenyl)methyl]-1-piperidinyl]-2-oxoethoxy]phenyl]urea

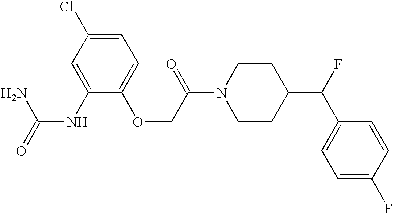 To a solution of Compound 98 (100 mg, 0.23 mmol) in dichloromethane (5 mL) at −78° C. was added (diethylamino)sulfur trifluoride (DAST, 0.10 mL, 0.68 mmol). The mixture was allowed to warm to ambient temperature and stirred overnight, then recooled to −78° C. and quenched by addition of triethylamine and methanol. Extraction and purification by reverse phase HPLC afforded Compound 155 as a light yellow solid. ¹H NMR (400 MHz, DMSO-d₆): δ/ppm=0.98-1.36 (m, 3H), 1.98 (m, 1H), 2.08 (m, 1H), 2.52 (m, 1H), 2.96 (m, 1H), 3.80 (m, 1H), 4.30 (m, 1H), 4.89 (m, 2H), 5.23 (s, 1H), 5.35 (s, 1H), 6.34 (br, 2H), 6.80 (m, 2H), 7.22 (m, 2H), 7.38 (m, 2H), 8.10 (d, 1H), 8.16 (br, 1H).
LRMS M+H, 438.1.

### Example 156

### N-[5-chloro-2-[2-[4-[fluoro(4-fluorophenyl)methyl]-4-methyl-1-piperidinyl]-2-oxoethoxy]phenyl]urea

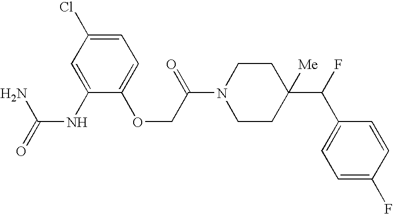 Prepared from Compound 18 in a similar manner to Compound 155. ¹H NMR (400 MHz, DMSO-d₆+TFA): δ/ppm=0.90 (m, 3H), 1.06 (m, 1H), 1.36-1.66 (m, 3H), 2.84 (m, 1H), 3.18 (m, 1H), 3.68 (m, 1H), 4.10 (m, 1H), 4.80-4.98 (m, 2H), 5.34 (s, 1H), 5.22 (s, 1H), 6.80 (m, 2H), 7.16 (m, 2H), 7.28 (m, 2H), 8.16 (br, 1H), 8.18 (m, 1H).
LRMS M+H, 452.1

### Example 157

### N-[5-chloro-2-[2-[4-(fluoromethyl)-4-[(4-fluorophenyl)methyl]-1-piperidinyl]-2-oxoethoxy]phenyl]urea

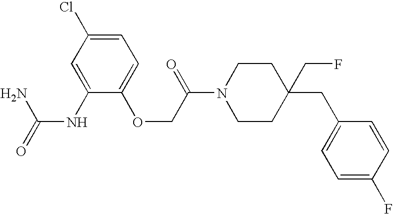 Prepared from Compound 32 in a similar manner to Compound 155. ¹H NMR (400 MHz, DMSO-d₆): δ/ppm=1.64 (m, 3H), 1.90 (m, 2H), 2.68 (m, 2H), 3.10 (m, 1H), 3.70 (m, 1H), 4.90 (m, 2H), 6.40 (s, 2H), 6.80 (m, 2H), 7.10 (m, 2H), 7.20 (m, 2H), 8.16 (m, 2H).
LRMS M+H, 452.2

### Example 158

### trifluoroacetic acid [1-[2-[2-[(aminocarbonyl)amino]-4-chlorophenoxy]acetyl]-4-piperidinyl](4-fluorophenyl)methyl ester

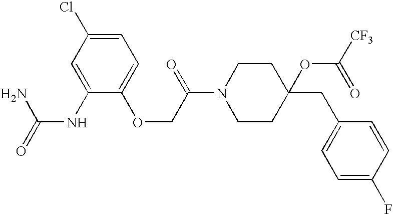 A solution of Compound 98 (170 mg, 0.39 mmol) in trifluoroacetic acid (10 mL) and dichloromethane (5 mL) was refluxed for 4 h. Concentration and purification by reverse phase HPLC afforded Compound 158 as a white solid. ¹H NMR (400 MHz, DMSO-d₆+TFA): δ/ppm=1.00-1.34 (m, 3H), 1.80 (m, 1H), 2.20 (m, 1H), 2.56 (m, 1H), 2.96 (m, 1H), 3.82 (m, 1H), 4.32 (m, 1H), 4.80-4.98 (m, 2H), 6.74 (m, 1H), 6.76-6.82 (m, 2H), 7.20 (m, 2H), 7.40 (m, 2H), 8.12 (br, 1H), 8.17 (br, 1H).
LRMS M+H, 532.1

### Example 159

### N-[5-chloro-2-[2-[4-[(4-fluorophenyl)methyl]-4-[(methylamino)methyl]-1-piperidinyl]-2-oxoethoxy]phenyl]urea

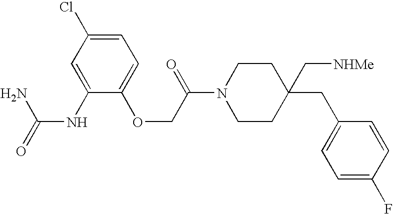 To a solution of Compound 36 (447 mg, 1.0 mmol) in 1,2-dichloroethane (5 mL) was added methylamine (2.0 M in THF, 0.6 mL, 1.2 mmol) and several drops of acetic acid. After stirring for 8 hours at ambient temperature, sodium triacetoxyborohydride (275 mg, 1.3 mmol) was added and the mixture stirred overnight. The reaction was diluted with dichloromethane (50 mL) and water (15 mL), and the aqueous layer adjusted to ca. pH 9. Extraction and purification by reverse phase HPLC afforded Compound 159 (62 mg). ¹H NMR (400 MHz, DMSO-d₆): δ/ppm=1.24 (m, 1H), 1.44 (m, 3H), 2.64 (m, 3H), 2.72 (s, 2H), 2.90 (m, 2H), 3.24 (m, 1H), 3.36 (m, 1H), 3.56 (m, 1H), 3.72 (m, 1H), 4.86 (m, 2H), 6.78 (m, 2H), 7.16 (m, 4H), 8.10 (s, 1H), 8.18 (s, 1H), 8.40 (m, 1H).
LRMS M+H, 463.1

### Example 160

### N-[5-chloro-2-[2-[4-[(4-fluorophenyl)methyl]4-(1-pyrrolidinylmethyl)-1-piperidinyl]-2-oxoethoxy]phenyl]urea

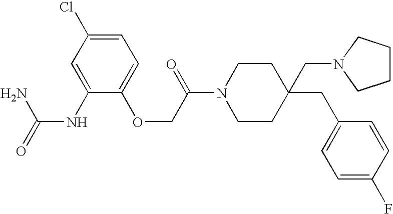 Prepared in a similar manner to Compound 159. ¹H NMR (400 MHz, DMSO-d₆): δ/ppm=1.36 (m, 2H), 1.54 (m, 2H), 1.92 (m, 2H), 2.04 (m, 2H), 2.88 (s, 2H), 3.12 (m, 2H), 3.24 (m, 2H), 3.44 (m, 2H), 4.88 (m, 2H), 6.38 (s, 1H), 6.78 (m, 2H), 7.18 (m, 4H), 8.10 (s, 1H), 8.18 (s, 1H), 9.10 (m, 1H).

### Example 161

### N-[5-chloro-2-[2-[4-[(4-fluorophenyl)methyl]4-[(4-methyl-1-piperazinyl)methyl]-1-piperidinyl]-2-oxoethoxy]phenyl]urea

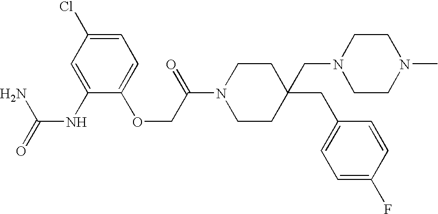 Prepared in a similar manner to Compound 159. ¹H NMR (400 MHz, DMSO-d₆): δ/ppm=1.22 (m, 2H), 1.46 (m, 2H), 2.26 (s, 2H), 2.70 (s, 2H), 2.78 (m, 5H), 3.00 (m, 2H), 3.34 (m, 2H), 3.50 (m, 6H), 4.86 (m, 2H), 6.36 (s, 1H), 6.78 (m, 2H), 7.12 (m, 4H), 8.10 (s, 1H), 8.18 (s, 1H), 8.30 (m, 1H).
LRMS M+H, 532.0

### Example 162

### N-[5-chloro-2-[2-[4-[(ethylamino)methyl]-4-[(4-fluorophenyl)methyl]-1-piperidinyl]-2-oxoethoxy]phenyl]urea

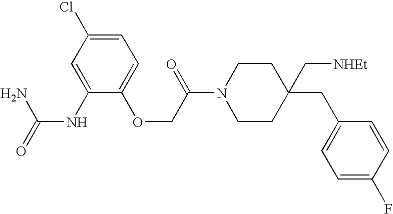 Prepared in a similar manner to Compound 159. ¹H NMR (400 MHz, DMSO-d₆): δ/ppm=1.25 (m, 4H), 1.35 (m, 1H), 1.49 (m, 2H), 2.76 (s, 2H), 2.89 (m, 2H), 3.00 (m, 2H), 3.25 (m, 1H), 3.38 (m, 1H), 3.59 (m, 1H), 3.70 (m, 1H), 4.86 (m, 2H), 6.35 (s, 2H), 6.78 (m, 2H), 7.17 (m, 4H), 8.09 (s, 1H), 8.16 (s, 3H).

### Example 163

### N-[5-chloro-2-[2-[4-[(4-fluorophenyl)methyl]-4-[[(2-hydroxyethyl)amino]methyl]-1-piperidinyl]-2-oxoethoxy]phenyl]urea

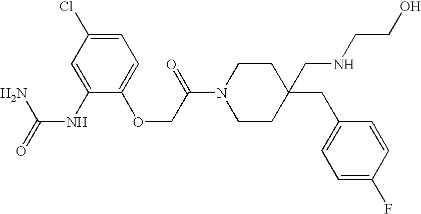 Prepared in a similar manner to Compound 159. ¹H NMR (400 MHz, DMSO-d₆): δ/ppm=1.24 (m, 1H), 1.38 (m, 1H), 1.48 (m, 2H), 2.77 (s, 2H), 2.96 (m, 2H), 3.06 (m, 2H), 3.36 (m, 2H), 3.58 (m, 1H), 3.70 (m, 3H), 4.86 (m, 2H), 6.78 (m, 2H), 7.18 (m, 4H), 8.09 (s, 1H), 8.16 (s, 1H), 8.26 (m, 1H).
LRMS M+H, 493.1

### Example 164

### 1-[[4-chloro-2-[(4-methyl-1-piperazinyl)methyl]phenoxy]acetyl]-4-[(4-fluorophenyl)methyl]-4-piperidinecarbonitrile

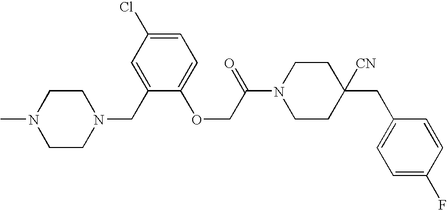 Prepared from Compound 25 in a similar manner to Compound 159. ¹H NMR (400 MHz, CDCl₃): δ/ppm=1.57-1.70 (m, 2H), 1.86-1.95 (m, 2H), 2.84-2.92 (m, 1H), 2.86 (s, 2H), 2.92 (s, 3H), 3.36-3.42 (m, 1H), 3.65-3.81 (m, 9H), 4.21 (d, 1H), 4.50-4.53 (m, 2H), 4.92 (d, 1H), 5.01 (d, 1H), 6.81 (d, 1H), 7.03-7.07 (m, 2H), 7.24-7.29 (m, 2H), 7.36 (d, 1H), 7.40 (dd, 1H).
LRMS M+H, 499.1

### Example 165

### 1-[[4-chloro-2-[(methylamino)methyl]phenoxy]acetyl]-4-[(4-fluorophenyl)methyl]-4-piperidinecarbonitrile

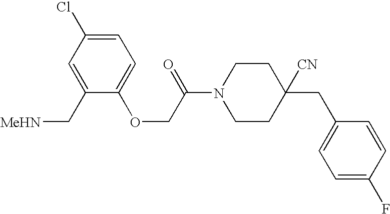 Prepared from Compound 25 in a similar manner to Compound 159. ¹H NMR (400 MHz, CDCl₃): δ/ppm=1.56 (dd, 1H), 1.66 (dd, 1H), 1.89-1.97 (m, 2H), 2.72-2.74 (m, 3H), 2.82-2.88 (m, 3H), 3.37-3.43 (m, 1H), 3.64 (d, 1H), 3.96-4.00 (m, 1H), 4.31-4.34 (m, 1H), 4.46 (br, 2H), 4.57 (d, 1H), 4.94 (d, 1H), 4.99 (d, 1H), 6.86 (d, 1H), 6.86 (d, 1H), 7.03-7.08 (m, 2H), 7.23-7.25 (m, 2H), 7.28 (d, 1H), 7.36 (dd, 1H), 9.6 (br, 1H), 10.0 (br, 1H).
LRMS M+H, 430.2

### Example 166

### 1-[[4-chloro-2-[[(2-hydroxyethyl)amino]methyl]phenoxy]acetyl]-4-[(4-fluorophenyl)methyl]-4-piperidinecarbonitrile

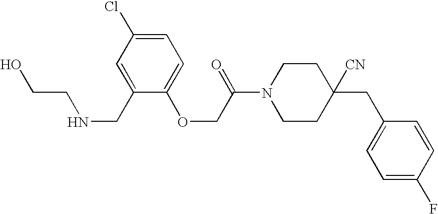 Prepared from Compound 25 in a similar manner to Compound 159. ¹H NMR (400 MHz, CDCl₃): δ/ppm=1.56-1.70 (m, 2H), 1.93 (t, 2H), 2.78-2.92 (m, 7H), 3.17 (br, 2H), 3.41 (t, 1H), 3.64 (d, 1H), 3.97 (br, 2H), 4.08-4.11 (m, 1H), 4.27 (d, 1H), 4.55 (d, 1H), 4.91 (d, 1H), 4.99 (d, 1H), 6.85 (d, 1H), 7.03-7.07 (m, 2H), 7.23-7.26 (m, 2H), 7.29 (d, 1H), 7.36 (dd, 1H), 9.60 (br, 1H), 10.00 (br, 1H).
LRMS M+H, 460.2

### Example 167

### 1-[[4-chloro-2-(4-morpholinylmethyl)phenoxy]acetyl]-4-[(4-fluorophenyl)methyl]-4-piperidinecarbonitrile

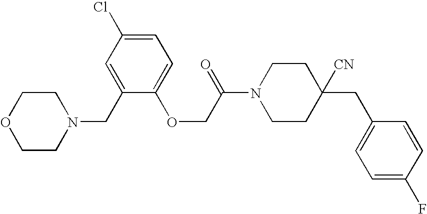 Prepared from Compound 25 in a similar manner to Compound 159. ¹H NMR (400 MHz, DMSO-d₆): δ/ppm=1.48-1.53 (m, 1H), 1.67-1.71 (m, 1H), 1.83 (t, 2H), 2.72 (t, 1H), 2.93 (s, 2H), 3.10-3.16 (m, 3H), 3.29 (m, 2H), 3.66 (t, 2H), 3.84 (d, 1H), 3.95 (d, 2H), 4.35-4.38 (m, 3H), 5.09 (d, 1H), 5.20 (d, 1H), 7.16-7.20 (m, 2H), 7.24 (d, 1H), 7.29-7.33 (m, 2H), 7.49-7.53 (m, 2H), 10.08 (br, 1H).
LRMS M+H, 486.1

### Example 168

### 1-[[4-chloro-2-[(dimethylamino)methyl]phenoxy]acetyl]-4-[(4-fluorophenyl)methyl]-4-piperidinecarbonitrile

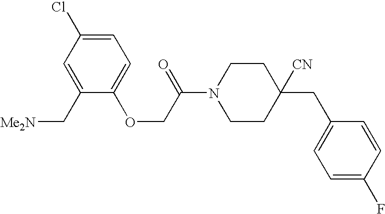 Prepared from Compound 25 in a similar manner to Compound 159. ¹H NMR (400 MHz, DMSO-d₆): δ/ppm=1.47-1.52 (m, 1H), 1.65-1.71 (m, 1H), 1.81 (t, 2H), 2.67-2.73 (m, 1H), 2.77 (s, 3H), 2.78 (s, 3H), 2.78 (s, 2H), 3.10-3.15 (m, 1H), 3.84 (d, 1H), 4.27 (d, 2H), 4.32 (d, 1H), 5.05 (d, 1H), 5.16 (d, 1H), 7.16-7.20 (m, 3H), 7.29-7.33 (m, 2H), 7.48 (dd, 1H), 7.52 (d, 1H).
LRMS M+H, 444.1

### Example 169

### [[[5-chloro-2-[2-[4-cyano-4-[(4-fluorophenyl)methyl]-1-piperidinyl]-2-oxoethoxy]phenyl]methyl]amino]acetic acid 1,1-dimethylethyl ester

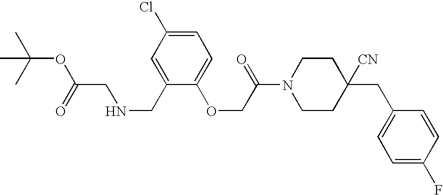 Prepared from Compound 25 in a similar manner to Compound 159. ¹H NMR (400 MHz, CDCl₃): δ/ppm=1.44 (s, 9H), 1.50-1.51 (m, 2H), 1.91 (d, 2H), 2.82 (s, 2H), 2.89 (t, 1H), 3.30 (s, 2H), 3.36 (t, 1H), 3.79 (s, 2H), 4.14-4.07 (m, 1H), 4.61-4.66 (m, 1H), 4.68 (d, 1H), 4.76 (d, 1H), 6.81 (d, 1H), 7.01-7.06 (m, 2H), 7.17 (dd, 1H), 7.20-7.24 (m, 2H), 7.30 (d, 1H).
LRMS M+H, 530.1

### Example 170

### 1-[[2-(aminomethyl)-4-chlorophenoxy]acetyl]-4-[(4-fluorophenyl)methyl]-4-piperidinecarbonitrile

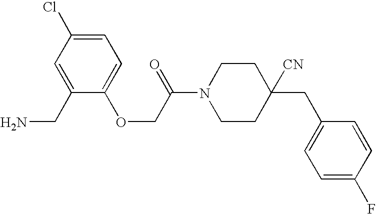 To a solution of Compound 25 (200 mg, 0.48 mmol) in methanol (50 mL) were added ammonium acetate (6.4 g, 83 mmol) and molecular sieves. After stirring overnight at ambient temperature, sodium triacetoxyborohydride (306 mg, 1.4 mmol) was added and the mixture stirred for 2 hours. The reaction was concentrated, diluted with dichloromethane and filtered. The filtrate was washed with water, dried and concentrated. Purification by reverse phase HPLC afforded Compound 170 (22 mg). ¹H NMR (400 MHz, DMSO-d₆): δ/ppm=1.46-1.51 (m, 1H), 1.65-1.71 (m, 1H), 1.81 (t, 2H), 2.65-2.73 (m, 1H), 2.92 (s, 2H), 3.12 (t, 1H), 3.84 (d, 1H), 4.02-4.04 (m, 2H), 4.32 (d, 1H), 5.05 (d, 1H), 5.15 (d, 1H), 7.16-7.20 (m, 3H), 7.29-7.32 (m, 2H), 7.41 (dd, 1H), 7.46 (d, 1H), 8.18 (br, 2H).
LRMS M+H, 416.1

### Example 171

### 1-[[4-chloro-2-(1H-1,2,4-triazol-1-ylmethyl)phenoxy]acetyl]-4-[(4-fluorophenyl)methyl]4-piperidinecarbonitrile

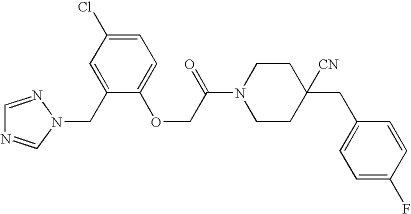

### A. 1-[[4-chloro-2-[[(methylsulfonyl)oxy]methyl]phenoxy]acetyl]-4-[(4-fluorophenyl)methyl]4-piperidinecarbonitrile

To a solution of Compound 99 (340 mg, 0.82 mmol) in dichloromethane (10 mL) at 0° C. was added triethylamine (0.17 mL, 1.2 mmol) followed by methanesulfonyl chloride (0.095 mL, 1.0 mmol) and the mixture stirred for 30 minutes. The reaction was quenched by addition of water and extracted with dichloromethane. The organic layer was washed with water, dried and concentrated to afford the methanesulfonate Intermediate 171a (360 mg) which was used without further purification.

### B. 1-[[4-chloro-2-(1H-1,2,4-triazol-1-ylmethyl)phenoxy]acetyl]-4-[(4-fluorophenyl)methyl]-4-piperidinecarbonitrile

To a solution of Intermediate 171a (280 mg, 0.57 mmol) in dimethylformamide (5 mL) were added potassium carbonate (235 mg, 1.7 mmol) and 1,2,4-triazole (59 mg, 0.85 mmol), and the mixture was heated at 60° C. overnight. After cooling to ambient temperature, extraction and purification by reverse phase HPLC afforded Compound 171 (31 mg). ¹H NMR (400 MHz, CDCl₃): δ/ppm=1.54-1.64 (m, 2H), 1.96 (d, 2H), 2.88-2.95 (m, 3H), 3.40 (t, 1H), 3.69 (d, 1H), 4.69 (d, 1H), 4.76 (s, 2H), 5.37 (d, 1H), 5.56 (d, 1H), 6.78 (d, 1H), 7.04-7.08 (m, 2H), 7.24-7.27 (m, 2H), 7.33 (dd, 1H), 7.42 (d, 1H), 8.16 (s, 1H), 9.4 (s, 1H).

LRMS M+H: 468

### Example 172

### 5-bromo-2-[2-[4-[(5-chloro-2-thienyl)methyl]-4-cyano-1-piperidinyl]-2-oxoethoxy]benzeneacetic acid methyl ester

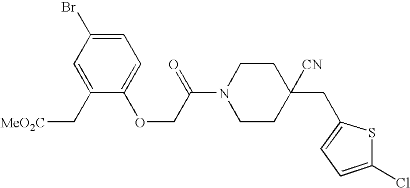 Prepared in a similar manner to Compound 59. ¹H NMR (400 MHz, CDCl₃) δ/ppm=1.40 (m, 2H), 1.80 (m, 2H), 2.80 (m, 1H), 2.85 (s, 2H), 3.20 (m, 1H), 3.50 (m, 2H), 3.55 (s, 3H), 3.90 (m, 1H), 4.50 (m, 3H), 6.65 (m, 3H), 7.22 (m, 2H).
LRMS M+H: 525

### Example 173

### 5-bromo-2-[2-[4-[(5-chloro-2-thienyl)methyl]-4-cyano-1-piperidinyl]-2-oxoethoxy]benzeneacetic acid

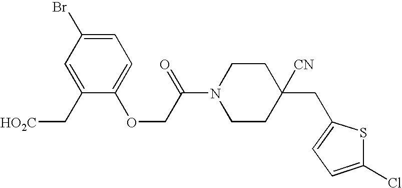 Prepared from Compound 172 in a similar manner to Compound 103. ¹H NMR (400 MHz, CDCl₃) δ/ppm=1.50 (m, 2H), 2.0 (m, 2H), 2.95 (m, 3H), 3.40 (m, 1H), 3.65 (s, 2H), 3.95 (m, 1H), 4.65 (m, 3H), 6.80 (m, 3H), 7.35 (m, 2H).
LRMS M+H: 511

### Example 174

### 5-chloro-2-[(1E)-3-[4-cyano-4-[(4-fluorophenyl)methyl]-1-piperidinyl]-3-oxo-1-propenyl]benzeneacetic acid methyl ester

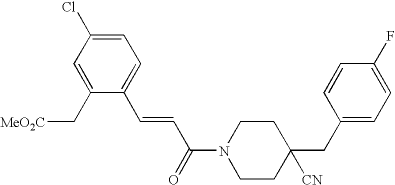

### A. 5-chloro-2-[(1E)-3-(1,1-dimethylethoxy)-3-oxo-1-propenyl]benzeneacetic acid methyl ester

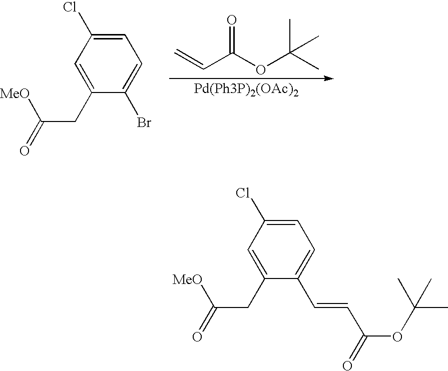 To a solution of 2-bromo-5-chlorobenzeneacetic acid methyl ester (2.0 g, 7.6 mmol) and 2-propenoic acid tert-butyl ester (1.2 mL, 8.4 mmol) in acetonitrile (5 mL) was added diisopropylethylamine (1.3 mL, 7.6 mmol), followed by bis(triphenylphosphine)palladium(II) acetate (1.3 g, 1.7 mmol), and the mixture heated at 110° C. in a sealed tube overnight. The mixture was diluted with ether (300 mL), filtered and concentrated in vacuo. Purified by chromatography on silica afforded Intermediate 174a (1.5 g) as a light yellow oil.

### B. 5-chloro-2-[(1E)-3-[4-cyano-4-[(4-fluorophenyl)methyl]-1-piperidinyl]-3-oxo-1-propenyl]benzeneacetic acid methyl ester

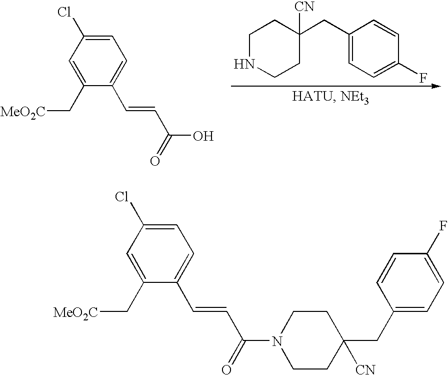 The tert-butoxycarbonyl protecting groups were removed from Intermediates 36a and 174a. The crude products were reacted in a similar manner as described for Compound 2. Purification by chromatography on silica afforded Compound 174 (1.5 g) as a white solid. ¹H NMR (400 MHz, CDCl₃): δ/ppm=1.50-1.70 (m, 2H), 1.90-2.00 (m, 2H), 2.86 (s, 2H), 2.94 (m, 1H), 3.42 (m, 1H), 3.70 (s, 3H), 3.74 (s, 2H), 4.12 (m, 1H), 4.80 (m, 1H), 6.76 (d, 1H), 7.04 (m, 2H), 7.22-7.30 (m, 4H), 7.48 (d, 1H), 7.80 (d, 1H).
LRMS M+H, 455.1

### Example 175

### 5-chloro-2-[(1E)-3-[4-cyano-4-[(4-fluorophenyl)methyl]-1-piperidinyl]-3-oxo-1-propenyl]benzeneacetic acid

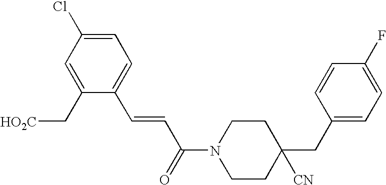 Prepared from Compound 174 in a similar manner to Compound 103, using barium hydroxide octahydrate. ¹H NMR (400 MHz, DMSO-d₆+TFA): δ/ppm=1.56 (m, 2H), 1.72-1.82 (m, 2H), 2.72 (m, 1H), 2.86 (s, 2H), 3.12 (m, 1H), 3.70 (s, 2H), 4.31 (m, 1H), 4.52 (m, 1H), 7.04-7.14 (m, 3H), 7.24-7.34 (m, 4H), 7.57 (d, 1H), 7.80 (d, 1H).
LRMS M+H, 440.9

### Example 176

### 5-bromo-2-[2-[4-[(4-fluorophenyl)methyl]-4-formyl-1-piperidinyl]-2-oxoethoxy]benzeneacetic acid methyl ester

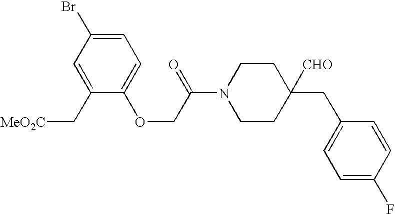 Prepared in a similar manner to Compound 36. ¹H NMR (400 MHz, DMSO-d₆): δ/ppm=1.38 (m, 1H), 1.50 (m, 1H), 1.82 (m, 2H), 2.80 (m, 2H), 2.94 (m, 1H), 3.58 (s, 2H), 3.64 (s, 2H), 4.00 (m, 1H), 4.76 (s, 2H), 6.82 (d, 1H), 7.02 (m, 3H), 7.44 (m, 2H), 9.60 (s, 1H).
LRMS M+H: 505

### Example 177

### 5-bromo-2-[2-[4-[(4-fluorophenyl)methyl]4-[(methylamino)methyl]-1-piperidinyl]-2-oxoethoxy]benzeneacetic acid methyl ester

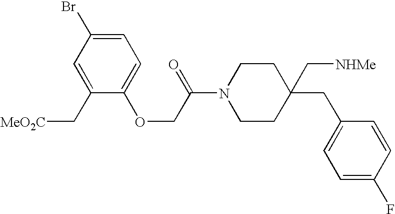 Prepared from Compound 176 in a similar manner to Compound 159. ¹H NMR (400 MHz, CDCl₃): δ/ppm=1.46 (m, 4H), 1.74 (m, 1H), 2.38 (s, 1H), 2.44 (s, 3H), 2.70 (s, 2H), 3.50 (m, 3H), 3.62 (s, 1H), 3.68 (s, 3H), 3.72 (m, 2H), 4.66 (s, 2H), 6.78 (d, 1H), 6.96 (m, 2H), 7.08 (m, 2H), 7.32 (m, 2H).
LRMS M+H: 520

### Example 178

### 5-bromo-2-[2-[4-[(4-fluorophenyl)methyl]-4-[(methylamino)methyl]-1-piperidinyl]-2-oxoethoxy]benzeneacetic acid

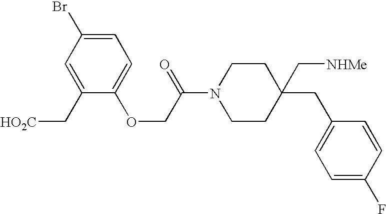 Prepared from Compound 177 in a similar manner to Compound 103. ¹H NMR (400 MHz, CDCl₃): δ/ppm=1.58 (m, 3H), 1.74 (m, 1H), 2.60 (s, 1H), 2.72 (s, 3H), 2.90 (m, 3H), 3.60 (m, 5H), 4.60 (d, 1H), 4.82 (d, 1H), 6.88 (d, 1H), 7.00 (m, 2H), 7.12 (m, 2H), 7.32 (m, 2H).
LRMS M+H: 506

### Example 179

### 5-bromo-2-[2-[4-cyano-4-[(4-fluorophenyl)methyl]-1-piperidinyl]-2-oxoethoxy]-4-(dimethylamino)benzeneacetic acid

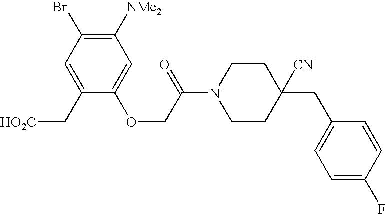

### A. 5-bromo-2-[(methoxycarbonyl)oxy]-4-nitrobenzeneacetic acid methyl ester

To a solution of 5-bromo-2-[(methoxycarbonyl)oxy]benzeneacetic acid methyl ester (1.3 g, 4.3 mmol) in chloroform (50 mL) at 0° C. was added dropwise a mixture of HNO3 (fuming, 1.0 mL) and H2SO4 (12 N, 5.0 mL). The mixture was stirred for 30 minutes, then poured onto ice water. Extraction with ethyl acetate and concentration in vacuo afforded Intermediate 179a (1.4 g).

### B. 5-bromo-2-hydroxy-4-nitrobenzeneacetic acid methyl ester

To a solution of Intermediate 179a (1.4 g, 4.0 mmol) in methylene chloride (20 mL) at 0° C. was added boron tribromide (1.0 M solution in hexane, 6.9 mL, 6.9 mmol). The mixture was warmed to ambient temperature and stirred for 2 hours, then quenched by addition of methanol. Extraction and concentration in vacuo afforded Intermediate 179b (0.76 g).

### C. 4-amino-5-bromo-2-hydroxybenzeneacetic acid methyl ester

To a solution of Intermediate 179b (0.22 g, 0.76 mmol) in methanol (10 mL) were added ammonium formate (0.48 g, 7.6 mmol) and 5% platinum on charcoal (0.6 mg). The mixture was heated at 90° C. for 1.5 hours, then cooled, filtered and concentrated. The residue was added to saturated sodium bicarbonate, extracted with methylene chloride and purified by chromatography on silica to afford Intermediate 179c (0.20 g).

### D. 5-bromo-4-(dimethylamino)-2-hydroxybenzeneacetic acid methyl ester

To a solution of Intermediate 179c (0.20 g, 0.76 mmol) in methanol (5 mL) was added formaldehyde (37% solution in water, 5.0 mL). After stirring for 20 minutes at ambient temperature, sodium cyanoborohydride (0.24 g, 3.8 mmol) was added and the mixture stirred overnight. Extraction and purification by reverse phase HPLC afforded Intermediate 179d (75 mg).

### E. 5-bromo-2-[2-[4-cyano-4-[(4-fluorophenyl)methlyl]-1-piperidinyl]-2-oxoethoxy]-4-(dimethylamino)benzeneacetic acid

Reaction of Intermediate 179d (70 mg, 0.25 mmol) with Intermediate 59b (120 mg, 0.40 mmol) in a similar manner to that described for Compound 59, followed by hydrolysis of the ester with lithium hydroxide and purification by reverse phase HPLC afforded Compound 178. ¹H NMR (400 MHz, DMSO-d₆): δ/ppm=1.48 (m, 1H), 1.65 (m, 1H), 1.78 (m, 2H), 2.67 (m, 1H), 2.82 (s, 4H dimethylamine rotamer), 2.90 (s, 2H dimethylamine rotamer), 3.00 (t, 0.3H rotamer), 3.10 (t, 0.7H rotamer), 3.50 (s, 2H), 3.73 (d, 0.3H rotamer), 3.90 (d, 0.7H rotamer), 4.05 (m, 2H), 4.40 (d, 1H), 4.84 (m, 2H), 6.90 (s, 1H), 7.15 (m, 2H), 7.28 (m, 2H), 7.44 (s, 1H).

LRMS M+H: 532

### Example 180

### [5-chloro-2-[2-[4-cyano-4-[(4-fluorophenyl)methyl]-1-piperidinyl]-2-oxoethoxy]phenoxy]acetic acid methyl ester

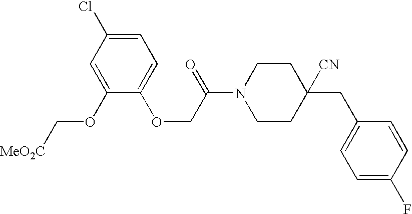 Prepared in a similar manner to Compound 59. LRMS ¹H NMR (400 MHz, CDCl₃) δ/ppm=1.50 (m, 2H), 1.90 (m, 2H), 2.80 (m, 3H), 3.35 (m, 1H), 3.75 (s, 3H), 4.20 (m, 1H), 4.65 (m, 3H), 4.75 (m, 2H), 6.80 (d, 1H), 6.95 (m, 2H), 7.05 (m, 2H), 7.22 (m, 2H).
LRMS M+H: 475

### Example 181

### [5-chloro-2-[2-[4-cyano-4-[(4-fluorophenyl)methyl]-1-piperidinyl]-2-oxoethoxy]phenoxy]acetic acid

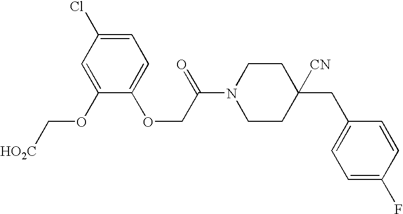 Prepared from Compound 180 in a similar manner to Compound 103. ¹H NMR (400 MHz, CDCl₃) δ/ppm=1.50 (m, 2H), 1.80 (m, 2H), 2.92 (s, 2H), 3.00 (m, 1H), 3.30 (m, 1H), 3.95 (m, 1H), 4.55 (m, 3H), 4.65 (m, 2H), 6.80 (m, 3H), 6.95 (m, 2H), 7.20 (m, 2H).
LRMS M+H: 461

### Example 182

### [[5-chloro-2-[2-[4-cyano-4-[(4-fluorophenyl)methyl]-1-piperidinyl]-2-oxoethoxy]phenyl]amino]acetic acid

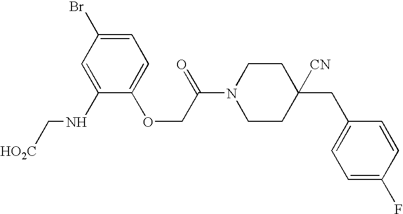 Prepared in a similar manner to Compound 121. ¹H NMR (400 MHz, DMSO-d₆): δ/ppm=1.55 (m, 1H), 1.80 (m, 3H), 2.72 (t, 1H), 2.95 (s, 2H), 3.18 (t, 1H), 3.90 (m, 3H), 4.21 (d, 1H), 4.80 (d, 1H), 4.92 (d, 1H), 6.60 (s, 1H), 6.74 (m, 2H), 7.20 (t, 2H), 7.35 (m, 2H).
LRMS M+H: 504

### Example 183

### 3-[5-chloro-2-[2-[4-cyano-4-[(4-fluorophenyl)methyl]-1-piperidinyl]-2-oxoethoxy]phenyl]-2-propenoic acid

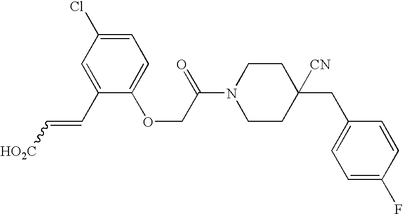 Prepared in a similar manner to Compound 2. Isolated as a 1:1 mixture of double bond isomers. ¹H NMR (400 MHz, CDCl₃) δ/ppm=1.50 (m, 2H), 1.90 (m, 2H), 2.85 (m, 3H), 3.35 (q, 1H), 3.98 (t, 1H), 4.6-4.8 (m, 3H), 6.04 (d, 0.5H), 6.53 (d, 0.5H), 6.85 (dd, 1H), 7.03 (m, 2H), 7.08 (d, 0.5H), 7.24 (m, 2.5H), 7.34 (dd, 0.5H), 7.50 (dd, 1H), 7.97 (d, 0.5H).
LRMS M+H: 456

### Example 184

### 5-bromo-2-[2-[4-[(4-fluorophenyl)methyl]-4-[[(2,2,2-trifluoroethyl)amino]methyl]-1-piperidinyl]-2-oxoethoxy]benzeneacetic acid

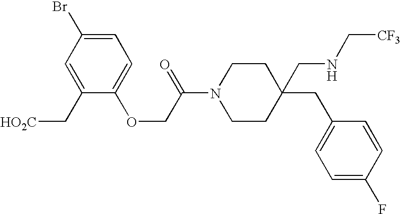 Prepared in a similar manner to Compound 103. ¹H NMR (400 MHz, CDCl₃): δ/ppm=1.34 (m, 1H), 1.58 (m, 2H), 1.73 (m, 1H), 2.65 (d, 1H), 2.77 (d, 1H), 2.96 (m, 2H), 3.29 (m, 1H), 3.46 (m, 1H), 3.56 (m, 3H), 3.68 (m, 1H), 3.77 (m, 1H), 4.13 (m, 1H), 4.52 (m, 1H), 4.87 (m, 1H), 6.88 (d, 1H), 7.06 (m, 4H), 7.33 (s, 1H), 7.38 (d, 1H).
LRMS M+H: 575

### Example 185

### 2-[2-[4-amino-4-[(4-fluorophenyl)methyl]-1-piperidinyl]-2-oxoethoxy]-5-bromobenzeneacetic acid

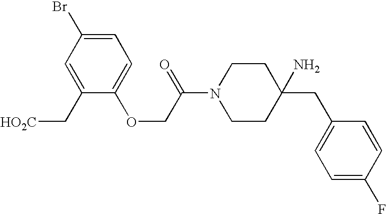 Prepared in a similar manner to Compound 103. ¹H NMR (400 MHz, CDCl₃): δ/ppm=1.20 (m, 1H), 1.78 (m, 3H), 1.98 (m, 1H), 2.60 (m, 1H), 3.02 (m, 2H), 3.55-3.77 (m, 6H), 4.51 (m, 1H), 4.75 (m, 1H), 6.82 (s, 1H), 7.02 (m, 2H), 7.18 (m.2H), 7.32 (m, 2H).
LRMS M+H: 479

### Example 186

### 5-chloro-2-[2-[4-cyano-4-[(4-fluorophenyl)methyl]-1-piperidinyl]-2-oxoethoxy]benzenepropanoic acid

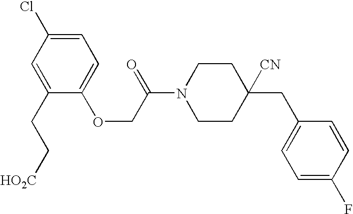 Prepared in a similar manner to Compound 103. ¹H NMR (400 MHz, CDCl₃): δ/ppm=1.50 (m, 2H), 1.95 (m, 2H), 2.65 (t, 2H), 2.85 (s, 2H), 2.90 (m, 3H), 3.40 (t, 1H), 4.00 (d, 1H), 4.05-4.75 (m, 3H), 6.75 (d, 1H), 7.05 (t, 2H), 7.16 (m, 2H), 7.25 (m, 2H).
LRMS M+H: 458

### Example 187

### 5-bromo-2-[2-[4-[(4-fluorophenyl)methyl]-4-hydroxy-1-piperidinyl]-2-oxoethoxy]benzeneacetic acid methyl ester

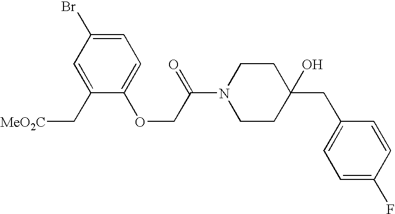 Prepared in a similar manner to Compound 1. ¹H NMR (400 MHz, CDCl₃): δ/ppm=1.60 (m, 3H), 1.85 (m, 1H), 2.75 (s, 2H), 3.0 (m, 1H), 3.45 (m, 1H), 3.65 (s, 2H), 3.70 (s, 3H), 3.75 (m, 1H), 4.00 (m, 1H), 4.35 (m, 1H), 4.70 (m, 2H), 6.80 (d, 1H), 7.02 (m, 2H), 7.15 (m, 2H), 7.35 (m, 2H).
LRMS M+H: 495

### Example 188

### 5-bromo-2-[2-[4-[(4-fluorophenyl)methyl]-4-hydroxy-1-piperidinyl]-2-oxoethoxy]benzeneacetic acid

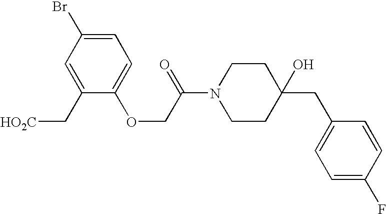 Prepared from Compound 187 in a similar manner to Compound 103. ¹H NMR (400 MHz, CDCl₃): δ/ppm=1.60 (m, 3H), 1.85 (m, 1H), 2.75 (s, 2H), 3.0 (m, 1H), 3.45 (m, 1H), 3.60 (m, 1H), 3.70 (s, 2H), 4.00 (m, 2H), 4.38 (m, 1H), 4.75 (m, 2H), 6.75 (d, 1H), 7.0 (t, 2H), 7.10 (m, 2H), 7.38 (m, 2H).
LRMS M+H: 481

### Example 189

### 5-bromo-2-[2-[4-[(4-fluorophenyl)methyl]-4-(1-piperidinylmethyl)-1-piperidinyl]-2-oxoethoxy]benzeneacetic acid methyl ester

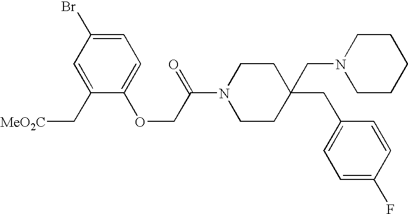 Prepared in a similar manner to Compound 2. ¹H NMR (400 MHz, CDCl₃): δ/ppm=1.26-1.41 (m, 3H), 1.47-1.62 (m, 9H), 2.14 (m, 2H), 2.38 (m, 3H), 2.68 (s, 2H), 3.50 (m, 3H), 3.62 (s, 2H), 3.68 (s, 3H), 4.66 (s, 2H), 6.78 (m, 1H), 6.94 (m, 2H), 7.04 (m, 2H), 7.32 (m, 2H).
LRMS M+H: 57

### Example 190

### 5-bromo-2-[2-[4-[(4-fluorophenyl)methyl]-4-(1-piperidinylmethyl)-1-piperidinyl]-2-oxoethoxy]benzeneacetic acid

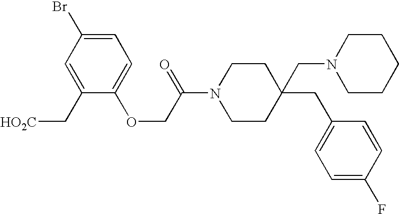 Prepared from Compound 189 in a similar manner to Compound 103. ¹H NMR (400 MHz, CDCl₃): δ/ppm=1.40 (m, 4H), 1.72 (m, 4H), 1.82 (m, 1H), 2.30 (m, 1H), 2.52 (m, 2H), 2.70 (m, 4H), 2.90 (m, 1H), 3.12 (m, 1H), 3.40 (m, 3H), 3.62 (m, 1H), 3.72 (m, 1H), 4.36 (m, 2H), 4.86 (m, 1H), 6.74 (m, 1H), 7.00 (m, 4H), 7.32 (m, 2H).
LRMS M+H: 561

### Example 191

### 2-[2-[4-(1-azetidinylmethyl)-4-[(4-fluorophenyl)methyl]-1-piperidinyl]-2-oxoethoxy]-5-bromobenzeneacetic acid methyl ester

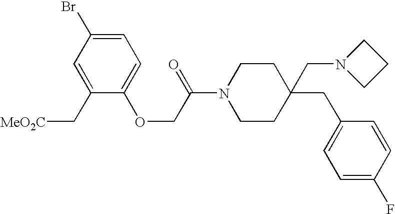 Prepared in a similar manner to Compound 2. ¹H NMR (400 MHz, CDCl₃): δ/ppm=1.36 (m, 4H), 2.10 (t, 1H), 2.22 (m, 1H), 2.92 (m, 2H), 3.01 (s, 2H), 3.30 (m, 4H), 3.50 (m, 3H), 3.64 (s, 2H), 3.71 (s, 3H), 4.68 (s, 2H), 6.76 (m, 1H), 6.94 (m, 2H), 7.06 (m, 2H), 7.32 (m, 2H), 8.01 (s, 1H).
LRMS M+H: 547

### Example 192

### 2-[2-[4-(1-azetidinylmethyl)-4-[(4-fluorophenyl)methyl]-1-piperidinyl]-2-oxoethoxy]-5-bromobenzeneacetic acid

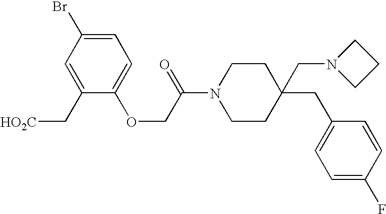 Prepared from Compound 191 in a similar manner to Compound 103. ¹H NMR (400 MHz, CDCl₃): δ/ppm=1.30 (m, 2H), 1.52 (m, 1H), 2.04 (m, 1H), 2.22 (m, 2H), 2.58 (m, 2H), 2.76 (m, 2H), 3.10 (m, 1H), 3.40 (m, 2H), 3.68 (m, 6H), 4.30 (m, 1H), 4.38 (m, 1H), 4.86 (m, 1H), 6.80 (m, 1H), 7.00 (m, 4H), 7.34 (m, 2H).
LRMS M+H: 533

### Example 193

### 5-bromo-2-[2-[4-[(4-fluorophenyl)methyl]-4-(1-pyrrolidinylmethyl)-1-piperidinyl]-2-oxoethoxy]benzeneacetic acid methyl ester

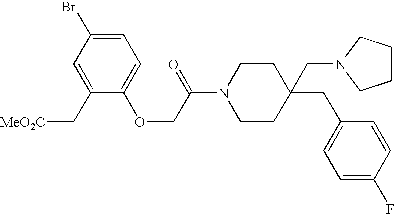 Prepared in a similar manner to Compound 2. ¹H NMR (400 MHz, DMSO-d₆): δ/ppm=1.20-1.40 (m, 2H), 1.52 (m, 2H), 1.88 (m, 2H), 2.00 (m, 1H), 2.34 (m, 2H), 2.86 (m, 2H), 3.30 (m, 4H), 3.44 (m, 2H), 3.55 (m, 5H), 3.62 (m, 2H), 4.80 (m, 2H), 6.80 (m, 1H), 7.08 (m, 2H), 7.18 (m, 2H), 7.37 (m, 2H), 7.88 (m, 1H).
LRMS M+H: 561

### Example 194

### 5-bromo-2-[2-[4-[(4-fluorophenyl)methyl]-4-(1-pyrrolidinylmethyl)-1-piperidinyl]-2-oxoethoxy]benzeneacetic acid

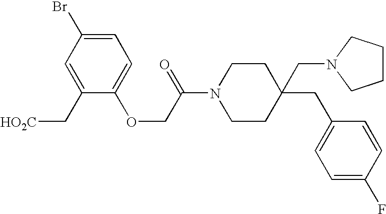 Prepared from Compound 193 in a similar manner to Compound 103. ¹H NMR (400 MHz, CDCl₃): δ/ppm=1.28-1.40 (m, 2H), 1.52 (m, 2H), 1.88 (m, 2H), 2.00 (m, 2H), 2.86 (m, 2H), 3.10 (m, 2H), 3.31-3.61 (m, 8H), 3.70 (m, 2H), 4.80 (m, 2H), 6.80 (m, 1H), 7.08 (m, 4H), 7.32 (m, 1H), 7.40 (m, 1H).
LRMS M+H: 547

### Example 195

### 5-bromo-2-[2-[4-cyano-4-[fluoro(4-fluorophenyl)methyl]-1-piperidinyl]-2-oxoethoxy]benzeneacetic acid methyl ester

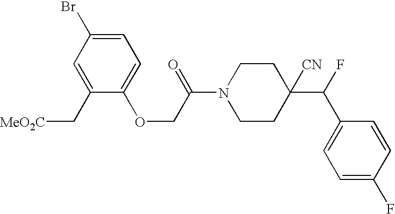 Prepared in a similar manner to Compound 2. ¹H NMR (400 MHz, DMSO-d₆): δ/ppm=1.27 (m, 1H), 1.66-1.88 (m, 2H), 2.18 (m, 1H), 2.64 (m, 1H), 3.08 (m, 1H), 3.56 (s, 3H), 3.66 (s, 2H), 3.90 (m, 1H), 4.40 (m, 1H), 4.84 (m, 2H), 5.70 (m, 1H), 6.88 (m, 1H), 7.36 (m, 4H), 7.48 (m, 2H).
LRMS M+H: 521

### Example 196

### 5-bromo-2-[2-[4-cyano-4-[fluoro(4-fluorophenyl)methyl]-1-piperidinyl]-2-oxoethoxy]benzeneacetic acid

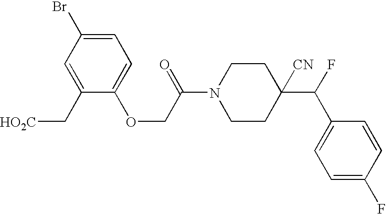 Prepared from Compound 195 in a similar manner to Compound 103. ¹H NMR (400 MHz, CDCl₃): δ/ppm=1.27 (m, 1H), 1.70 (m, 2H), 2.18 (m, 1H), 2.64 (m, 1H), 3.08 (m, 1H), 3.58 (s, 2H), 3.92 (m, 1H), 4.40 (m, 1H), 4.84 (m, 2H), 5.68 (m, 1H), 6.88 (m, 1H), 7.34 (m, 4H), 7.48 (m, 2H).
LRMS M+H: 507

### Example 197

### 5-bromo-2-[2-[4-cyano-4-[(4-fluorophenyl)hydroxymethyl]-1-piperidinyl]-2-oxoethoxy]benzeneacetic acid methyl ester

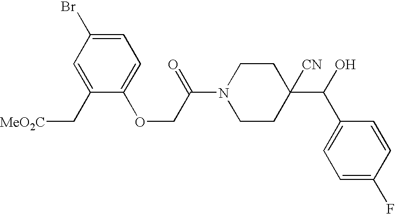 Prepared in a similar manner to Compound 2. ¹H NMR (400 MHz, DMSO-d₆): δ/ppm=1.34 (m, 2H), 1.47 (m, 1H), 1.65 (m, 1H), 2.14 (m, 1H), 2.62 (m, 1H), 3.04 (m, 1H), 3.56 (s, 3H), 3.62 (s, 2H), 3.84 (m, 1H), 4.38 (m, 1H), 4.54 (s, 1H), 4.84 (m, 2H), 6.21 (s, 1H), 6.86 (m, 1H), 7.18 (m, 2H), 7.36 (m, 2H), 7.44 (m, 1H).
LRMS M+H: 519

### Example 198

### 5-bromo-2-[2-[4-cyano-4-[(4-fluorophenyl)hydroxymethyl]-1-piperidinyl]-2-oxoethoxy]benzeneacetic acid

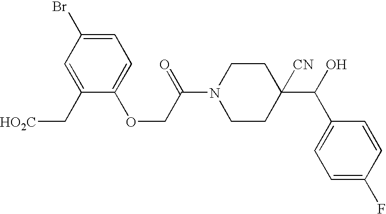 Prepared from Compound 197 in a similar manner to Compound 103. ¹H NMR (400 MHz, CDCl₃): δ/ppm=1.24 (m, 1H), 1.50 (m, 1H), 1.66 (m, 1H), 2.18 (m, 1H), 2.62 (m, 1H), 3.04 (m, 1H), 3.56 (s, 2H), 3.88 (m, 1H), 4.38 (m, 1H), 4.54 (s, 1H), 4.84 (m, 2H), 6.18 (s, 1H), 6.86 (m, 1H), 7.18 (m, 2H), 7.36 (m, 2H), 7.44 (m, 2H).
LRMS M+H: 505

### Example 199

### 5-chloro-2-[2-[4-cyano-4-[(4-fluorophenyl)methyl]-1-piperidinyl]-2-oxoethoxy]α-fluorobenzeneacetic acid methyl ester

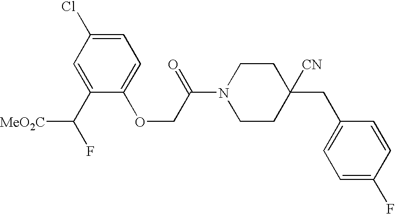 Prepared in a similar manner to Compound 2. ¹H NMR (400 MHz, CDCl₃): δ/ppm=1.30-1.70 (m, 1H), 1.70-1.90 (m, 3H), 2.80-2.96 (m, 3H), 3.26-3.42 (m, 1H), 3.76-3.82 (m, 3H), 3.92-4.08 (m, 1H), 4.48-4.86 (m, 3H), 5.80-6.08 (m, 1H), 6.88-7.06 (m, 3H), 7.18-7.30 (m, 2H), 7.32-7.42 (m, 2H).
LRMS M+H: 477

### Example 200

### 5-chloro-2-[2-[4-cyano-4-[(4-fluorophenyl)methyl]-1-piperidinyl]-2-oxoethoxy]-α-fluorobenzeneacetic acid

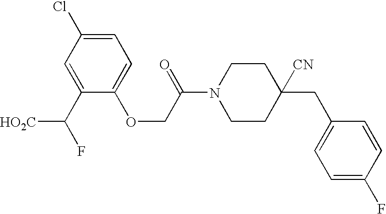 Prepared from Compound 199 in a similar manner to Compound 175. ¹H NMR (400 MHz, DMSO-d₆): δ/ppm=1.44-1.58 (m, 1H), 1.60-1.82 (m, 3H), 2.67 (m, 1H), 2.90 (s, 2H), 3.04-3.18 (m, 1H), 3.86 (m, 1H), 4.34 (m, 1H), 4.76-5.00 (m, 2H), 6.10 (d, 1H), 7.03 (m, 1H), 7.16 (m, 2H), 7.26-7.38 (m, 3H), 7.40-7.46 (m, 1H).
LRMS M+H: 463

### Example 201

### 5-bromo-2-[2-[4-(2-cyanoethyl)-4-[(4-fluorophenyl)methyl]-1-piperidinyl]-2-oxoethoxy]benzeneacetic acid methyl ester

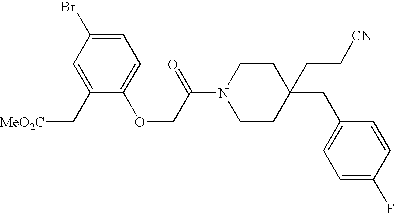 Prepared in a similar manner to Compound 2. ¹H NMR (400 MHz, CDCl₃): δ/ppm=1.36-1.50 (m, 2H), 1.72 (m, 2H), 2.32 (m, 2H), 2.62 (s, 2H), 3.40-3.52 (m, 2H), 3.56-3.68 (m, 1H), 3.62 (s, 2H), 3.68 (s, 3H), 3.76-3.86 (m, 1H), 4.66 (m, 2H), 6.74-6.80 (m, 1H), 6.94-7.06 (m, 4H), 7.30-7.34 (m, 2H).
LRMS M+H: 531

### Example 202

### 5-bromo-2-[2-[4-(2-cyanoethyl)-4-[(4-fluorophenyl)methyl]-1-piperidinyl]-2-oxoethoxy]benzeneacetic acid

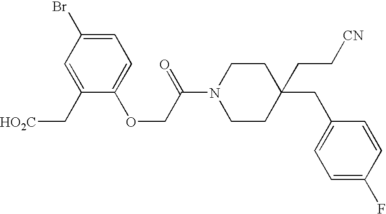 Prepared from Compound 201 in a similar manner to Compound 175. ¹H NMR (400 MHz, CDCl₃): δ/ppm=1.34-1.62 (m, 4H), 1.62-1.86 (m, 2H), 2.36 (m, 2H), 2.58-2.68 (m, 2H), 3.36-3.54 (m, 2H), 3.58-3.72 (m, 3H), 3.84-3.92 (m, 1H), 4.64-4.80 (m, 2H), 6.72-6.78 (m, 1H), 6.98-7.04 (m, 4H), 7.33-7.38 (m, 2H).
LRMS M+H: 517

### Example 204

### α-amino-5-chloro-2-[2-[4-cyano-4-[(4-fluorophenyl)methyl]-1-piperidinyl]-2-oxoethoxy]benzeneacetic acid

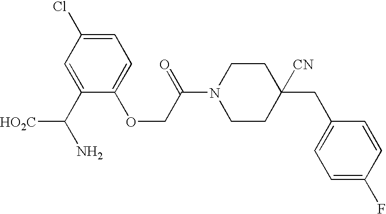 Prepared in a similar manner to Compound 103. ¹H NMR (400 MHz, CDCl₃): δ/ppm=1.38-1.64 (m, 2H), 1.66-1.90 (m, 2H), 2.76 (d, 3H), 3.14 (m, 1H), 3.58 (m, 1H), 4.38 (m, 1H), 4.88 (m, 2H), 5.10 (m, 1H), 6.82 (d, 1H), 7.02 (m, 2H), 7.20 (m, 2H), 7.34 (m, 2H), 8.58 (s, 2H).
LRMS M+H: 460

### Example 205

### 5-chloro-2-[2-[4-cyano-4-[(4-fluorophenyl)methyl]-1-piperidinyl]-2-oxoethoxy]benzeneacetic acid methyl ester

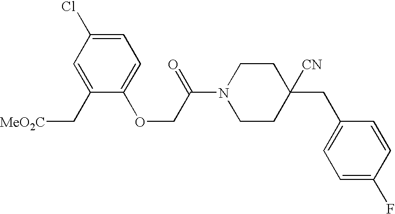 Prepared in a similar manner to Compound 59. ¹H NMR (400 MHz, CDCl₃): /ppm=1.50 (m, 2H), 1.90 (m, 2H), 2.85 (m, 3H), 3.30 (m, 1H), 3.60 (d, 2H), 3.68 (s, 3H), 4.00 (d, 1H), 4.65 (m, 2H), 4.75 (d, 1H), 6.85 (d, 1H), 7.05 (t, 2H), 7.22 (m, 4H).
LRMS M+H: 459

### Example 206

### 5-chloro-2-[2-[4-cyano-4-[(4-fluorophenyl)methyl]-1-piperidinyl]-2-oxoethoxy]benzeneacetic acid

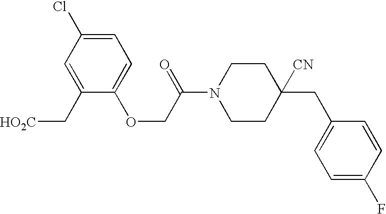 Prepared from Compound 205 in a similar manner to Compound 103. ¹H NMR (400 MHz, CDCl₃): δ/ppm=1.50 (m, 2H), 1.90 (m, 2H), 2.80 (m, 3H), 3.30 (m, 1H), 3.60 (m, 2H), 3.85 (m, 1H), 4.65 (m, 3H), 6.75 (m, 1H), 7.05 (m, 2H), 7.22 (m, 4H).
LRMS M+H: 445

### Example 207

### 5-chloro-2-[2-[4-cyano-4-[(4-fluorophenyl)methyl]-1-piperidinyl]-2-oxoethoxy]-4-(trifluoromethyl)benzoic acid methyl ester

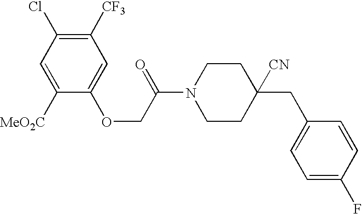 Prepared in a similar manner to Compound 59. ¹H NMR (400 MHz, CDCl₃): δ/ppm=1.40-1.55 (m, 2H), 1.85 (m, 2H), 2.75 (s, 2H), 2.80 (m, 1H), 3.30 (m, 1H), 3.82 (s, 3H), 4.05 (m, 1H), 4.55 (m, 1H), 4.70 (q, 2H), 6.95 (t, 2H), 7.20 (m, 2H), 7.25 (s, 1H), 7.85 (s, 1H).
LRMS M+H: 513

### Example 208

### 5-chloro-2-[2-[4-cyano-4-[(4-fluorophenyl)methyl]-1-piperidinyl]-2-oxoethoxy]-4-(trifluoromethyl)benzoic acid

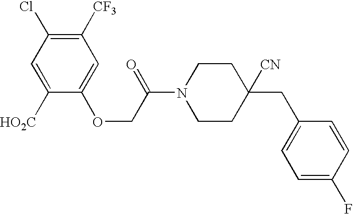 Prepared from Compound 207 in a similar manner to Compound 103. ¹H NMR (400 MHz, CDCl₃): δ/ppm=1.50 (m, 2H), 1.95 (m, 2H), 2.85 (s, 2H), 2.90 (m, 1H), 3.36 (m, 1H), 3.65 (m, 1H), 4.45 (m, 1H), 4.90 (q, 2H), 6.95 (m, 2H), 7.05 (m, 2H), 7.10 (s, 1H), 8.05 (s, 1H), 11.10 (s, 1H).
LRMS M+H: 499

### Example 209

### 5-bromo-2-[2-[4-[(4-fluorophenyl)methyl]-1-piperidinyl]-2-oxoethoxy]benzeneacetic acid methyl ester

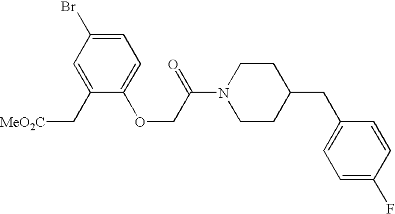 Prepared in a similar manner to Compound 59. ¹H NMR (400 MHz, CDCl₃): δ/ppm=1.10 (m, 2H), 1.68 (m, 3H), 2.50 (d, 2H), 2.55 (m, 1H), 2.98 (m, 1H), 3.60 (s, 2H), 3.65 (s, 3H), 3.90 (m, 1H), 4.55 (m, 1H), 4.65 (m, 2H), 6.80 (d, 1H), 6.95 (m, 2H), 7.05 (m, 2H), 7.35 (m, 2H).
LRMS M+H: 479

### Example 210

### 5-bromo-2-[2-[4-[(4-fluorophenyl)methyl]-1-piperidinyl]-2-oxoethoxy]benzeneacetic acid

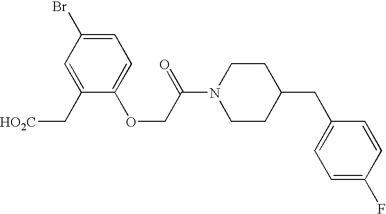 Prepared from Compound 209 in a similar manner to Compound 103. ¹H NMR (400 MHz, CDCl₃): δ/ppm=1.20 (m, 2H), 1.78 (m, 3H), 2.50 (m, 2H), 2.65 (m, 1H), 3.05 (m, 1H), 3.70 (s, 2H), 3.85 (m, 1H), 4.55 (m, 1H), 4.75 (m, 2H), 6.70 (d, 1H), 6.95 (t, 2H), 7.05 (m, 2H), 7.38 (m, 2H), 8.50 (s, 1H).
LRMS M+H: 465

### Example 211

### 5-bromo-2-[2-[4-[(4-chlorophenyl)methyl]-4-cyano-1-piperidinyl]-2-oxoethoxy]benzeneacetic acid methyl ester

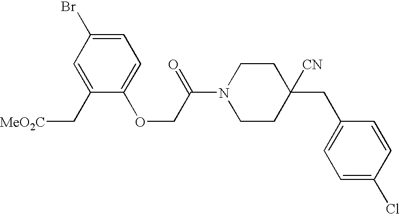 Prepared in a similar manner to Compound 59. ¹H NMR (400 MHz, CDCl₃): δ/ppm=1.50 (m, 2H), 1.90 (m, 2H), 2.85 (s, 2H), 2.90 (m, 1H), 3.30 (m, 1H), 3.60 (m, 2H), 3.65 (s, 3H), 4.00 (m, 1H), 4.60 (m, 2H), 4.70 (d, 1H), 6.80 (m, 1H), 7.20 (m, 2H), 7.35 (m, 4H).
LRMS M+H: 520

### Example 212

### 5-bromo-2-[2-[4-[(4-chlorophenyl)methyl]-4-cyano-1-piperidinyl]-2-oxoethoxy]benzeneacetic acid

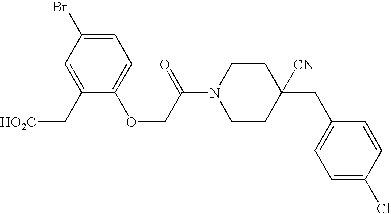 Prepared from Compound 211 in a similar manner to Compound 103. ¹H NMR (400 MHz, CDCl₃): δ/ppm=1.50 (m, 2H), 1.90 (d, 2H), 2.85 (s, 2H), 2.90 (m, 1H), 3.40 (m, 1H), 3.70 (s, 2H), 3.90 (m, 1H), 4.70 (m, 3H), 6.78 (d, 1H), 7.20 (d, 2H), 7.38 (m, 4H).
LRMS M+H: 506

### Example 213

### 5-chloro-2-[2-[4-cyano-4-[(4-fluorophenyl)methyl]-1-piperidinyl]-2-oxoethoxy]-α-hydroxybenzeneacetic acid methyl ester

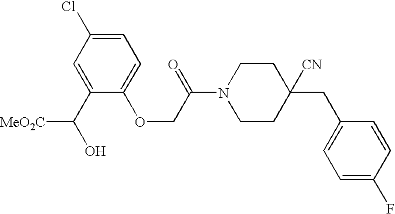 Prepared in a similar manner to Compound 59. ¹H NMR (400 MHz, CDCl₃): δ/ppm=1.46-1.66 (m, 2H), 1.86-2.00 (m, 2H), 2.78-2.98 (m, 3H), 3.32-3.44 (m, 1H), 3.74 (s, 3H), 3.78-3.92 (m, 1H), 4.58-4.86 (m, 3H), 5.18-5.28 (m, 1H), 6.84 (d, 1H), 7.00-7.08 (m, 2H), 7.20-7.32 (m, 4H).
LRMS M+H: 475

### Example 214

### 5-chloro-2-[2-[4-cyano-4-[(4-fluorophenyl)methyl]-1-piperidinyl]-2-oxoethoxy]-α-hydroxybenzeneacetic acid

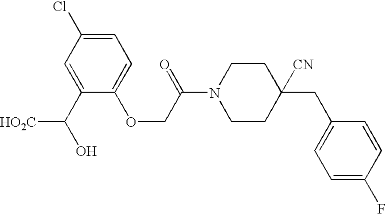 Prepared from Compound 213 in a similar manner to Compound 103. ¹H NMR (400 MHz, DMSO-d₆+TFA): δ/ppm=1.48 (m, 1H), 1.60-1.80 (m, 3H), 2.67 (m, 1H), 2.85 (s, 2H), 3.10 (m, 1H), 3.86 (m, 1H), 4.36 (m, 1H), 4.74-4.92 (m, 2H), 5.24 (s, 1H), 6.93 (m, 1H), 7.06 (m, 2H), 7.18-7.30 (m, 3H), 7.32 (m, 1H).
LRMS M+H: 461

### Example 215

### 5-chloro-2-[2-[4-cyano-4-[(4-fluorophenyl)methyl]-1-piperidinyl]-2-oxoethoxy]-4-methylbenzeneacetic acid methyl ester

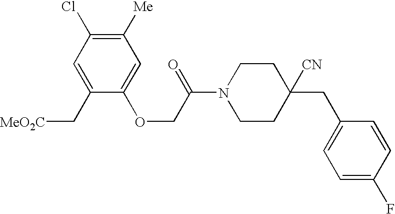 Prepared in a similar manner to Compound 59. ¹H NMR (400 MHz, CDCl₃): δ/ppm=1.44-1.58 (m, 2H), 1.84-1.96 (m, 2H), 2.34 (s, 3H), 2.84 (s, 2H), 2.85-2.94 (m, 1H), 3.26-3.38 (m, 1H), 3.52-3.64 (m, 2H), 3.68 (s, 3H), 3.98-4.06 (m, 1H), 4.56-4.76 (m, 3H), 6.77 (s, 1H), 7.00-7.07 (m, 2H), 7.18 (s, 1H), 7.21-7.26 (m, 2H).
LRMS M+H: 473

### Example 216

### 5-chloro-2-[2-[4-cyano-4-[(4-fluorophenyl)methyl]-1-piperidinyl]-2-oxoethoxy]-4-methylbenzeneacetic acid

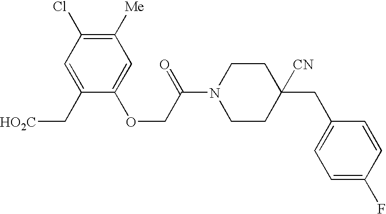 Prepared from Compound 215 in a similar manner to Compound 175. ¹H NMR (400 MHz, CDCl₃): δ/ppm=1.46-1.60 (m, 2H), 1.92 (m, 2H), 2.34 (s, 2H), 2.84 (s, 2H), 2.91 (m, 1H), 3.28 (m, 1H), 3.64 (s, 2H), 3.92 (m, 1H), 4.58-4.78 (m, 3H), 6.73 (s, 1H), 7.00-7.07 (m, 2H), 7.20-7.25 (m, 3H).
LRMS M+H: 459

### Example 217

### 5-bromo-2-[2-[4-cyano-4-[(4-fluorophenyl)methyl]-1-piperidinyl]-2-oxoethoxy]-α,α-difluorobenzeneacetic acid methyl ester

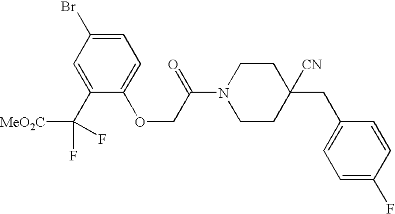 Prepared in a similar manner to Compound 59. ¹H NMR (400 MHz, CDCl₃): δ/ppm=1.62-1.73 (m, 1H), 1.82-1.89 (m, 2H), 1.90-1.98 (m, 1H), 2.82-2.98 (m, 3H), 3.30-3.42 (m, 1H), 3.82 (s, 3H), 3.88-3.96 (m, 1H), 4.46-4.84 (m, 3H), 6.85 (d, 1H), 7.00-7.08 (m, 2H), 7.24-7.30 (m, 2H), 7.60 (dd, 1H), 7.80 (d, 1H).
LRMS M+H: 540

### Example 218

### 5-bromo-2-[2-[4-cyano-4-[(4-fluorophenyl)methyl]-1-piperidinyl]-2-oxoethoxy]-α,α-difluorobenzeneacetic acid

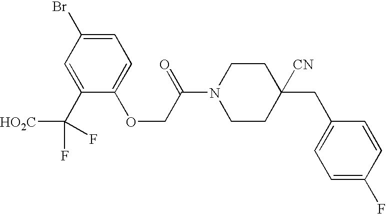 Prepared from Compound 217 in a similar manner to Compound 175. ¹H NMR (400 MHz, CDCl₃): δ/ppm=1.64-1.77 (m, 1H), 1.77-1.96 (m, 3H), 2.84-3.00 (m, 3H), 3.38 (m, 1H), 4.54 (m, 1H), 4.64 (m, 1H), 4.82 (m, 1H), 6.80 (m, 1H), 7.04 (m, 2H), 7.22-7.30 (m, 2H), 7.58 (m, 1H), 7.80-7.84 (m, 1H).
LRMS M+H: 525

### Example 219

### 5-bromo-2-[2-[4-cyano-4-[(4-fluorophenyl)methyl]-1-piperidinyl]-2-oxoethoxy]-α,α-dimethylbenzeneacetic acid methyl ester

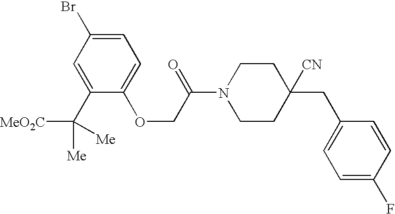 Prepared in a similar manner to Compound 59. ¹H NMR (400 MHz, DMSO-d₆): δ/ppm=1.43 (s, 6H), 1.54 (t, 1H), 1.75 (m, 3H), 2.68 (t, 1H), 2.93 (s, 2H), 3.12 (t, 1H), 3.50 (s, 3H), 3.84 (d, 1H), 4.36 (d, 1H), 4.80 (d, 2H), 6.80 (d, 1H), 7.19 (t, 2H), 7.34 (m, 4H).
LRMS M+H: 531

### Example 220

### 5-bromo-2-[2-[4-cyano-4-[(4-fluorophenyl)methyl]-1-piperidinyl]-2-oxoethoxy]-α,α-dimethylbenzeneacetic acid

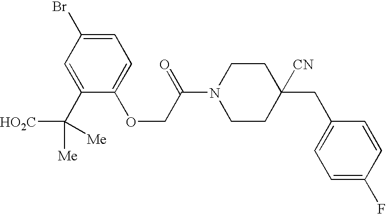 To a solution of Compound 219 (100 mg, 0.19 mmol) in pyridine (3 mL) under nitrogen was added lithium iodide (60 mg, 0.47 mmol), and the mixture was stirred at 140° C. After 48 h, the pyridine was removed under vacuum. Purification by reverse phase HPLC afforded Compound 220. ¹H NMR (400 MHz, DMSO-d₆): δ/ppm=1.42 (s, 6H), 1.53 (t, 1H), 1.76 (m, 3H), 2.68 (t, 1H), 2.92 (s, 2H), 3.14 (t, 1H), 3.86 (d, 1H), 4.37 (d, 1H), 4.78 (d, 2H), 6.81 (d, 1H), 7.19 (t, 2H), 7.33 (m, 4H).
LRMS M+H: 517

### Example 221

### 1-[(4-bromo-2-formyl phenoxy)acetyl]-4-[(4-fluorophenyl)methyl]-4-piperidinecarbonitrile

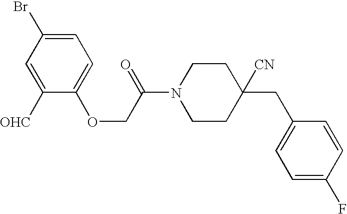 Prepared in a similar manner to Compound 59. ¹H NMR (400 MHz, CDCl₃): δ/ppm 1.46 (m, 2H), 1.95 (m, 2H), 2.84 (s, 2H), 2.91 (t, 1H), 3.40 (t, 1H), 3.98 (d, 1H), 4.62 (d, 1H), 4.80 (d, 1H), 4.84 (d, 1H), 6.92 (d, 1H), 7.02 (t, 2H), 7.22 (m, 2H), 7.62 (d, 1H), 7.95 (s, 1H), 10.40 (s, 1H).

### Example 222

### 1-[[4-bromo-2-(hydroxymethyl)phenoxy]acetyl]-4-[(4-fluorophenyl)methyl]-4-piperidinecarbonitrile

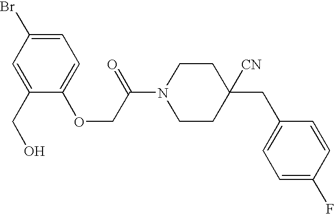 Prepared from Compound 221 in a similar manner to Compound 97. ¹H NMR (400 MHz, CDCl₃): δ/ppm=1.46 (m, 2H), 1.94 (m, 2H), 2.85 (m, 3H), 3.38 (t, 1H), 3.82 (d, 1H), 4.64 (m, 3H), 4.78 (s, 2H), 6.75 (d, 1H), 7.05 (t, 2H), 7.22 (m, 2H), 7.35 (m, 1H), 7.45 (m, 1H).
LRMS M+H: 461

### Example 223

### [[5-bromo-2-[2-[4-cyano-4-[(4-fluorophenyl)methyl]-1-piperidinyl]-2-oxoethoxy]phenyl]methyl]phosphonic acid diethyl ester

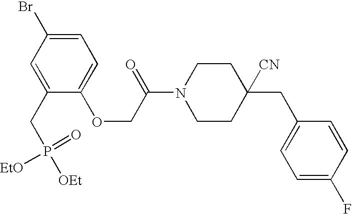

### A. 1-[[4-bromo-2-(chloromethyl)phenoxy]acetyl]-4-[(4-fluorophenyl)methyl]-4-piperidinecarbonitrile

To a solution of Compound 222 (460 mg, 1.0 mmol) in dichloromethane (10 mL) was added 2,6-lutidine (0.64 mL, 5.5 mmol) followed by thionyl chloride (0.60 mL, 5.0 mmol) and the mixture heated at reflux overnight. Additional 2,6-lutidine (0.5 mL, 4.3 mmol) and thionyl chloride (0.30 mL, 2.5 mmol) were added, and reflux continued for 3 hours. The reaction was diluted with dichloromethane and washed with aqueous hydrochloric acid (0.5 N). The organic layer was dried and concentrated to afford the chloro Intermediate 223a (470 mg) which was used without further purification.

### B. [[5-bromo-2-[2-[4-cyano-4-[(4-fluorophenyl)methyl]-1-piperidinyl]-2-oxoethoxy]phenyl]methyl]phosphonic acid diethyl ester

Intermediate 223a (480 mg, 1.0 mmol) was added to triethyl phosphite (0.25 mL, 1.5 mmol), and the mixture was heated at 80° C. for 3 hours. Purification by chromatography on silica afforded Compound 223 (470 mg). ¹H NMR (400 MHz, CDCl₃): δ/ppm=1.25 (t, 6H), 1.50 (m, 2H), 1.94 (m, 2H), 2.83 (s, 2H), 2.90 (t, 1H), 3.20 (dd, 2H), 3.35 (t, 1H), 4.05 (m, 5H), 4.70 (m, 3H), 6.80 (d, 1H), 7.03 (t, 2H), 7.24 (m, 2H), 7.30 (m, 1H), 7.44 (m, 1H).

LRMS M+H: 581

### Examples 224 and 225

### [[5-bromo-2-[2-[4-cyano-4-[(4-fluorophenyl)methyl]-1-piperidinyl]-2-oxoethoxy]phenyl]methyl]phosphonic acid and

### [[5-bromo-2-[2-[4-cyano-4-[(4-fluorophenyl)methyl]-1-piperidinyl]-2-oxoethoxy]phenyl]methyl]phosphonic acid ethyl ester

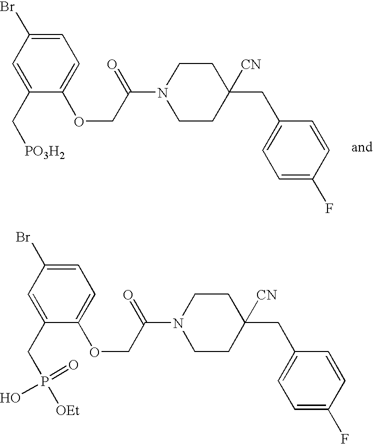 To a solution of Compound 223 (400 mg, 0.69 mmol) in dichloromethane (8 mL) were added anisole (0.15 mL, 1.4 mmol) and bromotrimethylsilane (0.14 mL, 1.0 mmol), and the mixture stirred at ambient temperature overnight. Concentration and separation of the products by reverse phase HPLC afforded Compounds 224 (130 mg) and 225 (49 mg).
Compound 224: ¹H NMR (400 MHz, DMSO-d₆): δ/ppm=1.46 (t, 1H), 1.65 (t, 1H), 1.76 (m, 2H), 2.65 (t, 1H), 2.90 (s, 2H), 3.02-3.15 (m, 3H), 3.90 (d, 1H), 4.35 (d, 1H), 4.82 (d, 1H), 4.84 (d, 1H), 6.85 (d, 1H), 7.15 (m, 2H), 7.30, (m, 3H), 7.42 (m, 1H).
LRMS M+H: 525
Compound 225: ¹H NMR (400 MHz, DMSO-d₆): δ/ppm=1.10 (t, 3H), 1.50 (m, 1H), 1.67 (m, 1H), 1.77 (m, 2H), 2.66 (m, 1H), 2.90 (s, 2H), 3.10 (m, 3H), 3.87 (m, 3H), 4.35 (d, 1H), 4.82 (d, 1H), 4.84 (d, 1H), 6.88 (d, 1H), 7.15 (t, 2H), 7.30 (m, 3H), 7.40 (m, 1H).
LRMS M+H: 553

### Example 226

### 5-chloro-2-[2-[4-cyano-4-[(4-fluorophenyl)methyl]-1-piperidinyl]-2-oxoethoxy]benzenemethanesulfonic acid

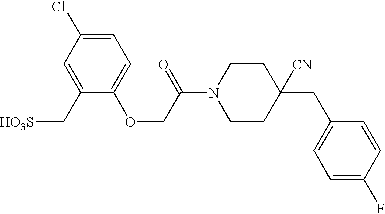

### A. 1-[[2-(bromomethyl)-4-chlorophenoxy]acetyl]-4-[(4-fluorophenyl)methyl]4-piperidinecarbonitrile

To a solution of Compound 99 (280 mg, 0.67 mmol) in dichloromethane (5 mL) were added carbon tetrabromide (230 mg, 0.74 mmol) and triphenylphosphine (190 mL, 0.74 mmol) and the mixture heated at 40° C. for 2 hours. Concentration and purification by chromatography on silica afforded the bromo Intermediate 226a (289 mg) as a colorless oil. ¹H NMR (400 MHz, CDCl₃): δ/ppm=1.42-1.58 (m, 2H), 1.90-1.98 (m, 2H), 2.80-2.96 (m, 3H), 3.34-3.44 (m, 1H), 4.16-4.22 (m, 1H), 4.44-4.54 (m, 2H), 4.60-4.86 (m, 3H), 6.88 (d, 1H), 6.98-7.06 (m, 2H), 7.18-7.26 (m, 3H), 7.34 (d, 1H).

LRMS M+H: 479

B. 5-chloro-2-[2-[4-cyano-4-[(4-fluorophenyl)methyl]-1-piperidinyl]-2-oxoethoxy]benzenemethanesulfonic acid

To a solution of Intermediate 226a (270 mg, 0.56 mmol) in aqueous ethanol (6 mL) was added sodium sulfite (283 mg, 2.2 mmol), and the mixture was heated at reflux for 2 hours. The mixture was cooled to ambient temperature and the solid removed by filtration. The filtrate was acidified to pH 1-2 by addition of 2 N hydrochloric acid and concentrated. Purification by reverse phase HPLC, followed by recrystallization afforded Compound 226 (103 mg) as a white solid. ¹H NMR (400 MHz, DMSO-d₆+D₂O): δ/ppm=1.44-1.56 (m, 1H), 1.64-1.82 (m, 3H), 2.86 (m, 1H), 2.88 (s, 2H), 3.08 (m, 1 n), 3.70-3.86 (m, 2H), 3.90-3.98 (m, 1H), 4.35 (m, 1H), 4.66-4.84 (m, 2H), 6.89 (m, 1H), 7.10-7.18 (m, 3H), 7.28-7.36 (m, 2H), 7.46 (m, 1H).

LRMS M+H: 481

### Example 227

### 5-chloro-2-[3-[4-cyano-4-[(4-fluorophenyl)methyl]-1-piperidinyl]-3-oxopropyl]benzeneacetic acid

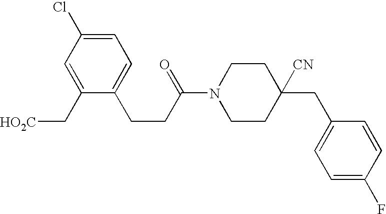

A mixture of Compound 175 (280 mg, 0.64 mmol) and 5% Pt-C (catalytic) in ethyl acetate and methanol (4:1 v/v, 50 mL) was hydrogenated at 45 psi overnight. The mixture was filtered and the filtrate concentrated to dryness. Purification by chromatography on silica afforded Compound 227 (40 mg) as a white solid. ¹H NMR (400 MHz, DMSO-d₆): δ/ppm=1.34-1.48 (m, 2H), 1.62-1.78 (m, 2H), 2.50-2.68 (m, 3H), 2.68-2.78 (m, 2H), 2.85 (s, 2H), 2.98 (m, 1H), 3.64 (s, 2H), 3.88 (m, 1H), 4.42 (m, 1H), 7.12-7.19 (m, 2H), 7.19-7.32 (m, 5H).

LRMS M+H: 443

### Example 228

### 5-chloro-2-[2-[4-cyano-4-[(4-fluorophenyl)methyl]-1-piperidinyl]-2-oxoethoxy]-α-hydroxybenzeneacetamide

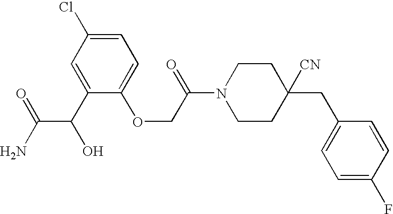

### A. 1-[[4-chloro-2-(cyanohydroxymethyl)phenoxy]acetyl]-4-[(4-fluorophenyl)methyl]-4-piperidinecarbonitrile

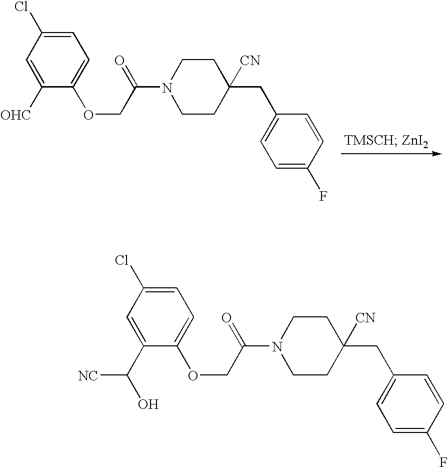 To a solution of Compound 25 (1000 mg, 2.4 mmol) in dichloromethane (10 mL) at −10° C. were added zinc iodide (100 mg, 0.31 mmol) and trimethylsilyl cyanide (0.45 mL, 3.4 mmol). The mixture was warmed to ambient temperature and stirred overnight. The reaction was quenched with water (1 mL), stirred for 15 minutes, then diluted with ethyl acetate, dried, concentrated and purified by chromatography on silica to afford Intermediate 228a (890 mg).

### B. 5-chloro-2-[2-[4-cyano-4-[(4-fluorophenyl)methyl]-1-piperidinyl]-2-oxoethoxy]-α-hydroxybenzeneacetamide

Intermediate 228a (400 mg, 0.9 mmol) was dissolved in a mixture of ether (5 mL), methanol (0.5 mL) and hydrochloric acid (4.0 N solution in dioxane, 3.5 mL, 14 mmol), and the solution stirred at ambient temperature for 24 hours. The resulting solid was collected by filtration and washed with ether. Purification by reverse phase HPLC afforded Compound 228 (220 mg) as a white solid. ¹H NMR (400 MHz, DMSO-d₆+D₂O): δ/ppm=1.42-1.56 (m, 1H), 1.62-1.84 (m, 3H), 2.68 (m, 1H), 2.87 (s, 2H), 3.10 (m, 1H), 3.84 (m, 1H), 4.34 (m, 1H), 4.78-5.00 (m, 2H), 5.20 (s, 1H), 6.97 (m, 1H), 7.13 (m, 2H), 7.20-7.32 (m, 4H).

LRMS M+H: 460

### Example 229

### N-[2-[5-bromo-2-[2-[4-cyano-4-[(4-fluorophenyl)methyl]-1-piperidinyl]-2-oxoethoxy]phenyl]acetyl]methanesulfonamide

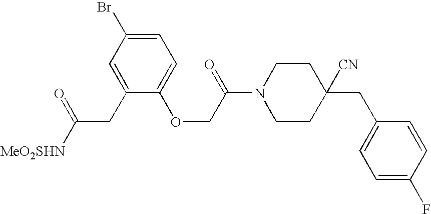 To a solution of Compound 105 (500 mg, 1.0 mmol) in dichloromethane (15 mL) and dimethylformamide (2 mL) were added dimethylaminopyridine (190 mg, 1.5 mmol) and 1-[3-(dimethylamino)propyl]-3-ethylcarbodiimide hydrochloride (280 mg, 1.4 mmol), followed by methanesulfonamide (130 mg, 1.3 mmol) and triethylamine (0.3 mL, 2.2 mmol). The mixture was stirred at ambient temperature for 5 days. The reaction was diluted with dichloromethane and washed with aqueous hydrochloric acid (0.2 N). The organic layer was dried, concentrated and purified by chromatography on silica. The resulting oil was dissolved in dichloromethane (10 mL) and ether (2 mL) was added. The mixture was concentrated and the resulting solid was washed with dichloromethane to afford Compound 229 (235 mg) as a white solid. ¹H NMR (400 MHz, DMSO-d₆): δ/ppm=1.49 (t, 1H), 1.67 (t, 1H), 1.79 (d, 2H), 2.67 (t, 1H), 2.92 (s, 2H), 3.10 (t, 1H), 3.21 (s, 3H), 3.64 (s, 2H), 3.87 (d, 1H), 4.35 (d, 1H), 4.85 (AB q, 2H), 6.88 (d, 1H), 7.18 (t, 2H), 7.32 (dd, 2H), 7.37 (d, 0.5H), 7.40 (s, 1.5H).
LRMS M+H: 566

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding U.S. Provisional Application Ser. No. 60/638,033, filed Dec. 20, 2004, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of the following general formulae I and II: 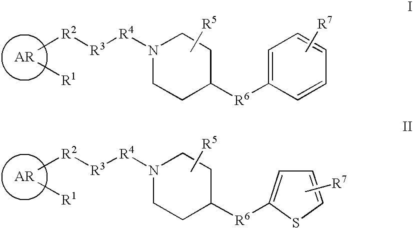
enantiomers, diastereomers, tautomers, salts, solvates and radio-labeled analogues thereof
wherein 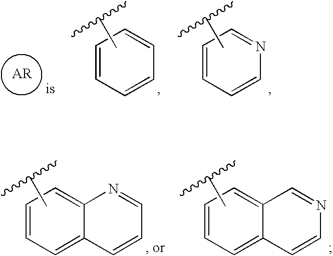
R¹ is one or more groups independently selected from
(c) hydrogen, halo, nitro, alkyl, alkoxy, haloalkyl, cycloalkyl, (cycloalkyl)alkyl, alkenyl, alkynyl, aryl, (aryl)alkyl, heteroaryl, (heteroaryl)alkyl, heterocyclo, or (heterocyclo)alkyl,
(d) —(O)_{q}—(Y)ₚ—CN,
—(O)_{q}—(Y)ₚ—OR¹⁰,
—(O)_{q*}—(Y)ₚ—C(═O)R¹⁰,
—(O)_{q*}—(Y)ₚ—C(═O)OR¹⁰,
—(O)_{q}—(Y)ₚ—C(═O)—C(═O)OR¹⁰,
—(O)_{q*}—(Y)ₚ—C(═O)—NR¹¹R¹²,
—(O)_{q*}—(Y)ₚ—C(═O)—NR^{11a}—S(═O)ₜR^{10a},
—(O)_{q}—(Y)ₚ—NH—OH,
—(O)_{q}—(Y)ₚ—NH—NH₂,
—(O)_{q}—(Y)ₚ—NR¹¹R¹²,
—(O)_{q}—(Y)ₚ—NR^{11a}—C(═O)R¹⁰,
—(O)_{q}—(Y)ₚ—NR^{11a}—C(═O)OR¹⁰,
—(O)_{q}—(Y)ₚ—NR^{11a}—SO₂R¹⁰,
—(O)_{q}—(Y)ₚ—N(R^{11a})—SO₂NR¹¹R¹²,
—(O)_{q}—(Y)ₚ—NR^{11a}—C(═O)—NR¹¹R¹²,
—(O)_{q}—(Y)ₚ—NR^{11a}—(Y)—C(═O)OR¹⁰,
—(O)_{q}—(Y)ₚ—NR^{11a}—C(═O)—(Y—NR¹¹R¹²,
—(O)_{q}—(Y)ₚ—NR^{11a}—C(═O)—(Y)—C(═O)—NR¹¹R¹²
—(O)_{q}—(Y)ₚ—SR¹⁰,
—(O)_{q}—(Y)ₚ—SO₃H,
—(O)_{q}—(Y)ₚ—S(═O)ₜR^{10a},
—(O)_{q}—(Y)ₚ—S(═O)ₜ—NR^{11a}—C(═O)R^{10a}, or
—(O)_{q}—(Y)ₚ—P(═O)(OH)OR¹⁰;
p is 0 or 1;
q* is or 1;
q is 0 or 1, provided that q is not 1 when p is 0;
t is 1 or 2;
Y at each occurrence is independently
c) alkylene optionally substituted independently with one or more halogen, —OH or —NR¹³R¹⁴ groups; or
d) alkenylene optionally substituted independently with one or more —OH or —NR¹³R¹⁴ groups, and;
R² is —O—, —S—, —N(R⁸)—, —N(R⁸)—C(═O)—, —C(R⁹)₂— or a bond;
R³ is alkylene or alkenylene either of which may be optionally independently substituted by one or more aryl, hydroxy, oxo, —C(═O)OR¹⁰, or —N(R⁸)₂;
R⁴ is —C(═O)—, —OC(═O)—, —C(═S)—, —CH₂— or a bond;
R⁵ is one or more groups independently selected from hydrogen, oxo, halo, alkyl, alkenyl, cycloalkyl, haloalkyl, (cycloalkyl)alkyl, (aryl)alkyl, (heterocyclo)alkyl, (heteroaryl)alkyl, -(alkyl)ₚ—CN, -(alkyl)ₚ—OR¹⁰, -(alkyl)ₚ—C(═O)R¹⁰, -(alkyl)ₚ—C(═O)OR¹⁰, -(alkyl)ₚ—S(═O)ₜR¹², -(alkyl)ₚ—C(═O)—NR¹¹R¹², -(alkyl)ₚ—NR^{11a}—(═O)NR¹¹R¹², -(alkyl)ₚ—NR^{11a}—C(═O)R¹⁰, or -(alkyl)ₚ—NR^{11a}—C(═O)OR¹⁰;
R⁶ is —C(═O)—, —C(═S)—, —C(R⁹)₂—, ═C(R⁹)—, —S—, —S(═O)ₜ—;
R⁷ is one or more groups independently selected from hydrogen, halo, alkyl, cycloalkyl, alkenyl, -(alkyl)ₚ—CN, -(alkyl)ₚ—OR¹⁰, -(alkyl)ₚ—C(═O)OR¹⁰, -(alkyl)ₚ—NR^{11a}—C(═O)R¹⁰, -(alkyl)ₚ—NR^{11a}—C(═O)OR¹⁰, -(alkyl)ₚ—C(═O)—NR¹¹R¹² or -(alkyl)ₚ—NR^{11a}—C(═O)—NR¹¹R¹²;
each R⁸ is independently selected from hydrogen, alkyl, cycloalkyl, (cycloalkyl)alkyl, aryl, (aryl)alkyl, heteroaryl, (heteroaryl)alkyl, heterocyclo, (heterocyclo)alkyl, -(alkyl)ₚ—C(═O)R¹⁰, -(alkyl)ₚ—C(═O)OR¹⁰, or -(alkyl)ₚ—C(═O)—NR¹¹R¹²;
each R⁹ is independently selected from hydrogen, halo, alkyl, cycloalkyl, haloalkyl, (cycloalkyl)alkyl, aryl, (aryl)alkyl, -(alkyl)ₚ—OR¹⁰, -(alkyl)ₚ—C(═O)R¹⁰, -(alkyl)ₚ—O—C(═O)R¹⁰, -(alkyl)ₚ-C(═O)OR¹⁰, -(alkyl)ₚ—NR¹¹R¹², -(alkyl)ₚ—NR^{11a}—C(═O)R¹⁰, -(alkyl)ₚ—NR^{11a}—C(═O)OR¹⁰, -(alkyl)ₚ—NR^{11a}—S(═O)ₜR^{10a} or -(alkyl)ₚ—C(═O)—NR¹¹R¹²;
each R¹⁰ is independently selected from
(c) hydrogen, or
(d) alkyl, haloalkyl, cycloalkyl, (cycloalkyl)alkyl, aryl, (aryl)alkyl, heterocyclo, (heterocyclo)alkyl, heteroaryl or (heteroaryl)alkyl any of which may be optionally independently substituted with one or more Z groups;
R^{10a} is alkyl, haloalkyl, cycloalkyl, (cycloalkyl)alkyl, aryl, (aryl)alkyl, heterocyclo, (heterocyclo)alkyl, heteroaryl or (heteroaryl)alkyl any of which may be optionally independently substituted with one or more Z groups;
each R¹¹, R^{11a} and R¹² is independently selected from
(d) hydrogen, hydroxy, NH₂ or
(e) —C(═NH)—NH₂, or
(f) alkyl, haloalkyl, (amino)alkyl, (hydroxy)alkyl, (alkoxy)alkyl, (aryloxy)alkyl, cycloalkyl, (cycloalkyl)alkyl, aryl, (aryl)alkyl, heterocyclo, (heterocyclo)alkyl, heteroaryl or (heteroaryl)alkyl any of which may be optionally independently substituted with one or more Z groups;
or R¹¹ and R¹² together with the nitrogen atom to which they are bonded may combine to form a heterocyclo ring optionally independently substituted with one or more Z groups;
R¹³ and R¹⁴ are independently hydrogen or alkyl;
Z at each occurrence is independently
(1) V, where V is
(i) alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocylco)alkyl, heteroaryl, or (heteroaryl)alkyl;
(ii) a group (i) which is itself substituted by one or more of the same or different groups (i); or
(iii) a group (i) or (ii) which is independently substituted by one or more (preferably 1 to 3) of the following groups (2) to (13),
(3) —SH or —SV,
(4) —C(O)H, —C(O)OH, —C(O)V, —C(O)OV, or —O—C(O)V,
(5) —SO₃H, —S(O)ₘV, or S(O)ₘN(V¹)V, where m is 1 or 2,
(6) halo,
(7) cyano,
(8) nitro,
(9) —U¹—NV²V³,
(10) —U¹—N(V¹)—U²—NV²V³,
(11) —U¹—N(V⁴)—U²—V,
(12) —U¹—N(V⁴)—U²—H,
(13) oxo;
U¹ and U² are each independently
(1) a single bond,
(2) —U³—S(O)ₜ—U⁴—,
(3) —U³—C(O) —U⁴—,
(4) —U³—C(S) —U⁴—,
(5) —U³—O—U⁴—,
(6) —U³—S—U⁴—,
(7) —U³—O—C(O) —U⁴—,
(8) —U³—C(O)—O—U⁴—,
(9) —U³—C(═NV^{1a}) —U⁴—, or
(10) —U³—C(O)—C(O) —U⁴—;
V¹, V^{1a}, V², V³ and V⁴
(1) are each independently hydrogen or a group provided in the definition of Z; or
(2) V² and V³ may together be alkylene or alkenylene, completing a 3- to 8-membered saturated or unsaturated ring together with the atoms to which they are attached, which ring is unsubstituted or substituted with one or more groups listed in the definition of Z, or
(3) V² or V³, together with V¹, may be alkylene or alkenylene completing a 3- to 8-membered saturated or unsaturated ring together with the nitrogen atoms to which they are attached, which ring is unsubstituted or substituted with one or more groups listed in the definition of Z; and
U³ and U⁴ are each independently
(1) a single bond,
(2) alkylene,
(3) alkenylene, or
(5) alkynylene.

2. A compound of claim 1 wherein 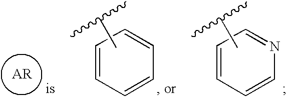
R¹ is one or more groups independently selected from hydrogen, alkyl, hydroxy, alkoxy, cyano, halo, -(alkyl)ₚ—OR¹⁰, -(alkyl)ₚ—NR^{11a}—C(═O)—NR¹¹R¹², -(alkyl)ₚ—C(═O)OR¹¹, -(alkyl)ₚ—C(═O)R¹⁰, -(alkyl)ₚ—CN, -(alkyl)ₚ—C(═O)—NR¹¹R¹², heteroaryl, (heteroaryl)alkyl, (heterocyclo)alkyl, -(alkyl)ₚ—NR¹¹R¹², -(alkyl)ₚ—NR^{11a}—C(═O)R¹⁰, -(alkyl)ₚ—NR^{11a}—CH₂—C(═O)OR¹⁰, or -(alkyl)ₚ—NR^{11a}—SO₂—R¹⁰;
R⁵ is one or more groups independently selected from hydrogen, alkyl, alkenyl, keto, —CN, —C(═O)OR¹⁰, haloalkyl, (heterocyclo)alkyl, —(CH₂)ₚ—OR¹⁰, —(CH₂)ₚ—NR¹¹R¹², or —C(═O)R¹⁰;
R⁶ is —CH₂—, —CHF—, —CH(OH)—, or —C(═O)—; and
R⁷ is one or two same or different halo groups; and
Y, p, q, R¹⁰, R¹¹, R^{11a}, and R¹² have the meaning as defined in claim 1.

3. A compound of claim 2 wherein
R² is —O—;
R³ is alkylene or alkenylene either of which may be optionally independently substituted by one or more aryl, hydroxy, oxo or —N(R⁸)₂; and
R⁴ is —C(═O)—; and
R⁸ has the meaning defined in claim 1.

4. A compound of claim 3 wherein

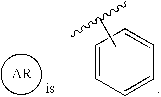

5. A compound of claim 4 wherein
R¹ is one or more groups independently selected from hydrogen, halo, -(alkyl)ₚ—NR^{11a}—C(═O)—NR¹¹R¹², -(alkyl)ₚ—C(═O)OR¹⁰, or -(alkyl)ₚ—C(═O)R¹⁰;
R⁶ is —CH₂—, —CH(OH)—, or —C(═O)—; and
p, q, R¹⁰, R¹¹, R^{11a}, and R¹² have the meaning as defined in claim 1.

6. Compounds of the following general formulae Ia and IIa: 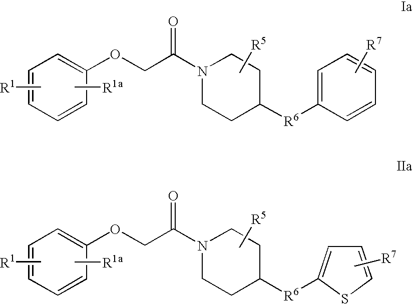
enantiomers, diastereomers, tautomers, salts, solvates and radio-labeled analogues thereof
wherein
R¹ is one or more groups independently selected from hydrogen, alkyl, haloalkyl, heterocyclo, hydroxy, alkoxy, cyano, halo or —(O)_{q}—(Y)ₚ—NR¹¹R¹²;
R^{1a} is heteroaryl, (heteroaryl)alkyl, (heterocyclo)alkyl,
—(O)_{q}—(Y)ₚ—CN,
—(O)_{q}—(Y)ₚ—OR¹⁰,
—(O)_{q*}—(Y)ₚ—C(═O)OR¹⁰,
—(O)_{q*}—(Y)ₚ—C(═O)R¹⁰,
—(O)_{q*}—(Y)ₚ—C(═O)—NR¹¹R¹²,
—(O)_{q}—(Y)ₚ—NR^{11a}—C(═O)—NR¹¹R¹²,
—(O)_{q}—(Y)ₚ—NR^{11a}—(Y)—C(═O)OR¹⁰,
—(O)_{q}—(Y)ₚ—NR^{11a}—SO₂R¹⁰, or
—(O)_{q}—(Y)ₚ—NR^{11a}—C(═O)—(Y)—NR¹¹R¹²
R⁵ is one or more groups independently selected from hydrogen, alkyl, alkenyl, keto, —CN, —C(═O)OR¹⁰, haloalkyl, (heterocyclo)alkyl, —(CH₂)ₚ—OR¹⁰, —(CH₂)ₚ—NR¹¹R¹², or —C(═O)R¹⁰;
R⁶ is —CH₂—, —CHF—, —CH(OH)—, or —C(═O)—; and
R⁷ is one or two same or different halo groups; and
Y, p, q, q*, R¹⁰, R¹¹, R^{11a}, and R¹² have the meaning as defined in claim 1.

7. A compound of claim 6 wherein
R¹ is halo; and
R^{1a} is —(CH₂)—OR¹⁰, —(CH₂)—NR¹¹R¹², —(CH₂)—NR^{11a}—C(═O)—NR¹¹R¹², —(CH₂)—C(═O)OR¹⁰, —(CH₂)—C(═O)R¹⁰, —(CH₂)—CN, —(CH₂)—C(═O)—NR¹¹R¹², —(CH₂)—NR^{11a}—C(═O)R¹⁰, —CH₂—NR^{11a}—CH₂—C(═O)OR¹⁰, —(CH₂)—NR^{11a}—SO₂—R¹⁰, —NR^{11a}—SO₂—R¹⁰, —C(═O)OR¹⁰, —CH═CH—C(═O)OR¹⁰, or —(CH₂)₂—C(═O)OR¹⁰, and
R¹⁰, R¹¹, R^{11a}, and R¹² have the meaning as defined in claim 1.

8. A compound of claim 6 and 7 wherein
q and q* is 0
p is 0 or 1
R¹⁰ is hydrogen or lower alkyl
R^{11a} is hydrogen
R¹¹ is hydrogen or lower alkyl
R¹² is hydrogen, lower alkyl, haloalkyl or alkyl-CO₂R¹⁰
or NR¹¹R¹² is pyrrolidine or piperidine

9. Compounds of the following general formulae Ib, Ic, and Id: 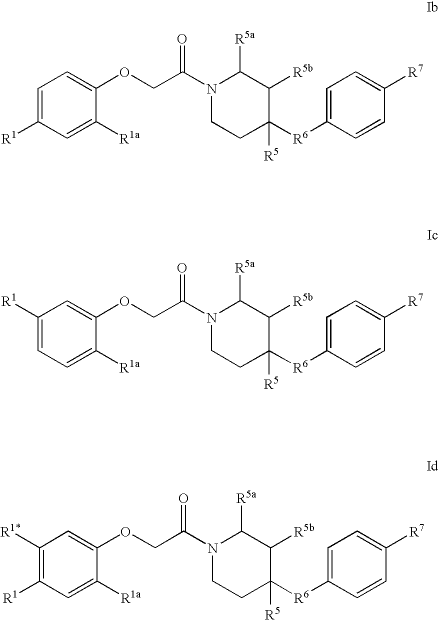
enantiomers, diastereomers, tautomers, salts, solvates and radio-labeled analogues thereof
wherein
R¹, R^{1a}, R⁵, R⁶, and R⁷ are as defined for formula Ia;
R¹* is hydrogen, alkyl, haloalkyl, heterocyclo, hydroxy, alkoxy, cyano, halo or —(O)_{q}—(Y)ₚ—NR¹¹R¹²;
R^{5a} is hydrogen or alkyl; and
R^{5b} is hydrogen, alkyl, keto, or hydroxyl;
Y, p, q, R¹¹, and R¹² have the meaning as defined in claim 1.

10. A compound of claim 9 wherein
R¹ is halo; and
R^{1a} is —(CH₂)—OR¹⁰, —(CH₂)—NR¹¹R¹², —(CH₂—NR^{11a}—C(═O)—NR¹¹R¹², —(CH₂)—C(═O)OR¹⁰, —(CH₂)—C(═O)R¹⁰, —(CH₂)CN, —(CH₂)—C(═O)—NR¹¹R¹², —(CH₂)—NR^{11a}—C(═O)R¹⁰, —CH₂—NR^{11a}—CH₂—C(═O)OR¹⁰, —(CH₂)—NR^{11a}—SO₂—R¹⁰; —NR^{11a}—SO₂—R¹⁰, —C(═O)OR¹⁰, —CH═CH—C(═O)OR¹⁰, or —(CH₂)₂—C(═O)OR¹⁰, and
R⁷ is fluoro or chloro; and
R¹⁰, R¹¹, R^{11a}, and R¹² have the meaning as defined in claim 1.

11. A compound of claim 9 and 10 wherein
q is 0
p is 0 or 1
Y is CH₂ or CH═CH or CH₂CH₂
R¹⁰ is hydrogen or lower alkyl
R^{11a} is hydrogen
R¹¹ is hydrogen or lower alkyl
R¹² is hydrogen, lower alkyl, haloalkyl or alkyl-CO₂R¹⁰
or NR¹¹R¹² is pyrrolidine or piperidine

12. Compounds of the following general formulae IIb, IIc, and IId: 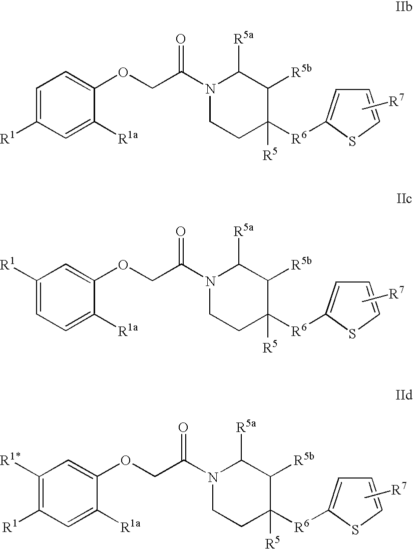
enantiomers, diastereomers, tautomers, salts, solvates and radio-labeled analogues thereof
wherein
R¹, R^{1a}, R⁵, R⁶, and R⁷ are as defined for formula Ia;
R¹* is hydrogen, alkyl, haloalkyl, heterocyclo, hydroxy, alkoxy, cyano, halo or —(O)_{q}—(Y)ₚ—NR¹¹R¹²;
R^{5a} is hydrogen or alkyl; and
R^{5b} is hydrogen, alkyl, keto, or hydroxyl; and
Y, p, q, R¹⁰, R¹¹, and R¹² have the meaning as defined in claim 1.

13. A compound of claim 12 wherein
R¹ is halo; and
R^{1a} is —(CH₂)—OR¹⁰, —(CH₂)—NR¹¹R¹², —(CH₂)—NR^{11a}C(═O)—NR¹¹R¹², —(CH₂)—C(═O)OR¹⁰, —(CH₂)—C(═O)R¹⁰, —(CH₂)CN, —(CH₂)—C(═O)—NR¹¹R¹², (CH₂)—NR^{11a}—C(═O)R¹⁰, —CH₂—NR^{11a}CH₂—C(═O)OR¹⁰, —(CH₂)—NR^{11a}—SO₂—R¹⁰; —NR^{11a}SO₂—R¹⁰, —C(═O)OR¹⁰, —CH═CH—C(═O)OR¹⁰, or —(CH₂)₂—C(═O)OR¹⁰, and
R⁷ is chloro or fluoro; and
R¹⁰, R¹¹, R^{11a}, and R¹² have the meaning as defined in claim 1.

14. A compound of claim 12 or 13 wherein
q is 0
p is 0 or 1
Y is CH₂ or CH═CH or CH₂CH₂
R¹⁰ is hydrogen or lower alkyl
R^{11a} is hydrogen
R¹¹ is hydrogen or lower alkyl
R¹² is hydrogen, lower alkyl, haloalkyl or alkyl-CO₂R¹⁰
or NR¹¹R¹² is pyrrolidine or piperidine

15. Compounds of formula:
N-[5-chloro-2-[2-oxo-2-[4-(phenylmethyl)-1-piperidinyl]ethoxy]phenyl]urea
1-[[2-[(aminocarbonyl)amino]-4-chlorophenoxy]acetyl]-4-[(4-fluorophenyl)methyl]-4-piperidinecarboxylic acid methyl ester
N-[5-chloro-2-[2-[4-[(4-fluorophenyl)methyl]-1-piperidinyl]-2-oxoethoxy]phenyl]urea
N-[5-chloro-2-[2-[4-cyano-4-[(4-fluorophenyl)methyl]-1-piperidinyl]-2-oxoethoxy]phenyl]urea
N-[5-chloro-2-[2-[4-cyano-4-[(4-fluorophenyl)methyl]-1-piperidinyl]-1-methyl-2-oxoethoxy]phenyl]urea
N-[5-chloro-2-[2-[4-[(4-fluorophenyl)methyl]-4-methyl-1-piperidinyl]-2-oxoethoxy]phenyl]urea
N-[5-chloro-2-[2-[cis-4-[(4-fluorophenyl)methyl]-2-methylpiperidinyl]-2-oxoethoxy]phenyl]urea
N-[5-chloro-2-[2-[4-[(4-fluorophenyl)methyl]-3-methyl-1-piperidinyl]-2-oxoethoxy]phenyl]urea
N-[5-chloro-2-[2-[4-[(4-fluorophenyl)methyl]-4-methoxy-1-piperidinyl]-2-oxoethoxy]phenyl]urea
N-[5-chloro-2-[2-[4-[(2,4-difluorophenyl)methyl]-4-hydroxy-1-piperidinyl]-2-oxoethoxy]phenyl]urea
1-[[2-[(aminocarbonyl)amino]-4-chlorophenoxy]acetyl]-4-[(4-fluorophenyl)methyl]-3-oxo-4-piperidinecarboxylic acid ethyl ester
N-[5-chloro-2-[2-[(3R,4R)-4-[(4-fluorophenyl)methyl]-3-hydroxypiperidinyl]-2-oxoethoxy]phenyl]urea
N-[5-chloro-2-[2-[4-[(4-fluorophenyl)methyl]-4-[(2-hydroxypropyl)amino]-1-piperidinyl]-2-oxoethoxy]phenyl]urea
N-[5-chloro-2-[2-[4-[(4-fluorophenyl)methyl]-4-[(2-hydroxypropyl)amino]-1-piperidinyl]-2-oxoethoxy]phenyl]urea
N-[5-chloro-2-[2-[4-(4-fluorobenzoyl)-4-methyl-1-piperidinyl]-2-oxoethoxy]phenyl]urea
N-[5-chloro-2-[2-[4-ethyl-4-(4-fluorobenzoyl)-1-piperidinyl]-2-oxoethoxy]phenyl]urea
N-[2-[2-[4-[(acetyloxy)(4-fluorophenyl)methyl]-4-methyl-1-piperidinyl]-2-oxoethoxy]-5-chlorophenyl]urea
N-[5-chloro-2-[2-[4-[(4-fluorophenyl)hydroxymethyl]-4-methyl-1-piperidinyl]-2-oxoethoxy]phenyl]urea
N-[5-chloro-2-[2-[4-cyano-4-[fluoro(4-fluorophenyl)methyl]-1-piperidinyl]-2-oxoethoxy]phenyl]urea
N-[5-chloro-2-[2-[4-cyano-4-(4-fluorobenzoyl)-1-piperidinyl]-2-oxoethoxy]phenyl]urea
N-[2-[2-[4-amino-4-[(4-fluorophenyl)hydroxymethyl]-1-piperidinyl]-2-oxoethoxy]-5-chlorophenyl]urea
5-chloro-2-[2-[4-cyano-4-[(4-fluorophenyl)methyl]-1-piperidinyl]-2-oxoethoxy]benzoic acid methyl ester
N-[2-[2-[4-cyano-4-[(4-fluorophenyl)methyl]-1-piperidinyl]-2-oxoethoxy]-4-methoxyphenyl]urea
4-[(4-fluorophenyl)methyl]-1-[(3,4,5-trimethoxyphenoxy)acetyl]-4-piperidinecarbonitrile
1-[(4-chloro-2-formylphenoxy)acetyl]-4-[(4-fluorophenyl)methyl]-4-piperidinecarbonitrile
2-[2-[4-amino-4-[(4-fluorophenyl)methyl]-1-piperidinyl]-2-oxoethoxy]-5-chlorobenzoic acid methyl ester
N-[5-chloro-2-[2-[4-(4-fluorobenzoyl)-1-piperidinyl]-2-oxoethoxy]phenyl]urea
N-[5-chloro-2-[2-[4-[(4-fluorophenyl)thio]-1-piperidinyl]-2-oxoethoxy]phenyl]urea
N-[5-chloro-2-[2-[4-(4-chlorobenzoyl)-1-piperidinyl]-2-oxoethoxy]phenyl]urea
N-[2-[2-[4-(4-bromobenzoyl)-1-piperidinyl]-2-oxoethoxy]-5-chlorophenyl]urea
N-[5-chloro-2-[2-[4-[(4-fluorophenyl)methylene]-1-piperidinyl]-2-oxoethoxy]phenyl]urea
N-[5-chloro-2-[2-[4-[(4-fluorophenyl)methyl]-4-(hydroxymethyl)-1-piperidinyl]-2-oxoethoxy]phenyl]urea
N-[[1-[2-[2-[(aminocarbonyl)amino]-4-chlorophenoxy]acetyl]-4-[(4-fluorophenyl)methyl]-4-piperidinyl]methyl]-2,2,2-trifluoroacetamide
N-[5-chloro-2-[2-[4-[(4-fluorophenyl)methyl]-4-hydroxy-1-piperidinyl]-2-oxoethoxy]phenyl]urea
N-[5-chloro-2-[2-[4-fluoro-4-[(4-fluorophenyl)methyl]-1-piperidinyl]-2-oxoethoxy]phenyl]urea
N-[5-chloro-2-[2-[4-[(4-fluorophenyl)methyl]-4-formyl-1-piperidinyl]-2-oxoethoxy]phenyl]urea
N-[5-chloro-2-[2-[4-[(dimethylamino)methyl]-4-[(4-fluorophenyl)methyl]-1-piperidinyl]-2-oxoethoxy]phenyl]urea
N-[5-chloro-2-[2-[4-(dimethylamino)-4-[(4-fluorophenyl)methyl]-1-piperidinyl]-2-oxoethoxy]phenyl]urea
N-[5-chloro-2-[2-[4-[(dipropylamino)methyl]-4-[(4-fluorophenyl)methyl]-1-piperidinyl]-2-oxoethoxy]phenyl]urea
N-[5-chloro-2-[2-[4-[(diethylamino)methyl]-4-[(4-fluorophenyl)methyl]-1-piperidinyl]-2-oxoethoxy]phenyl]urea
N-[1-[2-[2-[(aminocarbonyl)amino]-4-chlorophenoxy]acetyl]-4-[(4-fluoropheny)methyl]-4-piperidinyl]acetamide
N-[1-[[2-[(aminocarbonyl)amino]-4-chlorophenoxy]acetyl]-4-[(4-fluorophenyl)methyl]-4-piperidinyl]urea
[[1-[[2-[(aminocarbonyl)amino]-chlorophenoxy]acteyl]-4-[(4-fluorophenyl)methyl]-4-piperidinyl]amino]acetic acid ethyl ester
N-[5-chloro-2-[2-[4-[(4-fluorophenyl)methyl]-4-[(2-hydroxyethyl)amino]-1-piperidinyl]-2-oxoethoxy]phenyl]urea
[[[1-[[2-[(aminocarbonyl)amino]-4-chlorophenoxy]acetyl]-4-[(4-fluorophenyl)methyl]-4-piperidinyl]methyl]amino]acetic acid ethyl ester
N-[5-chloro-2-[2-[4-[(4-fluorophenyl)methyl]-4-(4-morpholinylmethyl)-1-piperidinyl]-2-oxoethoxy]phenyl]urea
N-[5-chloro-2-[2-[4-[(4-fluorophenyl)methyl]-4-(methylamino)-1-piperidinyl]-2-oxoethoxy]phenyl]urea
N-[5-chloro-2-[2-[4-ethenyl-4-[(4-fluorophenyl)methyl]-1-piperidinyl]-2-oxoethoxy]phenyl]urea
N-[5-chloro-2-[2-[4-ethyl-4-[(4-fluorophenyl)methyl]-1-piperidinyl]-2-oxoethoxy]phenyl]urea
N-[5-chloro-2-[2-[4-[(4-fluorophenyl)methyl]-4-(1-piperidinylmethyl)-1-piperidinyl]-2-oxoethoxy]phenyl]urea
N-[2-[2-[4-(1-azetidinylmethyl)-4-[(4-fluorophenyl)methyl]-1-piperidinyl]-2-oxoethoxy]-5-chlorophenyl]urea
N-[5-chloro-2-[2-[4-[fluoro(4-fluorophenyl)methyl]-4-(1-pyrrolidinylmethyl)-1-piperidinyl]-2-oxoethoxy]phenyl]urea
N-[5-chloro-2-[2-[4-[fluoro(4-fluorophenyl)methyl]-4-(1-piperidinylmethyl)-1-piperidinyl]-2-oxoethoxy]phenyl]urea
N-[5-chloro-2-[2-[4-[(dimethylamino)methyl]-4-[fluoro(4-fluorophenyl)methyl]-1-piperidinyl]-2-oxoethoxy]phenyl]urea
N-[5-chloro-2-[2-[4-[(4-fluorophenyl)methyl]-4-(1H-pyrrol-1-ylmethyl)-1-piperidinyl]-2-oxoethoxy]phenyl]urea
N-[5-chloro-2-[3-[4-(4-fluorobenzoyl)-1-piperidinyl]-2-hydroxypropoxy]phenyl]urea
[1-[3-(2-amino-4-chlorophenoxy)-2-hydroxypropyl]-4-piperidinyl](4-fluorophenyl)methanone
N-[5-chloro-2-[2-[4-(4-fluorobenzoyl)-1-piperidinyl]ethoxy]phenyl]urea
1-[(4-chloro-2-cyanophenoxy)acetyl]-4-[(4-fluorophenyl)methyl]-4-piperidinecarbonitrile
1-[[4-chloro-2-(1H-pyrazol-5-yl)phenoxy]acetyl]-4-[(4-fluorophenyl)methyl]-4-piperidinecarbonitrile
1-[[4-chloro-2-(5-isoxazolyl)phenoxy]acetyl]-4-[(4-fluorophenyl)methyl]-4-piperidinecarbonitrile
1-[[4-chloro-2-(cyanoacetyl)phenoxy]acetyl]-4-[(4-fluorophenyl)methyl]-4-piperidinecarbonitrile
N-[[5-chloro-2-[2-[4-cyano-4-[(4-fluorophenyl)methyl]-1-piperidinyl]-2-oxoethoxy]phenyl]methyl]methanesulfonamide
1-[(2-bromo-4-chlorophenoxy)acetyl]-4-[(4-fluorophenyl)methyl]-4-piperidinecarbonitrile
N-[2-[2-[4-cyano-4-[(4-fluorophenyl)methyl]-1-piperidinyl]-2-oxoethoxy]phenyl]urea
1-[[(5-chloro-8-quinolinyl)oxy]acetyl]-4-[(4-fluorophenyl)methyl]-4-piperidinecarbonitrile
5-chloro-2-[2-[4-(4-fluorobenzoyl)-1-piperidinyl]-2-oxoethoxy]benzamide
5-chloro-2-[2-[4-(4-fluorobenzoyl)-1-piperidinyl]-2-oxoethoxy]benzoic acid methyl ester
[1-[(2-amino-4-chlorophenoxy)acetyl]-4-piperidinyl](4-fluorophenyl)methanone
4-[(4-fluorophenyl)methyl]-1-[[(5-nitro-8-quinolinyl)oxy]acetyl]-4-piperidinecarbonitrile
1-[(4-chlorophenoxy)acetyl]-4-[(4-fluorophenyl)methyl]-4-piperidinecarbonitrile
4-[(4-fluorophenyl)methyl]-1-[(4-quinolinyloxy)acetyl]-4-piperidinecarbonitrile
4-[(4-fluorophenyl)methyl]-1-[(7-isoquinolinyloxy)acetyl]-4-piperidinecarbonitrile
4-[(4-fluorophenyl)methyl]-1-[[(2-hydroxy-8-quinolinyl)oxy]acetyl]4-piperidinecarbonitrile
4-[(4-fluorophenyl)methyl]-1-[(6-quinolinyloxy)acetyl]4-piperidinecarbonitrile
4-[(4-fluorophenyl)methyl]-1-[(5-isoquinolinyloxy)acetyl]-4-piperidinecarbonitrile
4-[(4-fluorophenyl)methyl]-1-[(6-isoquinolinyloxy)acetyl]-4-piperidinecarbonitrile
4-[(4-fluorophenyl)methyl]-1-[[(5-fluoro-8-quinolinyl)oxy]acetyl]-4-piperidinecarbonitrile
1-[[(2-amino-8-quinolinyl)oxy]acetyl]-4-[(4-fluorophenyl)methyl]-4-piperidinecarbonitrile
N-[[5-chloro-2-[2-[4-cyano-4-[(4-fluorophenyl)methyl]-1-piperidinyl]-2-oxoethoxy]phenyl]methyl]-1-propanesulfonamide
4-[(4-fluorophenyl)methyl]-1-[[4-(trifluoromethyl)phenoxy]acetyl]-4-piperidinecarbonitrile
5-chloro-2-[2-[4-cyano-4-[(4-fluorophenyl)methyl]-1-piperidinyl]-2-oxoethoxy]-N-methylbenzenesulfonamide
N-[5-chloro-2-[2-[4-cyano-4-[(4-fluorophenyl)methyl]-1-piperidinyl]-2-oxoethoxy]phenyl]methanesulfonamide
5-chloro-2-[2-[4-cyano-4-[(4-fluorophenyl)methyl]-1-piperidinyl]-2-oxoethoxy]benzenesulfonamide
5-bromo-2-[2-[4-cyano-4-[(4-fluorophenyl)methyl]-1-piperidinyl]-2-oxoethoxy]benzeneacetic acid methyl ester
4-[(4-fluorophenyl)methyl]-1-[(4-formyl-3,5-dimethoxyphenoxy)acetyl]-4-piperidinecarbonitrile
N-[5-chloro-2-[2-[4-cyano-4-[(4-fluorophenyl)methyl]-1-piperidinyl]-2-oxoethoxy]-4-methylphenyl]acetamide
N-[2-[2-[4-cyano-4-[(4-fluorophenyl)methyl]-1-piperidinyl]-2-oxoethoxy]-4-methylphenyl]acetamide
4-[(4-fluorophenyl)methyl]-1-[(4-nitrophenoxy)acetyl]-4-piperidinecarbonitrile
4-[(4-fluorophenyl)methyl]-1-[(2-methoxy-4-nitrophenoxy)acetyl]-4-piperidinecarbonitrile
4-[(4-fluorophenyl)methyl]-1-[(3-nitrophenoxy)acetyl]-4-piperidinecarbonitrile
1-[(4-chloro-3-nitrophenoxy)acetyl]-4-[(4-fluorophenyl)methyl]-4-piperidinecarbonitrile
4-[(4-fluorophenyl)methyl]-1-[(4-formyl-2-methyl phenoxy)acetyl]-4-piperidinecarbonitrile
2-[2-[4-cyano-4-[(4-fluorophenyl)methyl]-1-piperidinyl]-2-oxoethoxy]-benzenepropanoic acid methyl ester
1-[(4-cyano-3-fluorophenoxy)acetyl]-4-[(4-fluorophenyl)methyl]-4-piperidinecarbonitrile
1-[(2-amino-4-chlorophenoxy)acetyl]-α-(4-fluorophenyl)-4-methyl-4-piperidinemethanol
N-[5-chloro-2-[2-[4-cyano-4-[(4-fluorophenyl)hydroxymethyl]-1-piperidinyl]-2-oxoethoxy]phenyl]urea
N-[5-chloro-2-[2-[4-[(4-fluorophenyl)hydroxymethyl]-1-piperidinyl]-2-oxoethoxy]phenyl]urea
1-[[4-chloro-2-(hydroxymethyl)phenoxy]acetyl]-4-[(4-fluorophenyl)methyl]-4-piperidinecarbonitrile
5-chloro-2-[2-[4-cyano-4-[(4-fluorophenyl)methyl]-1-piperidinyl]-2-oxoethoxy]benzoic acid
5-chloro-2-[2-[4-(4-fluorobenzoyl)-1-piperidinyl]-2-oxoethoxy]-benzoic acid
2-[2-[4-amino-4-[(4-fluorophenyl)methyl]-1-piperidinyl]-2-oxoethoxy]-5-chlorobenzoic acid
[[[1-[[2-[(aminocarbonyl)amino]-4-chlorophenoxy]acetyl]-4-[(4-fluorophenyl)methyl]-4-piperidinyl]methyl]amino]acetic acid
[[1-[[2-[(aminocarbonyl)amino]-4-chlorophenoxy]acetyl]-4-[(4-fluorophenyl)methyl]-4-piperidinyl]amino]acetic acid
5-bromo-2-[2-[4-cyano-4-[(4-fluorophenyl)methyl]-1-piperidinyl]-2-oxoethoxy]benzeneacetic acid
N-[2-[2-[4-(aminomethyl)-4-[(4-fluorophenyl)methyl]-1-piperidinyl]-2-oxoethoxy]-5-chlorophenyl]urea
5-chloro-2-[2-[4-(4-fluorobenzoyl)-1-piperidinyl]-2-oxoethoxy]-N-[2-(2-hydroxyethoxy)ethyl]benzamide
5-chloro-N-(cyanomethyl)-2-[2-[4-(4-fluorobenzoyl)-1-piperidinyl]-2-oxoethoxy]benzamide
5-chloro-2-[2-[4-cyano-4-[(4-fluorophenyl)methyl]-1-piperidinyl]-2-oxoethoxy]-N-(1-methyl-4-piperidinyl)benzamide
5-chloro-2-[2-[4-cyano-4-[(4-fluorophenyl)methyl]-1-piperidinyl]-2-oxoethoxy]-N-[2-(1-pyrrolidinyl)ethyl]benzamide
5-chloro-2-[2-[4-cyano-4-[(4-fluorophenyl)methyl]-1-piperidinyl]-2-oxoethoxy]-N-[2-hydroxy-1-(hydroxymethyl)ethyl]benzamide
5-chloro-2-[2-[4-cyano-4-[(4-fluorophenyl)methyl]-1-piperidinyl]-2-oxoethoxy]-N-[2-(2-hydroxyethoxy)ethyl]benzamide
5-chloro-2-[2-[4-cyano-4-[(4-fluorophenyl)methyl]-1-piperidinyl]-2-oxoethoxy]-N-(1,2,2,6,6-pentamethyl-4-piperidinyl)benzamide
5-chloro-2-[2-[4-cyano-4-[(4-fluorophenyl)methyl]-1-piperidinyl]-2-oxoethoxy]-N-(6-methoxy-3-pyridinyl)benzamide
[[5-chloro-2-[2-[4-cyano-4-[(4-fluorophenyl)methyl]-1-piperidinyl]-2-oxoethoxy]benzoyl]amino]acetic acid 1,1-dimethylethyl ester
4-[[5-chloro-2-[2-[4-cyano-4-[(4-fluorophenyl)methyl]-1-piperidinyl]-2-oxoethoxy]benzoyl]amino]-1-piperidinecarboxylic acid 1,1-dimethylethyl ester
5-chloro-2-[2-[4-cyano-4-[(4-fluorophenyl)methyl]-1-piperidinyl]-2-oxoethoxy]-N-(4-piperidinyl)benzamide
[[5-chloro-2-[2-[4-(4-fluorobenzoyl)-1-piperidinyl]-2-oxoethoxy]benzoyl]amino]acetic acid
[[2-[2-[4-amino-4-[(4-fluorophenyl)methyl]-1-piperidinyl]-2-oxoethoxy]-5-chlorobenzoyl]amino]acetic acid
[[5-chloro-2-[2-[4-cyano-4-[(4-fluorophenyl)methyl]-1-piperidinyl]-2-oxoethoxy]benzoyl]amino]acetic acid
[[[5-chloro-2-[2-[4-cyano-4-[(4-fluorophenyl)methyl]-1-piperidinyl]-2-oxoethoxy]phenyl]methyl]amino]acetic acid
5-chloro-2-[2-[4-(4-fluorobenzoyl)-1-piperidinyl]-2-oxoethoxy]-N-(4-piperidinyl)benzamide
5-chloro-2-[2-[4-(4-fluorobenzoyl)-1-piperidinyl]-2-oxoethoxy]-N-(4-piperidinylmethyl)benzamide
[1-[[2-[(4-amino-1-piperidinyl)carbonyl]-4-chlorophenoxy]acetyl]-4-piperidinyl](4-fluorophenyl)methanone
N-(3-aminopropyl)-5-chloro-2-[2-[4-cyano-4-[(4-fluorophenyl)methyl]-1-piperidinyl]-2-oxoethoxy]benzamide
5-chloro-2-[2-[4-cyano-4-[(4-fluorophenyl)methyl]-1-piperidinyl]-2-oxoethoxy]-N-(3-piperidinylmethyl)benzamide
N-(3-aminopropyl)-5-chloro-2-[2-[4-(4-fluorobenzoyl)-1-piperidinyl]-2-oxoethoxy]benzamide
N-(2-aminoethyl)-5-chloro-2-[2-[4-(4-fluorobenzoyl)-1-piperidinyl]-2-oxoethoxy]benzamide
5-chloro-2-[2-[4-(4-fluorobenzoyl)-1-piperidinyl]-2-oxoethoxy]-N-(3-piperidinyl)benzamide
5-chloro-2-[2-[4-(4-fluorobenzoyl)-1-piperidinyl]-2-oxoethoxy]-N-(3-piperidinyl methyl)benzamide
5-chloro-2-[2-[4-(4-fluorobenzoyl)-1-piperidinyl]-2-oxoethoxy]-N-[2-(4-morpholinyl)ethyl]benzamide
5-chloro-2-[2-[4-(4-fluorobenzoyl)-1-piperidinyl]-2-oxoethoxy]-N-(1-methyl-4-piperidinyl)benzamide
5-chloro-2-[2-[4-(4-fluorobenzoyl)-1-piperidinyl]-2-oxoethoxy]-N-hydroxybenzamide
5-chloro-2-[2-[4-(4-fluorobenzoyl)-1-piperidinyl]-2-oxoethoxy]-N-(2-hydroxyethyl)benzamide
5-chloro-2-[2-[4-(4-fluorobenzoyl)-1-piperidinyl]-2-oxoethoxy]-N-(2-hydroxyethyl)-N-methylbenzamide
5-chloro-2-[2-[4-(4-fluorobenzoyl)-1-piperidinyl]-2-oxoethoxy]-N-[2-hydroxy-1-(hydroxymethyl)ethyl]benzamide
5-chloro-2-[2-[4-(4-fluorobenzoyl)-1-piperidinyl]-2-oxoethoxy]-N-[2-(1H-imidazol-4-yl)ethyl]benzamide
5-chloro-2-[2-[4-(4-fluorobenzoyl)-1-piperidinyl]-2-oxoethoxy]benzoic acid hydrazide
5-chloro-N-[2-(dimethylamino)ethyl]-2-[2-[4-(4-fluorobenzoyl)-1-piperidinyl]-2-oxoethoxy]benzamide
5-chloro-N-[3-(dimethylamino)propyl]-2-[2-[4-(4-fluorobenzoyl)-1-piperidinyl]-2-oxoethoxy]benzamide
2-amino-N-[5-chloro-2-[2-[4-(4-fluorobenzoyl)-1-piperidinyl]-2-oxoethoxy]phenyl]acetamide
3-amino-N-[5-chloro-2-[2-[4-(4-fluorobenzoyl)-1-piperidinyl]-2-oxoethoxy]phenyl]propanamide
(2S)-N-[5-chloro-2-[2-[4-(4-fluorobenzoyl)-1-piperidinyl]-2-oxoethoxy]phenyl]-2-pyrrolidinecarboxamide
(α⁴S)-α-amino-N-[5-chloro-2-[2-[4-(4-fluorobenzoyl)-1-piperidinyl]-2-oxoethoxy]phenyl]-1H-imidazole-4-propanamide
(2S)-2-amino-5-[(aminoiminomethyl)amino]-N-[5-chloro-2-[2-[4-(4-fluorobenzoyl)-1-piperidinyl]-2-oxoethoxy]phenyl]pentanamide
(2S)-2,5-diamino-N-[5-chloro-2-[2-[4-(4-fluorobenzoyl)-1-piperidinyl]-2-oxoethoxy]phenyl]pentanamide
(2S)-2-amino-N-[5-chloro-2-[2-[4-(4-fluorobenzoyl)-1-piperidinyl]-2-oxoethoxy]phenyl]-3-hydroxypropanamide
(2S)-2-amino-N′-[5-chloro-2-[2-[4-(4-fluorobenzoyl)-1-piperidinyl]-2-oxoethoxy]phenyl]pentanediamide
N-[5-chloro-2-[2-[4-[(4-fluorophenyl)hydroxymethyl]-4-methyl-1-piperidinyl]-2-oxoethoxy]phenyl]-4-piperidineacetamide
(α⁴S)-α-amino-N-[5-chloro-2-[2-[4-[(4-fluorophenyl)hydroxymethyl]-4-methyl-1-piperidinyl]-2-oxoethoxy]phenyl]-1H-imidazole-4-propanamide
N-[5-chloro-2-[2-[4-(4-fluorobenzoyl)-1-piperidinyl]-2-oxoethoxy]phenyl]-N′-[2-(1H-imidazol-4-yl)ethyl]urea
N-[5-chloro-2-[2-[4-(4-fluorobenzoyl)-1-piperidinyl]-2-oxoethoxy]phenyl]-N′-[(2R)-2-hydroxypropyl]urea
N-[5-chloro-2-[2-[4-(4-fluorobenzoyl)-1-piperidinyl]-2-oxoethoxy]phenyl]-N′-[(2S)-2-hydroxypropyl]urea
N-[5-chloro-2-[2-[4-[(4-fluorophenyl)hydroxymethyl]-4-methyl-1-piperidinyl]-2-oxoethoxy]phenyl]-N′-[2-(1H-imidazol-4-yl)ethyl]urea
N-[5-chloro-2-[2-[4-[fluoro(4-fluorophenyl)methyl]-1-piperidinyl]-2-oxoethoxy]phenyl]urea
N-[5-chloro-2-[2-[4-[fluoro(4-fluorophenyl)methyl]-4-methyl-1-piperidinyl]-2-oxoethoxy]phenyl]urea
N-[5-chloro-2-[2-[4-(fluoromethyl)-4-[(4-fluorophenyl)methyl]-1-piperidinyl]-2-oxoethoxy]phenyl]urea
trifluoroacetic acid [1-[2-[2-[(aminocarbonyl)amino]-4-chlorophenoxy]acetyl]-4-piperidinyl](4-fluorophenyl)methyl ester
N-[5-chloro-2-[2-[4-[(4-fluorophenyl)methyl]-4-[(methylamino)methyl]-1-piperidinyl]-2-oxoethoxy]phenyl]urea
N-[5-chloro-2-[2-[4-[(4-fluorophenyl)methyl]-4-(1-pyrrolidinylmethyl)-1-piperidinyl]-2-oxoethoxy]phenyl]urea
N-[5-chloro-2-[2-[4-[(4-fluorophenyl)methyl]-4-[(4-methyl-1-piperazinyl)methyl]-1-piperidinyl]-2-oxoethoxy]phenyl]urea
N-[5-chloro-2-[2-[4-[(ethylamino)methyl]-4-[(4-fluorophenyl)methyl]-1-piperidinyl]-2-oxoethoxy]phenyl]urea
N-[5-chloro-2-[2-[4-[(4-fluorophenyl)methyl]-4-[[(2-hydroxyethyl)amino]methyl]-1-piperidinyl]-2-oxoethoxy]phenyl]urea
1-[[4-chloro-2-[(4-methyl-1-piperazinyl)methyl]phenoxy]acetyl]-4-[(4-fluorophenyl)methyl]-4-piperidinecarbonitrile
1-[[4-chloro-2-[(methylamino)methyl]phenoxy]acetyl]-4-[(4-fluorophenyl)methyl]-4-piperidinecarbonitrile
1-[[4-chloro-2-[[(2-hydroxyethyl)amino]methyl]phenoxy]acetyl]4-[(4-fluorophenyl)methyl]-4-piperidinecarbonitrile
1-[[4-chloro-2-(4-morpholinylmethyl)phenoxy]acetyl]-4-[(4-fluorophenyl)methyl]4-piperidinecarbonitrile
1-[[4-chloro-2-[(dimethylamino)methyl]phenoxy]acetyl]-4-[(4-fluorophenyl)methyl]-4-piperidinecarbonitrile
[[[5-chloro-2-[2-[4-cyano-4-[(4-fluorophenyl)methyl]-1-piperidinyl]-2-oxoethoxy]phenyl]methyl]amino]acetic acid 1,1-dimethylethyl ester
1-[[2-(aminomethyl)-4-chlorophenoxy]acetyl]-4-[(4-fluorophenyl)methyl]-4-piperidinecarbonitrile
1-[[4-chloro-2-(1H-1,2,4-triazol-1-yl methyl)phenoxy]acetyl]-4-[(4-fluorophenyl)methyl]-4-piperidinecarbonitrile
5-bromo-2-[2-[4-[(5-chloro-2-thienyl)methyl]-4-cyano-1-piperidinyl]-2-oxoethoxy]benzeneacetic acid methyl ester
5-bromo-2-[2-[4-[(5-chloro-2-thienyl)methyl]-4-cyano-1-piperidinyl]-2-oxoethoxy]benzeneacetic acid
5-chloro-2-[(1E)-3-[4-cyano-4-[(4-fluorophenyl)methyl]-1-piperidinyl]-3-oxo-1-propenyl]benzeneacetic acid methyl ester
5-chloro-2-[(1E)-3-[4-cyano-4-[(4-fluorophenyl)methyl]-1-piperidinyl]-3-oxo-1-propenyl]benzeneacetic acid
5-bromo-2-[2-[4-[(4-fluorophenyl)methyl]-4-formyl-1-piperidinyl]-2-oxoethoxy]benzeneacetic acid methyl ester
5-bromo-2-[2-[4-[(4-fluorophenyl)methyl]-4-[(methylamino)methyl]-1-piperidinyl]-2-oxoethoxy]benzeneacetic acid methyl ester
5-bromo-2-[2-[4-[(4-fluorophenyl)methyl]-4-[(methylamino)methyl]-1-piperidinyl]-2-oxoethoxy]benzeneacetic acid
5-bromo-2-[2-[4-cyano-4-[(4-fluorophenyl)methyl]-1-piperidinyl]-2-oxoethoxy]-4-(dimethylamino)benzeneacetic acid
[5-chloro-2-[2-[4-cyano-4-[(4-fluorophenyl)methyl]-1-piperidinyl]-2-oxoethoxy]phenoxy]acetic acid methyl ester
[5-chloro-2-[2-[4-cyano-4-[(4-fluorophenyl)methyl]-1-piperidinyl]-2-oxoethoxy]phenoxy]acetic acid
[[5-chloro-2-[2-[4-cyano-4-[(4-fluorophenyl)methyl]-1-piperidinyl]-2-oxoethoxy]phenyl]amino]acetic acid
3-[5-chloro-2-[2-[4-cyano-4-[(4-fluorophenyl)methyl]-1-piperidinyl]-2-oxoethoxy]phenyl]-2-propenoic acid
5-bromo-2-[2-[4-[(4-fluorophenyl)methyl]-4-[[(2,2,2-trifluoroethyl)amino]methyl]-1-piperidinyl]-2-oxoethoxy]benzeneacetic acid
2-[2-[4-amino-4-[(4-fluorophenyl)methyl]-1-piperidinyl]-2-oxoethoxy]-5-bromobenzeneacetic acid
5-chloro-2-[2-[4-cyano-4-[(4-fluorophenyl)methyl]-1-piperidinyl]-2-oxoethoxy]benzenepropanoic acid
5-bromo-2-[2-[4-[(4-fluorophenyl)methyl]-4-hydroxy-1-piperidinyl]-2-oxoethoxy]benzeneacetic acid methyl ester
5-bromo-2-[2-[4-[(4-fluorophenyl)methyl]-4-hydroxy-1-piperidinyl]-2-oxoethoxy]benzeneacetic acid
5-bromo-2-[2-[4-[(4-fluorophenyl)methyl]-4-(1-piperidinylmethyl)-1-piperidinyl]-2-oxoethoxy]benzeneacetic acid methyl ester
5-bromo-2-[2-[4-[(4-fluorophenyl)methyl]-4-(1-piperidinylmethyl)-1-piperidinyl]-2-oxoethoxy]benzeneacetic acid
2-[2-[4-(1-azetidinylmethyl)-4-[(4-fluorophenyl)methyl]-1-piperidinyl]-2-oxoethoxy]-5-bromobenzeneacetic acid methyl ester
2-[2-[4-(1-azetidinylmethyl)-4-[(4-fluorophenyl)methyl]-1-piperidinyl]-2-oxoethoxy]-5-bromobenzeneacetic acid
5-bromo-2-[2-[4-[(4-fluorophenyl)methyl]-4-(1-pyrrolidinylmethyl)-1-piperidinyl]-2-oxoethoxy]benzeneacetic acid methyl ester
5-bromo-2-[2-[4-[(4-fluorophenyl)methyl]-4-(1-pyrrolidinylmethyl)-1-piperidinyl]-2-oxoethoxy]benzeneacetic acid
5-bromo-2-[2-[4-cyano-4-[fluoro(4-fluorophenyl)methyl]-1-piperidinyl]-2-oxoethoxy]benzeneacetic acid methyl ester
5-bromo-2-[2-[4-cyano-4-[fluoro(4-fluorophenyl)methyl]-1-piperidinyl]-2-oxoethoxy]benzeneacetic acid
5-bromo-2-[2-[4-cyano-4-[(4-fluorophenyl)hydroxymethyl]-1-piperidinyl]-2-oxoethoxy]benzeneacetic acid methyl ester
5-bromo-2-[2-[4-cyano-4-[(4-fluorophenyl)hydroxymethyl]-1-piperidinyl]-2-oxoethoxy]benzeneacetic acid
5-chloro-2-[2-[4-cyano-4-[(4-fluorophenyl)methyl]-1-piperidinyl]-2-oxoethoxy]-α-fluorobenzeneacetic acid methyl ester
5-chloro-2-[2-[4-cyano-4-[(4-fluorophenyl)methyl]-1-piperidinyl]-2-oxoethoxy]-α-fluorobenzeneacetic acid
5-bromo-2-[2-[4-(2-cyanoethyl)-4-[(4-fluorophenyl)methyl]-1-piperidinyl]-2-oxoethoxy]benzeneacetic acid methyl ester
5-bromo-2-[2-[4-(2-cyanoethyl)-4-[(4-fluorophenyl)methyl]-1-piperidinyl]-2-oxoethoxy]benzeneacetic acid
α-amino-5-chloro-2-[2-[4-cyano-4-[(4-fluorophenyl)methyl]-1-piperidinyl]-2-oxoethoxy]benzeneacetic acid
5-chloro-2-[2-[4-cyano-4-[(4-fluorophenyl)methyl]-1-piperidinyl]-2-oxoethoxy]benzeneacetic acid methyl ester
5-chloro-2-[2-[4-cyano-4-[(4-fluorophenyl)methyl]-1-piperidinyl]-2-oxoethoxy]benzeneacetic acid
5-chloro-2-[2-[4-cyano-4-[(4-fluorophenyl)methyl]-1-piperidinyl]-2-oxoethoxy]-4-(trifluoromethyl)benzoic acid methyl ester
5-chloro-2-[2-[4-cyano-4-[(4-fluorophenyl)methyl]-1-piperidinyl]-2-oxoethoxy]-4-(trifluoromethyl)benzoic acid
5-bromo-2-[2-[4-[(4-fluorophenyl)methyl]-1-piperidinyl]-2-oxoethoxy]benzeneacetic acid methyl ester
5-bromo-2-[2-[4-[(4-fluorophenyl)methyl]-1-piperidinyl]-2-oxoethoxy]benzeneacetic acid
5-bromo-2-[2-[4-[(4-chlorophenyl)methyl]-4-cyano-1-piperidinyl]-2-oxoethoxy]benzeneacetic acid methyl ester
5-bromo-2-[2-[4-[(4-chlorophenyl)methyl]-4-cyano-1-piperidinyl]-2-oxoethoxy]benzeneacetic acid
5-chloro-2-[2-[4-cyano-4-[(4-fluorophenyl)methyl]-1-piperidinyl]-2-oxoethoxy]-α-hydroxybenzeneacetic acid methyl ester
5-chloro-2-[2-[4-cyano-4-[(4-fluorophenyl)methyl]-1-piperidinyl]-2-oxoethoxy]-α-hydroxybenzeneacetic acid
5-chloro-2-[2-[4-cyano-4-[(4-fluorophenyl)methyl]-1-piperidinyl]-2-oxoethoxy]4-methylbenzeneacetic acid methyl ester
5-chloro-2-[2-[4-cyano-4-[(4-fluorophenyl)methyl]-1-piperidinyl]-2-oxoethoxy]-4-methylbenzeneacetic acid
5-bromo-2-[2-[4-cyano-4-[(4-fluorophenyl)methyl]-1-piperidinyl]-2-oxoethoxy]-α,α-difluorobenzeneacetic acid methyl ester
5-bromo-2-[2-[4-cyano-4-[(4-fluorophenyl)methyl]-1-piperidinyl]-2-oxoethoxy]-α,α-difluorobenzeneacetic acid
5-bromo-2-[2-[4-cyano-4-[(4-fluorophenyl)methyl]-1-piperidinyl]-2-oxoethoxy]-α,α-dimethylbenzeneacetic acid methyl ester
5-bromo-2-[2-[4-cyano-4-[(4-fluorophenyl)methyl]-1-piperidinyl]-2-oxoethoxy]-α,α-dimethylbenzeneacetic acid
1-[(4-bromo-2-formyl phenoxy)acetyl]-4-[(4-fluorophenyl)methyl]-4-piperidinecarbonitrile
1-[[4-bromo-2-(hydroxymethyl)phenoxy]acetyl]-4-[(4-fluorophenyl)methyl]-4-piperidinecarbonitrile
[[5-bromo-2-[2-[4-cyano-4-[(4-fluorophenyl)methyl]-1-piperidinyl]-2-oxoethoxy]phenyl]methyl]phosphonic acid
[[5-bromo-2-[2-[4-cyano-4-[(4-fluorophenyl)methyl]-1-piperidinyl]-2-oxoethoxy]phenyl]methyl]phosphonic acid ethyl ester
5-chloro-2-[2-[4-cyano-4-[(4-fluorophenyl)methyl]-1-piperidinyl]-2-oxoethoxy]benzenemethanesulfonic acid
5-chloro-2-[3-[4-cyano-4-[(4-fluorophenyl)methyl]-1-piperidinyl]-3-oxopropyl]benzeneacetic acid
5-chloro-2-[2-[4-cyano-4-[(4-fluorophenyl)methyl]-1-piperidinyl]-2-oxoethoxy]-α-hydroxybenzeneacetamide
N-[2-[5-bromo-2-[2-[4-cyano-4-[(4-fluorophenyl)methyl]-1-piperidinyl]-2-oxoethoxy]phenyl]acetyl]methanesulfonamide

16. Compounds according to claim 15 and preferably:
N-[5-chloro-2-[2-[4-cyano-4-[(4-fluorophenyl)methyl]-1-piperidinyl]-2-oxoethoxy]phenyl]urea
N-[5-chloro-2-[2-[4-[(4-fluorophenyl)methyl]-4-[(2-hydroxypropyl)amino]-1-piperidinyl]-2-oxoethoxy]phenyl]urea
N-[5-chloro-2-[2-[4-[(4-fluorophenyl)methyl]-4-hydroxy-1-piperidinyl]-2-oxoethoxy]phenyl]urea
[[[1-[[2-[(aminocarbonyl)amino]-4-chlorophenoxy]acetyl]-4-[(4-fluorophenyl)methyl]4-piperidinyl]methyl]amino]acetic acid
N-[5-chloro-2-[2-[4-cyano-4-(4-fluorobenzoyl)-1-piperidinyl]-2-oxoethoxy]phenyl]urea
N-[5-chloro-2-[2-[4-cyano-4-[fluoro(4-fluorophenyl)methyl]-1-piperidinyl]-2-oxoethoxy]phenyl]urea
5-chloro-2-[2-[4-cyano-4-[(4-fluorophenyl)methyl]-1-piperidinyl]-2-oxoethoxy]benzoic acid
N-[[5-chloro-2-[2-[4-cyano-4-[(4-fluorophenyl)methyl]-1-piperidinyl]-2-oxoethoxy]phenyl]methyl]methanesulfonamide
N-[5-chloro-2-[2-[4-cyano-4-[(4-fluorophenyl)methyl]-1-piperidinyl]-2-oxoethoxy]phenyl]methanesulfonamide
5-bromo-2-[2-[4-cyano-4-[(4-fluorophenyl)methyl]-1-piperidinyl]-2-oxoethoxy]benzeneacetic acid
5-bromo-2-[2-[4-[(4-fluorophenyl)methyl]-4-(1-piperidinylmethyl)-1-piperidinyl]-2-oxoethoxy]benzeneacetic acid
5-chloro-2-[2-[4-cyano-4-[(4-fluorophenyl)methyl]-1-piperidinyl]-2-oxoethoxy]benzeneacetic acid
5-bromo-2-[2-[4-[(4-fluorophenyl)methyl]-4-(1-pyrrolidinylmethyl)-1-piperidinyl]-2-oxoethoxy]benzeneacetic acid
3-[5-chloro-2-[2-[4-cyano-4-[(4-fluorophenyl)methyl]-1-piperidinyl]-2-oxoethoxy]phenyl]-2-propenoic acid
5-chloro-2-[2-[4-cyano-4-[(4-fluorophenyl)methyl]-1-piperidinyl]-2-oxoethoxy]benzenepropanoic acid
5-bromo-2-[2-[4-[(4-fluorophenyl)methyl]-4-[(methylamino)methyl]-1-piperidinyl]-2-oxoethoxy]benzeneacetic acid
[[5-bromo-2-[2-[4-cyano-4-[(4-fluorophenyl)methyl]-1-piperidinyl]-2-oxoethoxy]phenyl]methyl]phosphonic acid ethyl ester
5-bromo-2-[2-[4-(2-cyanoethyl)-4-[(4-fluorophenyl)methyl]-1-piperidinyl]-2-oxoethoxy]benzeneacetic acid
5-chloro-2-[2-[4-cyano-4-[(4-fluorophenyl)methyl]-1-piperidinyl]-2-oxoethoxy]benzenemethanesulfonic acid
5-bromo-2-[2-[4-[(4-fluorophenyl)methyl]-4-[[(2,2,2-trifluoroethyl)amino]methyl]-1-piperidinyl]-2-oxoethoxy]benzeneacetic acid
5-bromo-2-[2-[4-cyano-4-[(4-fluorophenyl)methyl]-1-piperidinyl]-2-oxoethoxy]-4-(dimethylamino)benzeneacetic acid
5-chloro-2-[2-[4-cyano-4-[(4-fluorophenyl)methyl]-1-piperidinyl]-2-oxoethoxy]benzeneacetic acid
5-chloro-2-[2-[4-cyano-4-[(4-fluorophenyl)methyl]-1-piperidinyl]-2-oxoethoxy]-4-methylbenzeneacetic acid
5-chloro-2-[(1E)-3-[4-cyano-4-[(4-fluorophenyl)methyl]-1-piperidinyl]-3-oxo-1-propenyl]benzeneacetic acid
5-bromo-2-[2-[4-cyano-4-[fluoro(4-fluorophenyl)methyl]-1-piperidinyl]-2-oxoethoxy]benzeneacetic acid
5-bromo-2-[2-[4-[(5-chloro-2-thienyl)methyl]-4-cyano-1-piperidinyl]-2-oxoethoxy]benzeneacetic acid
5-bromo-2-[2-[4-[(4-chlorophenyl)methyl]-4-cyano-1-piperidinyl]-2-oxoethoxy]benzeneacetic acid

17. A compound according to any one of the claims 1 to 16 for use as a medicament.

18. Use of a compound according to any one of the claims 1 to 16 for the production of a medicament for the treatment of inflammatory disorders.

19. Use of a compound according to any one of the claims 1 to 16 for the production of a medicament for the treatment of multiple sclerosis, leukoencephalopathy, encephalomyelitis, Alzheimer's disease, Guillian-Barre syndrome, acute cell-mediated renal transplant rejection, allograft rejection, rheumatoid arthritis, atherosclerosis, uricaria, angioderma, allergic conjunctivitis, atopic dermatitis, psoriasis, allergic contact dermatitis, drug or insect sting allergy or systemic anaphylaxis.

20. Use of a compound according to any one of the claims 1 to 16 for the production of a medicament for the treatment of fibrosis particularly renal fibrosis, heart transplant rejection, and myocarditis.

21. Use of a compound according to any one of the claims 1 to 16 for the production of a medicament for the treatment of endometriosis.

22. Use of a compound according to any one of the claims 1 to 16 for the production of a medicament for the treatment of multiple myeloma.

23. Use of a radio-labeled analog of a compound according to any one of the claims 1 to 16 as an imaging agents.

24. A pharmaceutical composition comprising a compound according to any one of claims 1 to 16 in association with a pharmaceutically acceptable diluent or carrier.

25. A method of treating inflammatory disorders, which method comprises administering to a human in need of such treatment a therapeutically effective amount of a compound according to any one of the claims 1 to 16.

26. A method of treating multiple sclerosis, leukoencephalopathy, encephalomyelitis, Alzheimer's disease, Guillian-Barre syndrome, acute cell-mediated renal transplant rejection, allograft rejection, rheumatoid arthritis, atherosclerosis, uricaria, angioderma, allergic conjunctivitis, atopic dermatitis, psoriasis, allergic contact dermatitis, drug or insect sting allergy or systemic anaphylaxis, which method comprises administering to a human in need of such treatment a therapeutically effective amount of a compound according to any one of the claims 1 to 16.

27. A method of treating fibrosis particularly renal fibrosis, heart transplant rejection, or myocarditis, which method comprises administering to a human in need of such treatment a therapeutically effective amount of a compound according to any one of the claims 1 to 16.

28. A method of treating endometriosis, which method comprises administering to a human in need of such treatment a therapeutically effective amount of a compound according to any one of the claims 1 to 16.

29. A method of treating multiple myeloma, which method comprises administering to a human in need of such treatment a therapeutically effective amount of a compound according to any one of the claims 1 to 16.

30. A process for the preparation of a compound according to any one of the claims 1 to 16 where in said compounds R² is —O—, R³ is alkylene, and R⁴ is —C(═O)—, according to the following scheme 1: 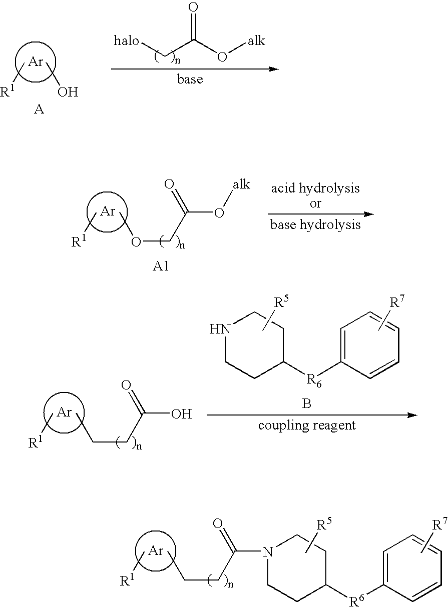
characterized in that precursor A is reacted with a haloalkylester to generate precursor A1, which is then hydrolyzed to its acid form and coupled with precursor B, or
according to the following scheme 2: 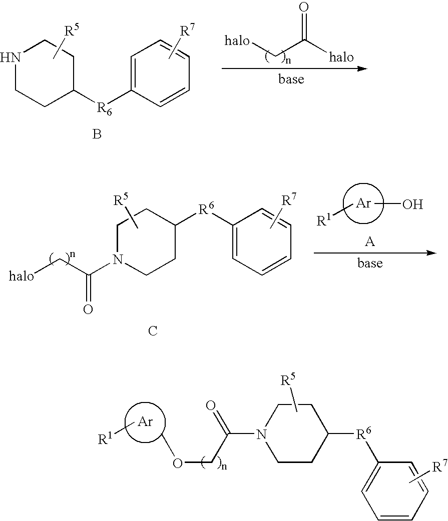
precursor B is reacted with the desired haloalkylcarbonylhalide to generate precursor C, which is then coupled with precursor A, and where all other substituents have the meaning as defined in claim 1.
